(19) United States
(12) Patent Application Publication
(10) Pub. No.: US 2004/0018528 A1
(43) Pub. Date: Jan. 29, 2004

(54) Novel biomarkers of tyrosine kinase inhibitor exposure and activity in mammals

(75) Inventors: Alyssa Morimoto, San Mateo, CA (US); Samuel DePrimo, Palo Alto, CA (US); Anne-Marie O'Farrell, Menlo Park, CA (US); Beverly D. Smolich, Mountain View, CA (US); William C. Manning, Redwood City, CA (US); Sarah A. Walter, Redwood City, CA (US); James W. Schilling JR., San Mateo, CA (US); Julie Cherrington, San Francisco, CA (US)

Correspondence Address: FOLEY AND LARDNER, SUITE 500, 3000 K STREET NW, WASHINGTON, DC, 20007, US

(73) Assignee: Sugen, Inc.

(21) Appl. No.: 10/440,464

(22) Filed: May 19, 2003

Related U.S. Application Data
(60) Provisional application No. 60/448,922, filed on Feb. 24, 2003.
(60) Provisional application No. 60/380,872, filed on May 17, 2002.
(60) Provisional application No. 60/448,874, filed on Feb. 24, 2003.

(51) Int. Cl. C12Q 1/68; G01N 33/53

(52) U.S. Cl. 435/6; 435/7.1

(57) ABSTRACT

The present invention describes novel methods that measure in a mammal the level of at least one biomarker, such as a protein and/or mRNA transcript. Based on the level of at least one biomarker in a mammal exposed to a test compound, compared to the level of the biomarker(s) in a mammal that has not been exposed to a test compound, the ability of the test compound to inhibit tyrosine kinase activity can be determined. The invention also relates to novel methods, wherein a change in the level of at least one biomarker in a mammal exposed to a compound, compared to the level of the biomarker(s) in a mammal that has not been exposed to the compound, indicates whether the mammal is being exposed to, or is experiencing or will experience a therapeutic or toxic effect in response to, a compound that inhibit tyrosine kinase activity.

This application claims benefit of priority from U.S. provisional application Ser. Nos 60/380,872, filed May 17, 2002, 60/448,922, filed Feb. 24, 2003, and 60/448,874, filed Feb. 24, 2003, all of which are incorporated by reference in their entirety.

### BACKGROUND OF THE INVENTION

A biomarker is a molecular marker of a biological event or phenomenon in a organism. Changes in the level of certain biomakers indicate a biological response to a chemical compound in an organism. Biological responses include events at the molecular, cellular or whole organism level. Changes in biomarker levels can be measured and used to indicate whether or not a particular effect has been achieved in the organism. Changes in biomarker levels can indicate that an organism has been exposed to a particular compound. Changes in biomarker levels also can indicate whether an organism is experiencing or will experience a therapeutic effect or even a toxic event in response to a compound.

### SUMMARY OF INVENTION

The present invention relates to novel methods comprising measuring in a mammal the level of at least one biomarker, such as a protein and/or mRNA transcript. In the novel methods, the level of at least one biomarker in a mammal exposed to a compound is compared to the level of the biomarker(s) in a mammal that has not been exposed to the compound.

The invention includes methods for determining whether a test compound inhibits the activity of a protein tyrosine kinase. The invention further relates to methods for determining whether a mammal has been exposed to a test compound that inhibits tyrosine kinase activity. The invention also discloses methods for determining if a mammal is responsive to the administration of a compound that inhibits tyrosine kinase activity. In addition, the invention relates to methods for identifying mammals that will respond therapeutically to a compound that inhibits VEGFR and/or PDGFR tyrosine kinases. The invention further discloses methods for testing or predicting, as well as kits for determining, whether a mammal will respond therapeutically to a compound that inhibits tyrosine kinase activity. The invention also relates to methods for testing or predicting whether a mammal will experience an adverse event, such as fatigue, in response to a method of treatment comprising adminstering a compound that inhibits tyrosine kinase activity.

### BRIEF DESCRIPTION OF THE FIGURES

FIG. 1 shows the levels of various plasma proteins in plasma from human patients, measured by ELISA, before and 24 hours after the first dose of Compound A (SU6668).

FIG. 2 shows the abundance of a protein (spot #5) in patient plasma, measured by 2D polyacrylamide gel analysis, before and 4 hours after the first dose of Compound A (SU6668).

FIG. 3 shows the identification by mass spectrometry analysis of spot #5 from the 2D gel analysis of patient plasma analyzed in FIG. 2.

FIG. 4A shows the change in level of various RNA transcripts, before versus 24 hours after the first dose of Compound A (SU6668), in patient whole blood, as measured by Taqman and DNA Array analysis. FIG. 4B shows the change in the level of vinculin RNA, before versus 24 hours after the first dose of Compound A (SU6668), in patient whole blood, as measured by Taqman and DNA Array analysis.

FIG. 5 shows the levels of various RNA transcripts, in patient blood samples, on treatment day 28 (27 days after the first dose of Compound A) versus the levels on treatment day 0 (before treatment with Compound A). Numbers shown indicate increase and/or decrease relative to baseline on day 0. No significant change is shown as ˜1. Levels decreased are less than 1 and levels increased are greater than 1.

FIG. 6 shows the differential expression of candidate biomarker transcripts in patient PBMC at day 56 relative to day 1 of therapy. The diagram is a depiction of the Affymetrix Difference Calls assigned to each day 56:day 1 expression comparison among the patient sample pairs analyzed via GeneChip hybridization analysis. Letters within blocks represent the Difference Call assigned to each relative expression comparison. The abbreviations are: I=Increase, MI=Marginally Increased, NC=Not Changed; MD=Marginally Decreased; D=Decreased. Cases in which an Increased or Marginally Increased call is assigned to a day 56:day 1 comparison are shaded in gray. Each column represents a different patient. Column headings in each grid represent patient response assessed at the end of first treatment cycle: PR=partial response, CR=complete response, PD=progressive disease.

FIGS. 7A and 7B show the percentage of patients with increased expression of biomarker transcripts following treatment with Compound B (SU5416). Differential expression of six transcripts as measured by microarray and quantitative RT-PCR is presented. The percentage of cases in 5-FU/LV (control) and 5-FU/LV+SU5416 trial arms with increased expression (at predose day 56 relative to predose day 1) of each transcript is displayed. FIG. 7A shows the results of the Affymetrix analysis and FIG. 7B shows the results from SYBR Green RT-PCR. For the SYBR Green data, an increased is defined as relative expression value of 2-fold or greater. A total of 31 sample pairs were used in RT-PCR analysis; 18 were from SU5416 arm (5 PR, 1 CR, 11 PD and 1 SD response at end of cycle 1), and 13 were from the control arm (9 PR, 3 PD and 1 SD).

FIG. 8 shows the percentage of patients with increased expression of four biomarker transcripts, following treatment with Compound B (SU5416). Differential expression of four transcripts as measured by quantitative RT-PCT is presented. Percentage of cases in CPT-11/5-FU/LV (control) and CPT-11/5-FU/LV+SU5416 trial arms with increased expression (at predose day 42 relative to predose day 1) of four candidate biomarker transcripts in a second SU5416 Phase III clinical trial is displayed. The convention is the same as in panel B in FIG. 7. A total of 36 sample pairs was included in this analysis; 18 from the Compound B arm and 18 from the control arm (8 PR and 10 SD responses at end of cycle 1 in each group).

FIG. 9 shows hierarchical clustering of relative expression ratios for four biomarker transcripts. This mosaic depicts association between patent samples and relative expression of the four potential biomarker transcripts. Natural log-transformed SYBR Green RT-PCR ratio data (relative expression of day 56:day 1) were used in analysis. In the color scheme, higher ratios are indicated in red, lower ones in green (scale ranges from −4 to +4). Results from individual patients are oriented as rows and transcripts are oriented as columns. Red bars on the right side of the map indicate cases from the SU4316 arm. The hierarchical clustering method is average linkage and the distance metric is Euclidean.

FIG. 10 shows PAI-1 levels on day 1 and day 56 in patient plasma samples. MR=minor response (cycle 1); PR=partial response (cycle 1); PD=progressive disease (cycle 1)

FIG. 11 shows the mRNA and protein sequences for lactoferrin (SEQ ID NOS 68-69, respectively), lipocalin-2 (SEQ ID NOS 70-71 and 180, respectively), MMP9 (SEQ ID NOS 72 & 66, respectively), and CD24 (SEQ ID NO: 73-74, respectively).

FIG. 12 shows mRNA and protein sequences for eucaryotic initiation factor 4A11 (SEQ ID NOS 75-76, respectively), human (clone 5) orphan G protein-coupled receptor (Genbank Accession No. L06792) (SEQ ID NOS 77-78, respectively), Homo sapiens thymosin beta-10 (SEQ ID NOS 79-80, respectively), Homo sapiens hnRNPcore protein A1 (SEQ ID NOS 81-82, respectively), human leucocyte antigen (CD37) (SEQ ID NOS 83-84, respectively), human MHC call II HLA-DR beta-1 (SEQ ID NOS 85-86, respectively), Homo sapiens translation initiation factor elF3 p66 subunit (SEQ ID NOS 87-88, respectively), Homo sapiens nm23-H2 gene (SEQ ID NOS 89-90, respectively), human acidic ribosomal phosphoprotein P0 (SEQ ID NOS 91-92, respectively), human cyclophillin (SEQ ID NOS 93-94, respectively), Genbank Accession No. AI541256 (cDNA) (SEQ ID NO: 95), human T-cell receptor active beta chain (SEQ ID NOS 96-97, respectively), human MHC class II lymphocyte antigen (HLA-DP) beta chain (SEQ ID NOS 98-99, respectively), human KIAA0195 (SEQ ID NOS 100-101, respectively), Homo sapiens MAP kinase kinase 3 (MKK3) (SEQ ID NOS 102-103, respectively), human beta-tubulin class III isotype (beta-3) (SEQ ID NOS 104-105, respectively), human tropomyosin (SEQ ID NOS 106-107, respectively), 1-phosphatidyl inositol-4-phosphate-5-kinase isoform C (SEQ ID NOS 108-109, respectively), human MLC emb gene for embryonic myosin alkaline light chain (SEQ ID NOS 110-111, respectively), Homo sapiens glyoxalase II (SEQ ID NOS 112-113, respectively), Homo sapiens trans-golgi network glycoprotein 48 (SEQ ID NOS 114-115, respectively), histone H2B (SEQ ID NOS 116-117, respectively), human RLIP76 protein (SEQ ID NOS 118-119, respectively), Genbank Accession No. W26677 (human retina cDNA) (SEQ ID NO: 120), human PMI gene for a putative receptor protein (SEQ ID NOS 121-122, respectively), human DNA-binding protein A (dbpA) (SEQ ID NOS 123-124, respectively), human ITIH4 (SEQ ID NOS 125-126, respectively), IL-8 (SEQ ID NOS 182-183, respectively) and C-reactive protein (SEQ ID NOS 184-185, respectively).

FIG. 13 shows the changes in VEGF plasma levels, as measured by ELISA, in patients receiving a malate salt of Compound 1 in Trial C.

FIG. 14 shows by hybrid ELISA that VEGF/PLGF heterodimers are detected in plasma of cancer patients and are induced in patients after treatment with a malate salt of Compound 1 in Trial C. The hybrid ELISA assay demonstrates that levels of heterodimers are increased in 3 of 3 patients tested, and follow a pattern of induction similar to that seen for VEGF and PLGF.

FIG. 15 shows that plasma levels of soluble VEGFR2 decrease in patients in Trial D following treatment with a malate salt of Compound 1 in a dose-dependent manner.

FIG. 16 shows that the decrease in sVEGFR2 following treatment with Compound 1 or malate salt thereof correlates with AUC values (end of C1 dosing, all trials). The scatter graph plots sVEGFR2 fold change (end of cycle 1 dosing over baseline) against AUC values from end of cycle 1 dosing. Results from the first 44 patients (representing 4 trials) are included.

FIG. 17 shows that chemokine MIG is induced in patients during treatment with a malate salt of Compound 1. MIG is a biomarker that also correlates with tumor responses as measured by ¹⁸FDG-PET imaging. Results are from Trial C.

FIG. 18 discloses the amino acid sequence of human vascular endothelial growth factor (VEGF) (SEQ ID NO: 127).

FIG. 19 discloses the amino acid sequence of human placenta growth factor (PLGF) (SEQ ID NO: 128).

FIG. 20 discloses the amino acid sequence of human vascular endothelial growth factor receptor 2 (VEGFR2) (SEQ ID NO: 129).

FIG. 21 discloses the amino acid sequence of human Monokine Induced by Interferon-Gamma (MIG) (SEQ ID NO: 55).

FIG. 22 discloses the amino acid sequence of human interferon-inducible cytokine IP-10 (SEQ ID NO: 130).

FIG. 23 discloses the amino acid sequence of human Interferon-inducible T-cell alpha chemoattractant (I-TAC) (SEQ ID NO: 131).

FIG. 24 shows cDNA or mRNA sequences for human vinculin (SEQ ID NOS 132 & 181, respectively), basic transcription factor 3 homologue (SEQ ID NO: 133), human c-jun proto oncogene (SEQ ID NO: 134), human c-fos proto-oncogen (SEQ ID NO: 135), Homo sapien PTP-nonreceptor type 2 (SEQ ID NO: 136), human cdc2-related protein kinase (SEQ ID NO: 137), human cyclin C (SEQ ID NO: 138), human DNA polymerase-gamma (SEQ ID NO: 139), protein kinase C-alpha (SEQ ID NO: 140), lipocortin II/annexin A2 (SEQ ID NO: 141), histone H2B member R (SEQ ID NO: 142), Homo sapien amphiregulin (SEQ ID NO: 143), human basic transcription factor 3 (SEQ ID NO: 144), Homo sapien phosphoinositide-3-kinase p110 subunit (SEQ ID NO: 145), human gelsolin (SEQ ID NO: 146), Homo sapien Cyclin D2 (SEQ ID NO: 147), ephrin receptor (EphB4) (SEQ ID NO: 148), human Hanukah factor/granzyme A (SEQ ID NO: 149), von Hippel-Lindau (VHL) tumor suppressor (SEQ ID NO: 150), human mRNA for OB-cadherin-1 (SEQ ID NO: 151), human mRNA for OB-cadherin-2 (SEQ ID NO: 152), phosphoinositol 3-phosphate-binding protein-3 (PEPP3) (SEQ ID NO: 153), human phosphoinositol 3-kinase p85 subunit (SEQ ID NO: 154), human mucin 1 (SEQ ID NO: 155), ErbB3/HER3 receptor tyrosine kinase (SEQ ID NO: 156), and Homo sapien gene for hepatitis C-associated microtubulear aggregate protein p44 (nine exons) (SEQ ID NOS 157-164, respectively).

FIG. 25 shows that FLT3 ligand (FL) is induced in patients during treatment with Compound 1.

FIG. 26 demonstrates that interleukin-6 (IL-6) is induced in patients during treatment with Compound 1, and that a greater than 2-fold increase in IL-6 plasma concentration after treatment with Compound 1 correlates with patient fatigue.

FIG. 27 demonstrates that C-reactive protein (CRP) is induced in patients during treatment with Compound 1, and that a greater than 2-fold increase in CRP plasma concentration after treatment with Compound 1 correlates with patient fatigue.

FIG. 28 shows that a higher baseline value of CRP in patients with GIST correlates with progressive disease, in Trial D.

FIG. 29 shows that protein expression of OB-cadherin 1 (cadherin 11) is up-regulated in Colo205 xenograph tumors after exposure to Compound 1 for 24 or 48 hours.

### BRIEF DESCRIPTION OF THE TABLES

Tables 1-22 appear following the Examples disclosed in this application, and specifically after Section K.

Table 1 shows Compound B (SU5416) PBMC sample processing history for Trial A.

Table 2 shows a list of biomarker transcripts as detected in Affymetrix analysis.

Table 3 shows primer sequences used in RT-PCR validation analysis.

Table 4 shows a Mann-Whitney U Test comparison of expression fold change data from Compound B and control arms (Trial A). This statistical analysis was performed to assess the significance of differences in expression change ratios (day 56 vs day 1) between the Compound B and control arms. Separate comparisons were performed of expression change values from Affymetrix analysis and from SYBR Green RT-PCR validation experiments. P-values≦0.05 were considered statistically significant.

Table 5 shows the Mann-Whitney U Test of Compound B expression data in Trial B.

Table 6 shows a summary of class prediction results for pooled data (3 gene predictor set).

Table 7 shows changes in plasma levels of PLGF in patients in Trial C receiving daily treatment with a malate salt of Compound 1.

Table 8 shows changes in plasma levels of MIG, IP-10, and I-TAC in patients receiving treatment with Compound 1 or a malate salt thereof. Levels of IP-10 and I-TAC at end cycle 1 dosing are estimated values in some cases (>500), as the amount of IP-10 or I-TAC in these samples was higher than the highest standard provided for standard curve generation. All patients represented in this table are from Trial C, except for patient 11 from Trial B and patient 9 from Trial A. Patients in Trial C received treatment with a malate salt of Compound 1, while patients from Trials A and B received treatment with Compound 1.

Table 9 shows changes in PLGF and/or sVEGFR2 plasma levels in cancer patients after receiving treatment with Compound 1 or a malate salt thereof. For PLGF, italics text indicates a fold-change of 3-fold or greater, end of cycle 1 dosing relative to day 1. For sVEGFR2, italics text indicates a decrease of 30% or more, end of cycle 1 dosing relative to day 1. Patients in Trials C and D received treatment with a malate salt of Compound 1, while patients from Trials A and B received treatment with Compound 1.

Table 10 shows an increase in MIG plasma levels in cancer patients after receiving treatment with Compound 1 or malate salt thereof. As with Table 2, results are from Trial C except for patient 11 from Trial B and patient 9 from Trial A.

Table 11A shows the change in levels of various mRNA transcripts isolated from Colo205 xenograft tumors, as measured by DNA Array analysis, before exposure to Compound 1, and 6 hours and 24 hours after exposure to the first dose of Compound 1.

Table 11B shows the change in levels of various mRNA transcripts isolated from SF767 xenograft tumors, measured by DNA Array analysis, before exposure to Compound 1, and 4 hours and 24 hours after exposure to the first dose of Compound 1.

Table 12 shows the change in the levels of protein expression and/or mRNA transcript abundance in Colo205 xenograft tumors, as measured by Taqman Real Time PCR, before exposure to Compound 1, and at 6 hours versus 24 hours after exposure to the first dose of Compound 1. The following transcripts were measured: Amphiregulin, Cdc2-related protein kinase, phosphoinositol 3-kinase, p110 subunit, cyclin C, OB-Cadherin1, OB-Cadherin2, p85 subunit, Mucin 1, von Hippel-Lindau (VHL) tumor suppressor, ephrin recetor (EphB4), and Gelsolin.

Table 13 shows the forward and reverse primer and probe sequences used in the TaqMan Real Time PCR Analysis of Colo205 xenograft tumor samples.

Table 14 lists three sets of 2D gels used in MALDI-TOF-MS and MALDI-MS/MS analysis.

Table 15 shows the quantification of Spot #1202 from 2D gel analysis. 2D gel analysis was performed on samples isolated from HUVECs that were stimulated with VEGF after pre-treatment with Compound 1 or vehicle control (DMSO).

Table 16 shows definitive identification of Spot #1202 as interstitial collagenase precursor (pro-MMP-1), as seen in MALDI-TOF-MS analysis.

Table 17 identifies Spot #1202 as interstitial collagenase precursor (pro-MMP-1), as seen in MALDI-MS/MS analysis.

Table 18 shows quantitative ELISA analysis of pro-MMP1 levels in HUVEC conditioned media, after stimulatation of HUVEC cells with VEGF after pre-treatment with Compound 1 at 10 nM, 100 nM or 1 μM concentrations, or vehicle control (DMSO).

Table 19 shows an increase pro-MMP1 levels in patient plasma after treatment with Compound 1. Results are from Study B.

Table 20 lists the analytes measured using Array 1.1 and Array 2.1 in an antibody chip microassay analysis.

Table 21 lists 23 biomarkers that show changes in plasma levels following treatment with Compound 1. An up arrow, down arrow or (−) denote relative increase, decrease or no change in detected level respectively, in samples for patients 1, 2 and 3. The accession numbers for markers, not previously described herein, are as follows: ENA-78 (epithelial derived neutrophil activating protein 78) (SEQ ID NO: 48), P42830; MPIF-1 (myeloid progenitor inhibitory factor 1) (SEQ ID NO: 49), P55773; GCP-2 (gamma tubulin complex component 2) (SEQ ID NO: 50), Q9BSJ2; Amphiregulin (Amphireg) (SEQ ID NO: 51), AAA51781; IL-1α (interleukin-1 alpha) (SEQ ID NO: 52), NP 000566 for preprotein; IL-1β (interleukin-1 beta) (SEQ ID NO: 53), NP 000567 for preprotein; IL-2 (interleukin-2) (SEQ ID NO: 54), NP 000577 for preprotein; MIG (mitogen inducible gene) (SEQ ID NO: 55), NP 061821; NT4 (neurotrophin 4/neurotrophic factor 5) (SEQ ID NO: 56), NP 006170; IGFBP-1 (insulin-like growth factor binding factor-1) (SEQ ID NO: 57), NP 000587; GRO-β (SEQ ID NO: 58), AAA63183; TNFR1 (tumor necrosis factor receptor 1) (SEQ ID NO: 59), P19438; FLT3 ligand (fms-like tyrosine kinase ligand/Flk 2 ligand) (SEQ ID NO: 60), I38440; IL-6 (interleukin-6) (SEQ ID NO: 61), NP-000591; MCP-1 (monocyte chemoattractant protein 1) (SEQ ID NO: 62), P13500; TNFα (tumor necrosis factor alpha) (SEQ ID NO: 63), NP 000585; TARC (thymus and activation regulated chemokine) (SEQ ID NO: 64), Q92583; MMP7 (SEQ ID NO: 65), NP 002414 for preprotein; MMP9 (SEQ ID NO: 66), NP 004985 for preprotein; and leptin (SEQ ID NO: 67), NP000221 for preprotein. Note that accession numbers and SEQ ID NOs in this specification are used to identify cDNAs, mRNAs or proteins of interest, rather limit the biomarkers to specific sequences.

Table 22 shows the relative fold change of six plasma biomarkers in three patients (denoted 1, 2 and 3) following Compound 1 treatment relative to predose, as measured by two methods: ELISA; and antibody chip technology (MSI).

### DETAILED DESCRIPTION OF THE INVENTION

The present invention relates to novel methods for determining whether a test compound inhibits tyrosine kinase activity and novel methods for determining whether a mammal has been exposed to a test compound that inhibits tyrosine kinase activity. The invention also relates to novel methods for determining whether a mammal is experiencing or will experience a particular biological phenomenon, such as a therapeutic effect, “responding” (as defined herein), or an adverse event, in response to a compound that inhibit tyrosine kinase activity.

The novel methods comprise measuring in a mammal the level of at least one biomarker, such as a protein and/or mRNA transcript. Based on the level of at least one biomarker in the mammal exposed with a test compound, as compared to the level of the biomarker(s) in a mammal that has not been exposed to a test compound, the ability of the test compound to inhibit tyrosine kinase activity can be determined. The tyrosine kinases of the novel methods include, but are not limited to, those selected from the group of Flk-1 (KDR), c-kit, FLT1, FLT3, PDGFR-alpha, PDGFR-beta, FGFR-1, FGFR-2 and c-fms/CSF-1 receptor.

In certain embodiments, the test compound is an inhibitor of VEGF-mediated signal transduction. In further embodiments, the test compound is an inhibitor of VEGF-mediated tyrosine phosphorylation of a protein kinase, such as Flk-1. In other embodiments, the test compound is an indolinone, as described herein, and also in U.S. Ser. No. 10/281,266. In other embodiments, the tyrosine kinase inhibitor comprises compounds described in U.S. Ser. No. 09/783,264, WO 01/60814, U.S. Ser. No. 10/076,140, U.S. Ser. No. 10/281,266, U.S. Ser. No. 10/281,985, U.S. Ser. No. 10/237,966 (now a U.S. provisional application), as well as a U.S. provisional application Ser. No. 60/448,861, filed Feb. 24, 2003 (entitled “Treatment of excessive osteolysis with indolinone compounds”), all of which are hereby incorporated by reference.

Identification of biomarkers that provide rapid and accessible readouts of efficacy, drug exposure, or clinical response is increasingly important in the clinical development of drug candidates. Embodiments of the invention include measuring changes in the expression levels of secreted proteins, or plasma markers, which represent one category of biomarker. In one embodiment, plasma samples, which represent a readily accessible source of material, serve as a surrogate tissue for biomarker analysis.

A. Definitions

Unless otherwise stated the following terms used in the specification and claims have the meanings discussed below.

“Test compound” refers to any pharmaceutical composition that inhibits or modulates a protein tyrosine kinase.

“Tyrosine kinase modulator” or “tyrosine kinase inhibitor” refers to any chemical composition that modulates, affects, alters, inhibits or reduces the enzymatic activity or tyrosine phosphation action of a tyrosine kinase.

B. Biomarkers Modulated in Mammals Exposed to Tyrosine Kinase Inhibitors

In one embodiment, the invention includes a method for determining whether a test compound inhibits tyrosine kinase activity in a mammal, comprising:

(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A 11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;

(b), exposing the mammal to the test compound; and

(c) following the exposing of step (b), measuring in the mammal the level of at least one of the proteins and/or mRNA transcripts measured in step (a),

wherein a difference in the level of said protein and/or mRNA transcript measured in (c), compared to the level of protein and/or mRNA transcript measured in step (a) indicates that the test compound is an inhibitor of tyrosine kinase in the mammal.

Alternatively, a method for determining whether a test compound inhibits tyrosine kinase activity in a mammal comprises:

(a) exposing the mammal to the test compound; and

(b) following the exposing of step (a), measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1,

wherein a difference in the level of said protein and/or mRNA measured in (b), compared to the level of protein and/or mRNA in a mammal that has not been exposed to said test compound, indicates that the compound is an inhibitor of tyrosine kinase in the mammal.

In an other embodiment, the invention includes a method for determining whether a mammal has been exposed to a test compound that inhibits tyrosine kinase activity, comprising:

(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;

(b), exposing the mammal to the test compound; and

(c) following the exposing of step (b), measuring in the mammal the level of at least one of the proteins and/or mRNA transcripts measured in step (a),

wherein a difference in the level of said protein and/or mRNA measured in (c), compared to the level of protein and/or mRNA in step (a) indicates that the mammal has been exposed to a test compound that inhibits tyrosine kinase activity.

Alternatively, a method for determining whether a mammal has been exposed to a test compound that inhibits tyrosine kinase activity comprises:

(a) exposing the mammal to the test compound; and

(b) following the exposing of step (a), measuring in a mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1,

wherein a difference in the level of said protein and/or mRNA measured in (b), compared to the level of protein and/or mRNA in a mammal that has not been exposed to said test compound, indicates that the mammal has been exposed to a test compound that is an inhibitor of tyrosine kinase.

In an other embodiment, the invention includes a method for measuring the level of exposure in a mammal to a test compound that inhibits tyrosine kinase activity, comprising:

(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;

(b), exposing the mammal to the test compound; and

(c) following the exposing of step (b), measuring in the mammal the level of at least one of the proteins and/or mRNA transcripts measured in step (a),

wherein a difference in the level of said protein and/or mRNA measured in (c), compared to the level of protein and/or mRNA in step (a) is indicative of the level of exposure in the mammal to the test compound that inhibits tyrosine kinase activity.

Alternatively, a method for measuring the level of exposure in a mammal to a test compound that inhibits tyrosine kinase activity comprises:

(a) exposing the mammal to the test compound; and

(b) following the exposing of step (a), measuring in a mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1,

wherein a difference in the level of said protein and/or mRNA measured in (b), compared to the level of protein and/or mRNA in a mammal that has not been exposed to said test compound, is indicative of the level of exposure in the mammal to the test compound that inhibits tyrosine kinase activity.

In another embodiment, the invention includes a method for determining whether a mammal is responding to a compound that inhibits tyrosine kinase activity, comprising:

(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;

(b), exposing the mammal to the compound; and

(c) following the exposing of step (b), measuring in the mammal the level of at least one of the proteins and/or mRNA transcripts measured in step (a),

wherein a difference in the level of said protein and/or mRNA transcripts measured in (c), compared to the level of protein and/or mRNA transcript for said protein in step (a) indicates that that the mammal is responding to the compound that inhibits tyrosine kinase activity.

Alternatively, a method for determining whether a mammal is responding to a compound that inhibits tyrosine kinase activity comprises:

(a) exposing the mammal to the compound; and

(b) following the exposing step (a), measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1,

wherein a difference in the level of said protein and/or mRNA measured in (b), compared to the level of protein and/or mRNA in a mammal that has not been exposed to said compound, indicates that the mammal is responding to the compound that inhibits tyrosine kinase.

The term “responding” encompasses responding by way of a biological and cellular response, as well as a clinical response (such as improved symptoms, a therapeutic effect or an adverse event), in a mammal.

In another embodiment, the invention includes a method for identifying a mammal that will respond therapeutically to a method of treating cancer comprising administering at least one inhibitor of VEGFR and/or PDGFR tyrosine kinases, wherein the method for identifying the mammal comprises:

(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;

(b) exposing the mammal to at least one inhibitor of VEGFR and/or PDGFR tyrosine kinases; and

(c) following the exposing of step (b), measuring in the mammal the level of at least one of the proteins and/or mRNA transcripts measured in step (a),

wherein a difference in the level of said protein and/or mRNA transcripts measured in (c), compared to the level of protein and/or mRNA transcript for said protein in step (a) indicates that that the mammal will respond therapeutically to a method of treating cancer comprising administering at least one inhibitor of VEGFR and/or PDGFR tyrosine kinases.

In another embodiment, the invention includes a method for testing or predicting whether a mammal will respond therapeutically to a method of treating cancer comprising administering at least one inhibitor of VEGFR and/or PDGFR tyrosine kinases, wherein the method for testing or predicting comprises:

(a) measuring in a mammal with cancer the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;

(b) measuring in a same type of mammal without cancer the level of at least one of the same proteins and/or mRNA transcripts measured in step (a);

(c) comparing levels of said proteins and/or mRNA transcripts measured in (a) and (b);

wherein a difference in the level of said protein and/or mRNA in the mammal with cancer as measured in step (a), compared to the level of said protein and/or mRNA in the mammal without cancer as measured in step (b), indicates that the mammal will respond therapeutically to at least one inhibitor of VEGFR and/or PDGFR tyrosine kinases.

As used throughout the specification, the term “respond therapeutically” refers to the alleviation or abrogation of a disease, such as cancer. This term means that the life expectancy of an individual affected with the disease will be increased or that one or more of the symptoms of the disease will be reduced or ameliorated. The term encompasses a reduction in cancerous cell growth or tumor volume. Whether a mammal responds therapeutically can be measured by many methods well known known in the art, such as PET imaging.

In another embodiment, the mammal is a human. In other embodiments, the mammal is a rat, mouse, dog, rabbit, pig, sheep, cow, horse, cat, primate, or monkey.

In other embodiments, any of the proceeding methods is an in vitro method, and the protein and/or mRNA biomarker is measured in at least one mammalian biological tissue. In other embodiments, the protein and/or mRNA biomarker is measured in at least one biological fluid, including but not limited to whole fresh blood, peripheral blood mononuclear cells, frozen whole blood, fresh plasma, frozen plasma, urine and saliva. In other embodiments, the protein and/or mRNA biomarker is measured in at least one biological tissue including but not limited to buccal mucosa tissue, skin, hair follicles, tumor tissue and bone marrow.

In yet other embodiments, the methods of the invention are carried out on mammals who have cancer. The cancer can be, for example, but is not limited to, prostate cancer, colorectal cancer (CRC), thyroid cancer, an advanced solid malignancy, pancreatic cancer, breast cancer, parotid cancer, synovial cell cancer or sarcoma, gastrointestinal stromal tumor (GIST), laryngeal cancer, testicular cancer, leiomyosarcoma, rectal cancer, gall-bladder cancer, hepatocellular cancer, melanoma, ovary cancer, lung cancer, colon cancer, renal cell carcinoma, sarcoma, retropero sarcoma, pelvis sarcoma, uterine cancer, pelvic angiosarcoma, pleural mesothelioma, neuroendocrine cancer, bronchial adenocarcinoma, head and neck cancer and/or thymic cancer.

In other embodiments, any of the preceeding methods also comprise a step wherein the mammal is also exposed to a cancer chemotherapeutic agent before, during and/or after exposure to the compound that inhibits tyrosine kinase activity.

Other embodiments also include any of the proceeding methods, wherein the “difference” refers to an increase in the level of at least one of the following protein(s) and/or mRNA transcript(s): PAI-1, TIMP-1, vinculin, VEGF, PLGF, VEGR/PLGR heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), histone H2B, human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ephrin receptor EphB4, OB-cadherin 1, phosphoinositol 3-kinase p85 subunit, mucin 1 and gelsolin, as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has at least one of prostate cancer, colon cancer, thyroid cancer and an advance solid malignancy, and wherein the “difference” refers to an increase in the level of VEGF protein and/or mRNA transcript as measured after exposure to a compound that inhibits tyrosine kinase activity, compared to the level of VEGF protein and/or mRNA transcript as measured before exposure to the compound.

Other embodiments also include any of the proceeding methods wherein the mammal has colon or colorectal cancer, and wherein the “difference” refers to an increase in the level of at least one of VEGF, MMP-9, lactoferrin, lipocalin-2, and/or CD24 antigen protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has at least one of synovial sarcoma, rectal cancer, gall-bladder cancer, hepatocellular cancer, melanoma, breast cancer, ovary cancer, small cell lung cancer, colon cancer, renal cell carcinoma, sarcoma, retropero sarcoma, pelvis sarcoma, parotid cancer, uterine cancer, pelvic angiosarcoma, colorectal cancer and gastrointestinal stromal tumor (GIST), and wherein the “difference” refers to an increase in the level of at least one of VEGF, PLGF and VEGF/PLGF heterodimers protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has an advanced solid malignancy, and wherein the “difference” refers to an increase in the level of VEGF and/or MMP-9 protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has at least one of pancreatic cancer, synovial sarcoma, colon cancer, non-small cell lung cancer (NSCLC), rectal cancer, pelvis sarcoma, and sarcoma and/or bronchial adenocarcinoma, and wherein the “difference” refers to an increase in the level of at least one of MIG, IP-10 and I-TAC protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has thryoid cancer, and wherein the “difference” refers to an increase in the level of at least one of VEGF, eucaryotic initiation factor 4A11, human (clone 5) orphan G protein-coupled receptor, Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophillin, Genbank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), histone H2b and human RLIP76 protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has pancreatic cancer, and wherein the “difference” refers to an increase in the level of at least one of eucaryotic initiation factor 4A11, human (clone 5) orphan G protein-coupled receptor, Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, and human MHC class II lymphocyte antigen (HLA-DP) beta chain protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has breast cancer, and wherein the “difference” refers to an increase in the level of at least one of human acidic ribosomal phosphoprotein P0, human cyclophillin, Genbank Accession No. AI541256 (Homo sapiens cDNA), human T-cell receptor active beta chain, and human MHC class II lymphocyte antigen (HLA-DP) beta chain protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has prostate cancer, and wherein the “difference” refers to an increase in the level of at least one of VEGF, eucaryotic initiation factor 4A11, human (clone 5) orphan G protein-coupled receptor, Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, Genbank Accession No. AI541256 (Homo sapiens cDNA), human T-cell receptor active beta chain, and human MHC class II lymphocyte antigen (HLA-DP) beta chain protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has parotid cancer, and wherein the “difference” refers to an increase in the level of at least one of Homo sapiens thymosin beta-10 gene, Homo sapiens MAP kinase kinase 3 (MKK3) and histone H2B member R protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has synovial cell cancer, and wherein the “difference” refers to an increase in the level of human RLIP76 protein and/or mRNA transcript as measured after exposure to a compound that inhibits tyrosine kinase activity, compared to the level of human RLIP76 protein and/or mRNA transcript as measured before exposure to the compound.

Other embodiments also include any of the proceeding methods, wherein the “difference” refers to a decrease in the level of at least one of the following protein(s) and/or mRNA transcript(s): ITIH4, PAI-1, soluble VEGF receptor 2 (sVEGFR2), Homo sapiens thymosin beta-10 gene, human leucocyte antigen (CD37), human MHC class II HLA-DR beta-1, Homo sapiens translation initiation factor elF3 p66 subunit, human MHC class II lymphocyte antigen (HLA-DP), human KIAA0195, human beta-tubulin class III isotype (beta-3), Homo sapiens MAP kinase kinase 3 (MKK3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, human RLIP76 protein, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78, MPIF-1, MMP7, MIG, cdc2 related protein kinase, and phosphoinositol 3-kinase p110 subunit, as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has is at least one of breast cancer, prostate cancer and thyroid cancer, and wherein the “difference” refers to a decrease in the level of ITIH4 protein and/or mRNA transcript as measured after exposure to a compound that inhibits tyrosine kinase activity, compared to the level of ITIH4 protein and/or mRNA transcript as measured before exposure to the compound.

Other embodiments also include any of the proceeding methods wherein the mammal has is at least one of synovial sarcoma, rectal cancer, gall-bladder cancer, hepatocellular cancer, melanoma, breast cancer, ovary cancer, small cell lung cancer, melanoma, colon cancer, renal cell carcinoma, non-small cell lung cancer (NSCLC), sarcoma, retropero sarcoma, pelvis sarcoma, squamous cell carcinoma parotid cancer, bronchial adenocarcinoma, uterine cancer, pelvic angiosarcoma, pleural mesothelioma, colorectal cancer (CRC), neuroendocrine cancer, gastrointestinal stromal tumor (GIST), head and neck cancer, thymic cancer and thyroid cancer, and wherein the “difference” refers to a decrease in the level of sVEGFR2 protein and/or mRNA transcript as measured after exposure to a compound that inhibits tyrosine kinase activity, compared to the level of sVEGFR2 protein and/or mRNA transcript as measured before exposure to the compound.

Other embodiments also include any of the proceeding methods wherein the mammal has parotid cancer, and wherein the “difference” refers to a decrease in the level of at least one of Homo sapiens thymosin beta-10 gene, human leucocyte antigen (CD37), human MHC class II HLA-DR beta-1, Homo sapiens translation initiation factor elF3 p66 subunit, human MHC class II lymphocyte antigen (HLA-DP), human beta-tubulin class III isotype (beta-3), and human RLIP76 protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has thyroid cancer, and wherein the “difference” refers to a decrease in the level of at least one of human KIAA0195, human beta-tubulin class III isotype (beta-3), Homo sapiens MAP kinase kinase 3 (MKK3), human tropomyosin, 1-phosphatidyl inositol-4-phosphate-5-kinase isoform C, human MLC1 emb gene for embryonic myosin alkaline light chain, Homo sapiens glyoxalase II, Homo sapiens trans-golgi network glycoprotein 48, histone H2B member R, human RLIP76 protein, Genbank Accession No. W26677 (human retina cDNA), human PMI gene for a putative receptor protein, and human DNA-binding protein A (dbpA) protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has pancreatic cancer, and wherein the “difference” refers to a decrease in the level of at least one of human KIAA0195, human beta-tubulin class III isotype (beta-3), Homo sapiens MAP kinase kinase 3 (MKK3), human tropomyosin, 1-phosphatidyl inositol-4-phosphate-5-kinase isoform C, human MCL1 emb gene for embryonic myosin alkaline light chain, Homo sapiens glyoxalase II, Genbank Accession No. W26677 (human retina cDNA), human PMI gene for a putative receptor protein, and human DNA-binding protein A (dbpA) protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has prostate cancer, and wherein the “difference” refers to a decrease in the level of at least one of human beta-tubulin class III isotype (beta-3), Homo sapiens MAP kinase kinase 3 (MKK3), human tropomyosin, 1-phosphatidyl inositol-4-phosphate-5-kinase isoform C, human MCL1 emb gene for embryonic myosin alkaline light chain, Homo sapiens glyoxalase II, Homo sapiens trans-golgi network glycoprotein 48, human RLIP76 protein, Genbank Accession No. W26677 (human retina cDNA), human PMI gene for a putative receptor protein, and human DNA-binding protein A (dbpA) protein(s) and/or mRNA transcript(s) as measured after exposure to a compound that inhibits tyrosine kinase activity, compared to the level of the same protein(s) and/or mRNA transcript(s) as measured before exposure to the compound.

Other embodiments also include any of the proceeding methods wherein the mammal has breast cancer, and wherein the “difference” refers to a decrease in the level of at least one of human KIAA0195, Homo sapiens trans-golgi network glycoprotein 48, histone H2B and human RLIP76 protein(s) and/or mRNA transcript(s) as measured after exposure to a compound that inhibits tyrosine kinase activity, compared to the level of the same protein(s) and/or mRNA transcript(s) as measured before exposure to the compound.

In another embodiment, the invention also includes a kit comprising:

(a) antibody and/or nucleic acid for detecting the presence of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1; and

(b) instructions for determining whether or not a mammal will respond therapeutically to a method of treating cancer comprising administering a compound that inhibits tyrosine kinase activity.

In another embodiment, the invention also includes the preceeding kit, wherein the instructions comprise the steps of:

(i) measuring in a mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B, Genbank Accession No. W26677 (human retina cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;

(ii) exposing the mammal to a compound that inhibits tyrosine kinase activity; and

(iii) following the exposing step of (ii), measuring in the mammal the level of at least one of the proteins and/or mRNA transcripts for such proteins measured in step (i);

wherein a difference in the level of said proteins and/or mRNA transcripts measured in (iii), compared to the level of proteins and or mRNA transcripts measured in step (i) indicates that the mammal will respond therapeutically to a method of treating cancer comprising administering the compound that inhibits tyrosine kinase activity.

In another embodiment, the invention also includes a method for testing or predicting whether a mammal will experience an adverse event in response to a method of treating cancer comprising administering a tyrosine kinase inhibitor, wherein the method for testing or predicting comprises:

(a) measuring in the mammal the level of IL-6 or C-reactive protein (CRP) protein and/or mRNA transcript for such protein and/or gene before administering the tyrosine kinase inhibitor;

(b) measuring in the mammal the level of IL-6 or CRP protein and/or mRNA transcript for such protein and/or gene after administering the tyrosine kinase inhibitor;

(c) comparing levels of said IL-6 or CRP protein and/or mRNA transcript measured in (a) and (b);

wherein a level of two-fold or greater of said protein and/or mRNA transcript as measured in step (b), compared to the level of said protein and/or mRNA transcript as measured in step (a), indicates that the mammal will experience fatigue in response to the method of treating cancer comprising administering the tyrosine kinase inhibitor.

As used in the specification, the term “adverse event” refers to a physiological effect in a mammal, such as fatigue or other side effect, that is severe enough to warrent altering, reducing or eliminating the mammal's exposure to a particular tyrosine kinase inhibitor. Exposure or adminstration can be altered, reduced or eliminated in terms of the amount or dosage of the tyrosine kinase inhibitor, as well as length of time and/or frequency of exposure. A determination as to whether a particular physiological effect is severe enough to be considered “an adverse event” falls within the judgment of those skilled in the art, such as a laboratory scientist, veterinarian or medical practitioner.

C. Further Embodiments of the Novel Methods

1. Measurement of Protein and mRNA

In other embodiments, the novel methods of Section B are carried out so that the step where the mammal is exposed to test compound includes administration of at least one dose of test compound, or at least two doses, or at least 5 doses or at least 10 doses, up to at least 55 or 56 doses. In certain embodiments, these doses are administered during a period of 4 hours, 6 hours, or 24 hours to about 100 days. In further embodiments, the doses are administered over a period of 24 hours, 2 days, or 28 days. In other embodiments, two doses are administered per every 24 hours, and in other embodiments, the doses are administered about every 12 hours. It will be understood by those of skill in the art that the administration of test compound, according to the exposure steps of the methods of Section B, can be varied to suit individual needs of the mammal being treated, the compound being administered, the method of administration and the disease being treated. For example, in a typical dosing regimen, the patient receives one dose per day of test compound, for a number of days, such as about 28 or about 56 days. In other dosing regimens, the test compound is administered about once per day, twice per week, or once per week.

The measurement of protein and/or RNA, following the exposure step in the methods of Section B, can be carried out on a sample from the mammal taken about 4 or 6 hours following the first dose (exposure) of the mammal to test compound. In other embodiments, this measurement is carried out on a sample taken 12 hours, 1 day, 2 days, up to about 100 days, after the first dose (exposure) of the mammal to test compound. In other embodiments, the protein and/or mRNA measurements are taken from samples from the mammals 4 or 6 hours after the first dose of test compound or 24 hours after the first dose of test compound, or 15 or 28 days after the first dose of test compound. Typically, dosing of test compound will be periodic between the first and last dose of test compound that precedes the sample taken for measurement of biomarker protein and/or mRNA. For example, the test compound is administered once a day, every day for 28 days. Typically, the mammal sample taken (for measurement of biomarker protein and/or mRNA) will be taken shortly following the most recent dose of test compound, for example within 24 of the most recent dose of test compound.

In other embodiments, the methods of Section B are carried out so that the measurement of protein and/or mRNA is carried out on a mammalian tissue selected from biological fluids, including but not limited to the group of whole fresh blood, peripheral blood mononuclear cells, frozen whole blood, fresh plasma, frozen plasma, urine, saliva, and other tissues including but not limited to buccal mucosa tissue, skin, hair follicles, tumor tissue, bone marrow.

In other embodiments, the methods of Section B are carried out on a mammal that is further exposed to other chemotherapeutic agents, including but not limited to 5-fluoro-uracil (5-FU), leucovorin, CPT11, aromasin, taxol, paclitaxel, other “standard of care” agents used in patients, COX-2 inibitors (such as celecoxcib), and other tyrosine kinase inhibitors. Such exposure to a cancer chemotherapeutic agent can be before, during and/or after exposure to test compound.

In other embodiments, the difference in the level of protein or mRNA measured in the methods of Sections B is an increase of at least about 10% or 15% or 20% or 25% or 30% or 35% or 50% or 75% or 100%. In another embodiment, the difference in the level of protein or mRNA measured in the methods of Sections B is an increase of at least 25%. In other embodiments, the difference in the level of protein or mRNA measured in the methods of Sections B is an increase of at least 2-, 3-, 5-, 10-, 15- or 24-fold. In still further embodiments, the difference in the level of protein or mRNA measured in the methods of Sections B is an increase of at least 1.3-, 1.4-, 1.5-, 1.6-, 1.7-, 2.0-, 2.1-, 2.2-, 2.3-, 2.5-, 3.0-, 3.5-, 4.0-, 4.2-, 4.5-, 5.0-, 5.5-, 6.0-, 6.1-, 6.5-, 7.0-, 7.3-, 10.0-, 15.0-, 19.0-, or 24-fold. In another embodiment, the difference in the level of protein or mRNA measured in the methods of Sections B is an increase of at least about 1.7- or 2.0-fold.

In other embodiments, the difference in the level of protein or mRNA measured in the methods of Sections B is a decrease of at least about 10% or 15% or 20% or 25% or 30% or 35% or 50% or 75% or 100%. In another embodiment, the difference in the level of protein or mRNA measured in the methods of Sections B is a decrease of at least about 25%. In still further embodiments, the difference in the level of protein or mRNA measured in the methods of Sections B is a decrease of at least 1.3-, 1.4-, 1.5-, 1.6-,1.7-, 2.0-, 2.1-, 2.2-, 2.3-, 2.5-, 3.0-, 3.5- or 3.7-fold. In another embodiment, the difference in the level of protein or mRNA measured in the methods of Sections B is a decrease of at least about 1.7- or 2.0-fold.

To quantify the protein and/or mRNA measured in the novel methods of Section B, methods well known to the skilled artisan are used. For example, quantification of protein can be carried out using methods such as ELISA, 2-dimensional SDS PAGE, Western Blot, immunoprecipitation, immunohistochemistry, fluorescense activated cell sorting (FACS), fow cytometry. Quantification of mRNA is measured using methods such as PCR, array hybridization, Northern blot, in-situ hybridization, dot-blot, Taqman, RNAse protection assay.

In further embodiments of the invention, the methods of Section B are carried out so that the level of at least two, or at least three, or at least four, or at least five, or at least 6, or at least seven or at least eight, or at least nine, up to 87 of the biomarkers are measured in a mammal. In other embodiments, the methods of Section B comprise measuring the level of at least two, up to 66 biomarkers of Section B that are increased upon exposure of a mammal to a compound that inhibits tyrosine kinase. In other embodiments, the methods of Section B comprise measuring the level of at least two, up to 39 biomarkers of Section B that are decreased upon exposure of a mammal to a compound that inhibits tyrosine kinase.

2. Tyrosine Kinase and Inhibitors of Tyrosine Kinase

In certain embodiments, the tyrosine kinases of the novel methods are selected from the group of Flk-1 (KDR), c-kit, FLT1, FLT3, PDGFR-alpha, PDGFR-beta, FGFR-1, FGFR-2 and c-fms/CSF-1 receptor. See, for example, U.S. Pat. No. 6,177,401 (Flk-1), WO 01/45689 (c-kit), GenBank Accession No. NM 002609 (PDGFR-beta), GenBank Accession No. NM 006206 (PDGFR-alpha), GenBank Accession No. NM 023109 (FGFR-1), GenBank Accession No. NM 023028 (FGFR-2) and GenBank Accession No. NP_{—}005202 (c-fms/CSF-1 receptor).

FLT3 (fms like tyrosine kinase 3) is a member of the class III receptor tyrosine kinases. Those of skill in the art will recognize that FLT3 has also been called “flk2” in the scientific literature. “FLT3” as used herein, refers to a polypeptide having, for example, the sequence set forth in accession number gi|4758396|ref|NP_{—}004110.1| fms-related tyrosine kinase 3 [Homo sapiens ], or gi|544320|sp|P36888|FLT3_HUMAN FL CYTOKINE RECEPTOR PRECURSOR (TYROSINE-PROTEIN KINASE RECEPTOR FLT3) (STEM CELL TYROSINE KINASE 1) (STK-1) (CD135 ANTIGEN), or gi|409573|gb|AAA18947.1| (U02687) serine/threonine protein kinase [Homo sapiens]. Corresponding mRNA accessions for the first two sequences are gi|4758395|ref|NM_{—}004119.1| Homo sapiens fms-related tyrosine kinase 3 (FLT3), mRNA gi|406322|emb|Z26652.1|HSFLT3RTK H.sapiens FLT3 mRNA for FLT3 receptor tyrosine kinase.

In other embodiments, the test compound is an inhibitor of VEGF-mediated signal transduction. In further embodiments, the test compound is an inhibitor of VEGF-mediated tyrosine phosphorylation of a protein kinase, such as Flk-1. In other embodiments, the test compound is an indolinone compound. In another embodiment, the test compound is a compound of Formula I. These, and other exemplary tyrosine kinase inhibitors, are shown below. The skilled artisan will recognize that the novel methods of the invention can be used to test any tyrosine kinase inhibitor, in addition to those listed below.

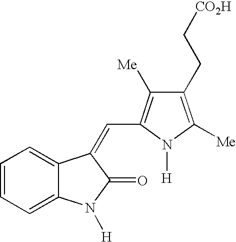

Compound A (SU6668): 3-[2,4-dimethyl-5-(2-oxo-1,2-dihydro-indol-3-ylidenemethyl)-1H-pyrrol-3-yl]-propionic acid.

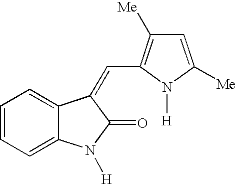

Compound B (SU5416): 3-(3,5-dimethyl-1H-pyrrol-2-ylmethylene)-1,3-dihdyro-indol-2-one.

A pyrrole substituted 2-indolinone having the formula:

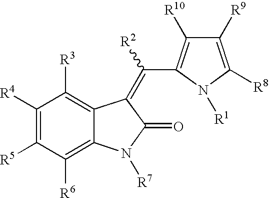

wherein:

R¹, R², and R⁷ are hydrogen;

R³, R⁴, R⁵, and R⁶ are independently selected from the group consisting of hydrogen, hydroxy, halo, unsubstituted lower alkyl, lower alkyl substituted with a carboxylic acid, unsubstituted lower alkoxy, carboxylic acid, unsubstituted aryl, aryl substituted with one or more unsubstituted lower alkyl alkoxy, and morpholino;

R⁸ is unsubstituted lower alkyl;

R⁹ is —(CH₂)(CH₂)C(═O)OH; and

R₁₀ is unsubstituted lower alkyl.

A compound having the formula:

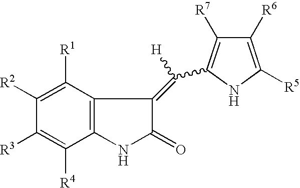

wherein:

R¹ is selected from the group consisting of hydrogen, halo, alkyl, cyclkoalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;

R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;

R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R¹⁴, —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);

R⁴ is selected from the group consisting of hydrogen, halogen, alkyl, hydroxy, alkoxy and —NR¹³R¹⁴;

R⁵ is selected from the group consisting of hydrogen, alkyl and —C(O)R₁₀;

R⁶ is selected from the group consisting of hydrogen, alkyl and —C(O)R₁₀;

R⁷ is selected from the group consisting of hydrogen, alkyl, aryl, heteroaryl, —C(O)R¹⁷ and —C(O)R¹⁰; or

R⁶ and R⁷ may combine to form a group selected from the group consisting of —(CH₂)₄—, —(CH₂)₅— and —(CH₂)₆—;

with the proviso that at least one of R⁵, R⁶ or R⁷ must be —C(O)R₁₀;

R⁸ and R⁹ are independently selected from the group consisting of hydrogen, alkyl and aryl;

R₁₀ is selected from the group consisting of hydroxy, alkoxy, aryloxy, —N(R¹¹)(CH₂)ₙR¹², and —NR¹³R¹⁴;

R¹¹ is selected from the group consisting of hydrogen and alkyl;

R¹² is selected from the group consisting of —NR¹³R¹⁴, hydroxy, —C(O)R¹⁵, aryl, heteroaryl, —N⁺(O⁻)R¹³R¹⁴, —N(OH)R¹³, and —NHC(O)R^{a} (wherein R^{a} is unsubstituted alkyl, haloalkyl, or aralkyl);

R¹³ and R¹⁴ are independently selected from the group consisting of hydrogen, alkyl, lower alkyl substituted with hydroxyalkylamino, cyanoalkyl, cycloalkyl, aryl and heteroaryl; or

R¹³ and R¹⁴ may combine to form a heterocyclo group;

R¹⁵ is selected from the group consisting of hydrogen, hydroxy, alkoxy and aryloxy;

R¹⁶ is selected from the group consisting of hydroxy, —C(O)R¹⁵, —NR¹³R¹⁴ and —C(O)NR¹³R¹⁴;

R¹⁷ is selected from the group consisting of alkyl, cycloalkyl, aryl and heteroaryl;

R²⁰ is alkyl, aryl, aralkyl or heteroaryl; and

n and r are independently 1, 2, 3, or 4;

or a pharmaceutically acceptable salt thereof.

A compound having the formula:

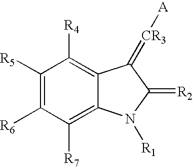

wherein:

R₁ is H;

R₂ is O or S;

R₃ is hydrogen;

R₄, R₅, R₆, and R₇ are each independently selected from the group consisting of hydrogen, alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R, and CONRR′;

A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole, optionally substituted at one or more positions with alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R or CONRR′;

n is 0-3;

R is H, alkyl or aryl; and

R′ is H, alkyl or aryl;

or a pharmaceutically acceptable salt thereof.

A compound having the formula:

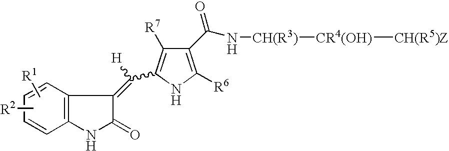

wherein:

R¹ is selected from the group consisting of hydrogen, halo, alkyl, haloalkoxy, cycloalkyl, heteroalicyclic, hydroxy, alkoxy, —C(O)R⁸, —NR⁹R¹⁰ and —C(O)NR¹²R¹³;

R² is selected from the group consisting of hydrogen, halo, alkyl, trihalomethyl, hydroxy, alkoxy, cyano, —NR⁹R¹⁰, —NR⁹C(O)R¹⁰, —C(O)R⁸, —S(O)₂NR⁹R¹⁰ and —SO₂R¹⁴ (wherein R¹⁴ is alkyl, aryl, aralkyl, heteroaryl and heteroaralkyl);

R³, R⁴ and R⁵ are independently hydrogen or alkyl;

Z is aryl, heteroaryl, heterocycle, or —NR¹⁵R¹⁶ wherein R¹⁵ and R¹⁶ are independently hydrogen or alkyl; or R¹⁵ and R¹⁶ together with the nitrogen atom to which they are attached from a heterocycloamino group;

R⁶ is selected from the group consisting of hydrogen or alkyl;

R⁷ is selected from the group consisting of hydrogen, alky, aryl, heteroaryl, and —C(O)R¹⁷ as defined below;

R⁸ is selected from the group consisting of hydroxy, alkoxy and aryloxy;

R⁹ and R¹⁰ are independently selected from the group consisting of hydrogen, alkyl, cyanoalkyl, cycloalkyl, aryl and heteroaryl; or

R⁹ and R¹⁰ combine to form a heterocycloamino group;

R¹² and R¹³ are independently selected from the group consisting of hydrogen, alkyl, hydroxyalkyl, and aryl; or R¹² and R¹³ together with the nitrogen atom to which they are attached form a heterocycloamino;

R¹⁷ is selected from the group consisting of alkyl, cycloalkyl, aryl, hydroxy and heteroaryl;

or a pharmaceutically acceptable salt thereof.

In other embodiments of the invention, a mammal is exposed to a compound of Formula I:

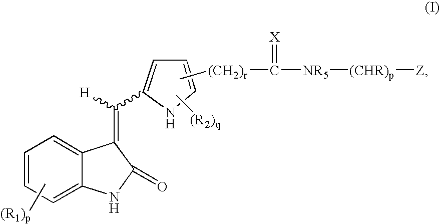

wherein:

R is independently H, OH, alkyl, aryl, cycloalkyl, heteroaryl, alkoxy, heterocyclic and amino;

each R₁ is independently selected from the group consisting of alkyl, halo, aryl, alkoxy, haloalkyl, haloalkoxy, cycloalkyl, heteroaryl, heterocyclic, hydroxy, —C(O)—R₈, —NR₉R₁₀, —NR₉C(O)—R₁₂ and —C(O)NR₉R₁₀;

each R₂ is independently selected from the group consisting of alkyl, aryl, heteroaryl, —C(O)—R₈, and SO₂R″, where R″ is alkyl, aryl, heteroaryl, NR₉N₁₀ or alkoxy;

each R₅ is independently selected from the group consisting of hydrogen, alkyl, aryl, haloalkyl, cycloalkyl, heteroaryl, heterocyclic, hydroxy, —C(O)—R₈ and (CHR)ᵣR₁₁;

X is O or S;

p is 0-3;

q is 0-2;

r is 0-3;

R₈ is selected from the group consisting of —OH, alkyl, aryl, heteroaryl, alkoxy, cycloalkyl and heterocyclic;

R₉ and R₁₀ are independently selected from the group consisting of H, alkyl, aryl, aminoalkyl, heteroaryl, cycloalkyl and heterocyclic, or R₉ and R₁₀ together with N may form a ring, where the ring atoms are selected from the group consisting of C, N, O and S;

R₁₁ is selected from the group consisting of —OH, amino, monosubstituted amino, disubstituted amino, alkyl, aryl, heteroaryl, alkoxy, cycloalkyl and heterocyclic

R₁₂ is selected from the group consisting of alkyl, aryl, heteroaryl, alkoxy, cycloalkyl and heterocyclic;

Z is OH, O-alkyl, or —NR₃R^{4,} where R₃ and R⁴ are independently selected from the group consisting of hydrogen, alkyl, aryl, heteroaryl, cycloalkyl, and heterocyclic, or R₃ and R₄ may combine with N to form a ring where the ring atoms are selected from the group consisting of CH₂, N, O and S or

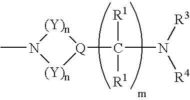

wherein Y is independently CH₂, O, N or S,

Q is C or N;

n is independently 0-4; and

m is 0-3;

or a pharmaceutically acceptable salt thereof.

In another embodiment, a mammal is exposed to a compound of Formula II:

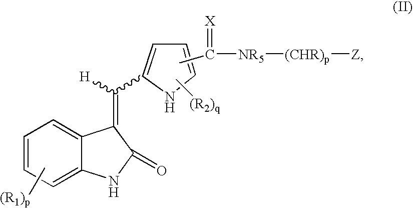

wherein:

R is independently H, OH, alkyl, aryl, cycloalkyl, heteroaryl, alkoxy, heterocyclic and amino;

each R₁ is independently selected from the group consisting of alkyl, halo, aryl, alkoxy, haloalkyl, haloalkoxy, cycloalkyl, heteroaryl, heterocyclic, hydroxy, —C(O)—R₈, —NR₉R₁₀, —NR₉C(O)—R₁₂ and —C(O)NR₉R₁₀;

each R₂ is independently selected from the group consisting of alkyl, aryl, heteroaryl, —C(O)—R₈, and SO₂R″, where R″ is alkyl, aryl, heteroaryl, NR₉N₁₀ or alkoxy;

each R₅ is independently selected from the group consisting of hydrogen, alkyl, aryl, haloalkyl, cycloalkyl, heteroaryl, heterocyclic, hydroxy, —C(O)—R₈ and (CHR)ᵣR₁₁;

X is O or S;

p is 0-3;

q is 0-2;

r is 0-3;

R₈ is selected from the group consisting of —OH, alkyl, aryl, heteroaryl, alkoxy, cycloalkyl and heterocyclic;

R₉ and R₁₀ are independently selected from the group consisting of H, alkyl, aryl, aminoalkyl, heteroaryl, cycloalkyl and heterocyclic, or R₉ and R₁₀ together with N may form a ring, where the ring atoms are selected from the group consisting of C, N, O and S;

R₁₁ is selected from the group consisting of —OH, amino, monosubstituted amino, disubstituted amino, alkyl, aryl, heteroaryl, alkoxy, cycloalkyl and heterocyclic

R₁₂ is selected from the group consisting of alkyl, aryl, heteroaryl, alkoxy, cycloalkyl and heterocyclic;

Z is OH, O-alkyl, or —NR₃R⁴, where R₃ and R⁴ are independently selected from the group consisting of hydrogen, alkyl, aryl, heteroaryl, cycloalkyl, and heterocyclic, or R₃ and R₄ may combine with N to form a ring where the ring atoms are selected from the group consisting of CH₂, N, O and S or

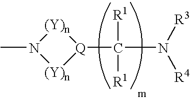

wherein Y is independently CH₂, O, N or S,

Q is C or N;

n is independently 0-4; and

m is 0-3;

or a pharmaceutically acceptable salt thereof.

In another embodiment of the invention, a mammal is exposed to a compound of Formula I or II, wherein R₁ is halo (e.g., F and Cl) and Z is —NR₃R₄ wherein R₃ and R₄ are independently H or alkyl.

In another embodiment, Z of Formula I or II is —NR₃R₄, wherein R₃ and R₄ form a morpholine ring.

In another embodiment, Z of Formula I or II is:

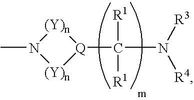

wherein each Y is CH₂, each n is 2, m is 0 and R₃ and R₄ form a morpholine ring.

In another embodiment of the invention, a mammal is exposed to a compound selected from the group consisting of

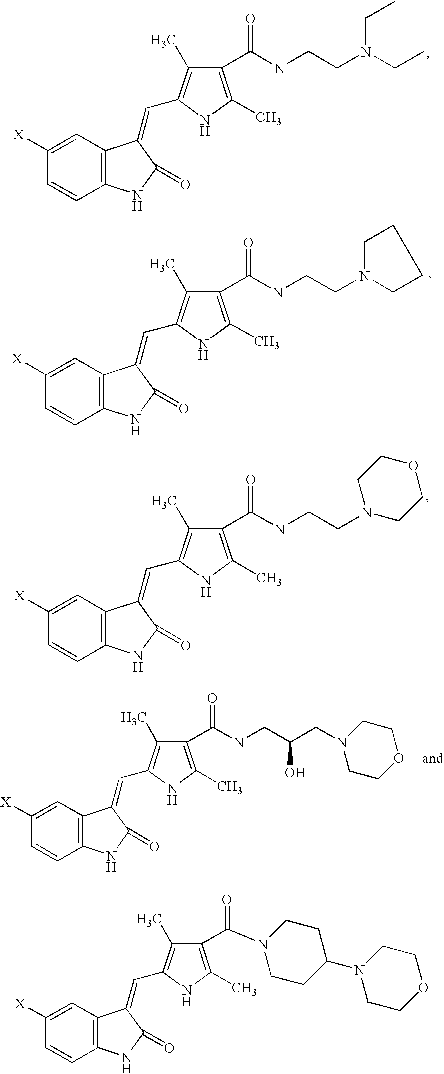

wherein X is F, Cl, I or Br; or a pharmaceutically acceptable salt thereof. In another embodiment, X is F.

In another embodiment of the invention, a mammal is exposed to a compound of Formula I selected from the group consisting of:

5-(5-Fluoro-2-oxo-1,2-dihydro-indol-3-ylidenemethyl)-2,4-dimethyl-1H-pyrrole-3-carboxylic acid (2-diethylamino-ethyl)-amide (Compound 1);

5-(5-Fluoro-2-oxo-1,2-dihydro-indol-3-ylidenemethyl)-2,4-dimethyl-1H-pyrrole-3-carboxylic acid (2-pyrrolidin-1-yl-ethyl)-amide (Compound 2);

5-(5-Fluoro-2-oxo-1,2-dihydro-indol-3-ylidenemethyl)-2,4-dimethyl-1H-pyrrole-3-carboxylic acid (2-morpholin-4-yl-ethyl)-amide (Compound 3);

(S)-5-(5-Fluoro-2-oxo-1,2-dihydro-indol-3-ylidenemethyl)-2,4-dimethyl-1H-pyrrole-3-carboxylic acid (2-hydroxy-3-morpholin-4-yl-propyl)-amide (Compound 4);

(R)-5-(5-Fluoro-2-oxo-1,2-dihydro-indol-3-ylidenemethyl)-2,4-dimethyl-1H-pyrrole-3-carboxylic acid (2-hydroxy-3-morpholin-4-yl-propyl)-amide (Compound 5);

5-(5-Fluoro-2-oxo-1,2-dihydro-indol-3-ylidenemethyl)-2,4-dimethyl-1H-pyrrole-3-carboxylic acid (2-hydroxy-3-morpholin-4-yl-propyl)-amide (Compound 6);

5-(5-Chloro-2-oxo-1,2-dihydro-indol-3-ylidenemethyl)-2,4-dimethyl-1H-pyrrole-3-carboxylic acid (2-hydroxy-3-morpholin-4-yl-propyl)-amide (Compound 7);

5-(5-Fluoro-2-oxo-1,2-dihydro-indol-3-ylidenemethyl)-2,4-dimethyl-1H-pyrrole-3-carboxylic acid (2-ethylamino-ethyl)-amide (Compound 8);

3-[3,5-dimethyl-4-(4-morpholin-4-yl-piperidine-1-carbonyl)-1H-pyrrol-2-methylene]-5-fluoro-1,3-dihydro-indol-2-one (Compound 9).

The above compounds are shown below:

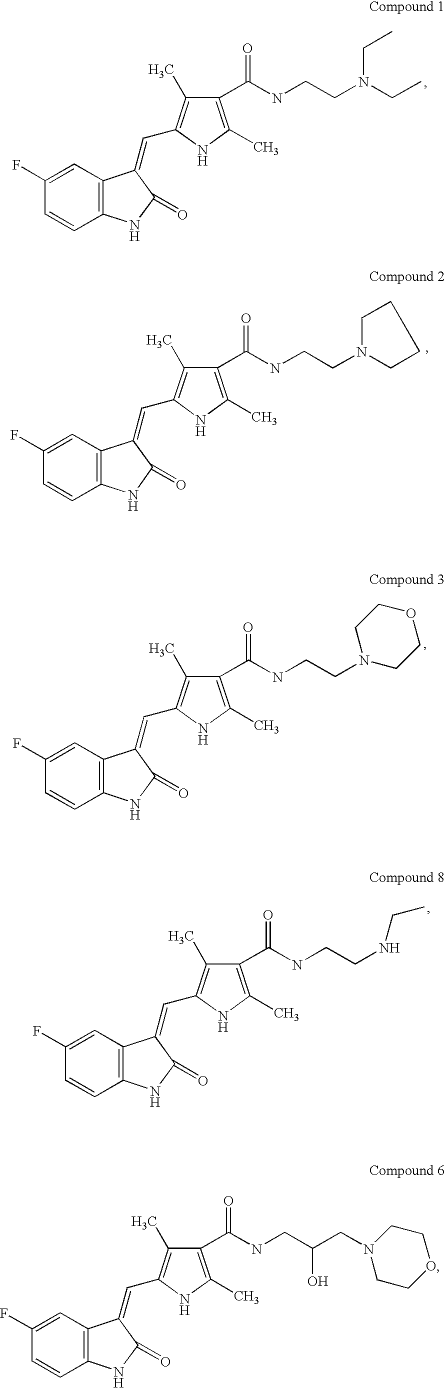

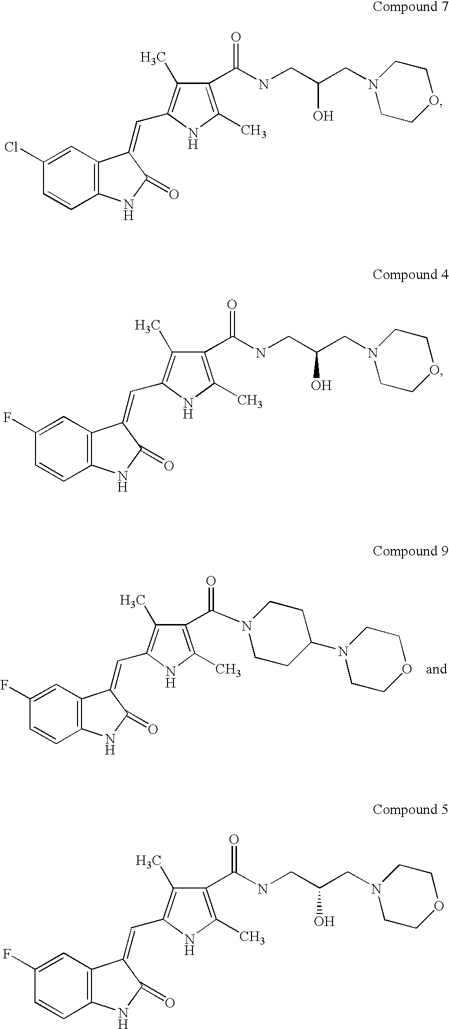

To clearly set forth the compounds of Formula I, Formula II and other compounds of the formulas described herein, useful in the inventive method, the following definitions are provided.

“Alkyl” refers to a saturated aliphatic hydrocarbon radical including straight chain and branched chain groups of 1 to 20 carbon atoms (whenever a numerical range; e.g. “1-20”, is stated herein, it means that the group, in this case the alkyl group, may contain 1 carbon atom, 2 carbon atoms, 3 carbon atoms, etc. up to and including 20 carbon atoms). Alkyl groups containing from 1 to 4 carbon atoms are referred to as lower alkyl groups. When said lower alkyl groups lack substituents, they are referred to as unsubstituted lower alkyl groups. More preferably, an alkyl group is a medium size alkyl having 1 to 10 carbon atoms e.g., methyl, ethyl, propyl, 2-propyl, n-butyl, iso-butyl, tert-butyl, pentyl, and the like. Most preferably, it is a lower alkyl having 1 to 4 carbon atoms e.g., methyl, ethyl, propyl, 2-propyl, n-butyl, iso-butyl, or tert-butyl, and the like. The alkyl group may be substituted or unsubstituted. When substituted, the substituent group(s) is preferably one or more, more preferably one to three, even more preferably one or two substituent(s) independently selected from the group consisting of halo, hydroxy, unsubstituted lower alkoxy, aryl optionally substituted with one or more groups, preferably one, two or three groups which are independently of each other halo, hydroxy, unsubstituted lower alkyl or unsubstituted lower alkoxy groups, aryloxy optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 5- or 6-member heterocyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo , hydroxy, unsubstituted lower alkyl or unsubstituted lower alkoxy groups, mercapto, (unsubstituted lower alkyl)thio, arylthio optionally substituted with one or more groups, preferably one, two or three groups which are independently of each other halo, hydroxy, unsubstituted lower alkyl or alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄, wherein R₁₃ and R₁₄ are independently selected from the group consisting of hydrogen, unsubstituted lower alkyl, trihalomethyl, cycloalkyl, heterocyclic and aryl optionally substituted with one or more, groups, preferably one, two or three groups which are independently of each other halo, hydroxy, unsubstituted lower alkyl or unsubstituted lower alkoxy groups.

Preferably, the alkyl group is substituted with one or two substituents independently selected from the group consisting of hydroxy, 5- or 6-member heterocyclic group having from 1 to 3 heteroatoms selected from the group consisting of nitrogen, oxygen and sulfur, the carbon and nitrogen (if present) atoms in the group being optionally substituted with one or more groups, preferably one, two or three groups which are independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and the nitrogen atoms in the group being optionally substituted with one or more groups, preferably one, two or three groups which are independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more groups, preferably one, two or three groups which are independently of each other halo, hydroxy, unsubstituted lower alkyl or unsubstituted lower alkoxy groups, or —NR₁₃R₁₄, wherein R₁₃ and R₁₄ are independently selected from the group consisting of hydrogen and alkyl. Even more preferably the alkyl group is substituted with one or two substituents which are independently of each other hydroxy, dimethylamino, ethylamino, diethylamino, dipropylamino, pyrrolidino, piperidino, morpholino, piperazino, 4-lower alkylpiperazino, phenyl, imidazolyl, pyridinyl, pyridazinyl, pyrimidinyl, oxazolyl, triazinyl, and the like.

“Cycloalkyl” refers to a 3 to 8 member all-carbon monocyclic ring, an all-carbon 5-member/6-member or 6-member/6-member fused bicyclic ring or a multicyclic fused ring (a “fused” ring system means that each ring in the system shares an adjacent pair of carbon atoms with each other ring in the system) group wherein one or more of the rings may contain one or more double bonds but none of the rings has a completely conjugated pi-electron system.

Examples, without limitation, of cycloalkyl groups are cyclopropane, cyclobutane, cyclopentane, cyclopentene, cyclohexane, cyclohexadiene, adamantane, cycloheptane, cycloheptatriene, and the like. A cycloalkyl group may be substituted or unsubstituted. When substituted, the substituent group(s) is preferably one or more, more preferably one or two substituents, independently selected from the group consisting of unsubstituted lower alkyl, trihaloalkyl, halo, hydroxy, unsubstituted lower alkoxy, aryl optionally substituted with one or more, preferably one or two groups independently of each other halo, hydroxy, unsubstituted lower alkyl or unsubstituted lower alkoxy groups, aryloxy optionally substituted with one or more, preferably one or two groups independently of each other halo, hydroxy, unsubstituted lower alkyl or unsubstituted lower alkoxy groups, 6-member heteroaryl having from 1 to 3 nitrogen atoms in the ring, the carbons in the ring being optionally substituted with one or more, preferably one or two groups independently of each other halo, hydroxy, unsubstituted lower alkyl or unsubstituted lower alkoxy groups, 5-member heteroaryl having from 1 to 3 heteroatoms selected from the group consisting of nitrogen, oxygen and sulfur, the carbon and nitrogen atoms of the group being optionally substituted with one or more, preferably one or two groups independently of each other halo, hydroxy, unsubstituted lower alkyl or unsubstituted lower alkoxy groups, 5- or 6-member heterocyclic group having from 1 to 3 heteroatoms selected from the group consisting of nitrogen, oxygen and sulfur, the carbon and nitogen (if present)atoms in the group being optionally substituted with one or more, preferably one or two groups independently of each other halo, hydroxy, unsubstituted lower alkyl or unsubstituted lower alkoxy groups, mercapto, (unsubstituted lower alkyl)thio, arylthio optionally substituted with one or more, preferably one or two groups independently of each other halo, hydroxy, unsubstituted lower alkyl or unsubstituted lower alkoxy groups, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄ are as defined above.

“Alkenyl” refers to a lower alkyl group, as defined herein, consisting of at least two carbon atoms and at least one carbon-carbon double bond. Representative examples include, but are not limited to, ethenyl, 1-propenyl, 2-propenyl, 1-, 2-, or 3-butenyl, and the like.

“Alkynyl” refers to a lower alkyl group, as defined herein, consisting of at least two carbon atoms and at least one carbon-carbon triple bond. Representative examples include, but are not limited to, ethynyl, 1-propynyl, 2-propynyl, 1-, 2-, or 3-butynyl, and the like.

“Aryl” refers to an all-carbon monocyclic or fused-ring polycyclic (i.e., rings which share adjacent pairs of carbon atoms) groups of 1 to 12 carbon atoms having a completely conjugated pi-electron system. Examples, without limitation, of aryl groups are phenyl, naphthalenyl and anthracenyl. The aryl group may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR¹³R₁₄, with R₁₃ and R₁₄ as defined above. Preferably, the aryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heteroaryl” refers to a monocyclic or fused ring (i.e., rings which share an adjacent pair of atoms) group of 5 to 12 ring atoms containing one, two, or three ring heteroatoms selected from N, O, or S, the remaining ring atoms being C, and, in addition, having a completely conjugated pi-electron system. Examples, without limitation, of unsubstituted heteroaryl groups are pyrrole, furan, thiophene, imidazole, oxazole, thiazole, pyrazole, pyridine, pyrimidine, quinoline, isoquinoline, purine and carbazole. The heteroaryl group may be substituted or unsubstituted. When substituted, the substituted group(s) is preferably one or more, more preferably one, two, or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄, with R₁₃ and R₁₄ as defined above. Preferably, the heteroaryl group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Heterocyclic” refers to a monocyclic or fused ring group having in the ring(s) of 5 to 9 ring atoms in which one or two ring atoms are heteroatoms selected from N, O, or S(O)n (where n is an integer from 0 to 2), the remaining ring atoms being C. The rings may also have one or more double bonds. However, the rings do not have a completely conjugated pi-electron system. Examples, without limitation, of unsubstituted heterocyclic groups are pyrrolidino, piperidino, piperazino, morpholino, thiomorpholino, homopiperazino, and the like. The heterocyclic ring may be substituted or unsubstituted. When substituted, the substituted group(s) is preferably one or more, more preferably one, two or three, even more preferably one or two, independently selected from the group consisting of unsubstituted lower alkyl, trihaloalkyl, halo, hydroxy, unsubstituted lower alkoxy, mercapto,(unsubstituted lower alkyl)thio, cyano, acyl, thioacyl, O-carbamyl, N-carbamyl, O-thiocarbamyl, N-thiocarbamyl, C-amido, N-amido, nitro, N-sulfonamido, S-sulfonamido, RS(O)—, RS(O)₂—, —C(O)OR, RC(O)O—, and —NR₁₃R₁₄, with R₁₃ and R₁₄ as defined above. Preferably, the heterocyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

Preferably, the heterocyclic group is optionally substituted with one or two substituents independently selected from halo, unsubstituted lower alkyl, trihaloalkyl, hydroxy, mercapto, cyano, N-amido, mono or dialkylamino, carboxy, or N-sulfonamido.

“Hydroxy” refers to an —OH group.

“Alkoxy” refers to both an —O-(unsubstituted alkyl) and an —O-(unsubstituted cycloalkyl) group. Representative examples include, but are not limited to, e.g., methoxy, ethoxy, propoxy, butoxy, cyclopropyloxy, cyclobutyloxy, cyclopentyloxy, cyclohexyloxy, and the like.

“Aryloxy” refers to both an —O-aryl and an —O-heteroaryl group, as defined herein. Representative examples include, but are not limited to, phenoxy, pyridinyloxy, furanyloxy, thienyloxy, pyrimidinyloxy, pyrazinyloxy, and the like, and derivatives thereof.

“Mercapto” refers to an —SH group.

“Alkylthio” refers to both an —S-(unsubstituted alkyl) and an —S-(unsubstituted cycloalkyl) group. Representative examples include, but are not limited to, e.g., methylthio, ethylthio, propylthio, butylthio, cyclopropylthio, cyclobutylthio, cyclopentylthio, cyclohexylthio, and the like.

“Arylthio” refers to both an —S-aryl and an —S-heteroaryl group, as defined herein. Representative examples include, but are not limited to, phenylthio, pyridinylthio, furanylthio, thientylthio, pyrimidinylthio, and the like and derivatives thereof.

“Acyl” refers to a —C(O)—R″ group, where R″ is selected from the group consisting of hydrogen, unsubstituted lower alkyl, trihalomethyl, unsubstituted cycloalkyl, aryl optionally substituted with one or more, preferably one, two, or three substituents selected from the group consisting of unsubstituted lower alkyl, trihalomethyl, unsubstituted lower alkoxy, halo and —NR₁₃R₁₄ groups, heteroaryl (bonded through a ring carbon) optionally substituted with one or more, preferably one, two, or three substituents selected from the group consisting of unsubstituted lower alkyl, trihaloalkyl, unsubstituted lower alkoxy, halo and —NR₁₃R₁₄ groups and heterocyclic (bonded through a ring carbon) optionally substituted with one or more, preferably one, two, or three substituents selected from the group consisting of unsubstituted lower alkyl, trihaloalkyl, unsubstituted lower alkoxy, halo and —NR¹³R₁₄ groups. Representative acyl groups include, but are not limited to, acetyl, trifluoroacetyl, benzoyl, and the like.

“Aldehyde” refers to an acyl group in which R″ is hydrogen.

“Thioacyl” refers to a —C(S)—R″ group, with R″ as defined herein.

“Ester” refers to a —C(O)O—R″ group with R″ as defined herein except that R″ cannot be hydrogen.

“Acetyl” group refers to a —C(O)CH₃ group.

“Halo” group refers to fluorine, chlorine, bromine or iodine, preferably fluorine or chlorine.

“Trihalomethyl” group refers to a —CX₃ group wherein X is a halo group as defined herein.

“Methylenedioxy” refers to a —OCH₂O— group where the two oxygen atoms are bonded to adjacent carbon atoms.

“Ethylenedioxy” group refers to a —OCH₂CH₂O— where the two oxygen atoms are bonded to adjacent carbon atoms.

“S-sulfonamido” refers to a —S(O)₂NR₁₃R₁₄ group, with R₁₃ and R₁₄ as defined herein.

“N-sulfonamido” refers to a —NR₁₃S(O)₂R group, with R₁₃ and R as defined herein.

“O-carbamyl” group refers to a —OC(O)NR₁₃R₁₄ group with R₁₃ and R₁₄ as defined herein.

“N-carbamyl” refers to an ROC(O)NR₁₄— group, with R and R₁₄ as defined herein.

“O-thiocarbamyl” refers to a —OC(S)NR₁₃R₁₄ group with R₁₃ and R₁₄ as defined herein.

“N-thiocarbamyl” refers to a ROC(S)NR₁₄— group, with R and R₁₄ as defined herein.

“Amino” refers to an —NR₁₃R₁₄ group, wherein R₁₃ and R₁₄ are both hydrogen.

“C-amido” refers to a —C(O)NR₁₃R₁₄ group with R₁₃ and R₁₄ as defined herein.

“N-amido” refers to a RC(O)NR₁₄— group, with R and R₁₄ as defined herein.

“Nitro” refers to a —NO₂ group.

“Haloalkyl” means an unsubstituted alkyl, preferably unsubstituted lower alkyl as defined above that is substituted with one or more same or different halo atoms, e.g., —CH₂Cl, —CF₃, —CH₂CF₃, —CH₂CCl₃, and the like.

“Aralkyl” means unsubstituted alkyl, preferably unsubstituted lower alkyl as defined above which is substituted with an aryl group as defined above, e.g., —CH₂phenyl, —(CH₂)₂phenyl, —(CH₂)₃phenyl, CH₃CH(CH₃)CH₂phenyl, and the like and derivatives thereof.

“Heteroaralkyl” group means unsubstituted alkyl, preferably unsubstituted lower alkyl as defined above which is substituted with a heteroaryl group, e.g., —CH₂pyridinyl, —(CH₂)₂pyrimidinyl, —(CH₂)₃imidazolyl, and the like, and derivatives thereof.

“Monoalkylamino” means a radical —NHR′ where R′ is an unsubstituted alkyl or unsubstituted cycloalkyl group as defined above, e.g., methylamino, (1-methylethyl)amino, cyclohexylamino, and the like.

“Dialkylamino” means a radical —NR′R′ where each R′ is independently an unsubstituted alkyl or unsubstituted cycloalkyl group as defined above, e.g., dimethylamino, diethylamino, (1-methylethyl)-ethylamino, cyclohexylmethylamino, cyclopentylmethylamino, and the like.

“Cyanoalkyl” means unsubstituted alkyl, preferably unsubstituted lower alkyl as defined above, which is substituted with 1 or 2 cyano groups.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocyclo group is not substituted with the alkyl group.

A “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

As used herein, a “physiologically/pharmaceutically acceptable carrier” refers to a carrier or diluent that does not cause significant irritation to an organism and does not abrogate the biological activity and properties of the administered compound.

An “pharmaceutically acceptable excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

As used herein, the term “salt” of a compound of Formula I, II or other formulas or compounds described in this specification refers to those salts which retain the biological effectiveness and properties of the parent compound. Such salts include:

(i) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic acid such as the L-malate salt of 5-(5-fluoro-2-oxo-1,2-dihydroindol-3-ylidenemethyl)-2,4-dimethyl-1H-pyrrole-3-carboxylic acid(2-diethylaminoethyl)amide; or

(ii) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Method” refers to manners, means, techniques and procedures for accomplishing a given task including, but not limited to, those manners, means, techniques and procedures either known to, or readily developed from known manners, means, techniques and procedures by, practitioners of the chemical, pharmaceutical, biological, biochemical and medical arts.

“In vivo” refers to procedures performed within a living organism such as, without limitation, a mouse, rat or rabbit.

“Treat”, “treating” and “treatment” refer to a method of alleviating, ameliorating, abrogating or relieving a disease condition and/or any of its attendant symptoms.

“Patient” refers to any living entity comprised of at least one cell. A living organism can be as simple as, for example, a single eukariotic cell or as complex as a mammal, including a human being.

“Therapeutically effective amount” refers to that amount of the compound being administered which will prevent, alleviate, ameliorate or relieve to some extent, one or more of the signs or symptoms of the disorder being treated.

Administration and Pharmaceutical Composition

In another embodiment of the invention, a human patient is exposed or administered a compound of Formula I, Formula II or other formulas or compounds described in this application, or a pharmaceutically acceptable salt thereof. Alternatively, the compounds of Formula I, Formula II or other formulas or compounds described herein can be administered in pharmaceutical compositions in which the foregoing materials are mixed with suitable carriers or excipient(s). Techniques for formulation and administration of drugs may be found in “Remington's Pharmacological Sciences,” Mack Publishing Co., Easton, Pa., latest edition.

As used herein, “exposing,” “administer” or “administration” refers to the delivery of a compound of Formula I, Formula II or other formulas or compounds described herein or a pharmaceutically acceptable salt thereof or of a pharmaceutical composition containing a compound of Formula I, Formula II or other formulas or compounds described herein or a pharmaceutically acceptable salt thereof of this invention to a mammal.

Suitable routes of administration may include, without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Furthermore, one administer the compound in a targeted drug delivery system, for example, in a liposome coated with tumor-specific antibody. The liposomes will be targeted to and taken up selectively by the tumor progenitor.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinyl pyrrolidone, agar, or alginic acid. A salt such as sodium alginate may also be used.

Dragee cores are provided with suitable coatings. For this purpose, concentrated sugar solutions may be used which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, or liquid polyethylene glycols. Stabilizers may be added in these formulations, also.

Pharmaceutical compositions which may also be used include hard gelatin capsules. As a non-limiting example, compound 1 in a capsule oral drug product formulation may be as 50 and 200 mg dose strengths. The two dose strengths are made from the same granules by filling into different size hard gelatin capsules, size 3 for the 50 mg capsule and size 0 for the 200 mg capsule.

The capsules may be packaged into brown glass or plastic bottles to protect the active compound from light. The containers containing the active compound capsule formulation must be stored at controlled room temperature (15-30° C.).

For administration by inhalation, the compounds for use according to the present invention are conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

A non-limiting example of a pharmaceutical carrier for the hydrophobic compounds of the invention is a cosolvent system comprising benzyl alcohol, a nonpolar surfactant, a water-miscible organic polymer and an aqueous phase such as the VPD co-solvent system. VPD is a solution of 3% w/v benzyl alcohol, 8% w/v of the nonpolar surfactant Polysorbate 80, and 65% w/v polyethylene glycol 300, made up to volume in absolute ethanol. The VPD co-solvent system (VPD:D5W) consists of VPD diluted 1:1 with a 5% dextrose in water solution. This co-solvent system dissolves hydrophobic compounds well, and itself produces low toxicity upon systemic administration. Naturally, the proportions of such a co-solvent system may be varied considerably without destroying its solubility and toxicity characteristics. Furthermore, the identity of the co-solvent components may be varied: for example, other low-toxicity nonpolar surfactants may be used instead of Polysorbate 80, the fraction size of polyethylene glycol may be varied, other biocompatible polymers may replace polyethylene glycol, e.g., polyvinyl pyrrolidone, and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional stratergies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Examples of formulations for use in the present invention are in Tables A-C:

TABLE A
Composition of 5-(5-fluoro-2-oxo-1,2-dihydro-indol-3-ylidenemethyl)-
2,4-dimethyl-1H-pyrrole-3-carboxylic acid (2-diethylamino-ethyl)-amide
hard gelatin capsules

| | | Amount | Amount | Amount |
|---|---|---|---|---|
| | Concentration | in 50 mg | in 75 mg | in 200 mg |
| | in Granulation | Capsule | Capsule | Capsule |
| Ingredient Name | (% w/w) | (mg) | (mg) | (mg) |
| API | 65.0 | 50.0 | 75.0 | 200.0 |
| Mannitol | 23.5 | 18.1 | 27.2 | 72.4 |
| Croscaramellose | 6.0 | 4.6 | 6.9 | 18.4 |
| Sodium^{e} | | | | |
| Povidone (K-25) | 5.0 | 3.8 | 5.7 | 15.2 |
| Magnesium | 0.5 | 0.38 | 0.57 | 1.52 |
| Stearate | | | | |
| Capsule | — | Size 1 | Size 3 | Size 0 |

TABLE B
Composition of 5-(5-fluoro-2-oxo-1,2-dihydro-indol-3-
ylidenemethyl)-2,4-dimethyl-1H-pyrrole-3-carboxylic acid
(2-diethylamino-ethyl)-amide L-malate hard gelatin capsules

| | Concentration | |
|---|---|---|
| Ingredient | in Granulation | Amount in 50 mg |
| Name/Grade | (% w/w) | Capsule (mg) |
| API | 75.0 | 66.800^{c} |
| Mannitol | 13.5 | 12.024 |
| Croscaramellose | 6.0 | 5.344 |
| Sodium^{e} | | |
| Povidone (K-25) | 5.0 | 4.453 |
| Magnesium Stearate | 0.5 | 1.445 |
| Capsule | — | Size 3 |

TABLE C
Composition of 5-(5-fluoro-2-oxo-1,2-dihydro-indol-3-ylidenemethyl)-
2,4-dimethyl-1H-pyrrole-3-carboxylic acid (2-diethylamino-ethyl)-amide
L-malate hard gelatin capsules

| | | Amount | Amount | Amount |
|---|---|---|---|---|
| | Concentration | in 25 mg | in 50 mg | in 100 mg |
| Ingredient | in Granulation | Capsule | Capsule | Capsule |
| Name/Grade | (% w/w) | (mg) | (mg) | (mg) |
| API^{a} | 40.0 | 33.400^{d} | 66.800^{c} | 200.0^{b} |
| Mannitol | 47.5 | 39.663 | 79.326 | 158.652 |
| Croscaramellose | 6.0 | 5.010 | 10.020 | 20.04 |
| Sodium^{e} | | | | |
| Povidone (K-25) | 5.0 | 4.175 | 8.350 | 16.700 |
| Magnesium | 1.5 | 1.252 | 2.504 | 5.008 |
| Stearate | | | | |
| Capsule | — | Size 3 | Size 1 | Size 0 |

^{a}Drug substance quantity required for the batch will be adjusted to have 100% of labeled strength for capsules. Appropriate adjustment will be made to mannitol quantity to keep the same fill weight for each strength.
^{b}Quantity equivalent to 100 mg free base.
^{c}Quantity equivalent to 50 mg free base.
^{d}Quantity equivalent to 25 mg free base.
^{e}Half intragranular half extragranular.

which can be found in U.S. patent application Ser. No. 10/237,966, filed Sep. 10, 2002, now a provisional application, which is expressly incorporated in its entirety by reference.

Many of the compounds of Formula I, Formula II or other formulas or compounds described herein may be provided as physiologically acceptable salts wherein the compound may form the negatively or the positively charged species. Examples of salts in which the compound forms the positively charged moiety include, without limitation, quaternary ammonium, salts such as the hydrochloride, sulfate, carbonate, lactate, tartrate, malate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), Calcium hydroxide (Ca(OH)₂), etc.).

Pharmaceutical compositions suitable for use in the present invention include compositions wherein the active ingredients are contained in an amount sufficient to achieve the intended purpose, i.e., a therapeutically effective amount.

Determination of a therapeutically effective amount is well within the capability of those skilled in the art, especially in light of the detailed disclosure provided herein.

For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of phosphorylation of CSF1R). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀, wherein the LD₅₀ is the concentration of test compound which achieves a half-maximal inhibition of lethality, for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%.

At present, the therapeutically effective amounts of compounds of Formula I, Formula II or other formulas or compounds described in this application may range from approximately 25 mg/m² to 1500 mg/m² per day; alternatively about approximately 25 mg/m² to 1000 mg/m² per day. In another embodiment, the therapeutically effective amounts may range from approximately 25 mg/m² to 400 mg/m² per day.

In cases of local administration or selective uptake, the effective local concentration of the drug may not be related to plasma concentration and other procedures known in the art may be employed to determine the correct dosage amount and interval.

The amount of a composition administered will, of course, be dependent on the subject being treated, the severity of the affliction, the manner of administration, the judgement of the prescribing physician, etc.

It is contemplated that the inventive method could be used in combination with other therapies, including chemotherapies, radiation therapies and surgical therapies for cancer. For combination therapies and pharmaceutical compositions described herein, the effective amounts of the compound of the invention and of the other agent can be determined by those of ordinary skill in the art, based on the effective amounts for the compounds described herein and those known or described for the other agent. The formulations and route of administration for such therapies and composition can be based on the information described herein for compositions and therapies comprising the compound of the invention as the sole active agent and on information provided for the chemotherapeutic and other agent in combination therewith.

Although all biomarkers disclosed in this specification are identified by specific sequences (and corresponding SEQ ID NOs), those skilled in the art will recognize that variants and alleles of these sequences also may function as biomarkers. Specific sequences, GenBank accession numbers and SEQ ID NOs in the specification are used to identify exemplary cDNAs, mRNAs and/or proteins of interest, and do not limit the invention to only those particular sequences. The biomarkers of the invention encompass variants and alleles of the disclosed sequences.

### D. EXAMPLES

### Studies Using Compound A (SU6668)

1. Studies Using Compound A—Materials and Methods ELISAs

Reagents for human tissue inhibitor of metalloproteinase 1 (TIMP-1), human active and pro-matrix metalloproteinase 9 (total MMP-9) and human vascular endothelial growth factor (VEGF) ELISA kits were obtained from R&D Systems, Inc. (Minneapolis, Minn.). Human plasminogen activator inhibitor-1 (PAI-1) and human tissue factor (TF) ELISA kits were obtained from American Diagnostica, Inc. (Greenwich, Conn.). All ELISAs were performed on plasma samples according to the manufacturers' instructions.

2D Gel Analysis

Patient plasma was analyzed by 2D gel electrophoresis by Kendrick Labs (Madison, Wis.) according to the method of O'Farrell (J. Biol. Chem. 250: 4007-4021, 1975). Briefly, 150 ug of plasma protein was separated by isoelectric focusing using pH 4-8 gradient IEF gels. A 10% SDS/PAGE gel was used for the second gel dimension. Limited computerized comparisons were carried out on duplicate silver-stained gels and the spot percentage was calculated according to the formula: Difference=(1-spot % sample x/spot % sample ref)(−100). Spots whose abundance appeared to differ after Compound A exposure were subsequently excised and MALDI-TOF analysis was carried out for identification purposes.

Isolation of RNA from Whole Frozen Blood

TRI Reagent® BD—RNA, DNA, protein isolation reagent was used according to the manufacturer's protocol, Molecular Research Center, Inc. (Cincinnati, Ohio) <www.mrcgene.com>.

Transcriptional Profiling Using Affymetrix DNA Arrays

RNA processing and hybridization protocols were carried out as recommended by Affymetrix, Inc. (Santa Clara, Calif.); protocols are available in the Genechip® Expression Analysis Technical Manual <www.affymetrix.com/support/technical/manual/expression_manual.affx>. In brief, double-stranded cDNA was synthesized from total blood RNA (8 μg) of patient samples using Invitrogen Life Technologies SuperScript Choice system reagents (Carlsbad, Calif.). A T7-(dT)₂₄ oligomer was used to prime first-strand cDNA synthesis. Double-stranded cDNA product was generated and purified via phenol-chloroform extraction, then used as template for in vitro transcription (IVT) of cRNA. The IVT reaction was performed using BioArray HighYield RNA Transcript Labeling Kit (Affymetrix) according to manufacturer's protocol. The cRNA product was then purified with Qiagen RNeasy Mini Kit spin columns according to the manufacturer's protocol (Qiagen, Valencia, Calif.). Purified cRNA was quantitated, chemically fragmented, and hybridized overnight on Human Genome U95A Arrays. Hybridized arrays were washed and stained with phycoerythrin-conjugated streptavidin detection chemistry in an Affymetrix Fluidics station. Images were scanned with a Hewlett-Packard GeneArray scanner.

Data Analysis

Data files were generated from scanned array images in the Affymetrix Microarray Suite Version 4.0 program. The two key parameters used in determining transcriptional changes are the Average Difference (AD) values, which serve as relative indicators of the expression level of transcripts represented on the arrays, and the Absolute Call (AC), which determines the presence or absence of each transcript. To enable comparison of all hybridization data, global scaling was applied by multiplying the output of each experiment by a scaling factor (SF) to make its average intensity equal to a user-defined Target Intensity (1000 for these experiments). For comparisons between time points from a single patient, the data were analyzed using Microsoft Access 97 software (Microsoft, Redmond, Wash.). To determine the fold change, the AD of the post-treatment sample was divided by the AD of the pre-dose samples. A data filtering step was carried out to identify transcripts with AC of “present” that showed a fold change ≧1.7 (increasing or decreasing).

TaqMan (qRT-PCR)

Primers and probes were designed using Primer Express 2.0 software, and purchased from Applied Biosystems (Foster City, Calif.). In all cases, primers and probes were designed to hybridize to sequences represented by the Affymetrix probe set (see Affymetrix NetAffx website for detail). All probes contained a reporter dye (FAM) and a dye quencher (MGB). QRT-PCR was performed using 20 ng of total RNA with TaqMan® One-Step RT-PCR Master Mix Reagents Kit (Applied Biosystems) following the manufacturer's protocol. The reactions were performed in 96-well optical plates and analyzed using the ABI PRISM® 7700 Sequence Detection System (Applied Biosystems). Thermal cycler conditions used are as follows: 48° C. for 30 minutes, 95° C. for 10 minutes, 95° C. for 15 seconds followed by 60° C. for 1 minute for 40 cycles, and 25° C. for 2 minutes. VEGF (Genbank accession number AF022375) transcripts were amplified using forward primer GCTCTCTTATTTGTACCGGTTTTTG (SEQ ID NO: 165), reverse primer AAGCTAGTGACTGTCACCGATCAG (SEQ ID NO: 166), and probe TCATGTTTCCAATCTC (SEQ ID NO: 167) to generate an 82-bp amplicon product. Vinculin (Genbank accession number M33308) transcripts were amplified using forward primer CCTGATATAAATGCAATATTAATGCCTTTA (SEQ ID NO: 168), reverse primer AAGAACCGGGAGAGCAAACAT (SEQ ID NO: 169), and probe ATCTATGCCAAAGATCACTT (SEQ ID NO: 170) to generate a 124-bp amplicon product. PECAM-1 (Genbank accession number L34657) transcripts were amplified using forward primer GGAGCACCGCCTGTGAA (SEQ ID NO: 171), reverse primer TGTGCGTTGCCTGAATGAAC (SEQ ID NO: 172), and probe ACCAACCTGAAGACAC (SEQ ID NO: 173) to generate a 56-bp amplicon product. MAPK Kinase 3 (Genbank accession number L36719) transcripts were amplified using forward primer TCTCGACTGAATGGACTTTGCA (SEQ ID NO: 174), reverse primer TTGTGTACCCCGCACCAA (SEQ ID NO: 175), and probe CACACCTCTATCCCGGC (SEQ ID NO: 176) to generate a 77-bp amplicon product. Hemoglobin, epsilon 1 (Genbank accession number AI349593) transcripts were amplified using forward primer GCTGCATGTGGATCCTGAGA (SEQ ID NO: 177), reverse primer TGAGTAGCCAGAATAATCACCATCA (SEQ ID NO: 178), and probe CTTCAAGCTCCTGGGTAA (SEQ ID NO: 179) to generate a 66-bp amplicon product. GAPDH and 18S were ordered as pre-developed assay reagents (PDARs) from Applied Biosystems and used as endogenous controls.

Data analysis of TaqMan (qRT-PCR): The Ct scores represent the cycle number at which fluorescence signal (ΔRₙ) crosses an arbitrary (user-defined) threshold. The Ct scores for genes of interest for each sample were normalized against Ct scores for the corresponding endogenous control gene (GAPDH or 18S). Relative expression of specific transcripts in the post-dose sample compared to pre-dose sample was determined by the following calculation, as described in the Applied Biosytems users bulletin on Relative Quantitation of Gene Expression:

Rel Exp −2^{-ΔΔCt},

Where ΔΔCt=(Ct_{target}−Ct_{control})_{post-dose}−(Ct_{target}−Ct_{control})_{pre-dose}.

2. Studies Using Compound A—Results ELISAs

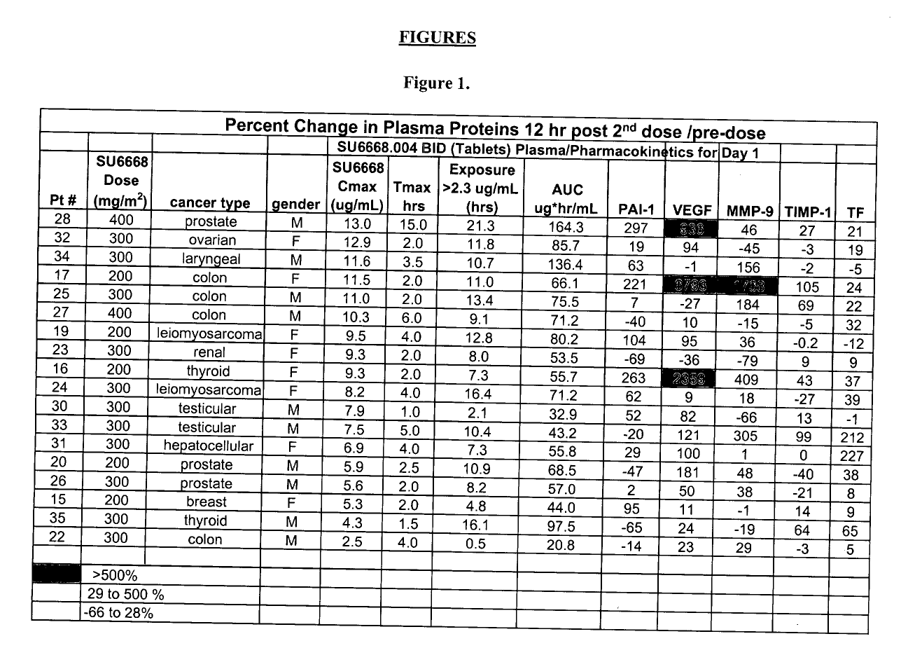

Samples of plasma from human patients were taken before and 24 hours after the first dose of Compound A (SU6668). The patients were dosed twice over 24 hours with Compound A. The results of the ELISA analysis are shown in FIG. 1, which shows that the levels of PAI-1, VEGF and TIMP-1 were increased in the plasma from patients exposed to Compound A. These proteins were therefore identified as biomarkers for a compound that inhibits tyrosine kinase, such as Compound A. These patients were suffering from various types of cancer.

Two Dimensional Polyacrylamide Gel Electrophoresis

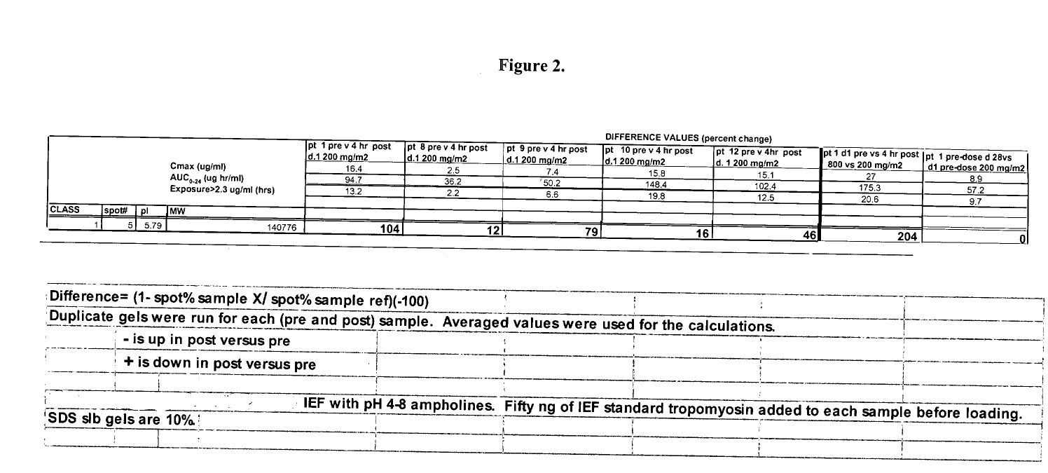

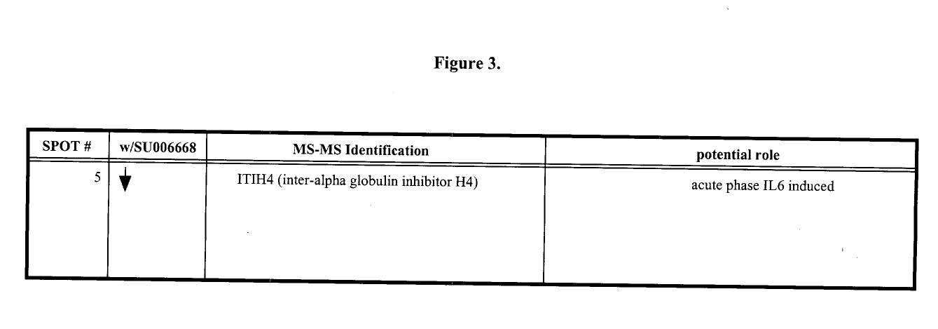

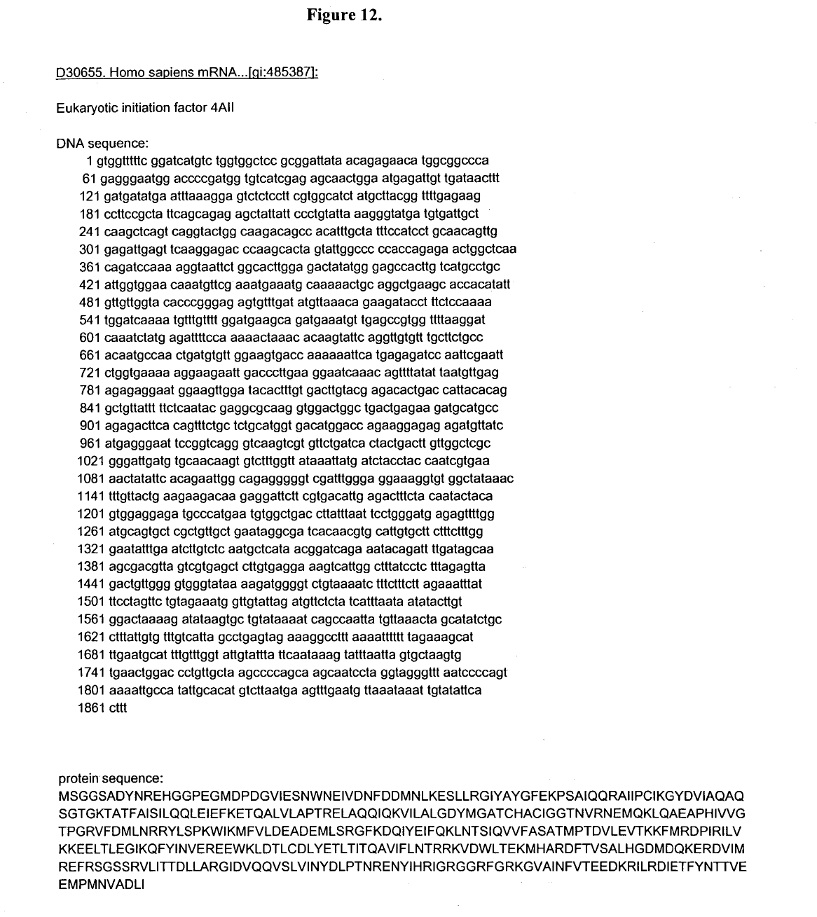

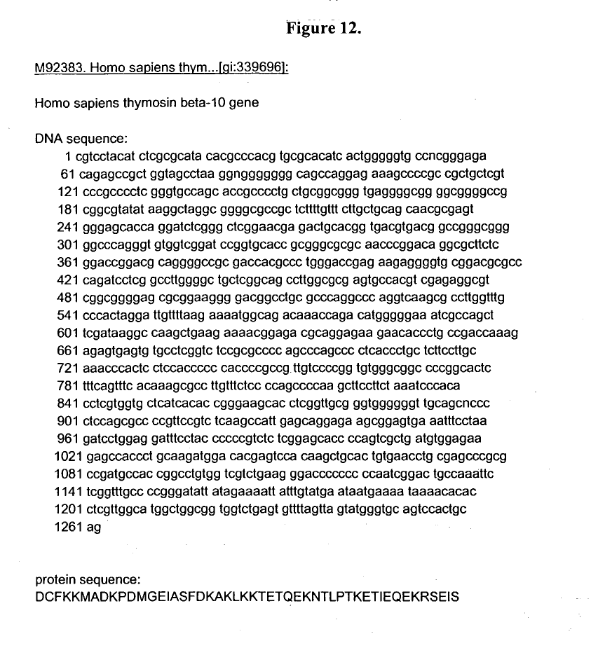

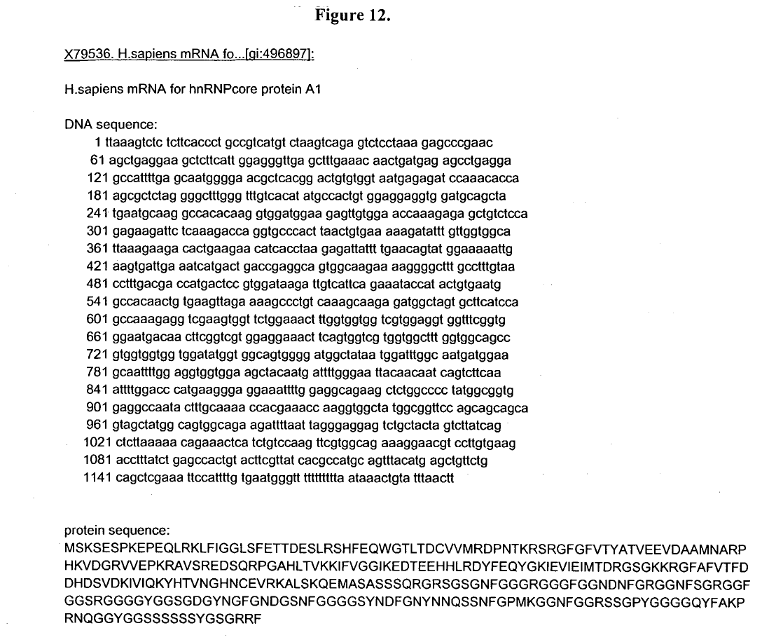

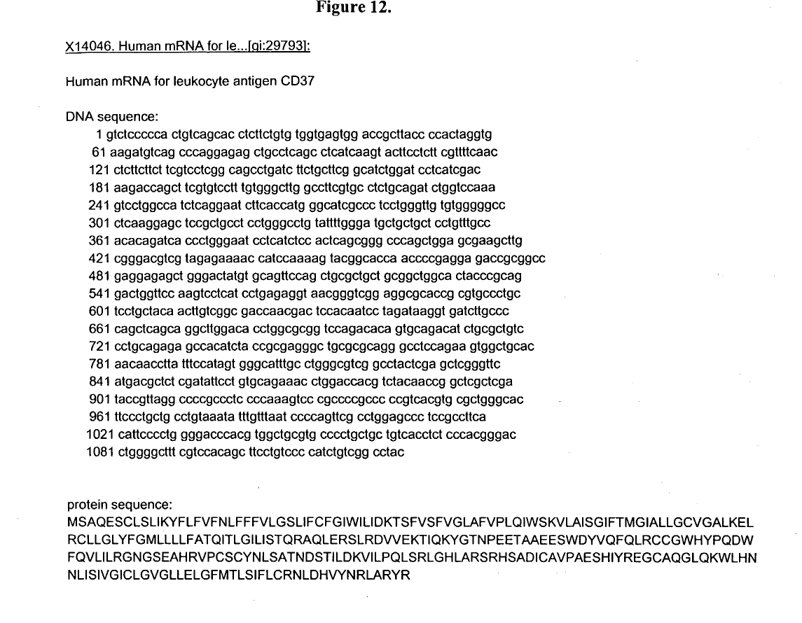

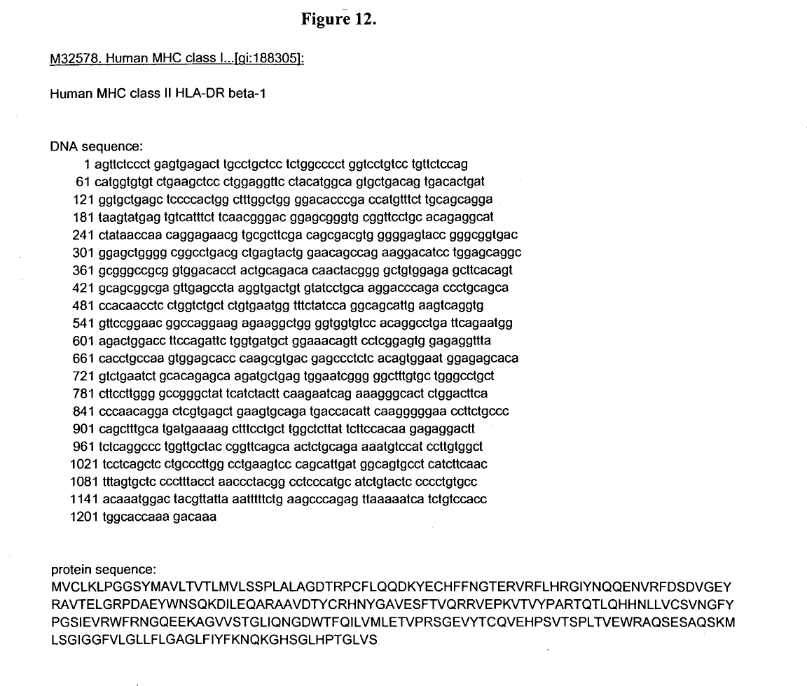

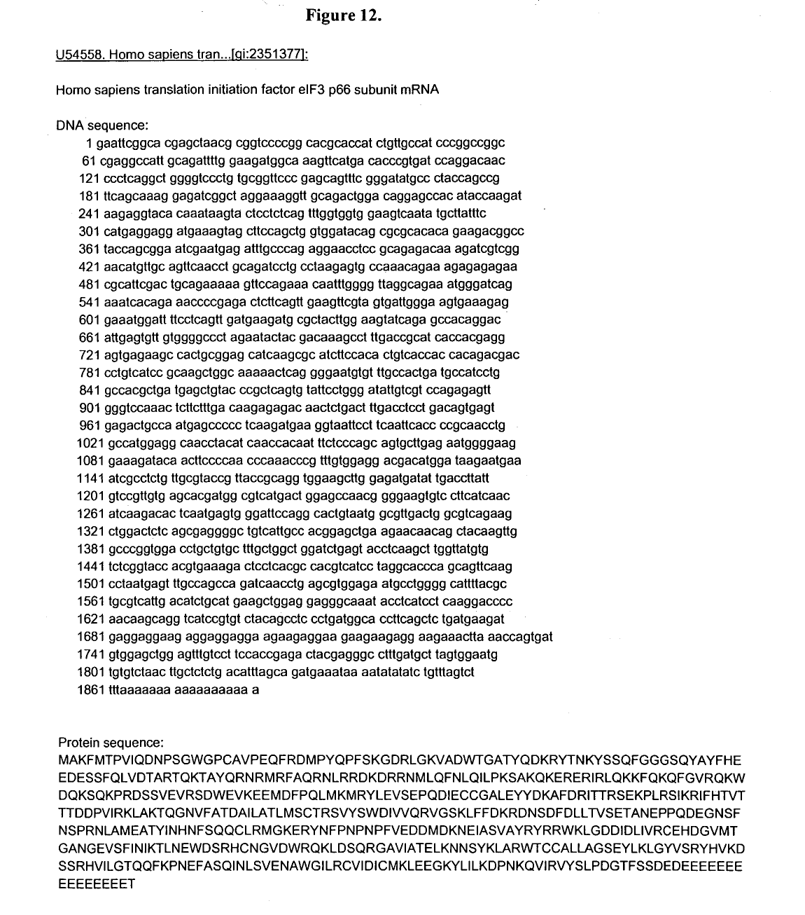

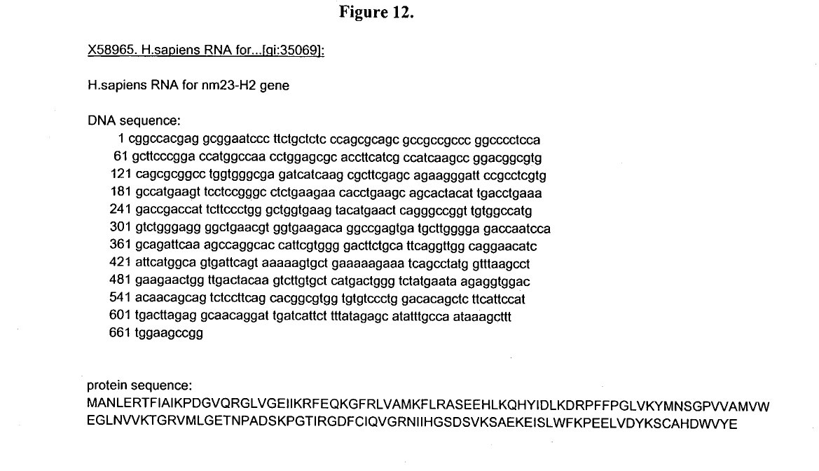

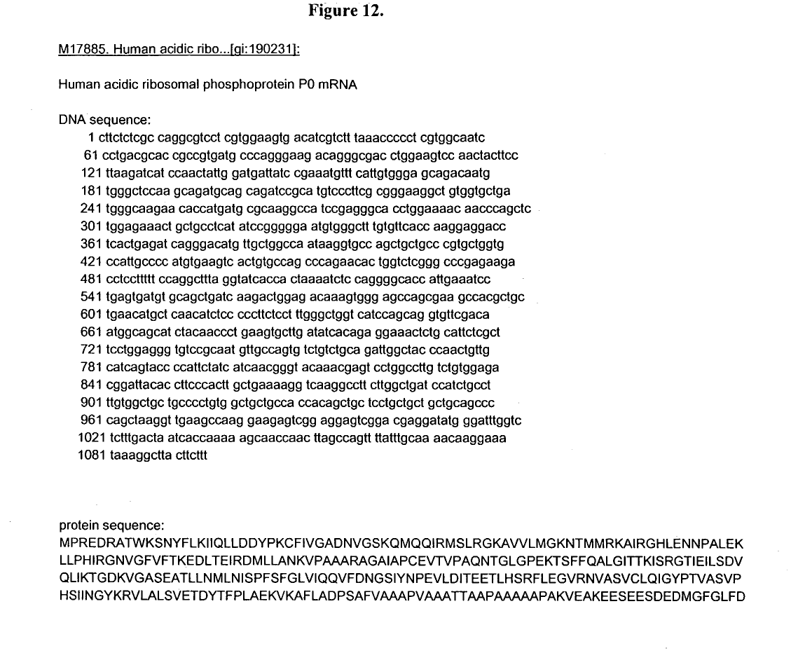

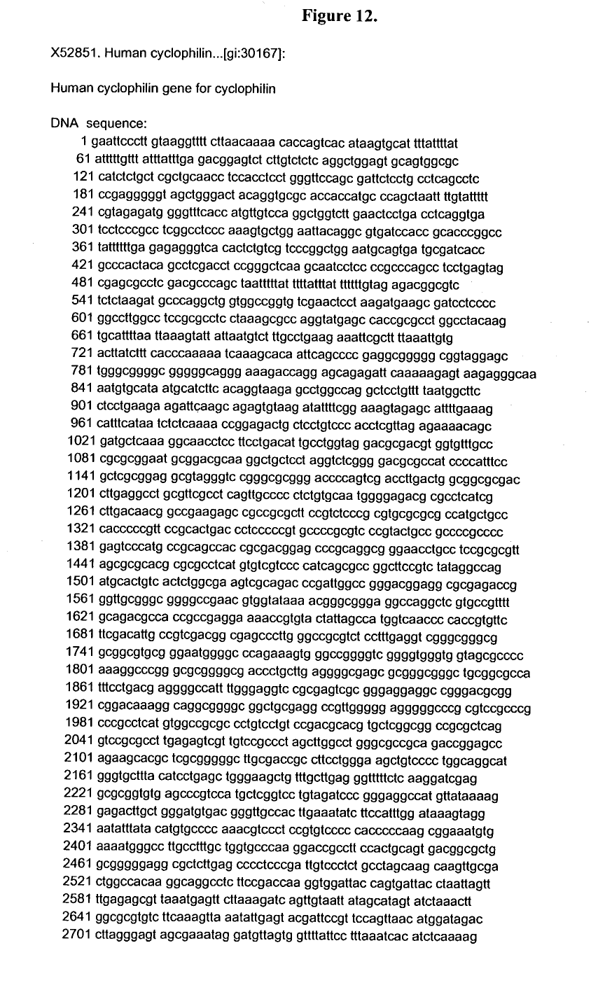

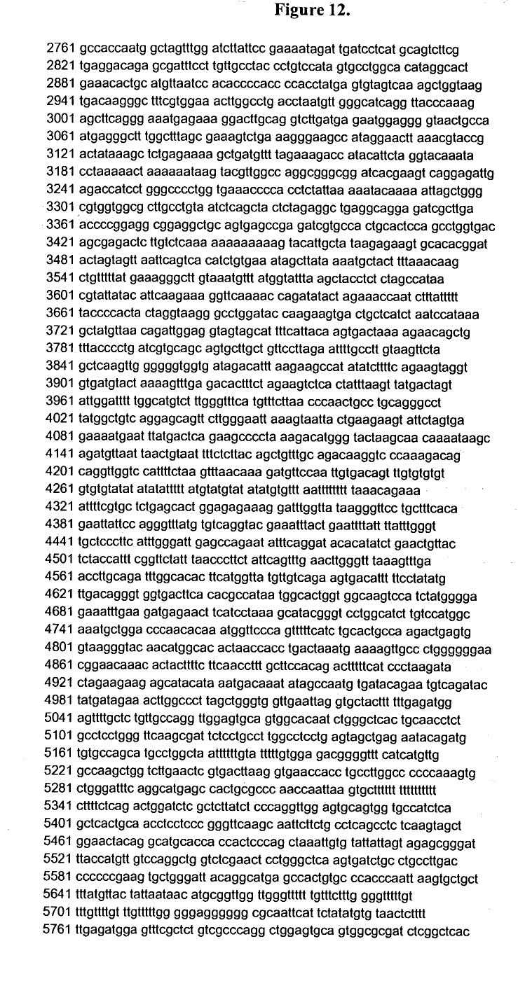

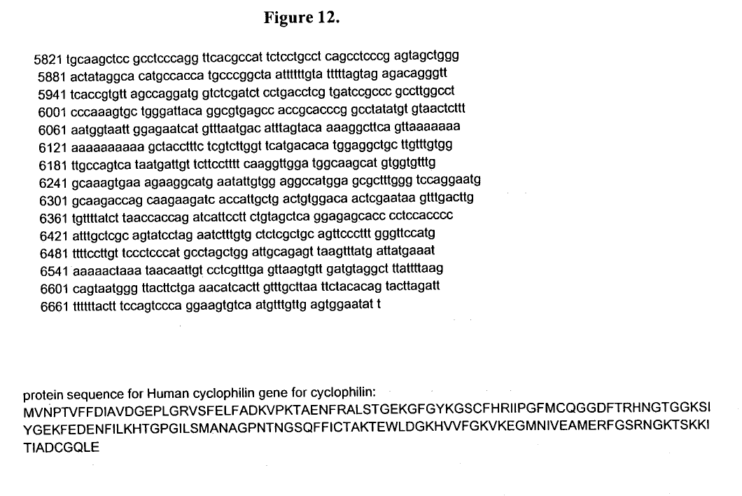

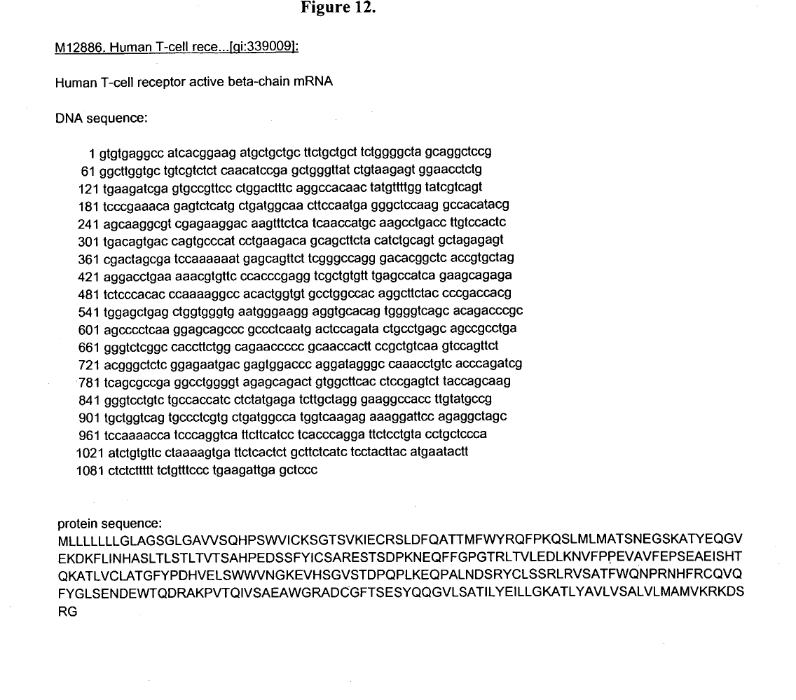

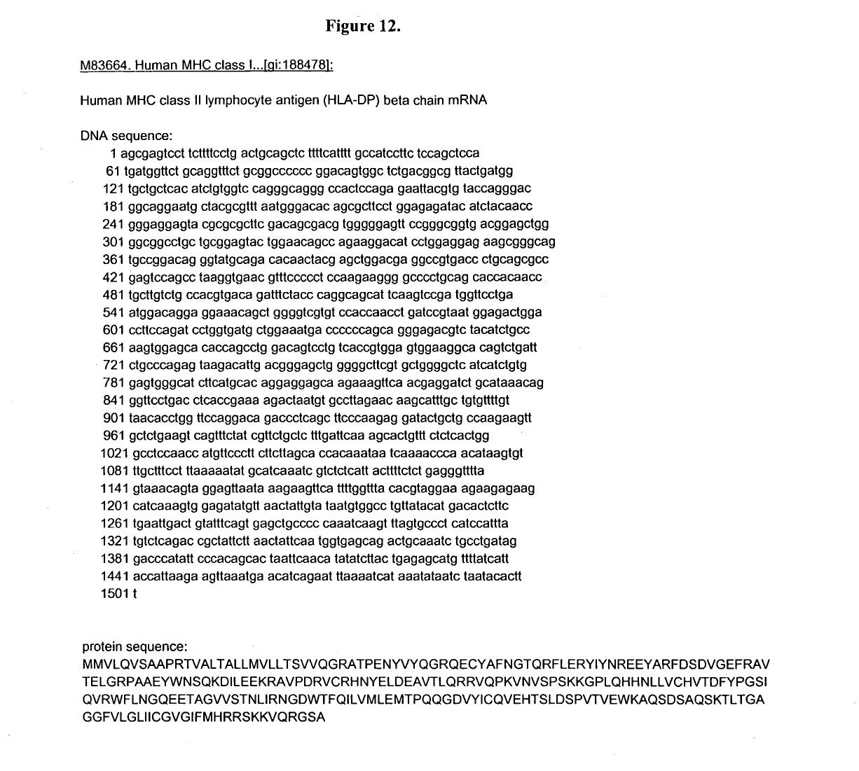

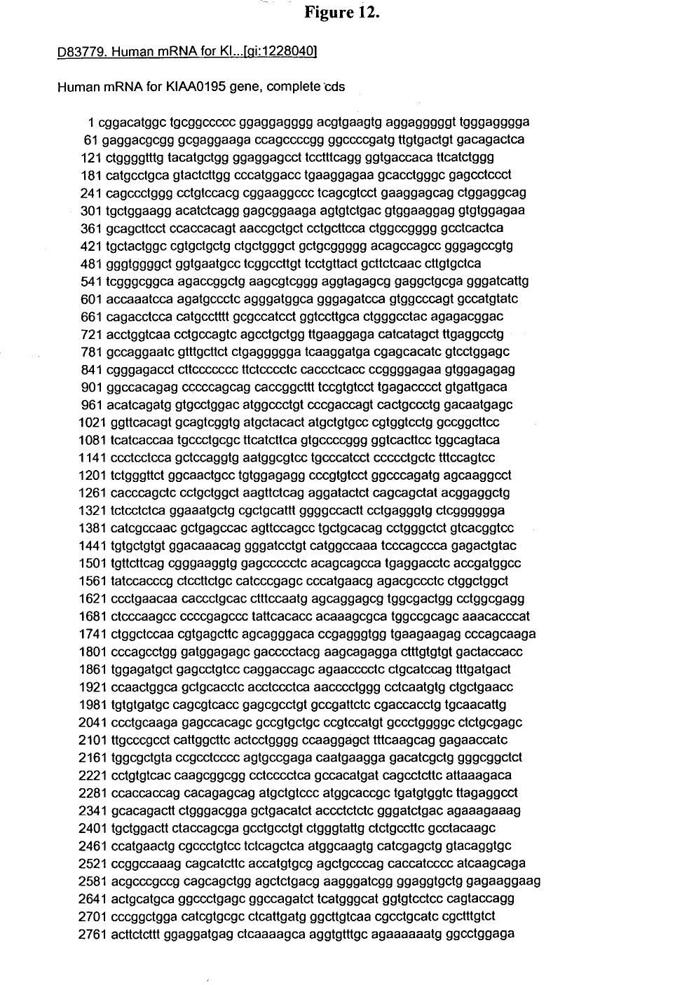

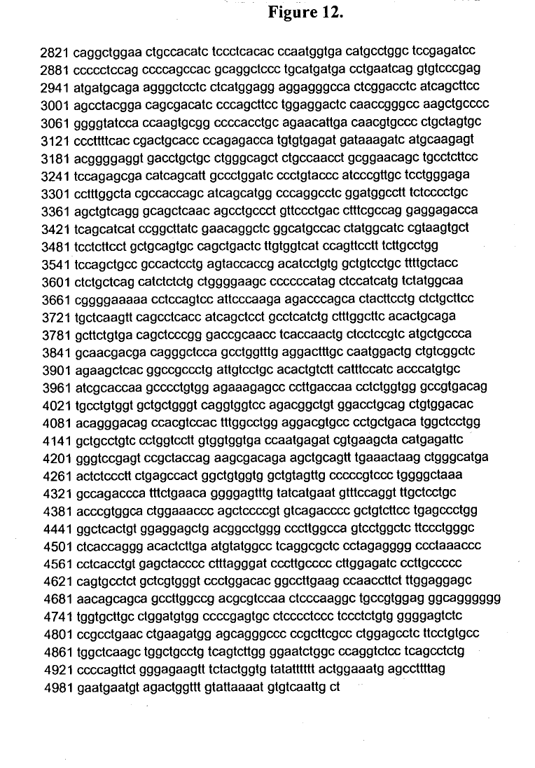

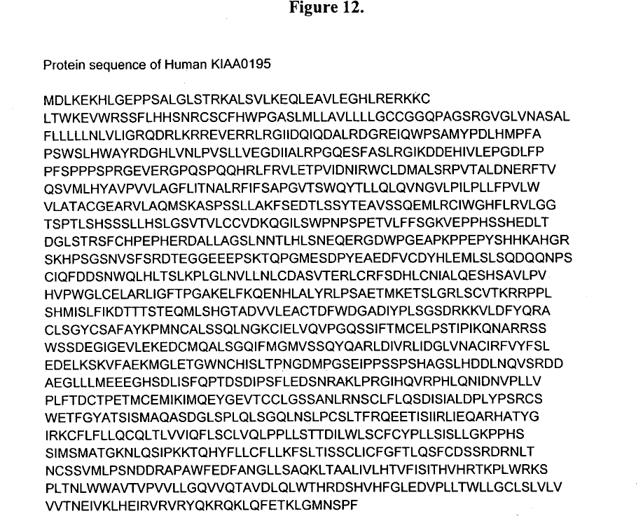

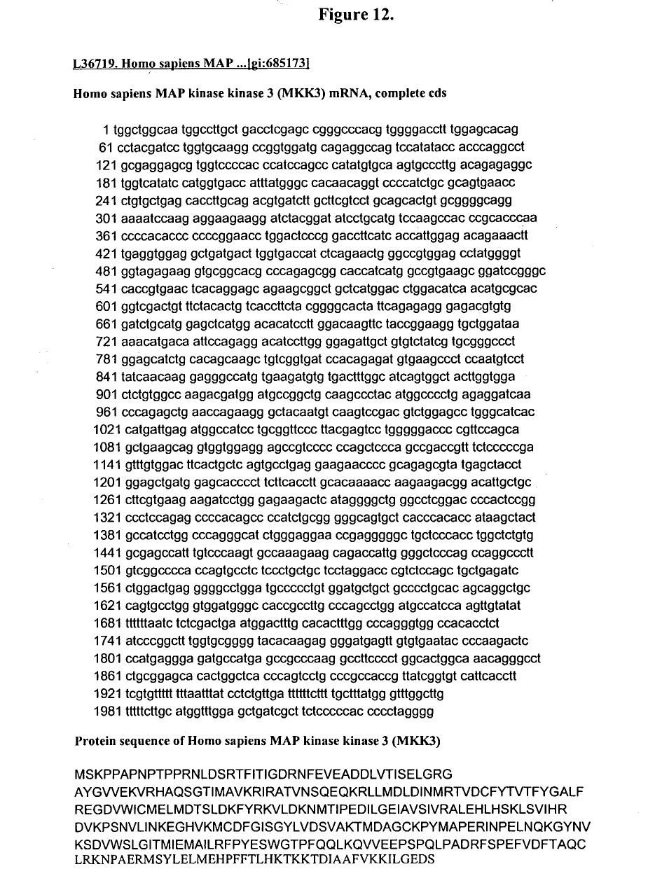

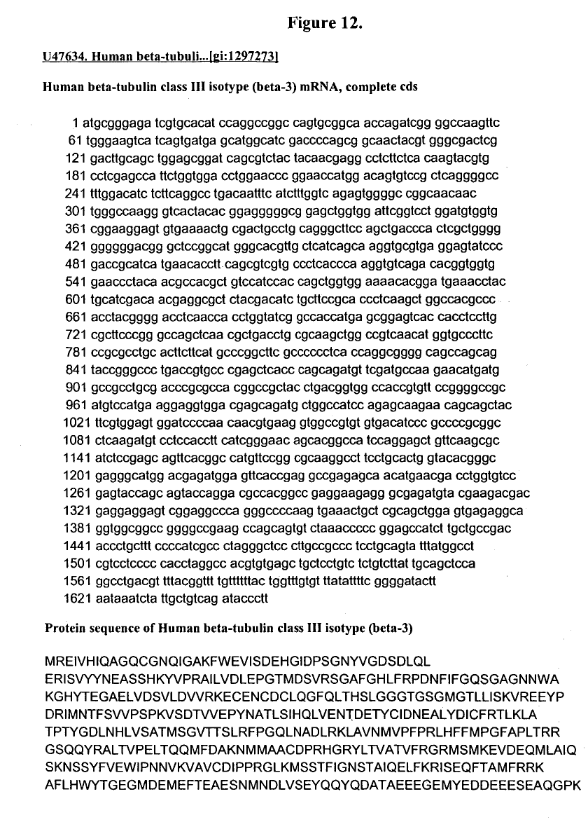

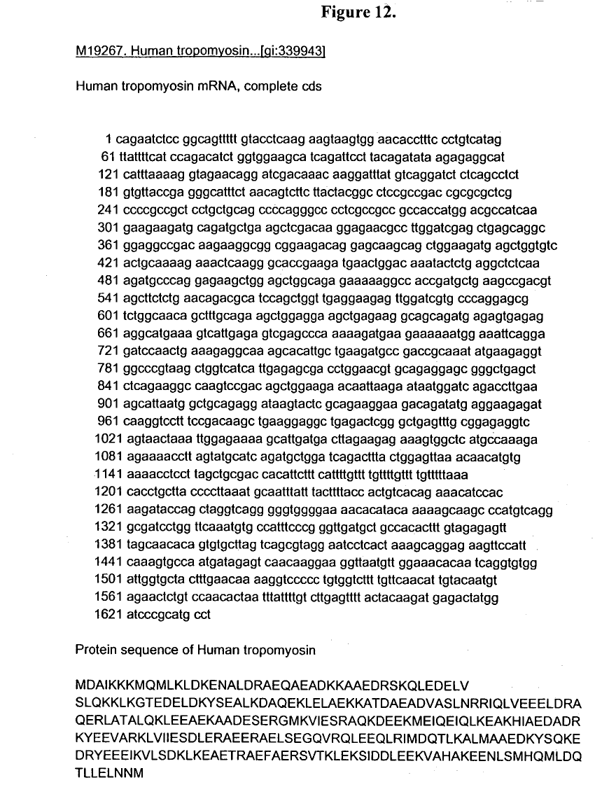

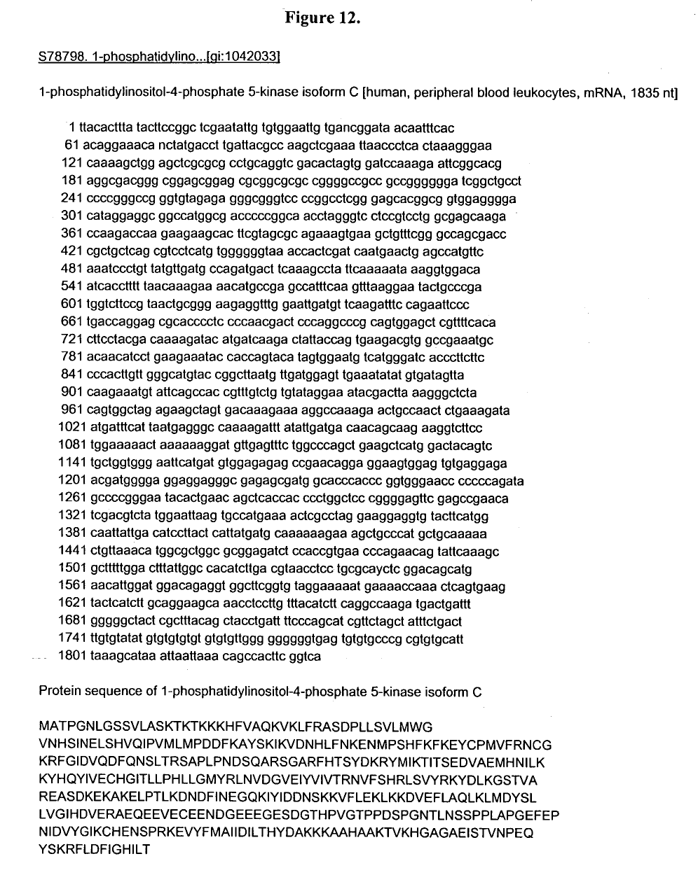

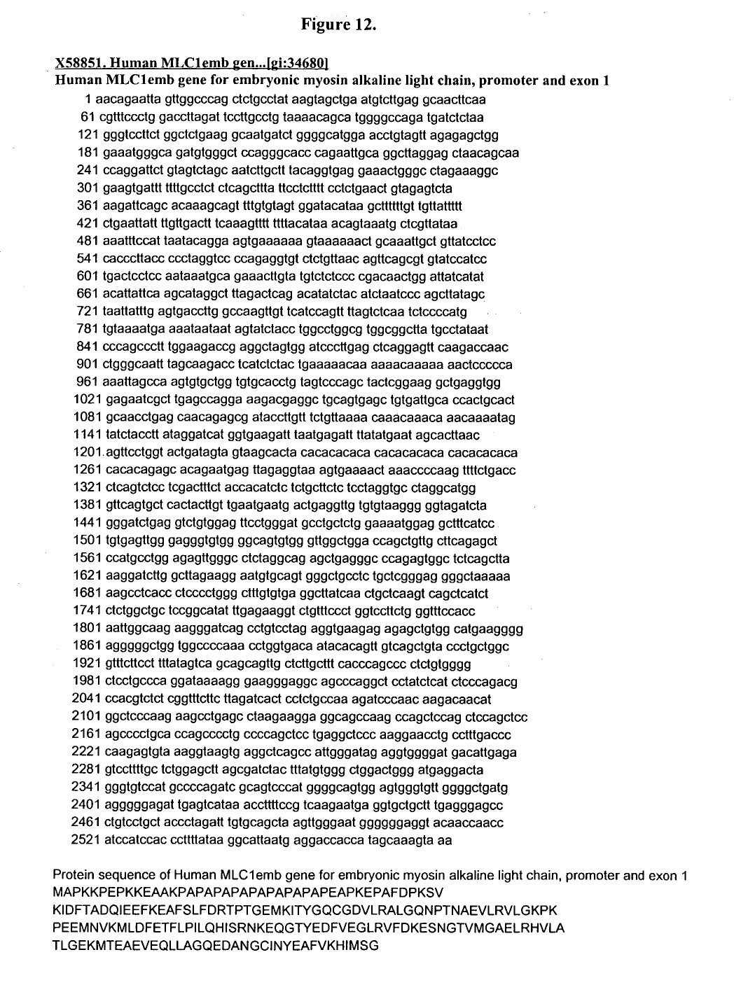

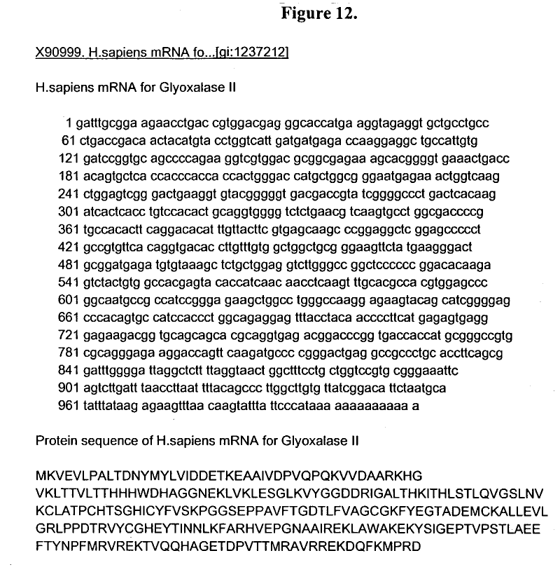

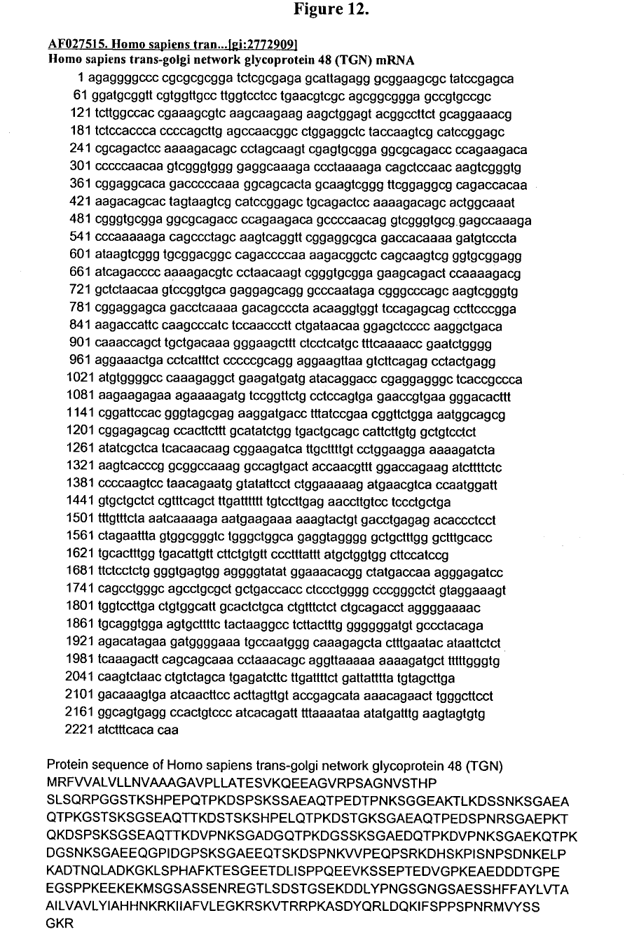

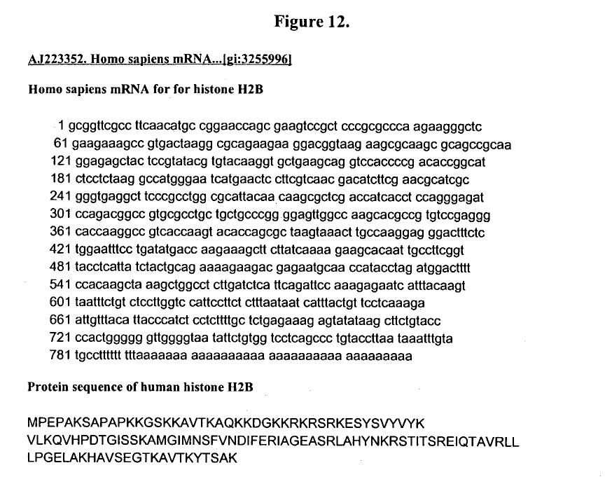

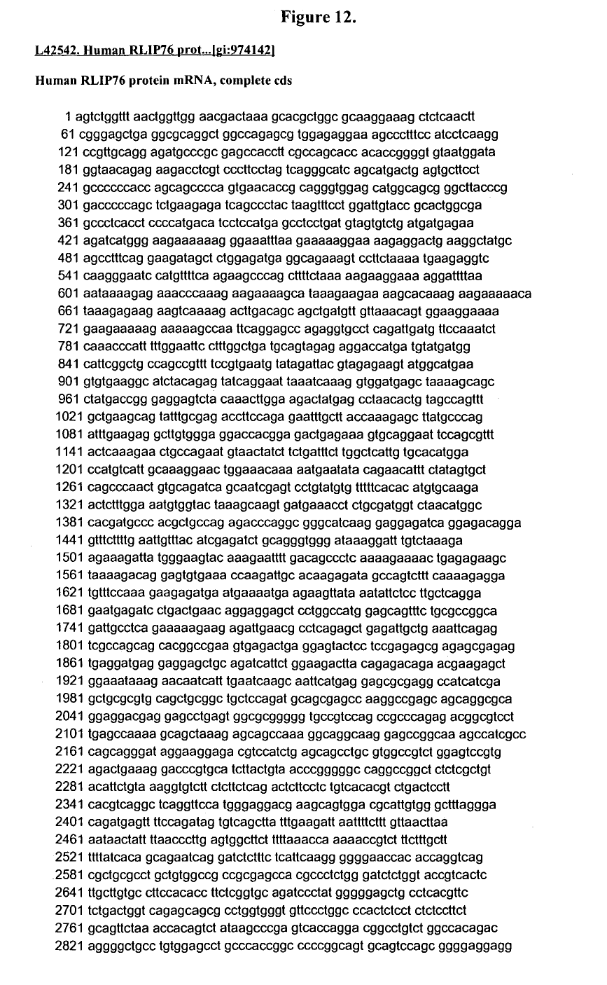

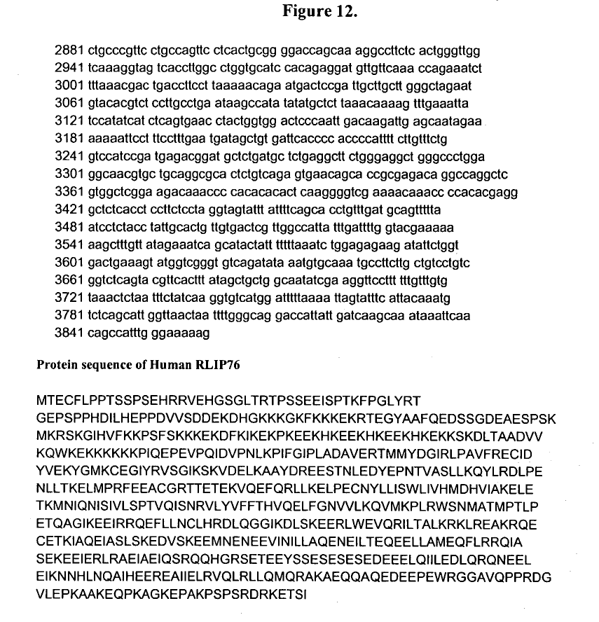

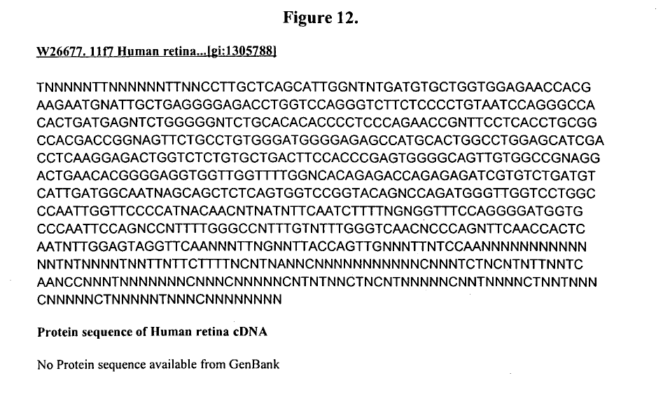

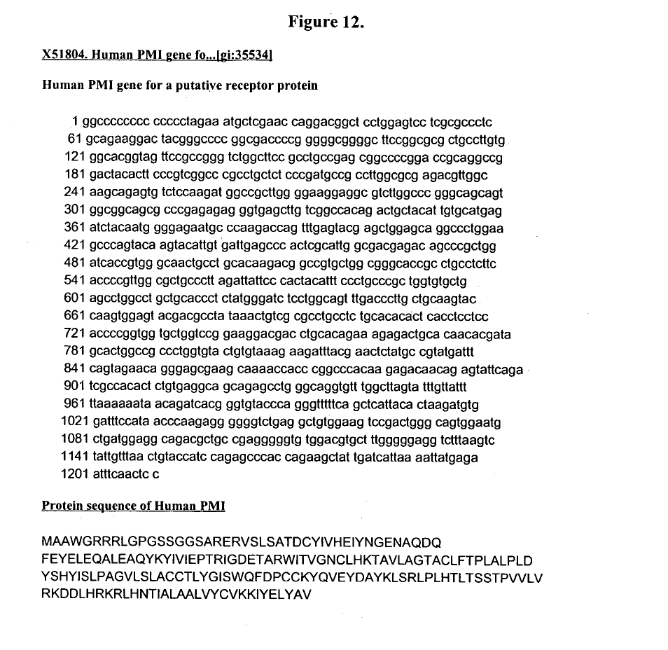

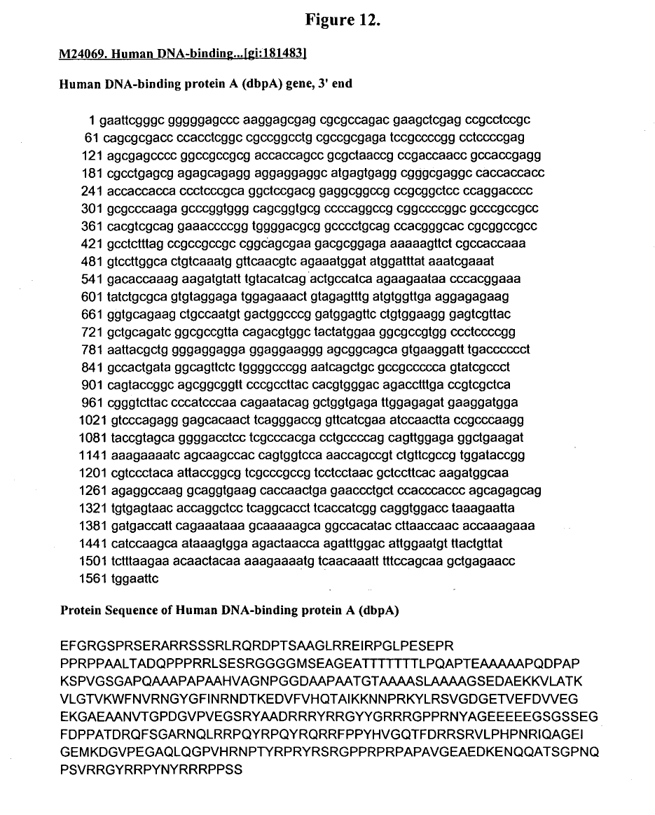

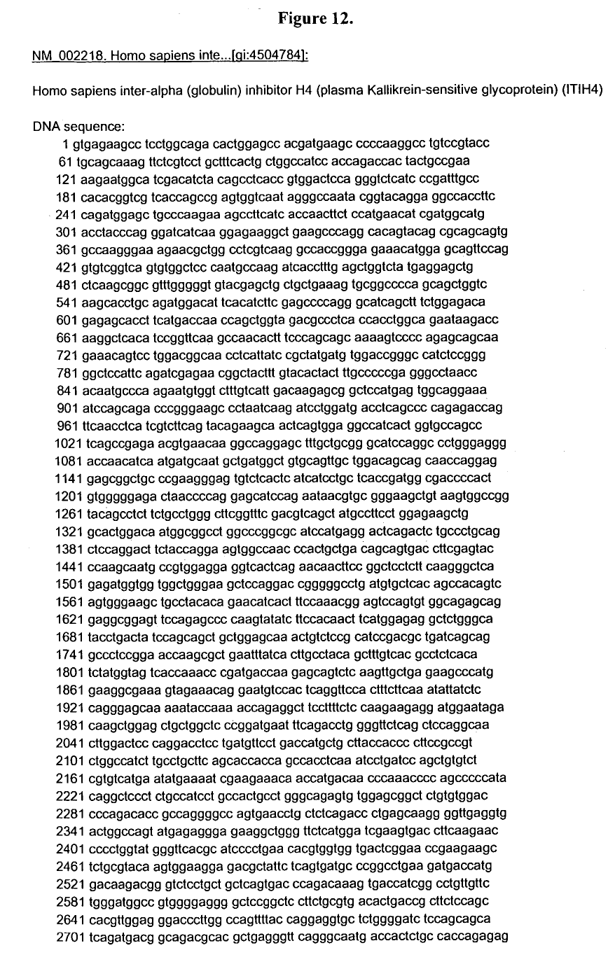

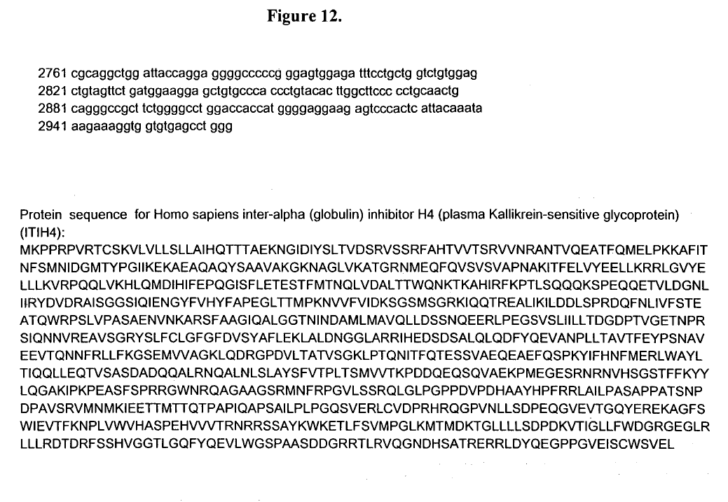

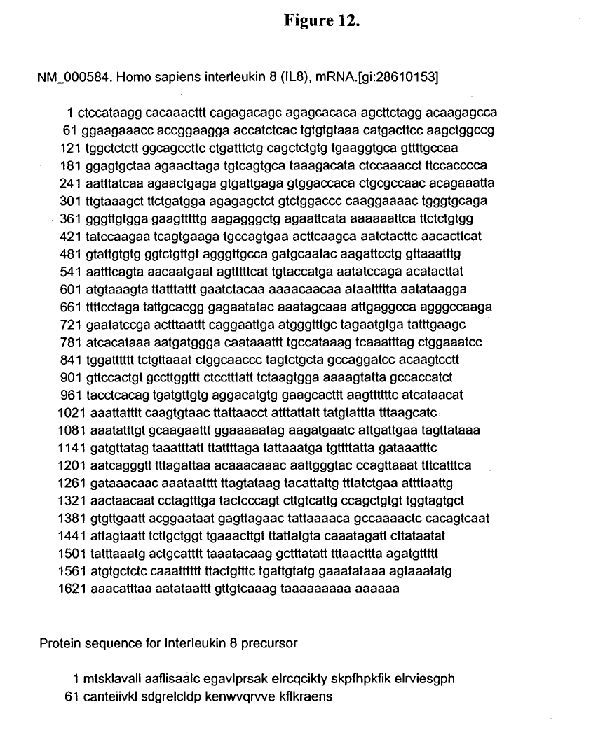

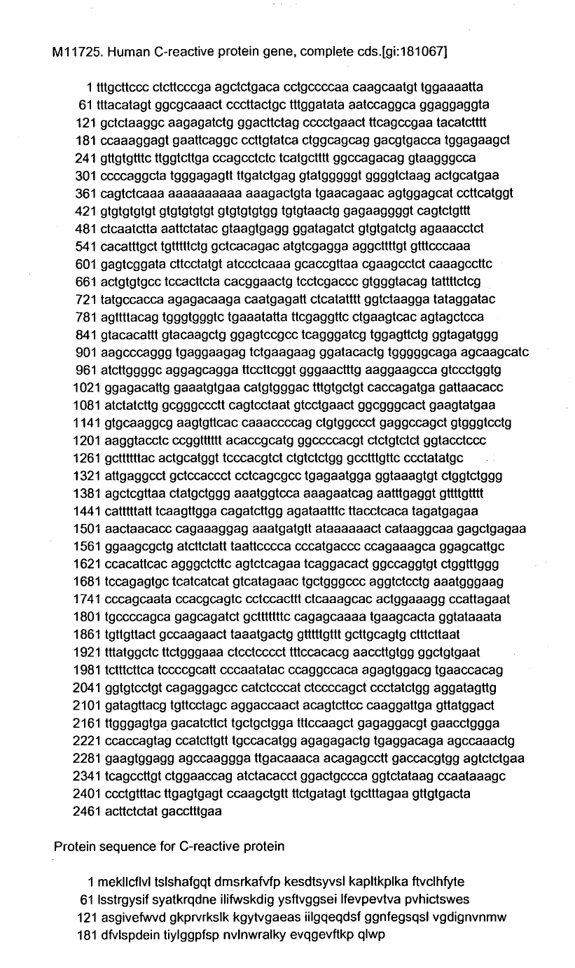

Samples of plasma from human patients suffering from advanced solid malignancies were taken before and 4 hours ater the first (and only) does of Compound A. A variety of proteins were increased and/or decreased in the plasma of patients treated with Compound A. As shown in FIGS. 2 and 3, mass spectrometry analysis identified one of these proteins (spot # 5) as ITIH4 (inter alpha (globulin) inhibitor H4). ITIH4 was therefore identified as a biomarker for a compound that inhibits tyrosine kinase, such as Compound A. See FIG. 12 for sequences for ITIH4.

Microarrays and RT-PCR Analysis

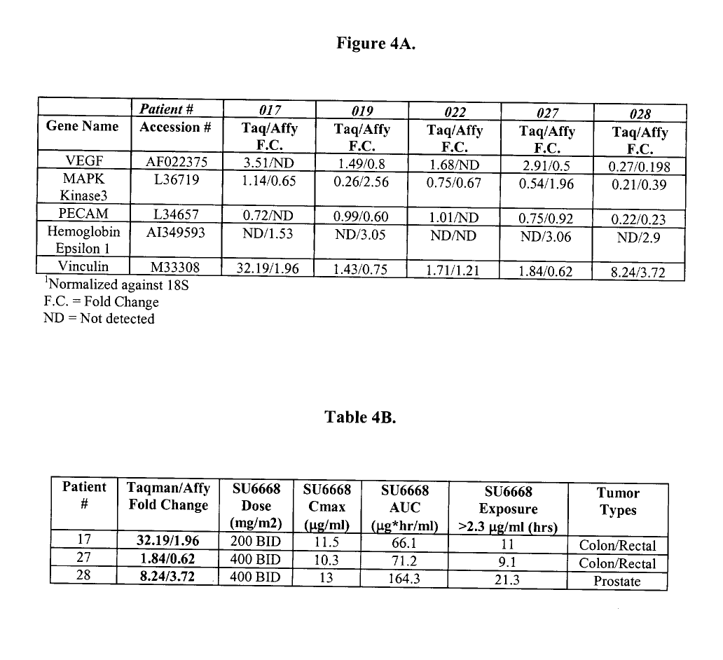

Samples of whole blood from human patients suffering from advanced solid malignancies were taken before and 24 hours after the first dose of Compound A. An Affymetrix GeneChip analysis of the RNA transcripts present in patient blood before and after exposure to Compound A indicated that the levels of vinculin and VEGF RNA increase after exposure to Compound A (see FIGS. 4A and 4B). Vinculin and VEGF were therefore identified as a biomarker for a compound that inhibits tyrosine kinase, such as Compound A.

Microarrays and RT-PCR Analysis

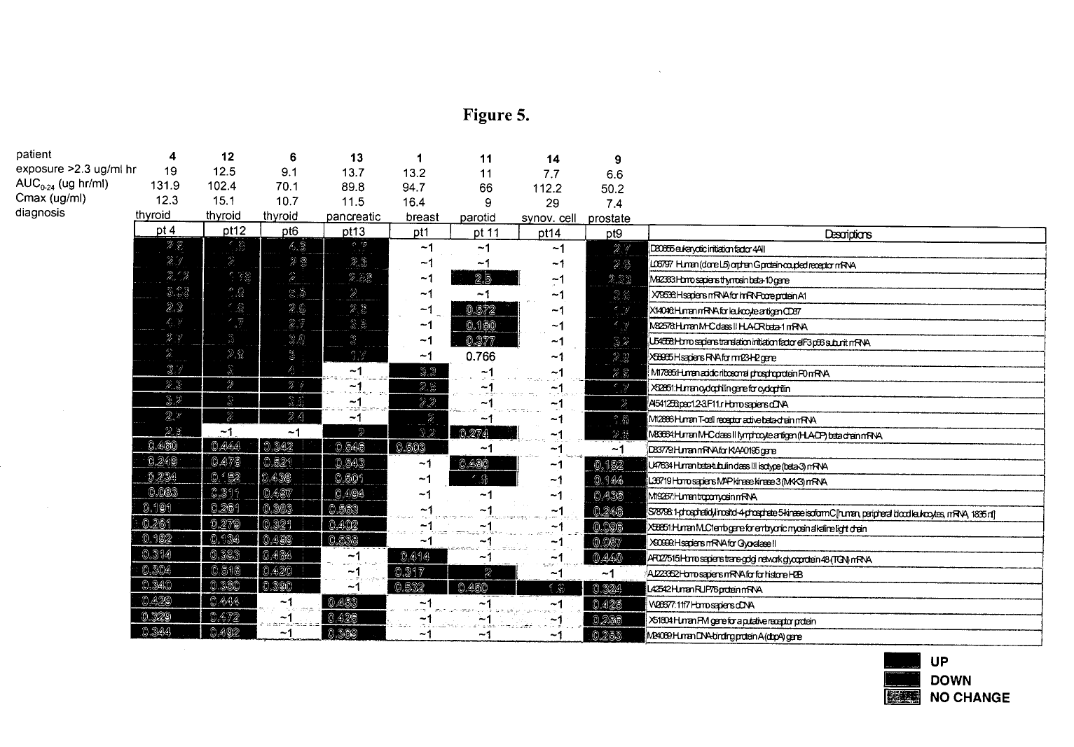

Samples of whole blood from human patients were taken before and 27 days after the first dose of Compound A (in other words, samples were taken on day 0 and day 28; patients were dosed about 2 times per day on day 1-day 27, and following the first dose on day 28, the sample of blood was taken to measure biomarker(s). An Affymetrix GeneChip analysis of the RNA transcripts present in patient plasma before and after exposure to Compound A indicated that the levels of 26 transcripts were increased and/or decreased after exposure to Compound A (see FIG. 5). Thus, 26 proteins/transcripts were identified as biomarkers for a compound that inhibits tyrosine kinase, such as Compound A: eucaryotic initiation factor 4A11, human (clone 5) orphan G protein-coupled receptor (Genbank Accession No. L06792), Homo sapiens thymosin beta-10, Homo sapiens hnRNPcore protein A1, human leucocyte antigen (CD37), human MHC call II HLA-DR beta-1, Homo sapiens translation initiation factor elF3 p66 subunit, Homo sapiens nm23-H2 gene, human acidic ribosomal phosphoprotein P0, human cyclophillin, Genbank Accession No. AI541256 (cDNA), human T-cell receptor active beta chain, human MHC class II lymphocyte antigen (HLA-DP) beta chain, human KIAA0195, Homo sapiens MAP kinase kinase 3 (MKK3), human beta-tubulin class III isotype (beta-3), human tropomyosin, 1-phosphatidyl inositol-4-phosphate-5-kinase isoform C, human MLC emb gene for embryonic myosin alkaline light chain, Homo sapiens glyoxalase II, Homo sapiens trans-golgi network glycoprotein 48, histone H2B member R, human RLIP76 protein, Genbank Accession No. W26677 (human retina cDNA), human PMI gene for a putative receptor protein, human DNA-binding protein A (dbpA). See FIG. 12 for sequences for these biomarkers.

### E. EXAMPLES

### Studies Using Compound B (SU5416)

1. Studies using Compound B—Materials and Methods

Study Population

Patient samples were derived from 2 randomized, open-label, multicenter Phase III clinical trials comparing standard of care chemotherapy alone or combined with Compound B in patients with metastatic colorectal cancer. In both trials Compound B was delivered twice weekly at a dose of 145 mg/m² via I.V. infusion. In the first trial (designated Trial A), the standard of care chemotherapy consisted of weekly administration of 5-FU and leucovorin (Rosewell Park regimen); in the second trial (designated Trial B), the standard of care chemotherapy consisted of weekly or bi-weekly administration of 5-FU, leucovorin and Irinotecan (CPT-11). A total of 23 patient sample pairs were included in Affymetrix microarray expression profiling analysis, 2 females and 9 males in the Compound B treatment arm, and 2 females and 10 males in the control arm. The median patient age was 66 and 65 years for the Compound B treatment arm and control arm, respectively. For RT-verification experiments, samples from 12 females and 24 males from the Compound B treatment arm, and 14 females and 17 males from the control arm were used. The median age for these patients was 62 and 60 years, respectively. Clinical response criteria were defined according to RECIST guidelines. Briefly, complete response (CR) is defined as complete disappearance of all measurable and evaluable clinical evidence of cancer; partial response (PR) is defined as at least a 50% reduction in the size of all measurable tumor areas; progressive disease (PD) is defined as an increase of ≧25% (compared to baseline or best response) in the size of all measurable tumor areas; and stable disease (SD) is defined as neither sufficient shrinkage to quantify for PR nor sufficient increase to qualify for PD.

Patient Samples

All clinical samples for biomarker analysis were harvested and handled in accordance with full Institutional Review Board-approved protocol, and study participants had signed the study informed consent prior to any study related procedures. All blood samples were collected into Vacutainer tubes containing sodium heparin. Ten 10 ml of blood was withdrawn from patients prior to receiving any treatment on day 1 and also prior to dosing at end of cycle 1 (day 56 in Trial A; day 42 in Trial B). For peripheral blood mononuclear cell (PBMC) preparations, blood samples were shipped overnight at ambient temperature to a central processing facility (Quest Diagnostics, Inc., Collegeville, Pa., USA) for PBMC isolation via Ficoll gradient method. Purified PBMCs were shipped in RNA lysis buffer (Clontech, Palo Alto, Calif., USA) to SUGEN where isolation of total RNA was performed. For Trial B, whole peripheral blood samples were directly frozen at the clinical sites and shipped on dry ice to SUGEN for RNA isolation.

RNA Sample Processing

Total RNA was purified from PBMC samples using Clontech Nucleospin RNA II kit reagents (Clontech, Palo Alto, Calif.) and from whole blood samples using MRC TRI Reagent BD (Molecular Research Center, Cincinnati, Ohio, USA), an adaptation of the Chomczynski single step method, according to the manufacturer's instructions. All sample preparations included a treatment with RNAse-free DNAse. RNA yields were measured by UV absorbance and RNA quality was assessed by agarose gel electrophoresis with ethidium bromide staining for visualization of ribosomal RNA band integrity.

Affymetrix High-Density Oligonucleotide Microarray Analysis of PBMC Expression Profiles

In general, the standard RNA processing and hybridization protocols as recommended by Affymetrix (Santa Clara, Calif., USA) were followed in this study; these protocols are available in the Genechip® Expression Analysis Technical Manual (viewable at <www.affymetrix.com/support/technical/manual/expression_manual.affx>. Yields of total RNA for PBMC samples were generally low and for the majority of patients it was not possible to use the standard amount of total RNA (≧5 μg) as recommended in the standard protocol. Therefore a double linear amplification approach was used in the generation of cRNA for hybridization. In these experiments, equal amounts of starting material were used for pre- and post-treatment samples from each donor (typically 2 μg). Briefly, the protocol was as follows: double-stranded cDNA was synthesized from total RNA (2 μg), with Invitrogen Life Technologies SuperScript Choice system reagents (Invitrogen, Carlsbad, Calif.). The T7-(dT)₂₄ oligomer was used for priming first-strand cDNA synthesis. Double-stranded cDNA product was purified via phenol-chloroform extraction, then used as template in first round of in vitro transcription (IVT) of cRNA. The IVT reaction was performed with BioArray High Yield RNA Transcript Labeling Kit (Affymetrix) according to manufacturer's protocol but with substitution of non-biotinylated ribonucleotides for biotinylated ribonucleotides. The cRNA product was then purified with Qiagen spin column clean-up protocol and used as template in second round of cDNA synthesis. This second round of synthesis was similar to the first round except that random hexamers were used in priming of first-strand synthesis, with T7-(dT)₂₄ oligomer priming the second-strand. Purification of the cDNA was as in the first round. The second round of IVT of cRNA was as in the first round but with biotinylated ribonucleotides rather than non-biotinylated ribonucleotides. Purified cRNA was quantitated, chemically fragmented according to Affymetrix protocol, and then hybridized overnight on Human Genome U95A Arrays (which contain probe sets for the detection of approximately 12,600 transcripts). Hybridized arrays were washed and stained with phyoerythrin-conjugated strepavidin detection chemistry in an Affymetrix Fluidics station, then images were scanned with a Hewlett-Packard GeneArray scanner.

Data Analysis

Data files were generated from scanned array images in the Affymetrix Microarray Suite Version 4.0 program. The key output from individual arrays are the Average Difference (AD) values, which serve as relative indicators of the expression level of transcripts represented on the arrays. Average Difference determination relies on difference between background-subtracted signal from perfect match (PM) oligos and corresponding mismatch control (MM) oligos within a probe set representing a given transcript. To enable comparison of all hybridization data, global scaling was applied by multiplying the output of each experiment by a Scaling factor (SF) to make its average intensity equal to a user-defined Target Intensity (which was set at 1500 for these experiments). For comparisons between time points from a single patient, batch files were generated with Microarray Suite. These files contain calculated fold change (FC) values, which represent differential expression ratios of day 56 compared to baseline, and also Difference Calls (DC), which represent a more conservative estimate of differential expression, with qualitative scores assigned to each transcript measurement according to the following system: Increased (I), Marginally Increased (MI), No Change (NC), Marginally Decreased (MD), and Decreased (D).

Subsequent data analysis was performed primarily with Spotfire DecisionSite for Functional Genomics software (version 7) package and its Array Explorer component (Spotfire, Somerville, Mass.). Hierarchical clustering analysis and statistical comparisons were included in this step. Further refinement of the data, including filtering by Difference Call scores, was done with the Microsoft Access 97 database analysis program.

SYBR Green quantitative RT-PCR verification of array results

Primers were designed with Primer Express 1.5 software (Applied Biosystems). In all cases, primers were designed to bind within the sequence that was used in Affymetrix probe set designs (target sequence information available on Affymterix NetAffx website). Total RNA samples (1 μg) were reverse transcribed to yield first-strand cDNA using the Applied Biosystems Reverse Transcription Reagents protocol (Applied Biosystems, Foster City, Calif.). The reverse transcription reactions were then diluted 1:5 in distilled H₂O. SYBR Green PCR reactions were performed in 96-well optical plates and run in an ABI PRISM® 7700 Sequence Detection System (SDS) machine. For individual reactions, 10 μl of each sample were combined with 15 μl of SYBR Green PCR Master Mix (Applied Biosystems) containing the appropriate primer pair at 350 nM. Data was extracted and amplification plots generated with ABI SDS software. All amplifications were done in duplicate and threshold cycle (Cₜ) scores were averaged for subsequent calculations of relative expression values. The Cₜ scores represent the cycle number at which fluorescence signal (ΔRₙ) crosses an arbitrary (user-defined) threshold. Heat dissociation curve analysis was performed after each SYBR Green run as a test of whether a single product had been generated in each PCR reaction; multiple peaks in the dissociation curves are indicative of multiple PCR products and thus reduced specificity and sensitivity.

Quantitation and Statistical Analysis of SYBR Green PCR Data

The Cₜ scores for genes of interest for each sample were normalized against Cₜ scores for the corresponding endogenous control gene, which was the β-glucoronidase (GUS) gene in these experiments. Relative expression for day 56 compared to day 1 was determined by the following calculation, as described in the Applied Biosytems users bulletin on Relative Quantitation of Gene Expression:

Rel Exp−2^{-ΔΔCt},

Where ΔΔCt=(Ct_{Target}−Ct_{GUS})_{day 56}−(Ct_{Target}−Ct_{GUS})_{day 1}.

The relative expression data for a select subset of potential biomarkers were tested for differences between the Compound B (treatment) and the standard of care (control) arms. The Mann-Whitney U Test with a critical alpha level of 0.05 was used for statistical significance. Individual genes observed to be significantly different by Affymetrix analysis and in both sets of SYBR Green RT-PCR experiments were screened as potential biomarker candidates. This subset of potential biomarker candidates was tested subsequently for utility as class predictors to discriminate between the Compound B and standard of care arms. Discriminant analysis, a multivariate statistical technique, was used for this purpose. The genes were tested individually, using all possible combinations, by reducing dimensions (Principal Component Analysis) in order to determine the subset of genes (predictor variables) that yielded highest classification accuracy. Cross-validation was used to test the robustness of classification accuracy. Results from three different cross-validations were evaluated to select the best set of predictable biomarkers: (1) jackknife method (dropping one case at a time); (2) randomly splitting the pooled data into two halves, prediction (for building model) and validation (for testing model); and (3) using the first trial as prediction and the later trial as validation sets, respectively. All statistical analyses were carried out after natural-log transformation on the data; SYSTAT 9.01 (SPSS, Inc., Chicago, Ill., USA) software was used in statistical analysis.

2. Studies Using Compound B—Results

Affymetrix Expression Profiling of Pre- and Post-Treatment Matched PBMC Samples

Expression profiling using Affymetrix high-density oligonucleotide microarrays was applied to PBMC samples harvested from patients in a Phase III clinical trial of Compound B in Trial A. The PBMC samples were harvested at baseline (day 1) and at end of cycle 1 (day 56) from patients receiving standard-of-care (5-FU/leucovorin) treatment and from those receiving standard-of-care plus Compound B. Sample pairs from 23 patients were processed and the dataset was filtered for expression changes that consistently correlated with the treatment arm (Compound B). Of 13 genes that met the initial requirement, 6 were further tested by quantitative RT-PCR analysis of additional patient samples from patients.

Table 1 includes a summary of the total samples processed. As RNA yields rarely exceeded 2 μg, a double amplification step was used in cRNA generation for the samples that were used (see Materials and Methods). Only samples from patients with cycle 1 responses of either PR/CR or PD were used in the final dataset.

Batch comparison files were generated for each day 1/day 56 sample pair after hybridization. Batch comparisons included both fold change (FC) values as calculated by Affymetrix Microarray Suite software as well as difference calls (DC). DC offer a more stringent but non-numerical measure of whether levels of a transcript are different in the 2 samples. Batch comparison results for the 23 cases were analyzed with Spotfire Decision Site software tools. Initial analysis suggested there was more similarity among patient samples of the same treatment arm than among samples of the same response category (PR/CR or PD) independent of treatment arm. Therefore, subsequent analysis focused on identification of transcripts that were differentially expressed in the Compound B arm but not in the control arm.

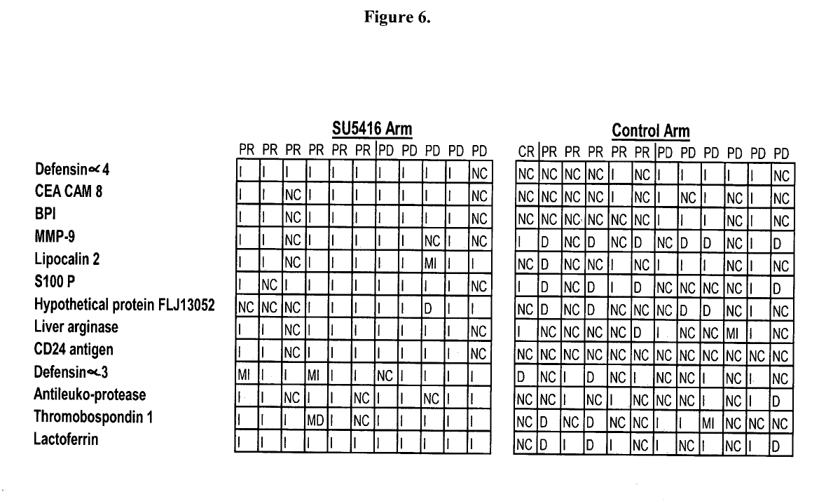

The Treatment Comparison tool in Spotfire was used to identify transcripts that were statistically significantly different in the two treatment arms; this tool uses t-test analysis of averaged fold changes for each group. To further refine this subset of genes, queries based on DC status were performed with Microsoft Access. The data were filtered to identify those genes that were called ‘Increased’ (I) or ‘Decreased’ (D) in a majority of the Compound B arm cases. A group of 13 genes that frequently showed increased expression was identified. FIG. 6 displays a schema of the DC scores assigned to each gene for each patient sample pair. All cases from the Compound B arm show induction in at least 6 of the 13 genes.

Table 2 includes a brief summary of putative biological function for each of the 13 gene products, as well as an ID number assigned by Affymetrix to each transcript-specific probe. The last two columns in Table 2 list the number of patients in which transcript levels were increased at day 56 relative to day 1 (i.e., an ‘Increase’ call was assigned). Total number of patients is 11 for the Compound B (SU5416) arm and 12 for the control arm. The average fold change of all of these transcripts was higher in the Compound B (SU5416) arm (the lowest average fold change was 2.6 for hypothetical protein FLJ13052, the highest was 33 for lactoferrin); the range of fold changes was also broader in this category, presumably representing variability among patients.

Quantitative RT-PCR Validation of Differentially Expressed Transcripts

To validate the microarray results, a subset of these transcripts was chosen for quantitative RT-PCR analysis. Primer sets were designed for 6 of the 13 genes; matrix metalloproteinase-9 (MMP-9), thrombospondin-1 (TSP-1), CD24, defensin α 3, lipocalin 2 (LCN2), and lactoferrin. These 6 genes were chosen based on potential roles of encoded proteins (for example, thrombospondin-1 and MMP-9 have known roles in angiogenesis) or because of the degree to which they appeared to be differentially regulated between treatment arms. The lipocalin-2 gene (LCN2) has been reported to be inducible by dexamethasone (Science, 293: 829-34 (2001)). Dexamethasone is one of the premedications administered to patients in the Compound B arm. Table 3 describes the forward and reverse primers that were used in validation of these transcripts.

SYBR Green chemistry was used to validate the microarray expression profiling data. SYBR Green is a dye that fluoresces when bound to double-stranded DNA, thus signal is directly proportional to the amount of product formed during PCR amplification. This method allows rapid and inexpensive comparison of gene expression across a large number of samples. The qRT-PCR validation was performed with a total of 31 Compound B patient sample pairs, 8 of which had previously been analyzed on Affymetrix U95A arrays and thus allowed a comparison of the correlation between the 2 transcript profiling methods. Of the 31 samples, 18 were from the Compound B arm and 13 were from the control arm.

Data for each gene was normalized to expression of a housekeeping gene, β-glucoronidase (GUS). By direct comparison of SYBR Green RT-PCR results and Affymetrix results from the same cases, the overall qualitative correlation (i.e., same trend of induction or no change detected in both samples) was greater than 70%. This number is perhaps an underestimate since results for one patient were completely discordant between methods and thus potentially due to experimental artifact.

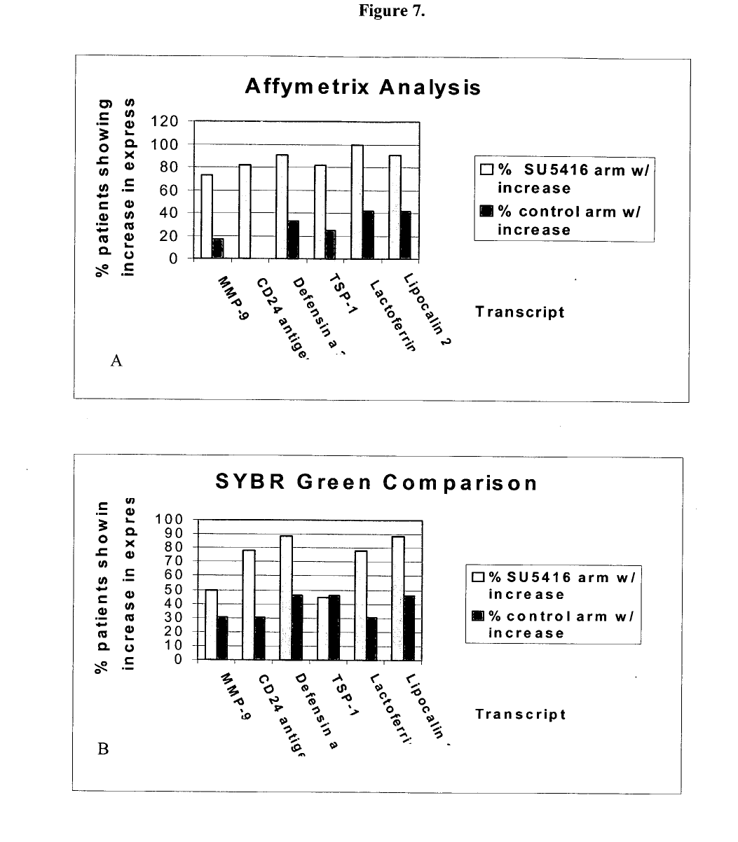

FIG. 7 summarizes the results from the RT-PCR validation and compares them with those from the Affymetrix analysis. It is clear that there are some differences in the trends displayed in the 2 datasets. This is further demonstrated by statistical analysis, as Mann-Whitney U test comparison of Compound B and control results from both analyses indicates that only 4 of the 6 genes display statistical significance (Table 4). These 4 genes are CD24, lactoferrin, LCN2, and MMP-9. (MMP-9 exhibited a p-value that was close to the significance cutoff and thus was also selected for further analysis.)

Qualitative RT-PCR Validation of Differentially Expressed Transcripts with Samples from a Second Phase III Compound B Trial

To further confirm these transcripts as biomarkers of Compound B administration, SYBR Green RT-PCR analysis of these 4 transcripts was carried out in a collection of samples from a second Phase III trial (Trial B). In this randomized metastatic colorectal cancer study, 5-FU/leucovorin/CPT-11 was administered as the standard of care, and compared to the standard of care plus Compound B. RNA samples from patients in this trial were derived from frozen whole blood (rather than purified PBMCs), and harvested at the beginning (pre-dose day 1) and at the end (day 42) of cycle 1. To test if similar results occurred, analysis was performed on 36 sample pairs, 18 from Compound B arm and 18 from control arm. Due to limited numbers of available samples, many of the cases analyzed in this analysis were from patients with stable disease (SD) at cycle 1 assessment rather than PR/CR and PD as in the previous approaches.

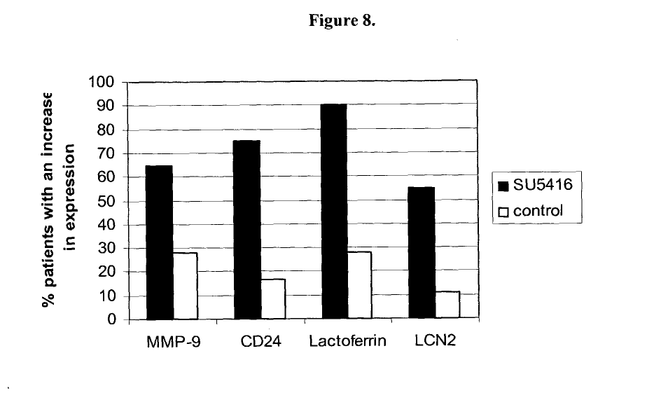

FIG. 8 summarizes the overall behavior of the transcript levels in both trial arms in terms of the frequency with which the transcripts showed an induction (here defined as relative expression, day 42 vs day 1) of 2-fold or greater in each arm. It is clear that there is more induction of these transcripts at day 42 in the Compound B arm than in the control arm. This is also reflected in statistical analysis, as indicated in results of the Mann-Whitney U Test of this dataset (Table 5).

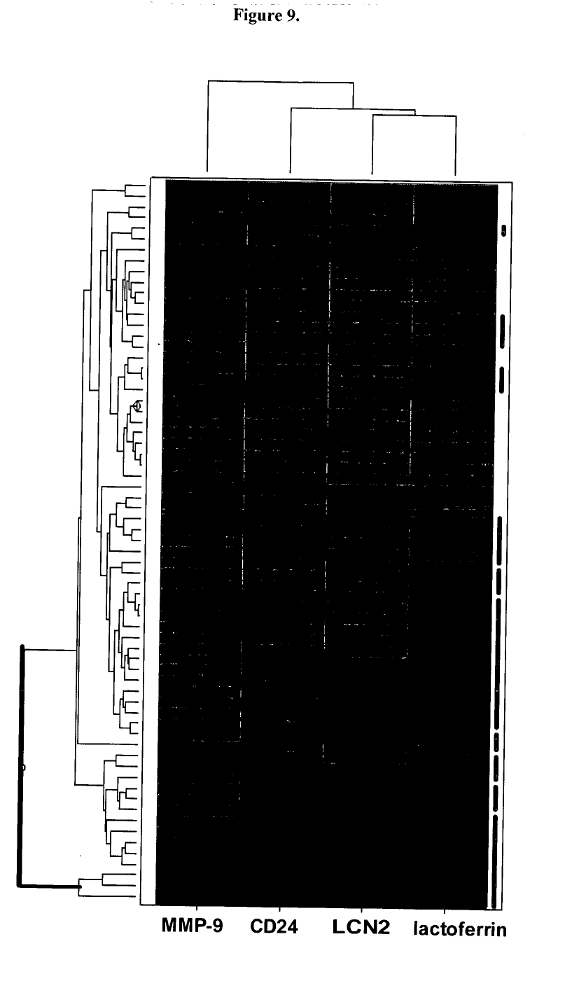

A visual representation of hierarchical clustering analysis of the qRT-PCR relative expression values from both trials for each of the transcripts is displayed in FIG. 9. This clustering pattern displays the distinction between the Compound B and control arms based on relative expression data, and also indicates further distinctions among subsets of patients as well as the degree of overlap between trial arms in the clustering pattern. The extent of similarity between the relative expression patterns for each transcript (represented in columns) is also indicated; the pattern of MMP-9 is distinct from the others as it appears in a separate branch in the dendrogram structure.

Discriminant Analysis of the Classification Power of Biomarkers

We tested whether relative expression data from these samples could be used in a predictive fashion to classify samples to the appropriate trial arm. To test this, discriminant analysis of the SYBR Green RT-PCR data was performed. Relative expression values from both the first and the second dataset were combined, after comparison of mean relative expression ratios and standard deviations indicated greater similarity between respective trial arms rather than between control and Compound B arm in either trial alone. The relative expression ratios were then natural log-transformed to reduce the scale of the values and thus make control and treated arms more comparable. When the samples were pooled (67 cases altogether) and subjected to classification prediction, a total prediction accuracy of 84% was achieved. Further cross-validation was performed by the jack-knife method (which does a series of predictions, randomly removing 1 case from the total each time), and by splitting the data set into 2 random halves (one a ‘training’ set and the other a ‘testing’ set).

The results from each of these steps are summarized in Table 6 for a set of 3 of the 4 transcripts that gave the best accuracy percentage (including MMP-9 slightly reduced the accuracy of cross-validation). Thus, it is predicted that expression data from these 3 genes would accurately distinguish Compound B arm patients from control arm in between 67% to 84% of cases. When the first trial data was used as the ‘training’ set and the second trial data as the ‘testing’, as opposed to randomly selecting the data, the % accuracy in cross-validation was 86% and 77% for the training and testing set, respectively. Cross-validation results are displayed for two different approaches. In section 2 of Table 6, one case is dropped at a time and its group membership predicted from the other cases. In sections 3 and 4, cross-validation is carried out by using a randomly selected half of the cases as a training set and the remaining half as a test set. Section 4 summarizes the prediction accuracy achieved when the group in section 3 is used as a training set.

Conclusions: Compound B Studies

Large-scale gene expression analysis was applied to blood RNA samples from a clinical trial of Compound B to investigate changes in gene expression that might correlate with exposure to cancer therapy. Independent quantitative RT-PCR validation of initial array hybridization results was performed on larger sample populations from two conceptually similar Phase III clinical trials using Compound B. A set of 4 transcripts (CD24, lactoferrin, LCN2, and MMP-9) was identified whose expression was significantly induced at the end of one treatment cycle relative to baseline following Compound B administration. Discriminant analysis indicates that data derived from the RT-PCR study would have a class prediction accuracy of at least 70%.

These 4 transcripts are considered to be biomarkers of Compound B administration and other compounds that inhibit tyrosine kinase. These results also demonstrate that human blood samples can serve as surrogate tissues for biomarker investigations and that large-scale gene expression analysis is a useful approach for characterization of clinical trial samples.

### F. EXAMPLES

### Further Studies Using Compound B (SU5416)

Baseline and Post-Treatment Levels of PAI-1 in Compound B Patient Plasma

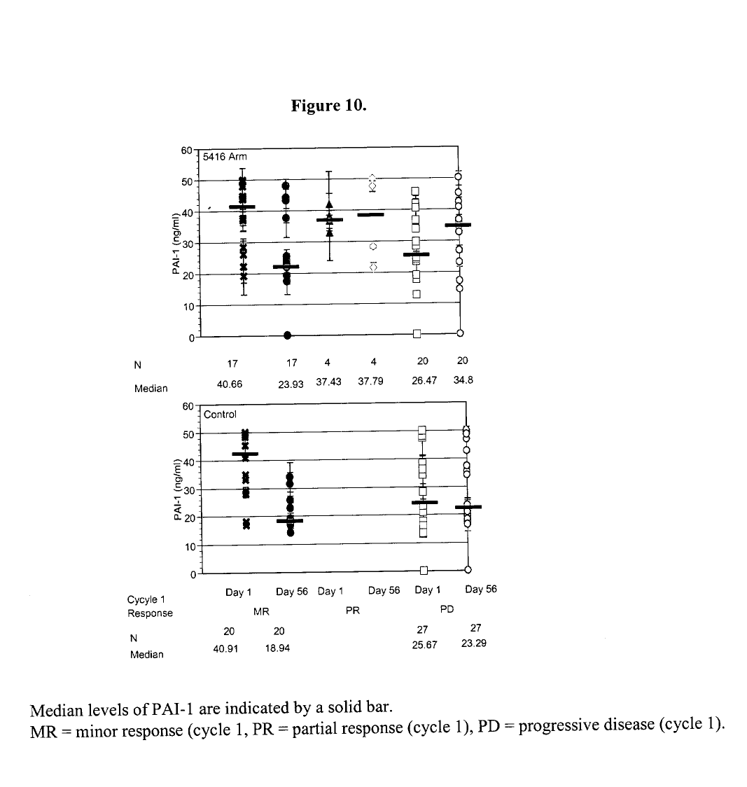

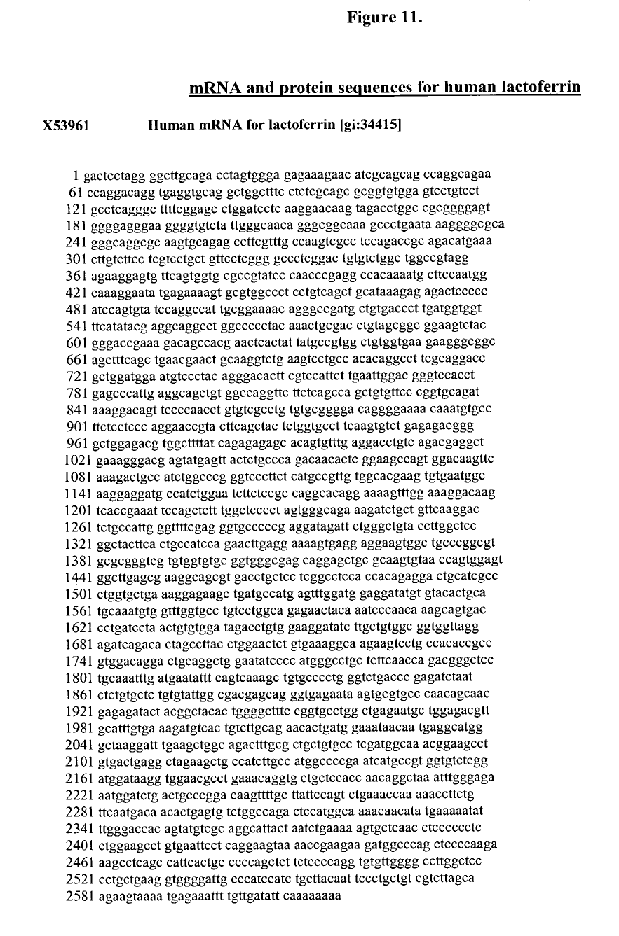

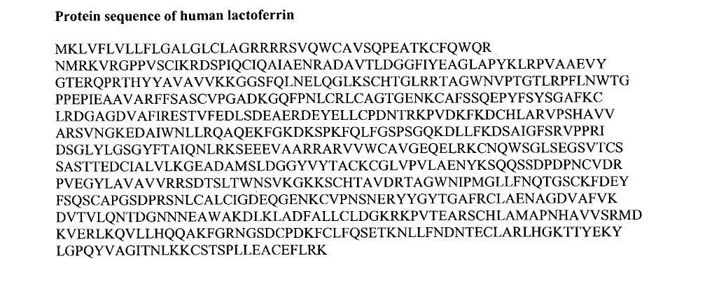

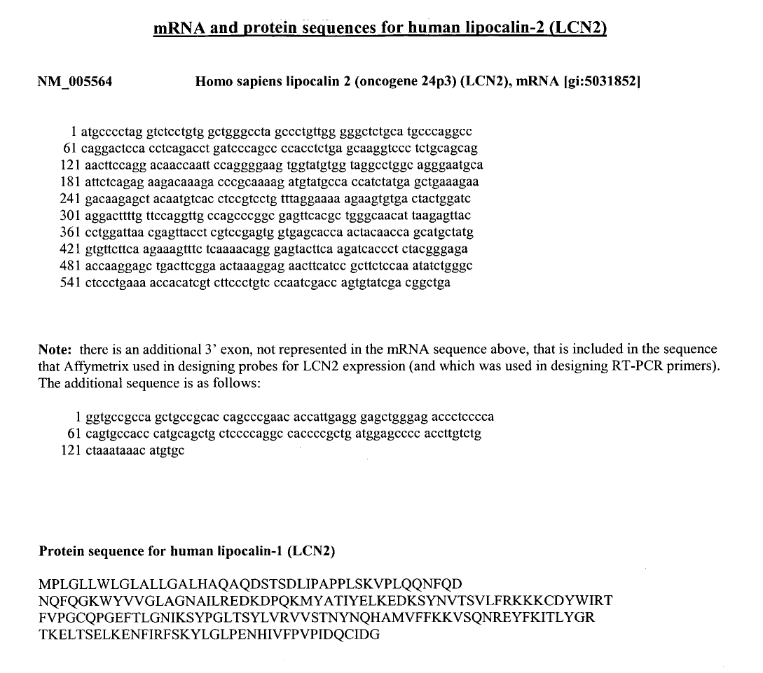

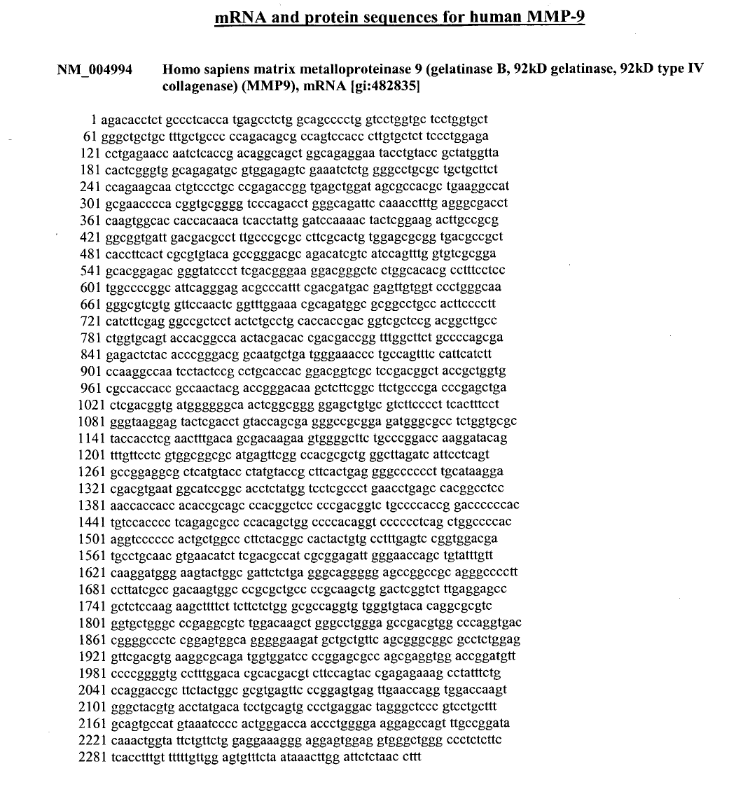

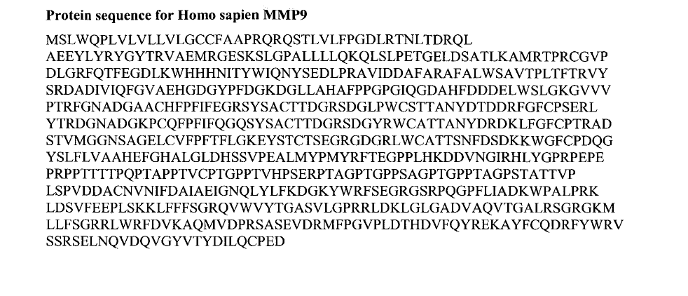

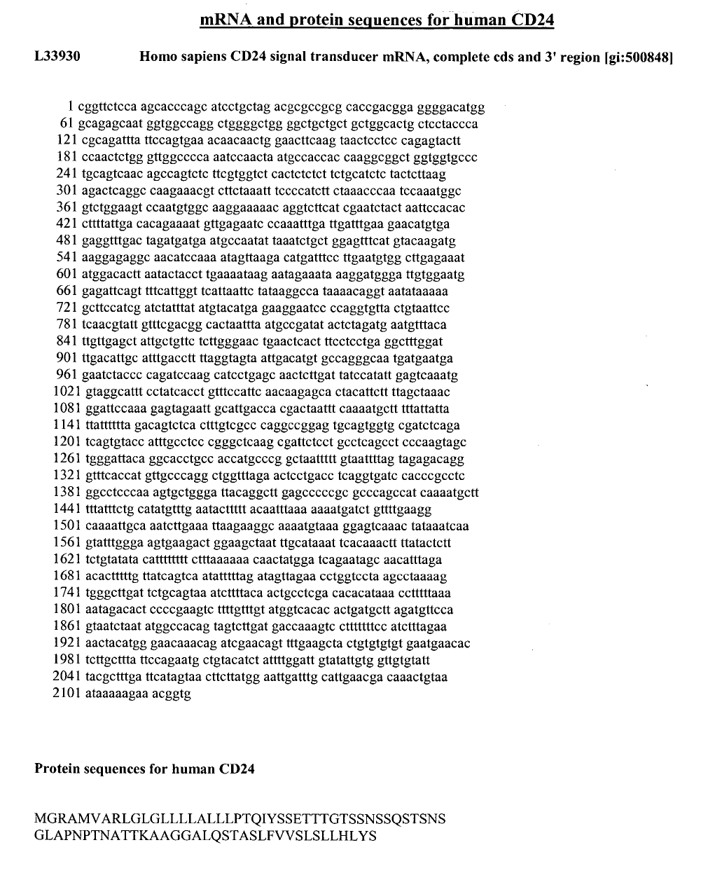

PAI-1 plasma levels were examined in samples from Compound B patients. Interestingly, median PAI-1 levels decreased after 56 days of treatment in samples from all patients examined with a MR (minor response) at the end of cycle 1 (FIG. 10, n=37; Compound B arm day 1 median 40.66 ng/ml, day 56 median 23.93 ng/ml, 5FU/LV arm day 1 median 40.91 ng/ml, day 56 median 18.94 ng/ml). In contrast, median PAI-1 levels in samples from all patients examined with a PD (progressive disease) response at the end of cycle 1 did not appear to change significantly (FIG. 10, n=47; Compound B arm day 1 median 26.47 ng/ml, day 56 median 34.8 ng/ml, 5FU/LV arm day 1 median 25.67 ng/ml, day 56 median 23.29 ng/ml). Furthermore, the decrease in PAI-1 plasma levels in the control arm MR patients after 56 days of treatment was statistically significant (day 1 median 40.91 ng/ml, day 56 median 18.94 ng/ml, P=0.0003; n=20). The decrease in PAI-1 levels of Compound B arm patients was not statistically significant (P=0.095; n=17). These data indicate that changes in plasma PAI-1 levels after one cycle of treatment correlate with cycle one clinical response of both the experimental and control arm regimens.

Pre-Treatment Levels of PAI-1

An analysis of the pre-treatment plasma levels of plasminogen activator inhibitor-1 (PAI-1) shows that pre-treatment levels also correlate with clinical response (on day 56) in either arm, indicating that PAI-1 is a biomarker predictive of response to tyrosine kinase inhibitor in advanced colorectal cancer.

An analysis of the pre-treatment levels of PAI-1 indicated that patients with an MR response (cycle 1) had a statistically significantly (P=0.001) higher level of plasma PAI-1 (median 41 ng/ml; n=37) than that of patients with a PD response (median 26 ng/ml; n=47) regardless of the regimen subsequently received. Thus far, only 4 patients that had a partial response (PR) at the end of cycle 1 have been examined for PAI-1 plasma levels. These patients have pre-treatment levels (median 37.4 ng/ml) similar to the MR patients (median 40 ng/ml), however PAI-1 levels did not decrease significantly in these patients samples after 56 days of treatment. These results (see FIG. 10) indicate that the pre-treatment levels of plasma PAI-1 are predictive of MR response (as compared to a PD response) to either the experimental or the control arm regimen.

The present invention includes a method for predicting the probability of whether a patient will respond positively to administration of a tyrosine kinase inhibitor, comprising measuring the level of PAI-1 in patient plasma, wherein a level of greater than 30 nanograms/per ml of plasma, or greater than at least 35 nanograms, or greater than at least 37 nanograms per ml, indicates a positive probability that the patient will respond positively to administration of a tyrosine kinase inhibitor.

### G. EXAMPLES

### Studies Using Compound 1

1. Studies Using Compound 1—Materials and Methods

A panel of proteins were investigated for their utility as biomarkers of Compound 1 in cancer patients receiving the compound in Phase I trials. The patient samples were from a total of four Phase I trials, 3 of which were open to patients with any advanced solid malignancy (these were Trials A, B and C) and one of which (Trial D) was a trial in patients with Gleevec-refractory, resistant, or intolerant gastrointestinal stromal tumors (GIST). In all cases, plasma samples were available from just before first Compound 1, or malate salt thereof, dose (baseline) and at various time points during dosing. In Trials A and B, patients received Compound 1. In Trials C and D, patients received a malate salt of Compound 1. For methods of making Compound 1, see U.S. Ser. No. 09/783,264 or WO 01/60814, U.S. Ser. No. 10/076,140 or U.S. Ser. No. 10/281,985, the disclosures of which are incorporated by reference. For methods of formulating Compound 1, see U.S. Ser. No. 10/237,966 (now a U.S. provisional application), the disclosure of which is incorporated by reference.

All of the ELISA-based screening of candidate proteins were performed with commercially available ELISA kits; the kits for the biomarkers described in this report are all available from R&D Systems (Minneapolis, Minn.). A commercially available membrane array containing antibodies for the detection of 42 human cytokines was also used in screening of a patient's plasma samples before and after treatment. The antibody array used in cytokine screening (RayBio Human Cytokine Array III) was from RayBiotech (Norcross, Ga.).

All clinical plasma samples were harvested and handled in accordance with full Institutional Review Board-approved protocol. Study participants signed the appropriate informed consent prior to any study related procedures. Plasma was separated from blood samples collected into Vacutainer tubes containing sodium heparin and shipped frozen to the SUGEN site. The time points for which plasma samples are available in each trial are as follows:

Trial A (4 weeks on/2 weeks off dosing schedule):

plasma—Day 1 (0, 6, 24 hr); Day 28 (0, 6, 24 hr)

Trial B (2 weeks on/2 weeks off):

Plasma—Day 1 (0, 6, 12, 24 hr); Day 13 (0, 6, 12, 24 hr)

Trial C (4 weeks on/2 weeks off):

Plasma—Day 1 (0, 6 hr); Day 15, 29, 42* (Cycle 1); Day 1, 15, 29 (Cycle 2)

Trial D (2 weeks on/2 weeks off):

Plasma—Day 1, 7, 14, 28* (Cycle 1); Day 1 only, in subsequent cycles

Trial E (4 weeks on/2 weeks off):

Plasma—Day 1, 3, 28 (Cycle 1)

* ‘washout’ sample

Plasma samples were also collected from a set of 10 SUGEN healthy donors; plasma was collected at 3 time points for each donor (day 1, 14, and 28) to mimic time points used in the Phase I trials and thus serve as controls for the normal level of fluctuation of plasma markers in the absence of Compound 1 treatment.

Data analysis was performed for each marker. This was done by generating ratios of plasma levels at various time points during treatment versus the plasma levels at baseline (pre-dose on day 1, cycle 1), or by comparing absolute plasma concentrations at times during treatment to the baseline absolute plasma concentrations. For correlative analysis, scatter plots were drawn and linear regressions were calculated comparing fold change (end of cycle 1 dosing to baseline) of each marker to corresponding values assigned to clinical parameters such as pharmacokinetics, drug dosage, and ¹⁸FDG-PET functional imaging.

2. Studies Using Compound 1—Results

A panel of candidate proteins was evaluated by ELISA analysis in plasma samples from cancer patients receiving Compound 1 or malate salt thereof. Of those investigated, a subset was observed to change consistently in patients receiving Compound 1 or malate salt thereof. One of the proteins was Vascular Endothelial Growth Factor (VEGF); large increases (greater than 3-fold) in plasma levels were seen in approximately 70% of patients in Trials A, B and C, and in a small proportion of patients in Trial D.

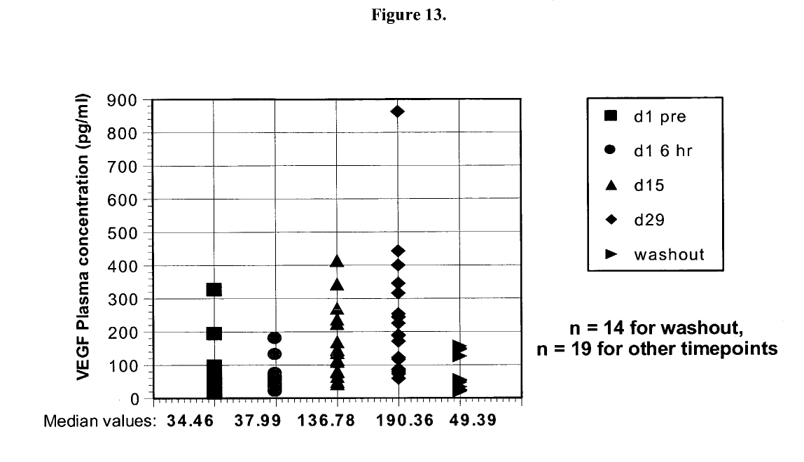

FIG. 13 displays typical pattern of VEGF plasma levels seen in Trial C. VEGF levels are observed to rise by day 15 of cycle 1 and typically peak at day 29, then tend to subside to near baseline levels by day 42, which is the end of the 2-week drug rest period, or ‘washout’, in these patients.

To further investigate this, levels of a related angiogenic factor, Placenta Growth Factor (PLGF), were measured in some of the same patients as in the VEGF tests. As shown in Table 7, levels of PLGF are induced in a majority of patient samples that were tested, and follow a similar pattern as VEGF in that levels are most induced at day 29 and decline by day 42.

A further question regarding VEGF and PLGF was whether the presence of VEGF/PLGF heterodimers in patients' plasma could be detected, and whether levels of the heterodimer could be modulated by treatment with Compound 1 or malate salt thereof. Heterodimers of VEGF and PLGF have been reported in the scientific literature. To measure heterodimers, a hybrid ELISA assay was used, combining reagents from both the R&D Systems VEGF and PLGF ELISA kits (where VEGF antibodies are used in capture step and PLGF antibodies are used in detection step).

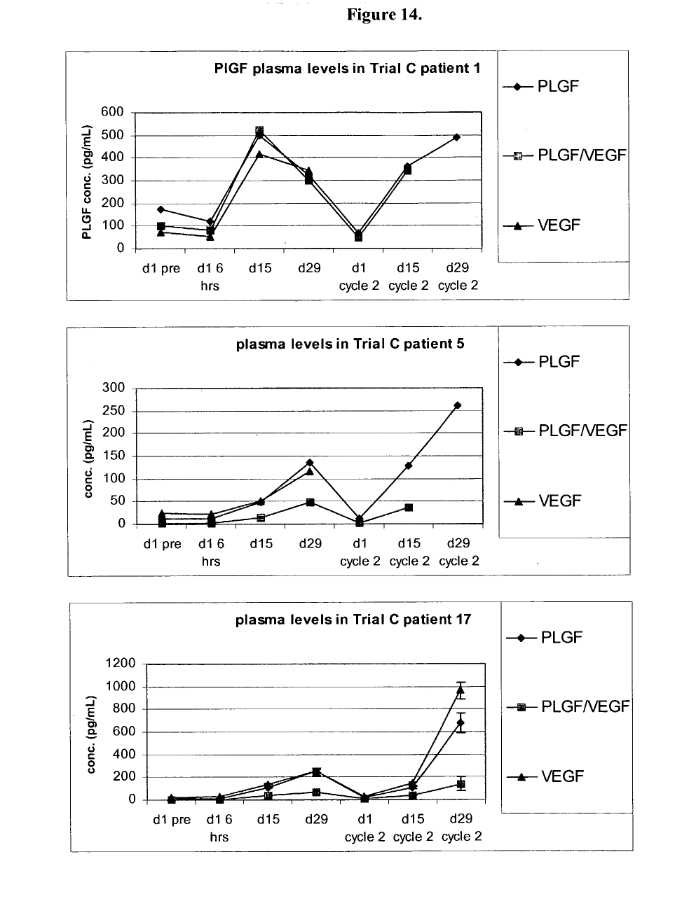

The results of applying this assay to plasma samples from 3 patients are shown in FIG. 14. Data from the same samples for VEGF and PLGF are also shown in the graphs in FIG. 14. A similar pattern of induction of the VEGF/PLGF heterodimer as was seen for VEGF and PLGF was observed. In 3 of 3 patients tested, an increase in plasma levels of VEGF/PLGF heterodimer is observed, indicating that both PLGF and the VEGF/PLGF heterodimer are novel biomarkers of Compound 1 activity in patients.

Another protein, VEGF receptor 2 (VEGFR2) was investigated. VEGFR2 is one of the targets of Compound 1 and is important in angiogenesis. Whether soluble VEGFR2 is detectable via ELISA in plasma samples from cancer patients was investigated, as well as whether levels of the protein would change in response to treatment with Compound 1 or malate salt thereof.

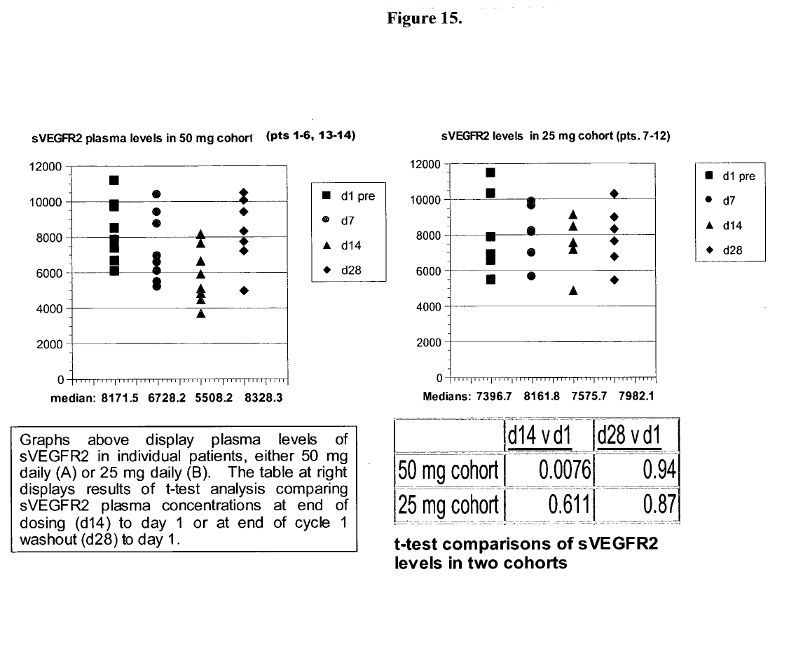

Intriguingly, levels of the plasma soluble form of VEGFR2 were observed to decrease in the vast majority of patients (greater than 90%) in Trials A, B and C at chronic time points (13 days or more) after the start of treatment with Compound 1 or malate salt thereof. Also, in Trial D, a dose-dependency of the sVEGFR2 decrease was seen, as changes were clearly observed in a cohort of patients in that trial receiving 50 mg daily doses of a malate salt of Compound 1, but not observed in a cohort of patients receiving 25 mg daily doses (FIG. 15). The difference between the dose cohorts was statistically significant as judged by t-test. Also, levels of sVEGFR2 typically increased to near baseline levels at the end of the 2-week drug rest period in patients from all 4 trials, thus exhibiting a pattern similar in timing but opposite in direction to that seen for VEGF and PLGF (Table 9). Table 9 displays results for sVEGFR2 in individual patients, and also includes results for PLGF where available. Also included in Table 9 is information on the types of cancers found in the patients.

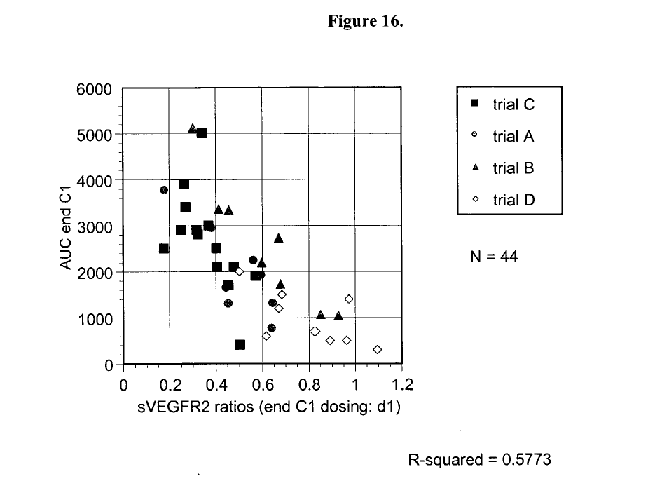

Further, data suggests that there exists some correlation between the extent of decrease in plasma sVEGFR2 and pharmacokinetics measurements of drug exposure in patients. This is demonstrated in FIG. 16, which shows a scatter graph plotting change in sVEGFR2 plasma level (ratio of level on last day of cycle 1 dosing to baseline level) against area under curve (AUC) drug exposure measurements (from last day of cycle 1 dosing). The graph is a composite of data from all 4 trials, and the R-squared value indicates there is some association between decrease in sVEGFR2 and drug exposure. Thus, soluble VEGFR2 is a novel marker of Compound 1 treatment and may be a marker of both drug exposure and biological activity of the compound.

Another potential biomarker of Compound 1 was identified first in an array-based screen of plasma samples, before and after Compound 1 treatment, from a patient in Trial B. The array screen utilized a commercially available antibody membrane array, which in principle allows for simultaneous measurement of 42 different human cytokines. Results of the screen indicated that levels of a protein called Monokine Induced by Interferon-gamma, or MIG, were significantly higher after treatment with Compound 1 than in baseline samples. This result was confirmed via an MIG ELISA assay on the same patient samples. Following confirmation, levels of MIG in plasma were assessed for a number of patients from Trial C. These results showed that MIG was induced more than 3-fold in 30-40% of the patients tested (data not shown).

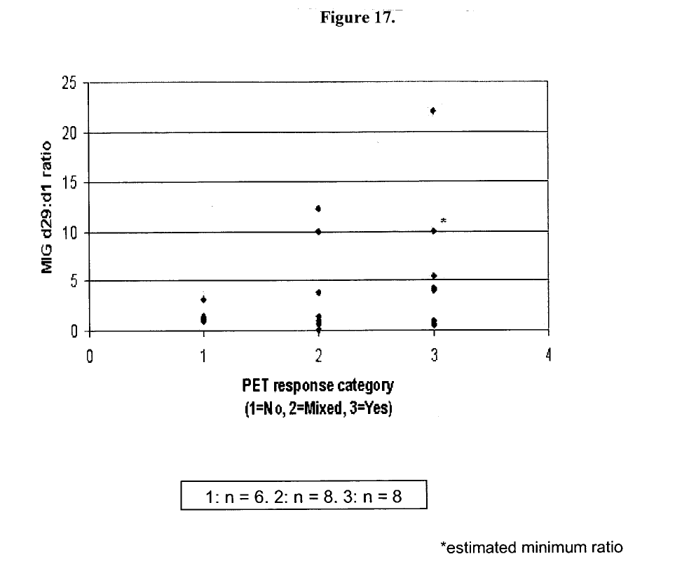

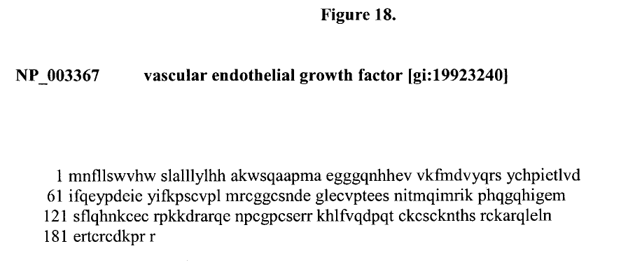

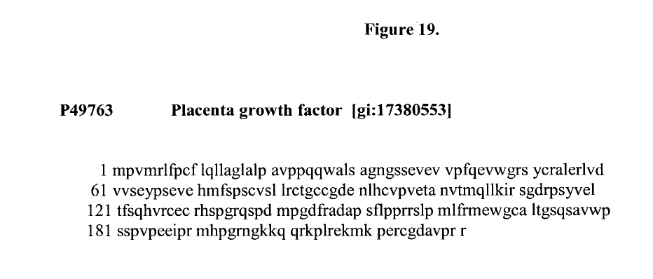

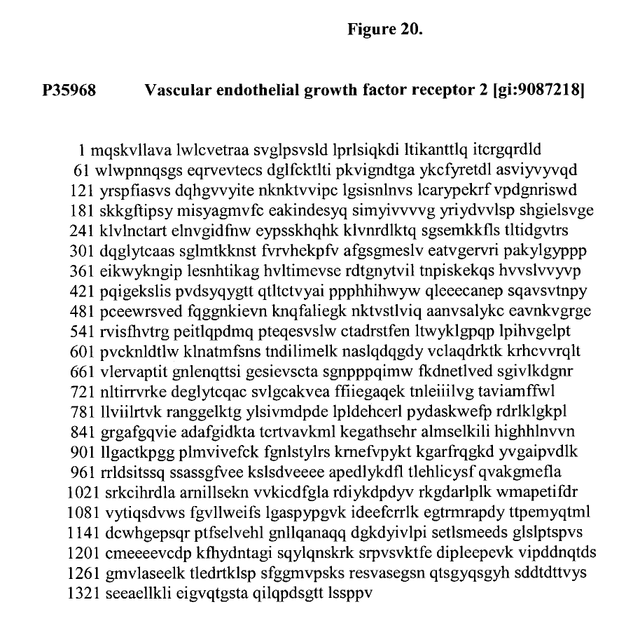

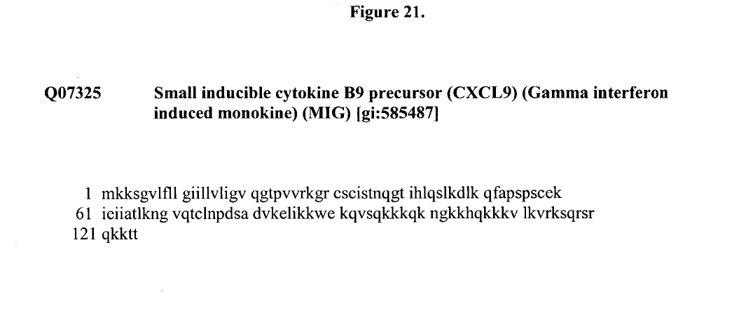

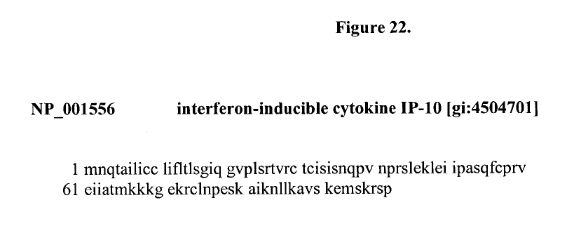

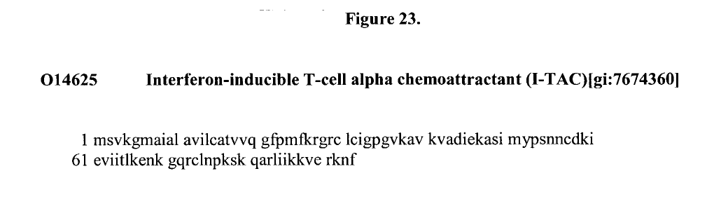

There is evidence of a correlation between increased MIG levels and a positive response in the functional imaging assay of ¹⁸FDG-PET (a feature of Trials C and D). This is illustrated in FIG. 17; those patients with at least a mixed response based on PET imaging tended to have higher folds of induction of secreted MIG protein. To further investigate the induction of MIG observed in patients, we have also measured the plasma levels of IP-10 and I-TAC before and after treatment with Compound 1 or malate salt thereof. IP-10 and I-TAC, like MIG, are regulated at the expression level by interferon-gamma, and both IP-10 and MIG have roles in chemoattraction of immune cells and exhibit angiostatic (anti-angiogenic) activity. Interestingly, evidence suggests that MIG and IP-10 are induced in tandem in 6 of 6 patients checked for both proteins while MIG and I-TAC are induced in tandem in 5 of 5 (Table 8). Similarly, all 3 proteins are induced in the 2 patients where all of the 3 were checked (Table 8). Table 10 indicates the types of cancer found in patients where MIG is induced. Thus, evidence indicates that MIG, IP-10 and I-TAC are novel biomarkers that are modulated in Compound 1 patients and are markers that correlate with an anti-tumor response as measured by PET imaging.

In summary, ELISA-based screening of plasma samples from Phase I clinical trials using Compound 1, or malate salt thereof, has yielded a set of circulating proteins that are novel surrogate markers for Compound 1 drug exposure and/or biological activity. Soluble VEGFR2 has been identified in plasma as a marker of drug exposure, while VEGF, PLGF, and VEGF/PLGF heterodimers have been frequently observed to increase in a majority of patients and appear to be correlates of biological activity and (to a lesser extent than sVEGFR2) drug exposure. MIG, IP-10 and I-TAC are additional biomarkers that appear to correlate with anti-tumor activity as measured by ¹⁸FDG-PET functional imaging.

### H. EXAMPLES

### Further Studies Using Compound 1

1. Further Studies Using Compound 1—Materials and Methods In Vivo Animal Studies

Female athymic-nu/nu mice (Charles River, Hollister, Calif.) were injected with Colo205 human colon cells (5×10⁶ cells) subcutaneously. The animals were treated with a single dose of either citrate vehicle or Compound 1 at 40 mg/kg when the tumors are approximately 350-400 mm3 in size. For biomarker studies, tumors were harvested at six and 24 hours post-treatment and snap frozen for RNA extraction.

Transcriptional Profiling Using Affymetrix DNA Arrays

RNA processing and hybridization protocols were carried out as recommended by Affymetrix, Inc. (Santa Clara, Calif.); protocols are available in the Genechip® Expression Analysis Technical Manual <www.affymetrix.com/support/technical/manual/expression_manual.affx>. In brief, total RNA from tumor samples was prepared using Nucleospin RNA II Kit in accordance with the manufacturer's recommendation (Clontech, Palo Alto, Calif.). RNA processing and hybridization protocols were carried out as recommended by Affymetrix, Inc. (Santa Clara, Calif.); protocols are available in the Genechip® Expression Analysis Technical Manual <www.affymetrix.com/support/technical/manual/expression_manual.affx>. In brief, double-stranded cDNA was synthesized from total RNA (8 μg) of tumor samples using Invitrogen Life Technologies SuperScript Choice system reagents (Carlsbad, Calif.). A T7-(dT)₂₄ oligomer was used to prime first-strand cDNA synthesis. Double-stranded cDNA product was generated and purified via phenol-chloroform extraction, then used as template for in vitro transcription (IVT) of cRNA. The IVT reaction was performed using BioArray High Yield RNA Transcript Labeling Kit (Affymetrix) according to manufacturer's protocol. The cRNA product was then purified with Qiagen RNeasy Mini Kit spin columns according to the manufacturer's protocol (Qiagen, Valencia, Calif.). Purified cRNA was quantitated, chemically fragmented, and hybridized overnight on Human Genome U95A Arrays. Hybridized arrays were washed and stained with phycoerythrin-conjugated streptavidin detection chemistry in an Affymetrix Fluidics'station. Images were scanned with a Hewlett-Packard GeneArray scanner. All techniques were performed on xenograft tissue samples according to the manufacturers' instructions.

Data Analysis of DNA Microarray

Data files were generated from scanned array images in the Affymetrix Microarray Suite Version 4.0 program. The two key parameters used in determining transcriptional changes are the Average Difference (AD) values, which serve as relative indicators of the expression level of transcripts represented on the arrays, and the Absolute Call (AC), which determines the presence or absence of each transcript. To enable comparison of all hybridization data, global scaling was applied by multiplying the output of each experiment by a scaling factor (SF) to make its average intensity equal to a user-defined Target Intensity (1500 for these experiments). For comparisons between different treatments from a single time point, the data were analyzed using Microsoft Access 97 software (Microsoft, Redmond, Wash.). To determine the fold change, the AD of the drug-treated samples was divided by the AD of the vehicle-treated samples. A data filtering step was carried out to identify transcripts with AC of “present” that showed a fold change ≧2.0 (increasing or decreasing).

Taqman Real-Time RT-PCR Assay

Primers and probes were designed using Primer Express 2.0 software (Applied Biosystems, Foster City, Calif.). All primers and probes were designed to hybridize to sequences represented by the Affymetrix probe set (see Affymetrix NetAffx website for detail). Taqman probes were labeled with reporter dye, 6-carboxy-fluorescein phosphoamidite (FAM), at the 5′ end and dye quencher, minor groove binder (MGB), at the 3′ end. Each 25-μl reaction consisted of 500 nm forward primer, 500 nm reverse primer, 100 nm of Taqman probe, cDNA (20 ng of total RNA from tumor samples), and 1× (final concentration) of Taqman® One-Step RT-PCR Master Mix Reagents Kit (Applied Biosystems). The reactions were performed in 96-well optical plates and analyzed using the ABI PRISM® 7700 Sequence Detection System (Applied Biosystems). Thermal cycler conditions used are as follows: 48° C. for 30 minutes, 95° C. for 10 minutes, 95° C. for 15 seconds followed by 60° C. for 1 minute for 40 cycles, and 25° C. for 2 minutes. 18S ribosomal gene's primers and probe pairs were purchased from Applied Biosystems and used according to manufacturer's recommendation as an endogenous control. All techniques were performed on the tissue samples according to the manufacturers' instructions.

Data Analysis of Taqman Assay

The Ct scores represent the cycle number at which fluorescence signal (ΔRₙ) crosses an arbitrary (user-defined) threshold. The Ct score for genes of interest for each sample were normalized against Ct score for the corresponding endogenous control gene (18S). Relative expression of specific transcripts in the drug-treated sample compared to vehicle-treated sample was determined by the following calculation, as described in the Applied Biosytems users bulletin on Relative Quantitation of Gene Expression:

Relative Expression=2^{-ΔΔCt},

where ΔΔCt=(Ct_{target}−Ct_{18s control})_{drug treatment}−(Ct_{target}−Ct_{18s control})_{vehicle treatment}.

2. Further Studies Using Compound 1—Results

Microarrays and RT-PCR Analysis

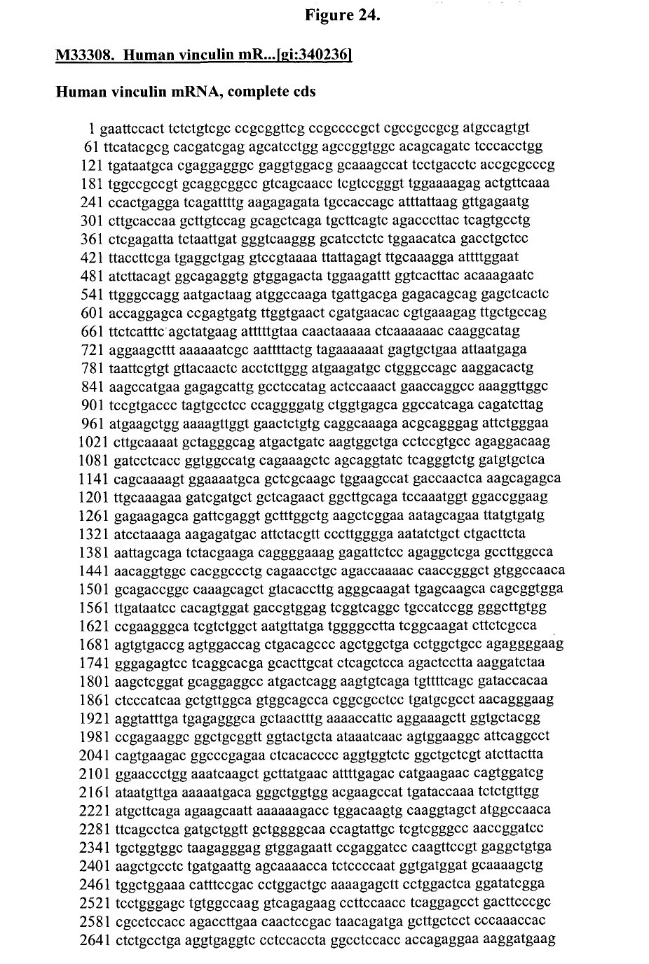

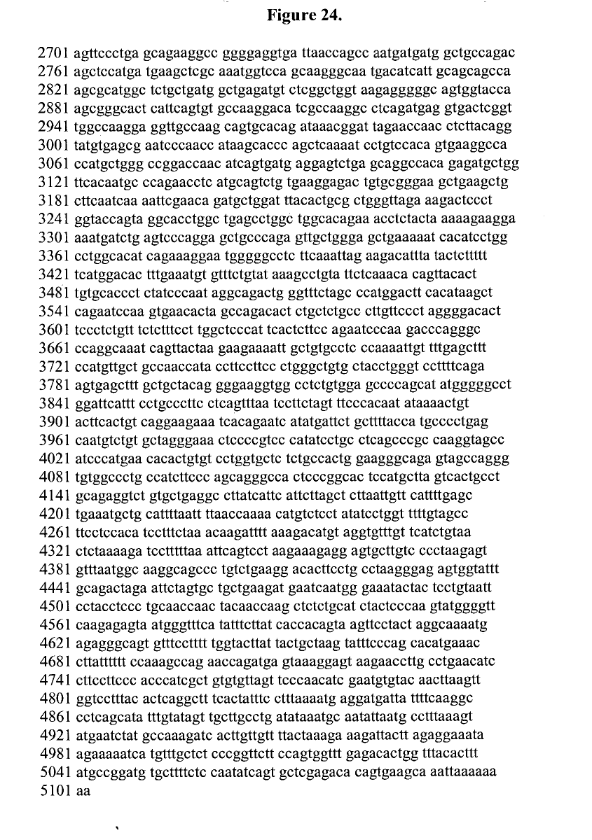

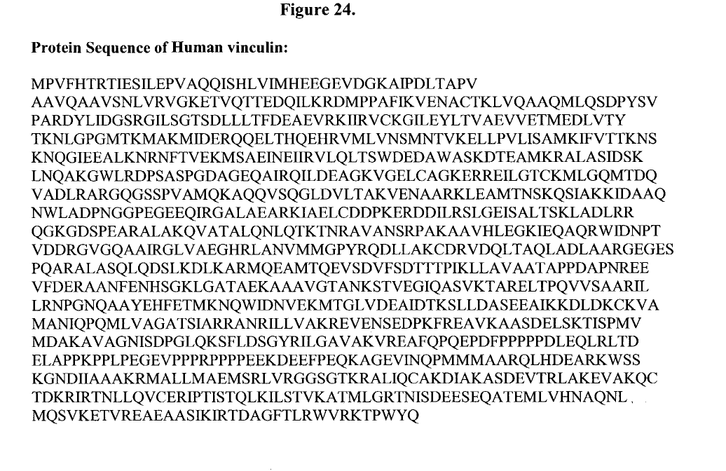

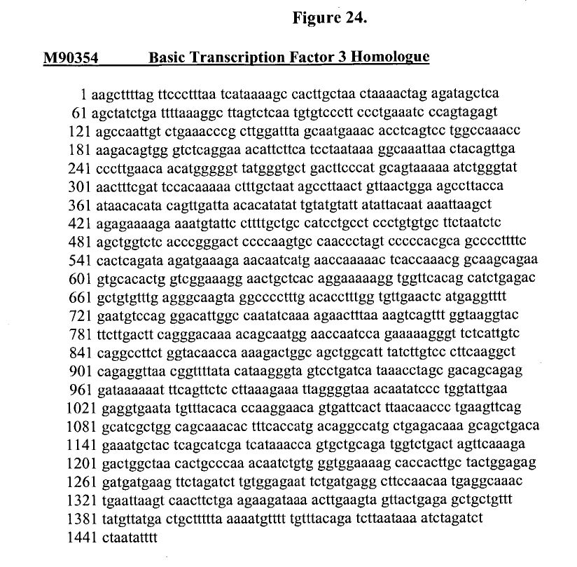

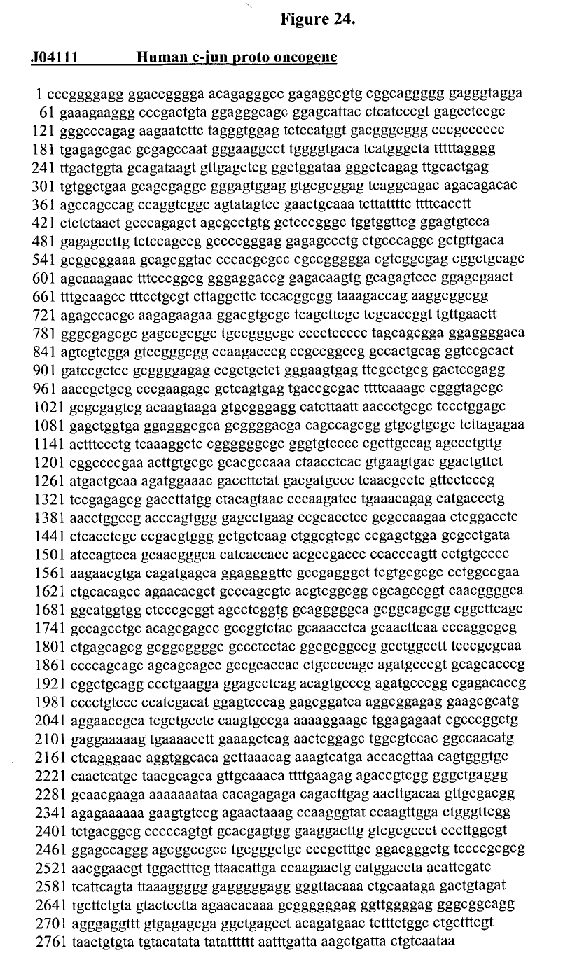

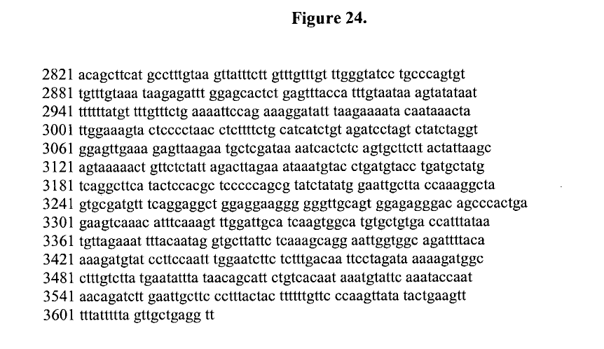

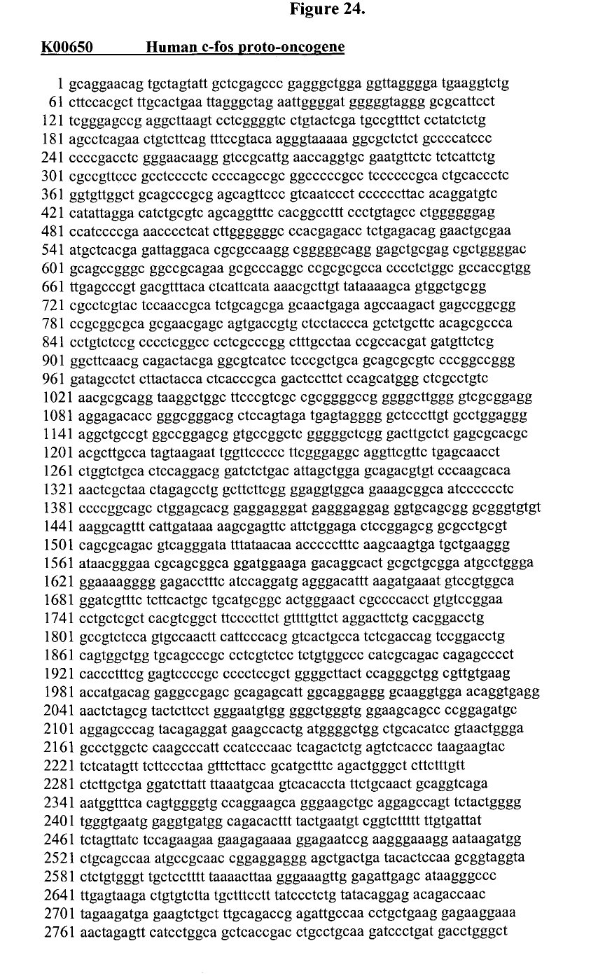

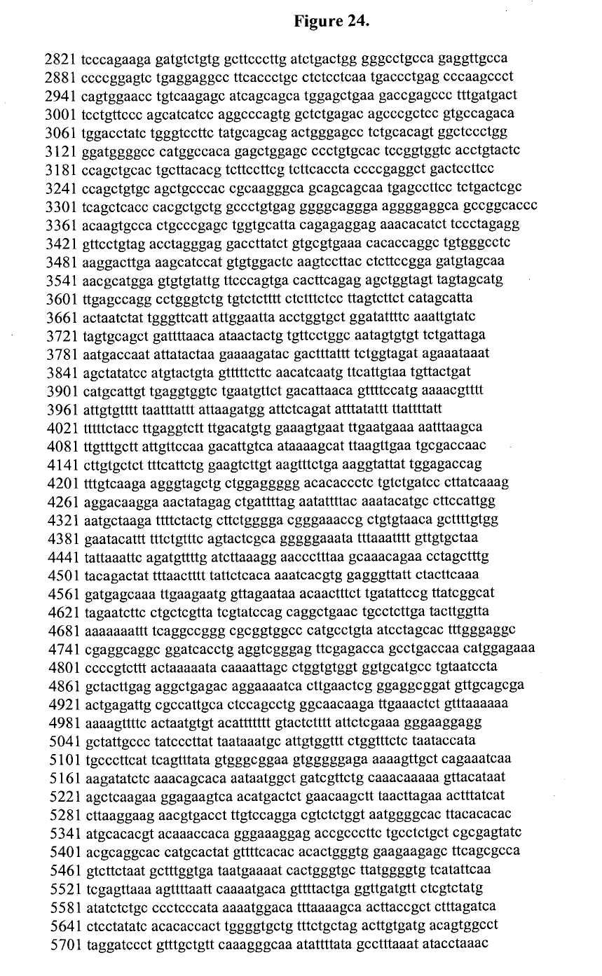

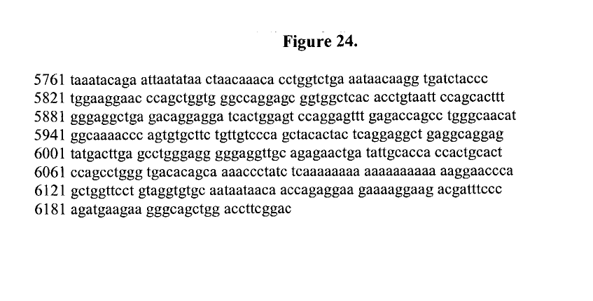

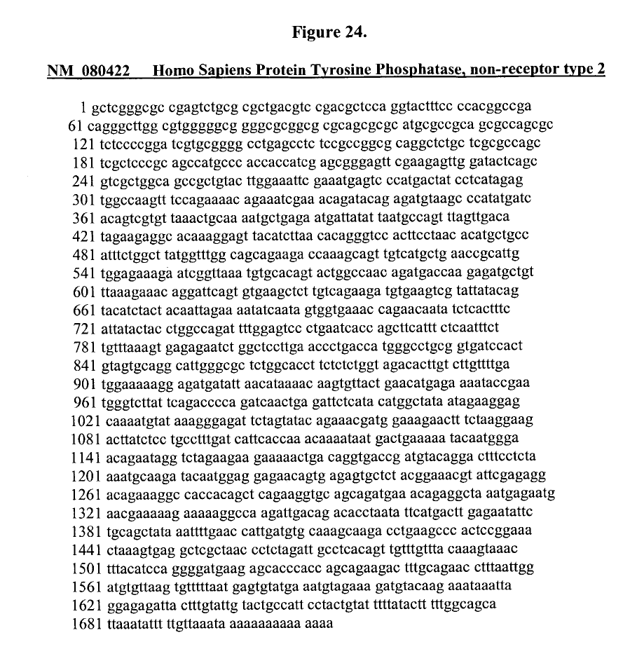

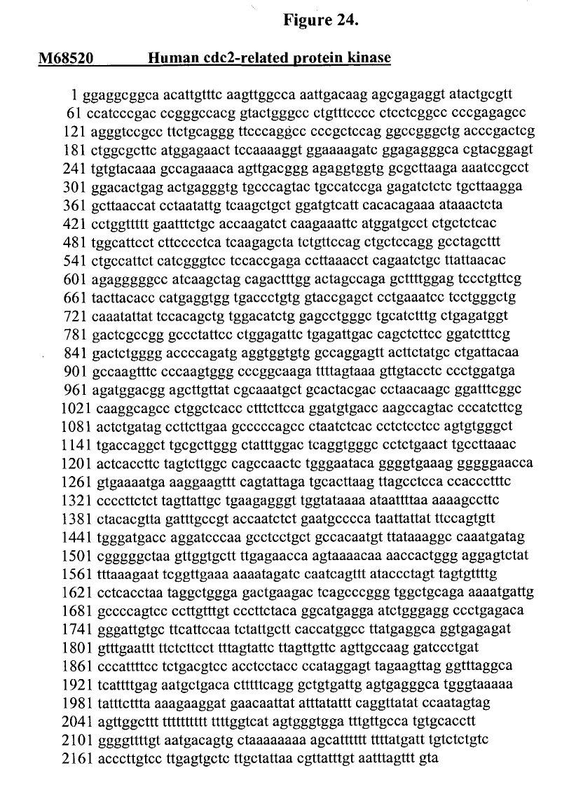

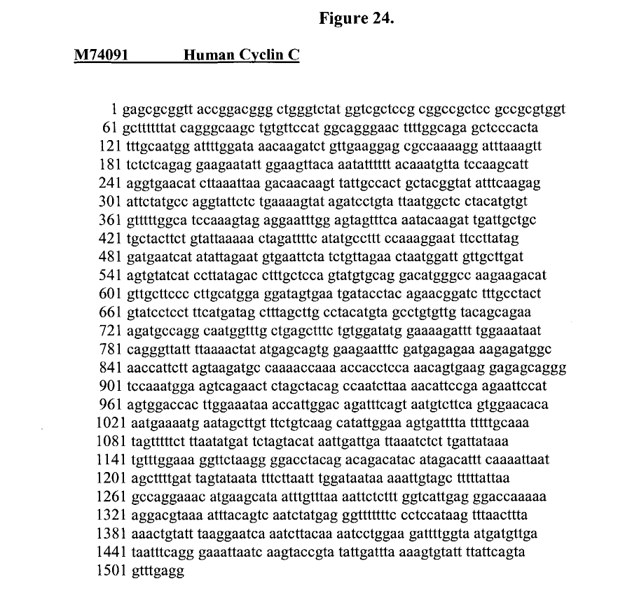

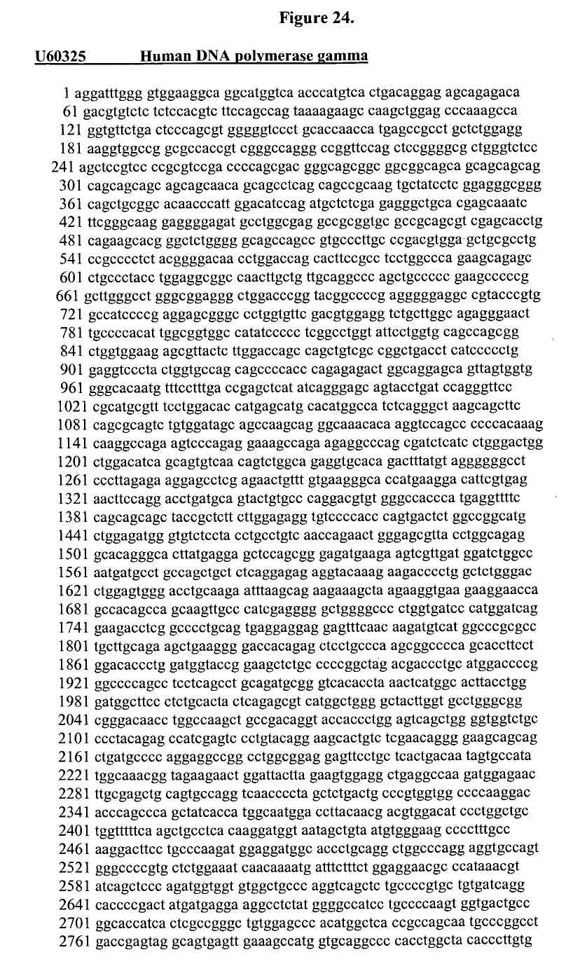

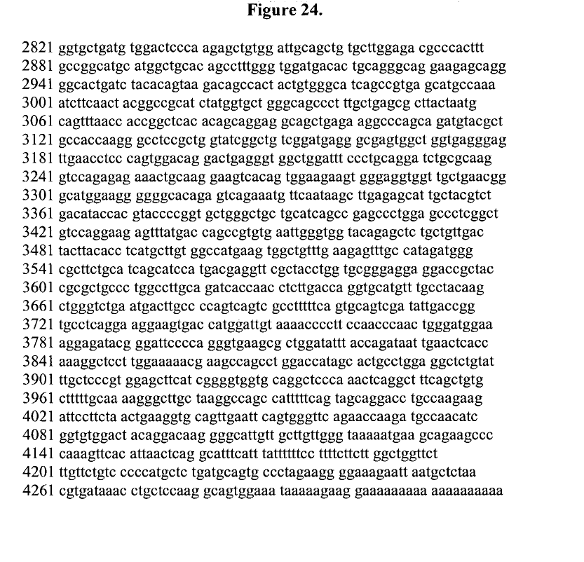

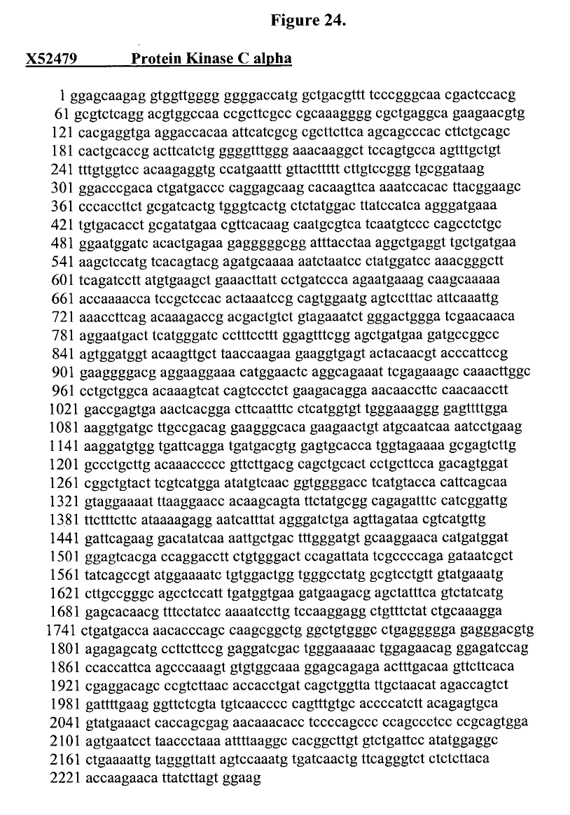

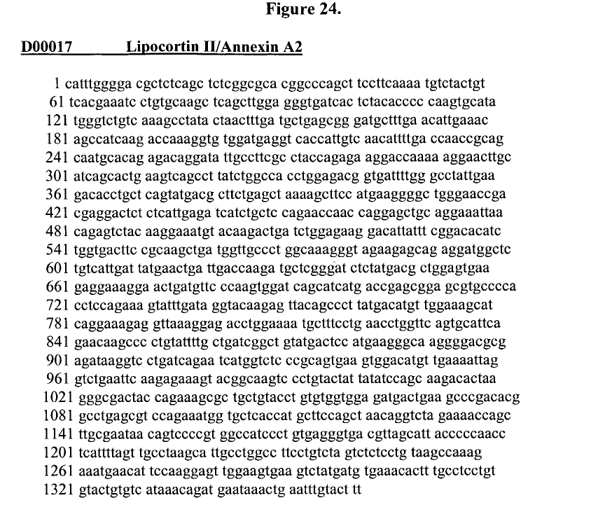

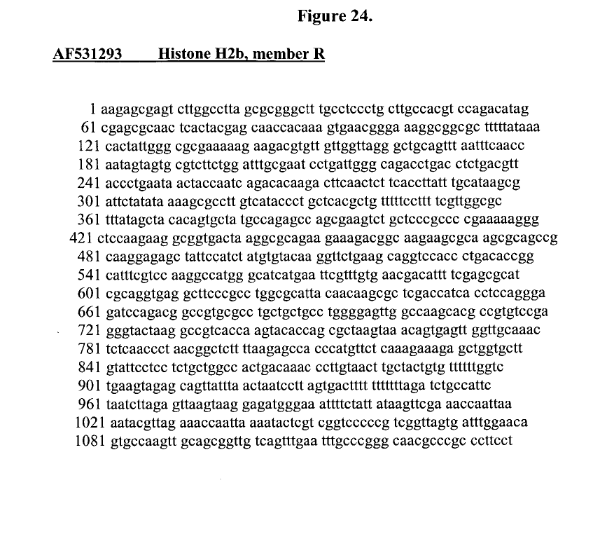

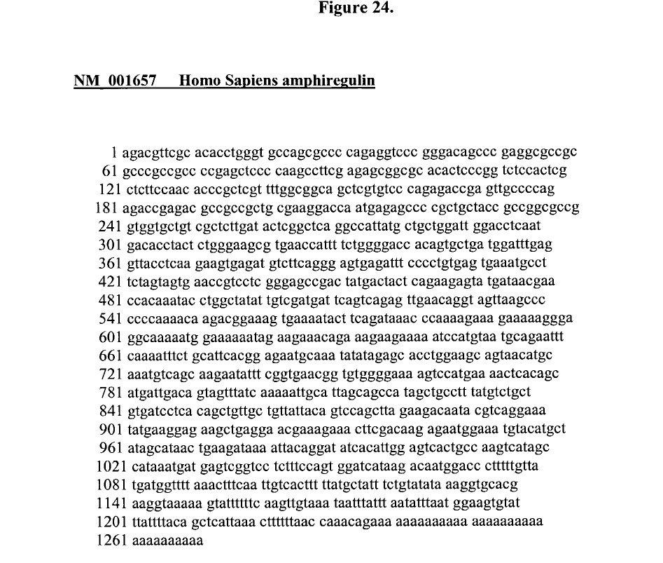

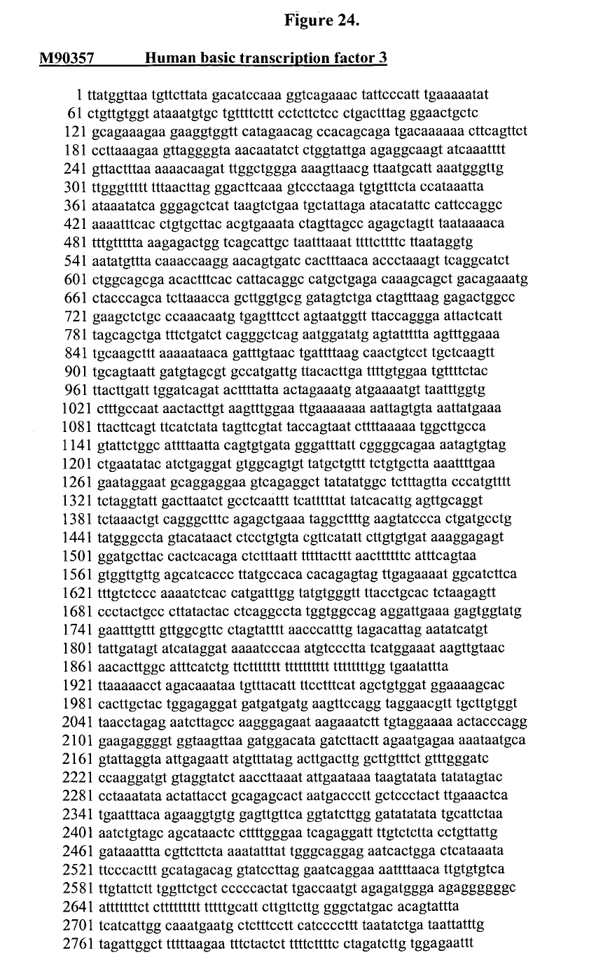

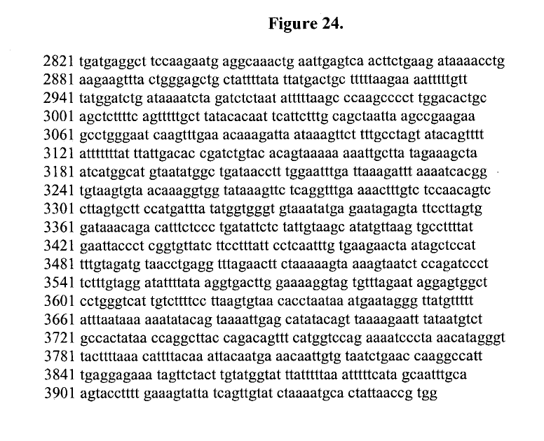

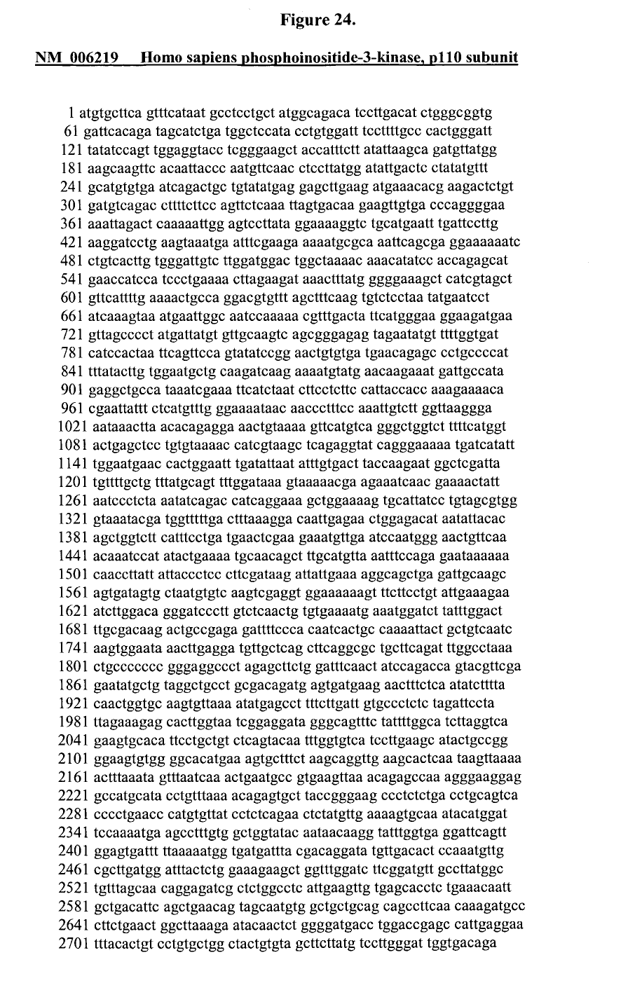

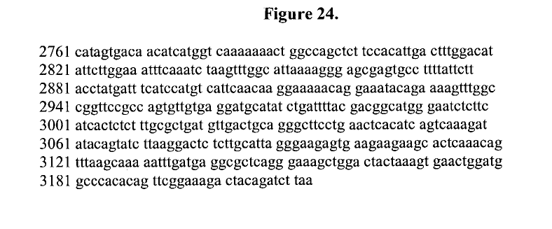

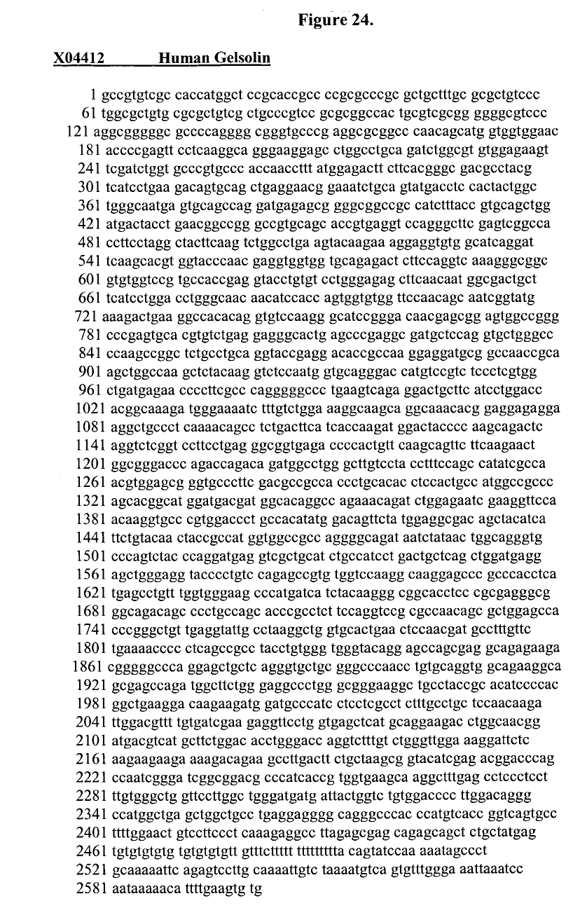

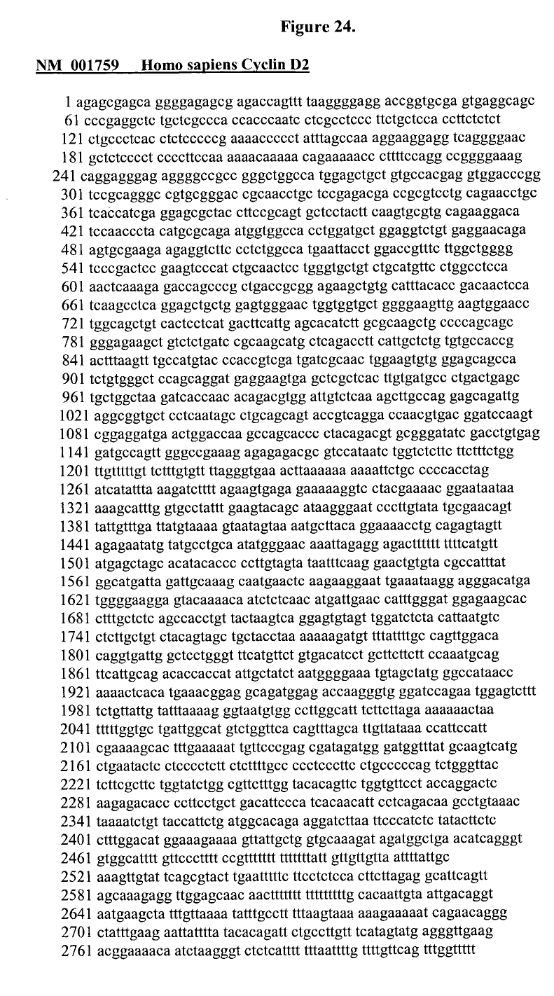

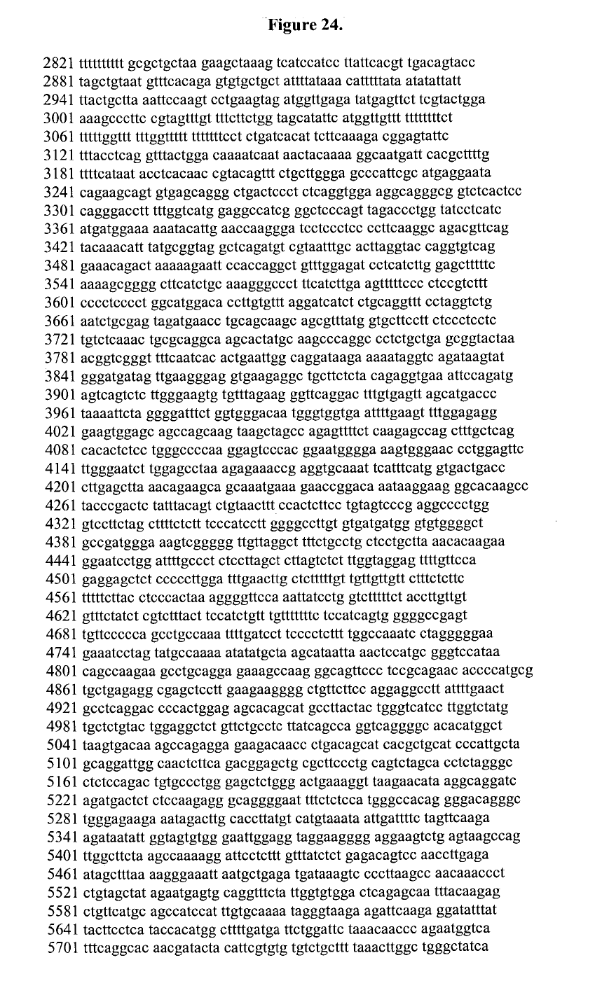

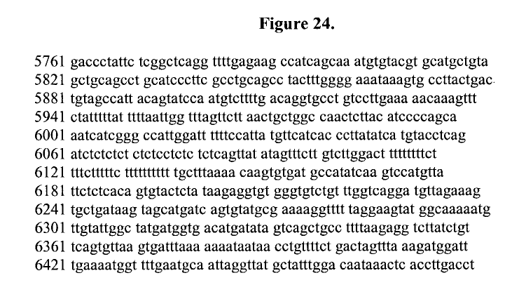

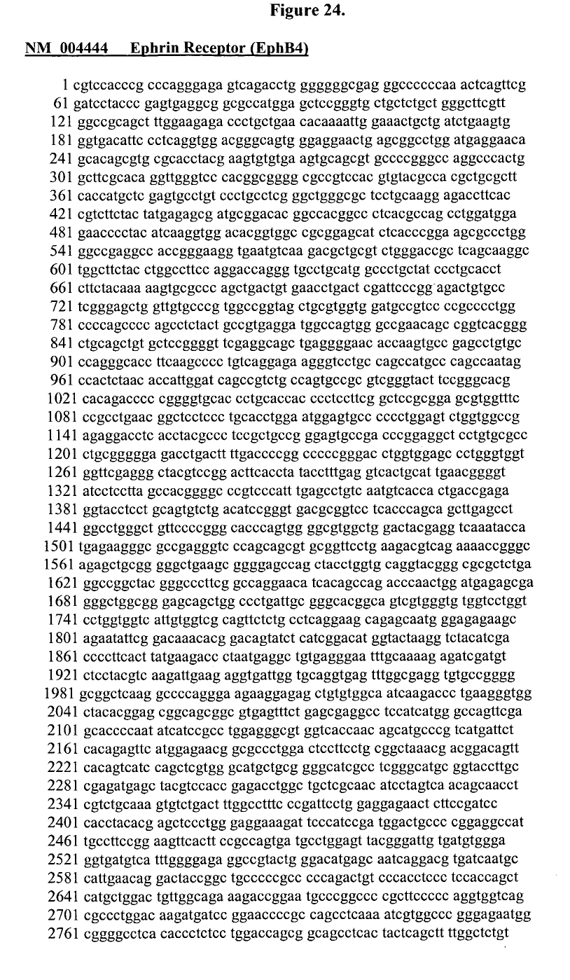

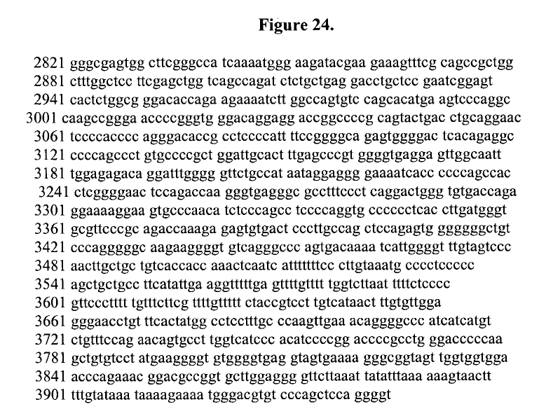

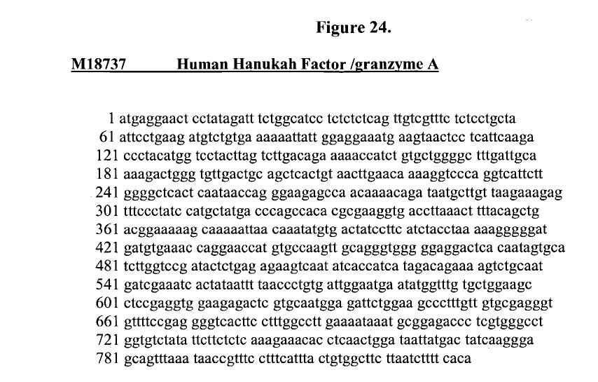

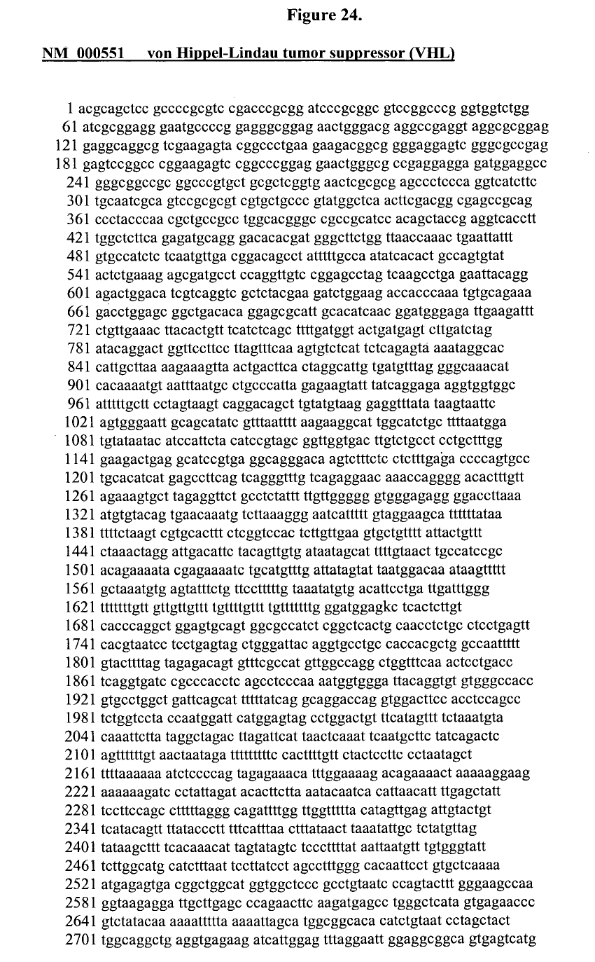

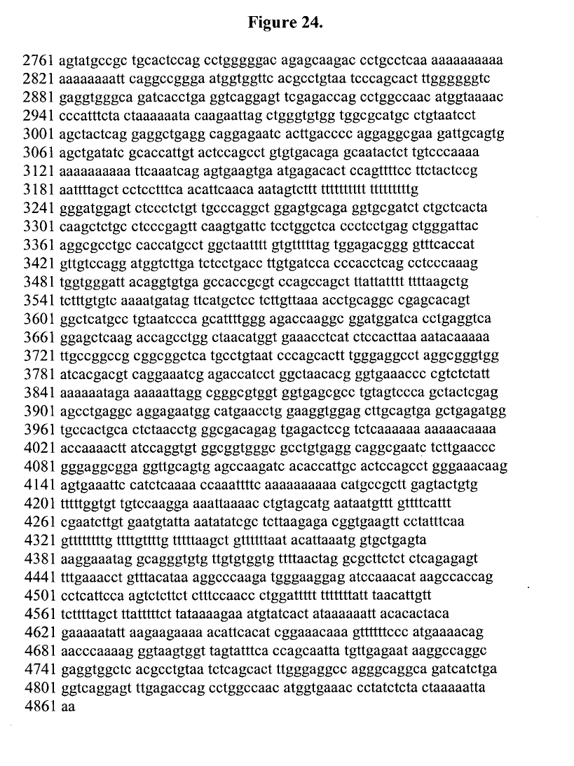

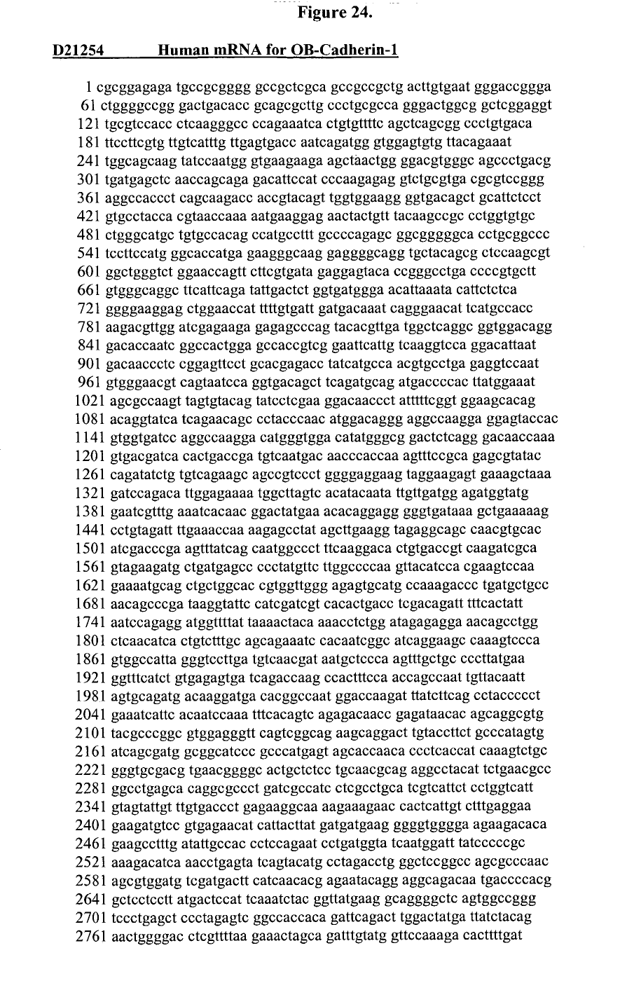

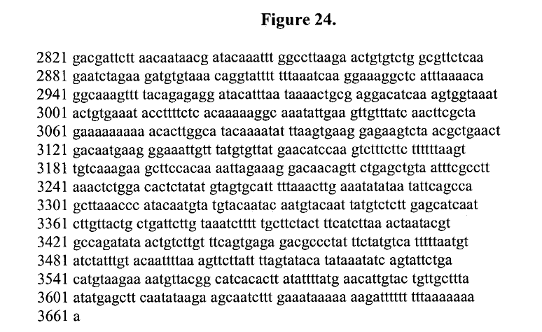

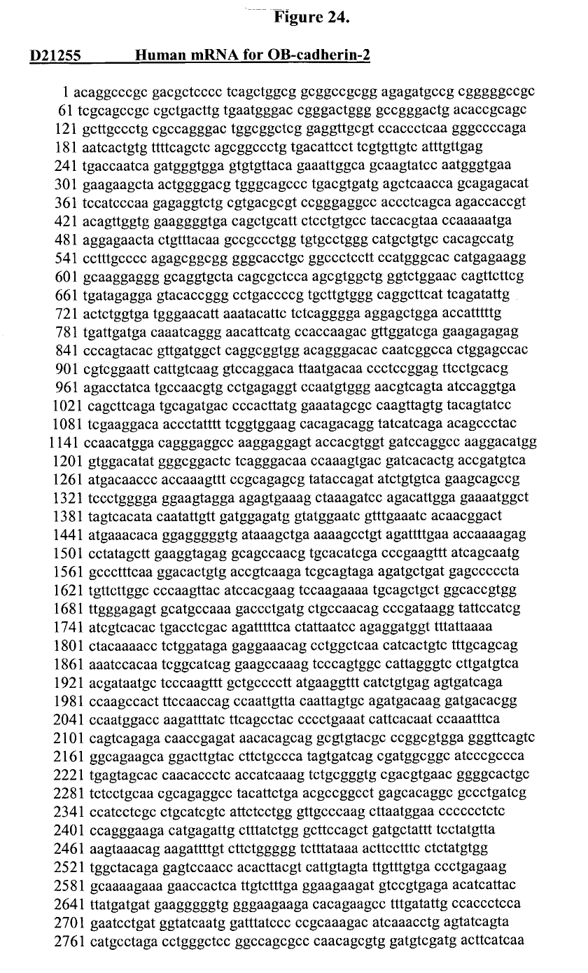

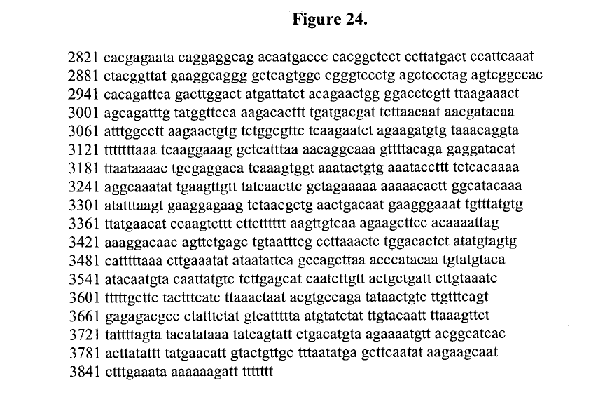

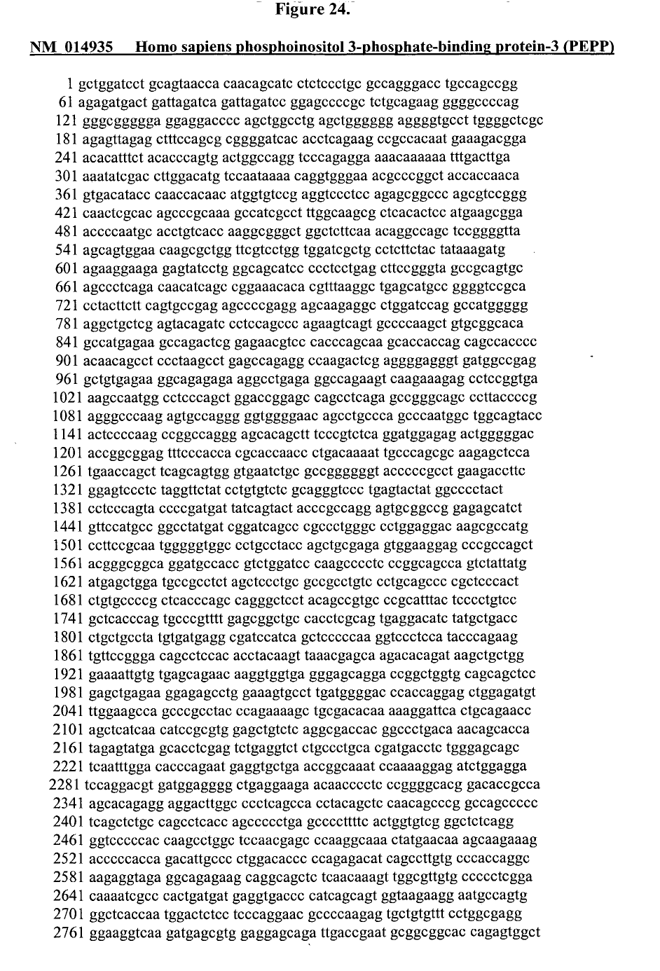

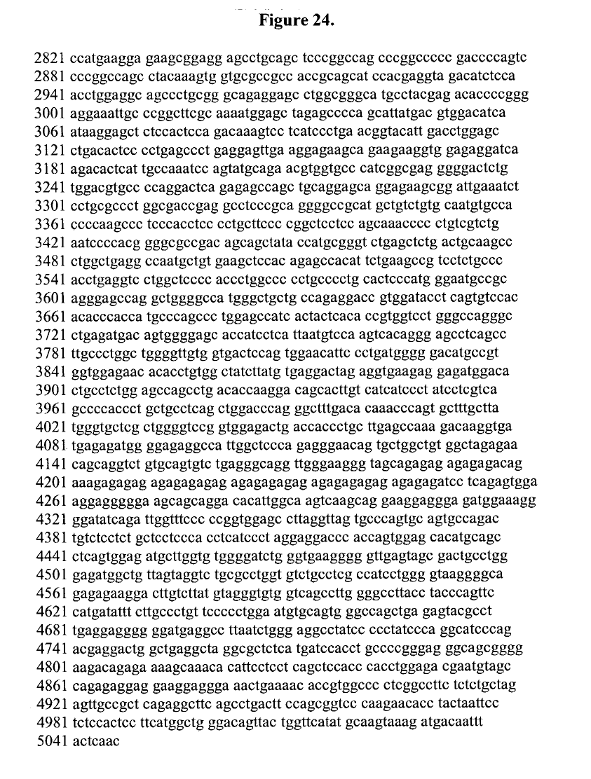

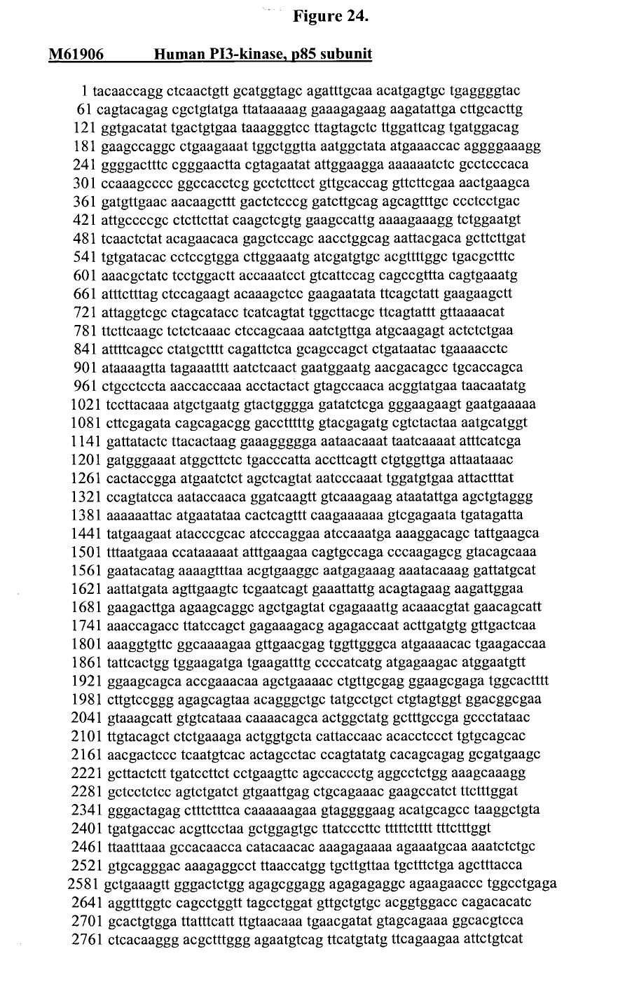

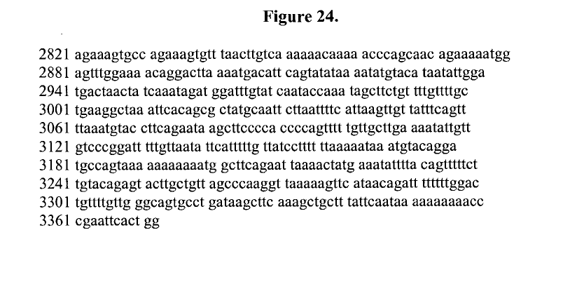

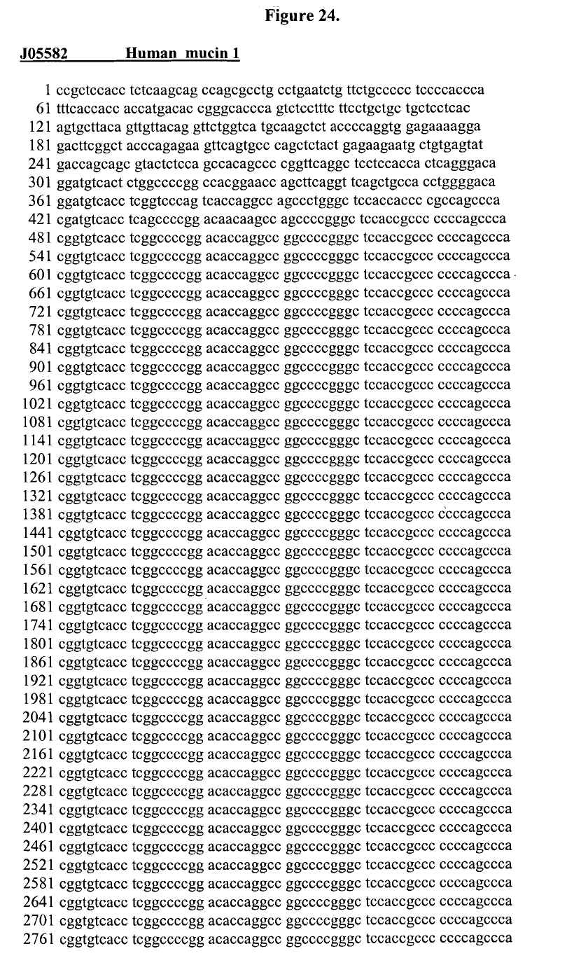

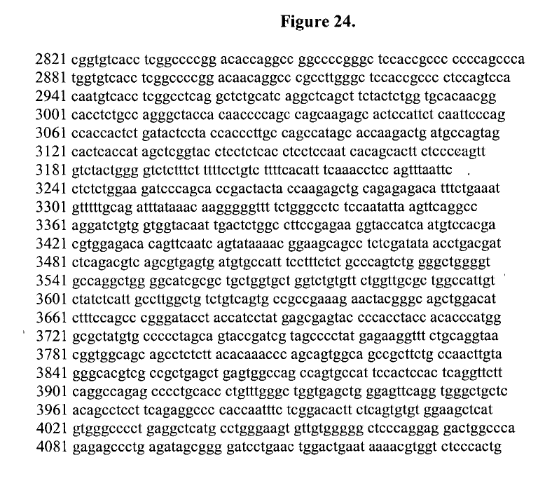

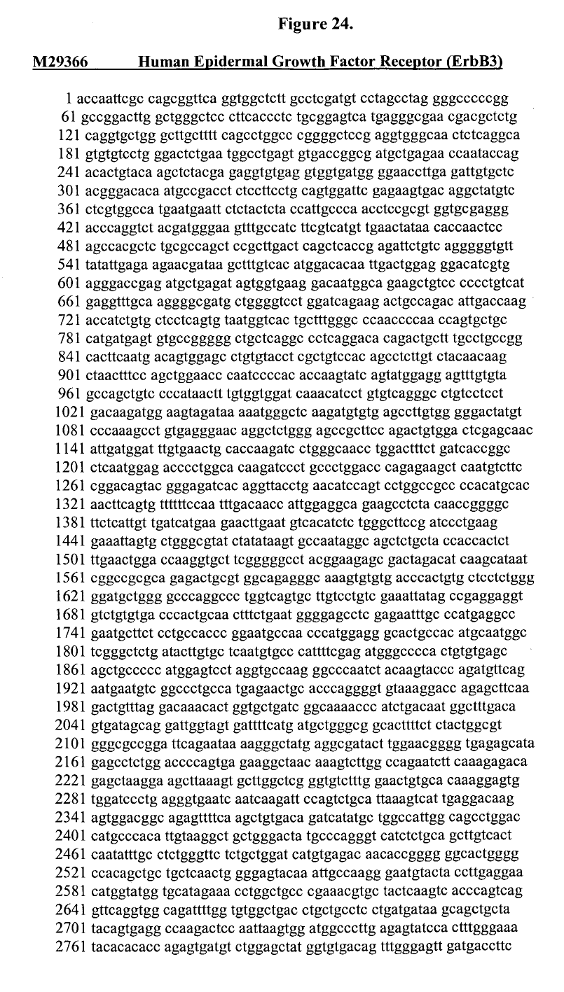

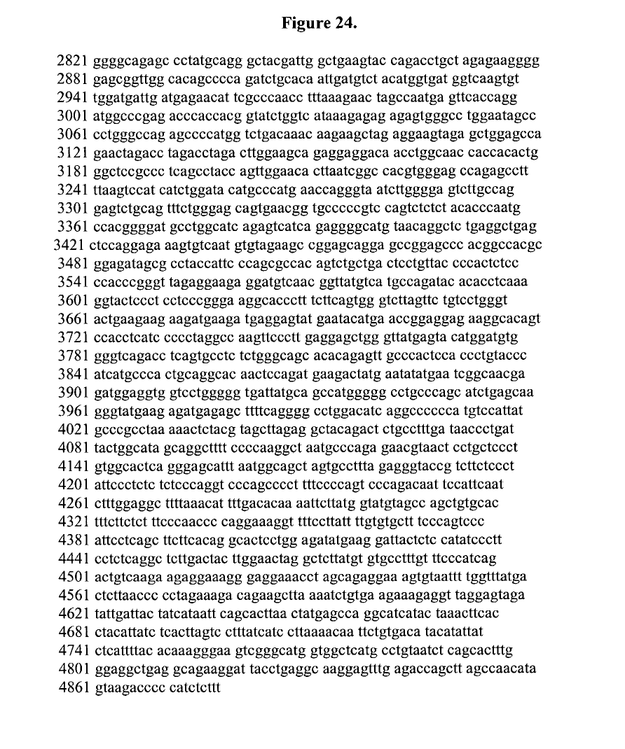

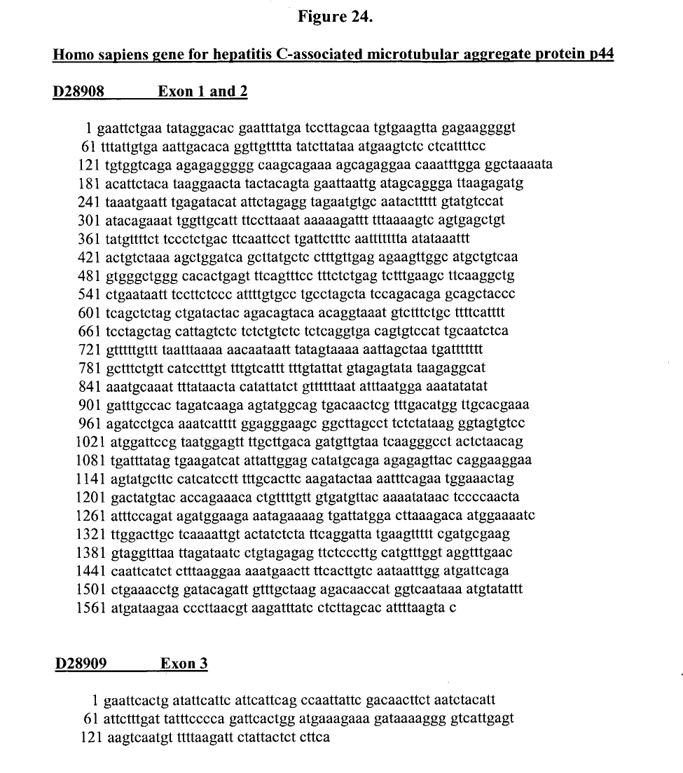

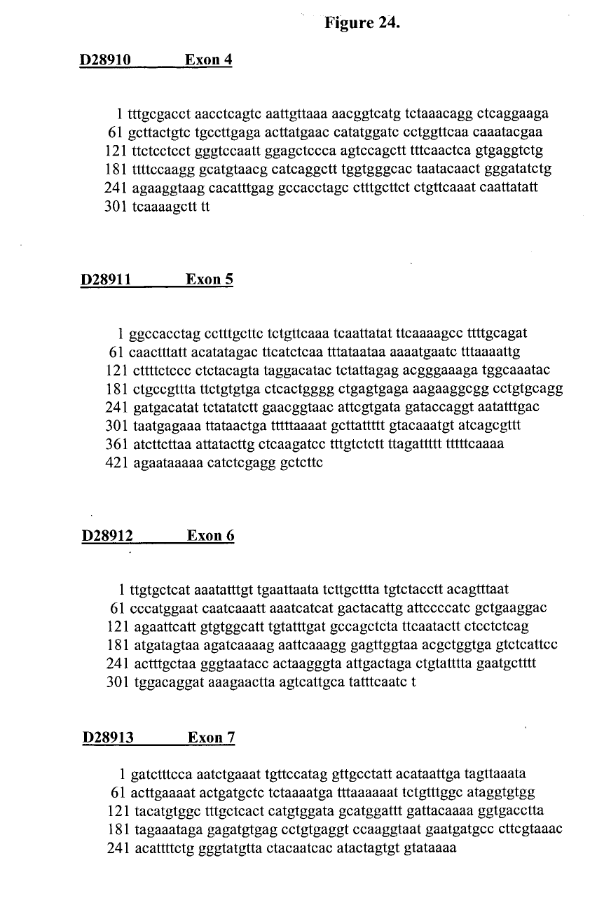

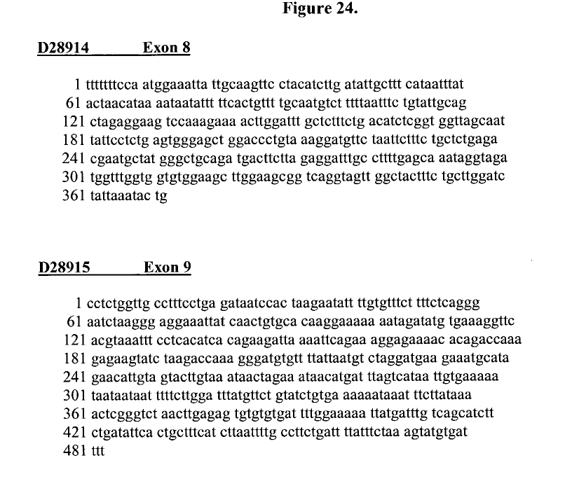

To identify biomarker(s), samples of tissue from the tumors were taken before and after the first dose of Compound 1. An Affymetrix GeneChip analysis of the RNA transcripts present in xenograft tissue before and after exposure to Compound 1 indicated that the levels of 28 transcripts increased and/or decreased after exposure to Compound 1 (see Table 11A and 11B). Thus, the following 26 proteins/trasnscripts were identified as biomarkers for a compound that inhibits tyrosine kinase, such as Compound 1: basic transcription factor 3 homologue, human c-jun proto-oncogene, human c-fos proto-oncogen, tyrosine phosphatase non-receptor type 2, cdc2 related protein kinase, cyclin C, DNA polymerase gamma, protein kinase C alpha, lipocortin II/annexin A2, histone H2B member R, amphiregulin, ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, vinculin, basic transcription factor 3, phosphoinositol 3-kinase p110 subunit, gelsolin and cyclin D2. See FIG. 24 for sequences for these biomarkers.

To validate the Affymetrix GeneChip results, a subset of 11 of these 26 transcripts was chosen for quantitative RT-PCR analysis. These 11 transcripts were chosen based on potential roles of encoded proteins. Table 13 describes the forward and reverse primers that that were designed and used in the RT-PCR experiments. The results of the quantitative RT-PCR analysis for these 11 transcripts are shown in Table 12. The RT-PCR analysis confirms the findings with the Affymetrix GeneChip analysis for these 11 transcripts.

### I. EXAMPLES

### Additional Studies Using Compound 1

1. Additional Studies Using Compound 1—Materials and Methods

Human Umbilical Vein Endothelial Cells (HUVECs)

HUVECs were obtained from Clonetics (San Diego, Calif. catalog#CC-2517) and were maintained in EGM media (Clonetics, catalog#CC-3121) containing EGM BulletKit (Clonetics, catalog#CC-4133: 2% Fetal Bovine Serum, 0.1% Epidermal Growth Factor, 0.1% Hydrocortisone, 0.1% Gentamicin Sulfate Amphotericin B, 0.4% Bovine Brain Extract). Cells were propagated at 37° C. in a humidifed atmosphere of 5% CO₂ using standard cell culture techniques. Cells were plated in 10-cm tissue culture plates at 8.5×10⁵ cells/ml. After 6 hours the cells were quiesced by serum starvation overnight in starvation medium (EBM containing 0.5% FBS). DMSO (Sigma Chemicals, St. Louis, Mo. #D2650) or Compound 1 (to a final concentration of 10 nM, 100 nM, and 1 μM) were added to cells. After 2 hours of exposure to Compound 1 or DMSO, VEGF₁₆₅ (R&D Systems, Minneapolis, Minn.; catalog#293VE050) was added to a final concentration of 100 ng/ml; no VEGF was added to samples that are subsequently referred to as the “baseline” samples. After a 10-min, 8 hr, 24 hr and 48 h VEGF stimulation the conditioned medium was filtered through 0.45 μM syringe filter from Pall Gelman Laboratory (Ann Arbor, Mich. catalog#4560) and immediately frozen on dry ice. Conditioned media was stored at −70° C. until subsequent analysis.

Analysis of Conditioned Media by 2D Gel Electrophoresis

Thawed conditioned media samples were precipitated with three volumes of acetone for 2 hours at −20° C., then centrifuged at 13000 RPM for 15 minutes. Pellets were washed with the 2D Clean-Up Kit (Amersham, Cat. #80-6484-51) as per protocol, air dried for three minutes, then resuspended in 8M urea (Amersham), 100 mM dithiothreitol (Fisher), 4% CHAPS (3[(cholamidopropyl)dimethylammonio]propanesulfonate from Calbiochem), and placed in a thermomixer (Eppindorf) at 600 RPM and 25° C. for 2 hours. Protein was quantitated with Bio-Rad Protein Assay (cat#500-0006) using the microassay for cuvettes protocol.

Samples were diluted to 0.3 μg/μl with IEF Buffer containing 1% IPG Buffer pH 3-10 (Amersham). Eighteen centimeter IPG strips pH 3-10 (Amersham) were rehydrated with 120 μg sample (400 μL) under Drystrip Cover Fluid (Amersham) on the IPGphor (Amersham) at 20° C. for 18 hours. Strips were focused with the following program: 200 volts for 1 hour, ramped from 200 volts to 1000 volts over two hours, held at 1000 volts for 1 hour, ramped from 1000 volts to 8000 volts over 6 hours, then held at 8000 volts for 10 hours. Polyacrylamide gels were hand cast in the Hoeffer DALT multi-gel casting chamber (Amersham) at 10% Acrylamide (Bio-Rad 40% Acrylamide Solution), 2.67% piperazine diacrylamide (Bio-Rad), 0.375 M tris, pH 8.8 (Bio-Rad), 0.075% ammonium persulfate (Bio-Rad), and 0.075% TEMED (N,N,N′, N′-tetramethylethylenediamine). Gels were over-layed with water-saturated butanol (Fisher), and left to polymerize at room temperature overnight.

Focused strips were equilibrated for ten minutes with gentle shaking in 10 milliliters Equilibration Buffer: 6 M Urea (Fisher), 50 mM tris-HCl pH 8.8 (Fisher), 30% glycerol (Fisher), 2% SDS (Fisher) with 1% dithiothreitol followed by ten minutes in Equilibration Buffer with 4% iodoacetamide.

The equilibrated strips were loaded onto the gel surfaces and sealed with hot agarose overlay solution containing 0.5% agarose in 50 mM tris-HCl pH 6.8, 2% SDS.

Gels were run in the Hoeffer DALT tank (Amersham) in 25 mM tris (Fisher), 192 mM glycine (Fisher), and 0.1% SDS overnight at 100 volts and 8° C.

The gels were washed three times in 500 mL Fixative (10% methanol and 7% glacial acetic acid ) for one hour each with gentle agitiation. The gels were then stained overnight in 500 mL Sypro Ruby Protein Gel Stain (Molecular Probes). Gels were again washed three times in 500 mL fixative for an hour each with gentle agitiation. Images were obtained on the Fluor S MultiImager (Bio-Rad) using transilluminated ultraviolet light for 45 seconds with the 520LP emission filter. Image analysis was done using PDQuest version 7.0.1 (Bio-Rad).

2D Gel Spot Cutting

The automated gel cutting was performed using the Proteome Works Spot Cutter (BioRad, Hercules, Calif.) and PDQUEST (v.7.0.1) software. Three sets of 2D gels were cut (Table 14). Based on the gel imaging analysis, the same spots of all three gels were combined in the same well of a 96-well plate.

Protein In-Gel Digestion

The automated digestion was performed using Investigator ProGest Digestion Station (Genomic Solutions). The sample plate (96-well pink plate) was placed onto the reaction block. A white sample collection plate was placed onto the collection block. The method used, Ruby48proGestv1, was based on the software ProGest Method Editor (v.1.1.0.29). Then the samples were digested automatically with trypsin (0.19 ag/well) at 37° C. for overnight.

MALDI-TOF-MS Analysis

After in-gel digestion, the digest was concentrated and desalted by using C18 reversed phase Ziptip (Millipore, Bedford, Mass.). Bound peptides were eluted with 4 μL matrix solution (a-cyano-4-hydroxycinnamic acid in acetonitrile/0.1%TFA 1:1 v/v).

1 μL eluted solution was spotted onto the MALDI target. Peptide mass mapping was performed on an ABI Voyager STR matrix-assisted laser desorption/ionization (MALDI) time-of-flight mass spectrometer (Applied Biosystems, Framingham, Mass.). The acceleration voltage was 20 kv, the grid voltage was 14 kv, the extraction delay time was 300 nsecexternal calibration during mass spectrometry data acquisition was used. The acquired peptide mass mapping spectra was processed and analyzed by Data Explorer software (Version 4.0.0.0.). The internal calibration was performed by using trypsin autolysis peptide mass 842.5099 and 2211.1046.

MALDI-MS/MS Analysis

The MALDI-MS/MS analysis was performed using API Qstar Pulsar equipped with oMALDI Source (PE Sciex). The curtain gas was 25, the declustering potential was 45, the focusing potential was set from range 220 to 250 V various by samples. CAD gas was 7 and collision energy was at 35 to 100 depending on samples. The ion energy was set at 1 kV. Data acquisition and processing was done using Analyst QS and oMALDI Server (v. 2.2) softwares. The biomaker identification was obtained with MASCOT database search using MS/MS spectra. The publically accessible link to the “MASCOT” tool for protein identification using peptide data is: <www.matrixscience.com/cgi/index.pl?page=/search_form_select.html>.

ELISA Analysis

Reagents for human pro-Matrix Metalloproteinase 1 (pro-MMP-1) ELISA kits were obtained from R&D Systems, Inc. (Minneapolis, Minn.; catalog #DMP100). ELISAs were performed on conditioned media samples according to the manufacturers' instructions. The optical density of each well was determined using a universal microplate spectrophotometer (μQuant) from Bio-Tek Instruments, Inc. (Winooski, Vt.). KC-4 software from Bio-Tek Instruments, Inc. was used to extrapolate cytokine concentrations from the standard curves.

2. Additional Studies Using Compound 1—Results

2D Gel Analysis of Conditioned Media from VEGF +/− Compound 1 Treated HUVECs.

Conditioned media isolated from HUVECs pre-treated with vehicle (DMSO) or Compound 1 (1 uM) and subsequently stimulated with VEGF for 24 and 48 hours or baseline, untreated samples were analyzed by 2D gel analysis (see Materials and Methods). This analysis identified 1 spot (#1202) whose abundance consistently increased with addition of VEGF in two separate gel runs and appeared to decreased with Compound 1 pre-treatment, although not consistently using this technology (Table 15). These spots were excised and underwent MALDI and MALDI-MS/MS analysis for subsequent protein identification.

Identification of Interstitial Collagenase Precursor/Pro-MMP1 by Database Search Based on Peptide Mass Fingerprint Spectra.

Peptide mass fingerprint data sets were analyzed by searching SwissProt protein database with ProteinProspector MS-Fit (Version 3.2.1). The searches were set with the following parameters, Human Mouse (Species), 1-66 kDa (molecular weight range), trpysin used for digest, maximum one missed cleavage, mass tolerance 50 ppm. Methionine was set as modified by oxidation and cysteine was set as modified by carbamidomethylation. Peptides were considered with hydrogen at N terminus and free acid at C terminus. The peptide masses were monoisotopic. The database search result was significant if the protein was ranked as the first hit and the sequence coverage was more than 30%, in addition a MOWSE score higher than 1e+003 (MS-Fit) was required. As summarized in Table 16 and Table 17, Spot 1202 was definitively identified as interstitial collagenase precusor (pro-MMP1).

ELISA Analysis of Pro-MMP1 Levels in HUVEC Conditioned Media

Because the quantitation of pro-MMP1levels in 2D gels is only semi-quantitative (and therefore less consistent), the levels of pro-MMP-1 in HUVEC conditioned media were also assayed using a quantitative ELISA assay. The ELISA analysis indicated that levels of pro-MMP1 increase quantitatively when HUVEC cells are treated with VEGF and are decreased with pre-incubation of Compound 1 at 10 nM, 100 nM or 1 uM concentrations (Table 18).

Pro-MMP1 Levels in Plasma from Compound 1 Treated Patients in Study B

Pro-MMP1 levels in the plasma of Study B patients after treatment with Compound 1 (day 1 pre-treatment, day 1 24 hr post-treatment, day 13 pre-treatment, day 13 12 hr post-treatment, and day 13 24 hr post-treatment) was analyzed. The results (see Table 19) demonstrate that pro-MMP1 levels increased in the plasma of patients after they received Compound 1.

### J. EXAMPLES

### More Studies Using Compound 1

1. More Studies Using Compound 1—Materials and Methods

Plasma Samples

All clinical plasma samples were harvested and handled in accordance with full Institutional Review Board-approved protocol, and study participants had signed the appropriate informed consent prior to any study related procedures. Plasma was separated from blood samples collected into Vacutainer tubes containing sodium heparin and shipped frozen to the SUGEN site.

Plasma samples were then thawed and centrifuged to remove particulate matter (10 min @ 5000×g). The resulting supernatants were collected and split into aliquots and were re-frozen at −80° C. Prior to assay, samples were thawed, Immunoglobulin Inhibiting Reagent (IIR, Bioreclamation Inc) was added to a final concentration 0.25 mg/mL, and Tween 20 was added to final concentration of 0.1%.

Antibody Chip Microarray Manufacture

Glass slides were cleaned and derivatized with 3-cyanopropyltriethoxysilane. The slides were equipped with a Teflon mask, which divided the slide into sixteen 0.65 cm diameter wells or circular analysis sites called subarrays. Printing was accomplished with a Perkin-Elmer Spotarray Enterprise non-contact arrayer equipped with piezoelectric tips, which dispense a droplet (˜350 pL) for each microarray spot. Antibodies were applied at a concentration of 0.5 mg/mL at defined positions. Each chip was printed with sixteen copies of one type of array, either Array 1.1 or Array 2.1 (see below). Both arrays consist of capture antibodies against different analytes and are defined by the antibody set contained. Analytes measured using both arrays are listed in Table 20.

| | |
|---|---|
| Analyte | Name |

Array 1.1 detector set.

| | |
|---|---|
| ANG | Angiogenin |
| BLC (BCA-1) | B-lymphocyte chemoattractant |
| EGF | Epidermal growth factor |
| ENA-78 | Epithelial cell-derived neutrophil-activating peptide |
| Eot | Eotaxin |
| Eot-2 | Eotaxin-2 |
| Fas | Fas (CD95) |
| FGF-7 | Fibroblast growth factor-7 |
| FGF-9 | Fibroblast growth factor-9 |
| GDNF | Glial cell line derived neurotrophic factor |
| GM-CSF | Granulocyte macrophage colony stimulating factor |
| IL-1ra | Interleukin 1 receptor antagonist |
| IL-2 sRα | Interleukin 2 soluble receptor alpha |
| IL-3 | Interleukin 3 |
| IL-4 | Interleukin 4 |
| IL-5 | Interleukin 5 |
| IL-6 | Interleukin 6 |
| IL-7 | Interleukin 7 |
| IL-8 | Interleukin 8 |
| IL-13 | Interleukin 13 |
| IL-15 | Interleukin 15 |
| MCP-2 | Monocyte chemotactic protein 2 |
| MCP-3 | Monocyte chemotactic protein 3 |
| MIP-1α | Macrophage inflammatory protein 1 alpha |
| MPIF | Myeloid progenitor inhibitory factor 1 |
| OSM | Oncostatin M |
| P1GF | Placental growth factor |

Array 2.1 detector set.

| | |
|---|---|
| AR | Amphiregulin |
| BDNF | Brain-derived neurotrophic factor |
| FLT-3 Lig | fms-like tyrosine kinase-3 ligand |
| GCP-2 | Granulocyte chemotactic protein 2 |
| HCC4 (NCC4) | Hemofiltrate CC chemokine 4 |
| I-309 | I-309 |
| IL-1α | Interleukin 1 alpha |
| IL-1β | Interleukin 1 beta |
| IL-2 | Interleukin 2 |
| IL-17 | Interleukin 17 |
| MCP-1 | Monocyte chemotactic protein 1 |
| M-CSF | Macrophage colony stimulating factor |
| MIG | Monokine induced by interferon gamma |
| MIP-1β | Macrophage inflammatory protein 1 beta |
| MIP-1γ | Macrophage inflammatory protein 1 delta |
| NT-3 | Neurotrophin 3 |
| NT-4 | Neurotrophin 4 |
| PARC | Pulmonary and activation-regulated chemokine |
| RANTES | Regulated upon activation, normal T expressed and |
| | presumably secreted |
| SCF | Stem cell factor |
| sgp130 | Soluble glycoprotein 130 |
| TARC | Thymus and activation regulated chemokine |
| TNF-RI | Tumor necrosis factor receptor I |
| TNF-α | Tumor necrosis factor alpha |
| TNF-β | Tumor necrosis factor beta |
| VEGF | Vascular endothelial growth factor |

Microarray Chip Physical Quality Measures

Each print run of microarray chips was assigned a unique Production Sheet Number, and the RCAT immunoassay run for this print run was documented. For each print run, printed slides were subjected to the following control measures: (1) two slides, one from the start and one from the end of the run, were inspected using light microscopy. If the percentage of missing spots observed was greater than 5%, then the batch failed and the slides were discarded immediately. For all print runs described herein, 100% of the printed spots were present on slides selected for this examination; and (2) for each print run, two of the printed slides were examined by a Cy5-labeled goat-anti-mouse antibody (GAM-Cy5). Since the majority of capture antibodies in these arrays were of mouse origin, this procedure examined total antibody attachment and provided a rapid measure of surface and binding uniformity. To account for differences in binding efficiency for different capture antibodies, the intensities of all spots for each individual capture antibody were measured across the chip (4 spots/subarray, 64 spots/chip) and a %CV was calculated for that feature. The average of these %CVs for all quantified capture antibodies must be below 20% for the print batch to pass. Chips treated with GAM-Cy5 were also checked for missing spots after the assay and if the percentage of missing spots was greater than 5%, then the batch failed (for these studies 100% of the printed spots were still present after this assay). Following these QC measures, qualified slides were stored at 4° C. until used.

Reagent Quality Control Measures

The assay suite was considered as consisting of the microarray chips, detector antibodies and the reagents required for the RCAT portion of the assay. There were validation procedures for these reagents individually as well as a functional validation of the entire set. Reagents used in the RCA portion of the assay were from reserved vendor lots where possible. Materials produced in-house were subjected to QC procedures and qualified on microarray chips before release. If lot numbers changed for a particular reagent that is supplied by an outside vendor, the new lots were qualified by comparison with existing qualified stocks.

For each array type, a concentrated batch of detectors was prepared which consisted of a mixture of biotinylated antibodies directed against all analytes represented by an array. A functional QC was then performed for each detector antibody batch by carrying out the standard RCAT assay on a specially prepared sample set. Mixtures of 2-3 different cytokines were prepared so as to provide a high intensity signal and applied to 14 wells of a chip (with each well being treated with a different mixture up to the total complement of detector antibodies) and two arrays were used as blank controls. The chips were developed and scanned and the resulting signals were compared to the positional map of the particular array. This examination demonstrated that the stock detector mixture was complete and the features were active. Once a detector batch had passed this QC, it was distributed into smaller volumes and released for use in the assay.

Positional and Functional Quality Measures

Following printing, a set of microarray chips was validated in concert with the qualified reagents discussed above. This was a two-part quality control measure. The first portion was identical to the detector antibody qualification procedure just described. In this case, the high intensity signals were compared to the array map and the proper positioning of capture antibody replicates was verified. The second test was a functional QC for all analytes of a specified array using known sample matrices. Normal human serum (Jackson ImmunoResearch Laboratories, Code#009-000-121) and heparinized plasma were assayed neat or spiked with purified recombinant cytokines representing all analytes in the array. Spiked mixtures were then titrated down the subarrays of a slide from 5,000 pg/ml to 20 pg/mL of spiked cytokine concentrations along with three subarrays for each un-spiked control sample. The data was quantified and for every analyte in the array a titration curve was generated to show that the feature intensity was above background and exhibiting increasing intensity with increasing analyte concentrations.

RCA Immunoassay

Prior to assay, the slides were removed from storage at room temperature in sealed containers and opened in a humidity controlled chamber (35-40%). Blocking was done by submerging the slides in a Coplin jar filled with blocking buffer (Seablock, Pierce Chemical Co., 1:1 dilution with 1× PBS) pre-chilled to 4° C., and placing the Coplin jar in a 37° C. incubator for 1 hour. The slides were then washed twice (2 min per wash) in 60 mL of 1× PBS/0.5% Brj-35 washing buffer. On each slide, control serum (Jackson ImmunoResearch Laboratories) was applied to one subarray, plasma control applied to two subarrays, and a negative control with PBS buffer applied to two subarrays. The test samples were assayed on the remaining 11 subarrays. Twenty microliters of the treated sample were then applied to each subarray. The basics of performing immunoassays with RCA signal amplification has been described (Nat. Biotechol. (2002) 20:359-65) and we are using SOPs derived from the protocols used in that study. Slides were scanned (GenePix 4000B, Axon Instruments Inc.) at 10 μm resolution with a laser setting of 100% and a PMT setting of 550 V. Mean pixel fluorescence values were quantified using the fixed circle method in GenePix Pro 4.0 (Axon Instruments). Using proprietary software, the fluorescence intensity of microarray spots was analyzed for each feature and sample, and the resulting mean intensity values were determined. Dose-response curves for selected cytokines were examined, ensuring that feature intensity is above background and exhibiting increasing intensity with increasing analyte concentration.

ELISA Analysis

Reagents for FLT3 ligand (FL) and IL-6 ELISA kits were obtained from R&D Systems, Inc. (Minneapolis, Minn.; catalog #s DFK00, Q6000). C-reactive protein (CRP) (accession ID AAA 52075) ELISA kits were obtained from KMI Diagnostics (Minneapolis, Minn.; catalog #EU59131). ELISAs were performed on patient plasma according to the manufacturers' instructions. The FL and CRP kits relied on a colorimetric readout; the optical density of each well was determined using a microplate spectrophotometer and data was analyzed using KC-4 software from Bio-Tek Instruments, Inc. The IL-6 kit was a chemiluminescent sandwich ELISA; luminescence values were determined on a microplate luminometer. SOFTmaxPRO software was used to extrapolate cytokine concentrations from the standard curves.

2. More Studies Using Compound 1—Results

Plasma Markers Identified Using Antibody Chip Technology

A multiplex antibody chip based approach (MSI, Molecular Staging Inc.) was used to identify plasma biomarkers of compound 1. Plasma samples harvested from 3 advanced malignancy patients pre and post Compound 1 treatment (Phase I trial A) were used for this analysis. Twenty three of 108 markers tested, showed changes following Compound 1 treatment (day 28). These are listed in Table 21. Controls included normal donor plasma which did not show significant changes in these markers. Each of these is a potential biomarker of Compound 1, and may reflect drug exposure, biological activity or efficacy.

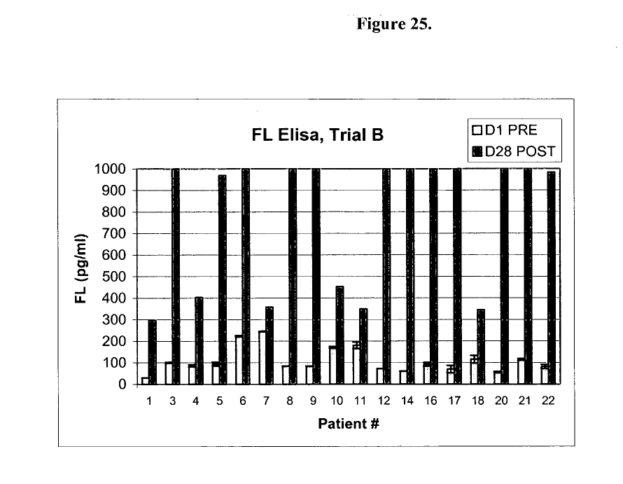

A number of markers showing the most dramatic changes and/or of known biological significance were further investigated (specifically VEGF, PLGF, IL-6, IL-8 and MCP-1). The relative changes were validated by ELISA on the same patient samples assessed in the antibody chip screen, and both methods showed good concordance (Table 22). Several of these markers had previously been identified by ELISA analysis on compound 1 treated samples, (PLGF, VEGF, IL-6), and several were novel (FLT3 ligand and MCP-1). Additional data on FLT3 ligand levels tested in an expanded set of patients is provided in FIG. 25. Dramatic induction was observed following Compound 1 treatment in all cases.

Plasma ELISA Studies

In an effort to identify novel biomarkers of exposure to Compound 1, plasma samples were analyzed from 18 patients enrolled in Trial B. Plasma was taken both before study (D1 PRE) as well as at the end of the first cycle of treatment (Day 28 POST). Each time point was measured in triplicate and the standard deviation from the mean was calculated. Both the mean value and standard deviation for each patient at each time point is shown graphically in FIG. 25. It was found that 100% of the patients exhibited an increase in FLT3 ligand (FL) concentration from day 1 to day 28. In 14 out of 18 patients, the increase was more than four-fold. The increase in FLT3 ligand concentration is attributed to treatment with Compound 1.

Plasma ELISA Studies—Fatigue Corrolation

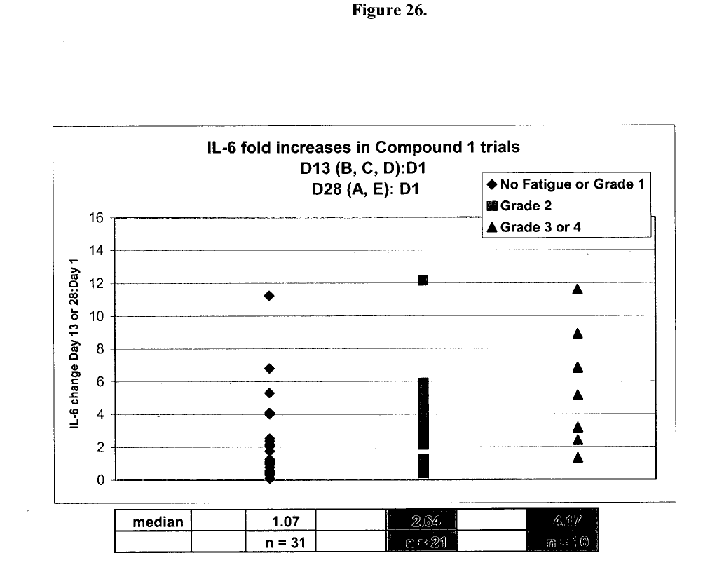

To find biomarkers that correlated with fatigue, plasma samples were analyzed from 62 patients enrolled in trials for Compound 1. Samples were taken before study (D1) and either two or four weeks after the start of cycle 1 dosing (Day 13 for trials B, C and D and Day 28 for A and E). The patients are grouped according to their highest recorded fatigue grade (0-4 scale from the NCI Common Toxicity Criteria). As seen in FIG. 26, there is a statistically significant difference between the increases in IL-6 seen in patients with low fatigue (Grade 1 or 0) and those with moderate to high fatigue (Grade 3 or 4), p=0.001. Thus, a patient who exhibits a large change in IL-6 plasma concentration (greater than two-fold) after treatment with Compound 1 has a much higher chance of experiencing a high degree of fatigue (Grade 3 or 4) than a patient whose IL-6 level remains more stable.

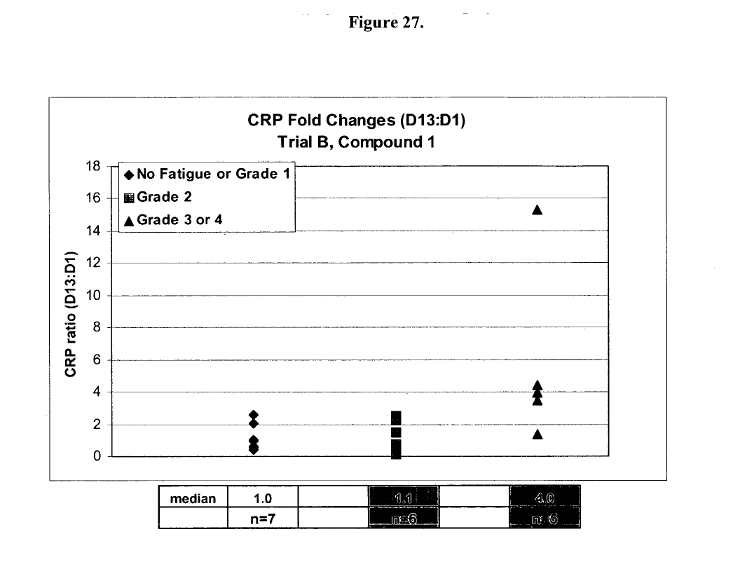

Plasma samples were further analyzed from 18 patients enrolled in Trial B for Compound 1. Samples were taken before study (D1) and two weeks after the start of cycle 1 dosing (D13). As shown with IL-6 levels, the patients are grouped according to their highest recorded fatigue grade (0-4). See FIG. 27. It was determined there is a statistically significant difference in C-reactive protein (CRP) (accession ID AAA 52075) induction between patients with little fatigue (Grade 0, 1, or 2) and those with moderate to severe fatigue (Grade 3 or 4), p=0.0088. Therefore, patients with a greater than two-fold increase in C-reactive protein after treatment with Compound 1 are more prone to experiencing high fatigue than those who have smaller fold changes in CRP.

Plasma ELISA Studies—Corrolation to Biological Response and/or Clinical Efficacy

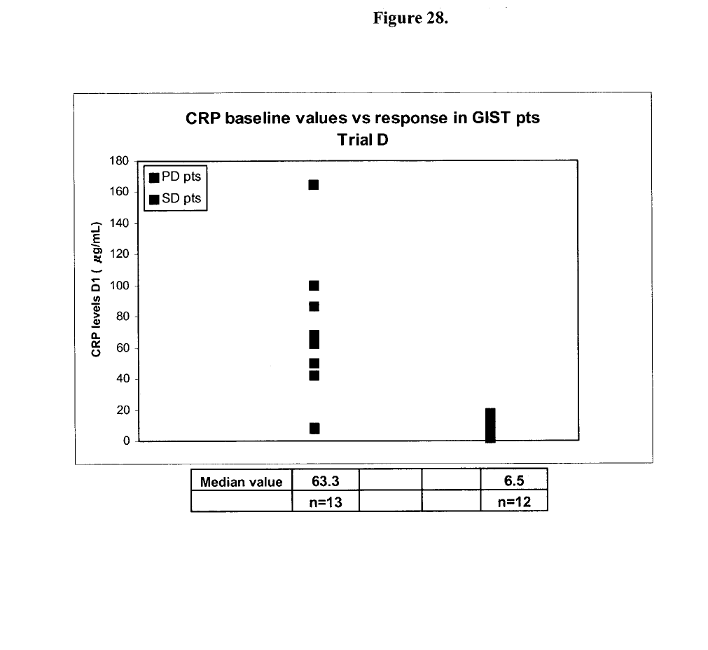

Levels of C-reactive protein were measured as described above for the experiments involving CRP and fatigue. ELISAs were performed on plasma samples from patients before treatment (i.e., baseline values). The patients' samples and results were divided into two groups based upon observed clinical outcome. Patients with stable disease (SD pts) were defined as patients on study for over 6 months. Patients with progressive disease (PD pts) were defined as patients who had come off study due to disease progression or lack of efficacy in fewer than 6 months. This separation of patients demonstrated that patients with progressive disease had much higher baseline levels of CRP than those patients who were stable (median values of 63.8 μg/mL vs. 6.5 μg/mL, respectively) (FIG. 28). If a patient were to have a baseline level of CRP of above 20 μg/mL before treatment, that patient has a greater chance of rapidly progressing than if the level of CRP were below 20 μg/mL. Thus, CRP is a baseline marker of biological response and/or clinical efficacy.

### K. EXAMPLES

### Compound 1 Studies of OB-Cadherin 1 Protein

1. Compound 1 Studies of OB-Cadherin 1 Protein—Materials and Methods

Tumor Samples

Colo205 human colon xenograft tumors were isolated and fixed in Streck Tissue Fixative (Streck Laboratories, Inc., La Vista, Nev.). Samples used in immunohistochemistry were sent out to BioPathology Sciences Medical Corporation (South San Francisco, Calif.) for paraffin embedding and sectioning.

Antibodies

A rabbit polyclonal antibody recognizing the cytoplasmic tail region of OB-cadherin 1 (cadherin 11) was purchased from Zymed Laboratories, Inc. (Zymed reagent #71-7600; South San Francisco, Calif.).

Immunohistochemistry

Sections (4-5 μm) stained using an automated immunohistochemistry system (Benchmark System, Ventana Medical Systems, Inc., Tucson, Ariz.). In brief, slides were deparaffinized using heat at 75° C. and Ventana's EZ Prep product (Ventana reagent #950-102). Antigen retrieval was performed by incubating the slides for 30 min with Ventana's CC2 product (Ventana reagent #950-123), a citrate-based solution with pH 6.0. Primary antibody (5 μg/ml) was incubated for 24 min at room temperature, followed by a secondary detection system, using biotinylated secondary antibody (Vector anti-rabbit secondary, BA-1000, at 2.5 μg/ml; Vector Laboratories, Burlingame, Calif.) with incubation time of 8 min. Streptavadin-horseradish peroxidase with 3, 3′ diaminobenzidine as a substrate were used in conjunction with the secondary detection system. All samples analyzed for OB-cadherin 1 expression were also stained with the omission of primary antibody as a negative control.

Compound 1 Studies of OB-Cadherin 1 Protein—Data Summary

As expression of OB-cadherin 1 (cadherin 11) RNA was found to be up-regulated at 24 hour post-Compound 1 treatment (see Table 12), effects on OB-cadherin 1 expression at the protein level was also examined. Colo205 xenograft tumors were isolated from Compound 1-treated mice at 24 and 48 hours post treatment. Tumors were fixed in formalin and sections were isolated and processed for immunochemistry (IHC).

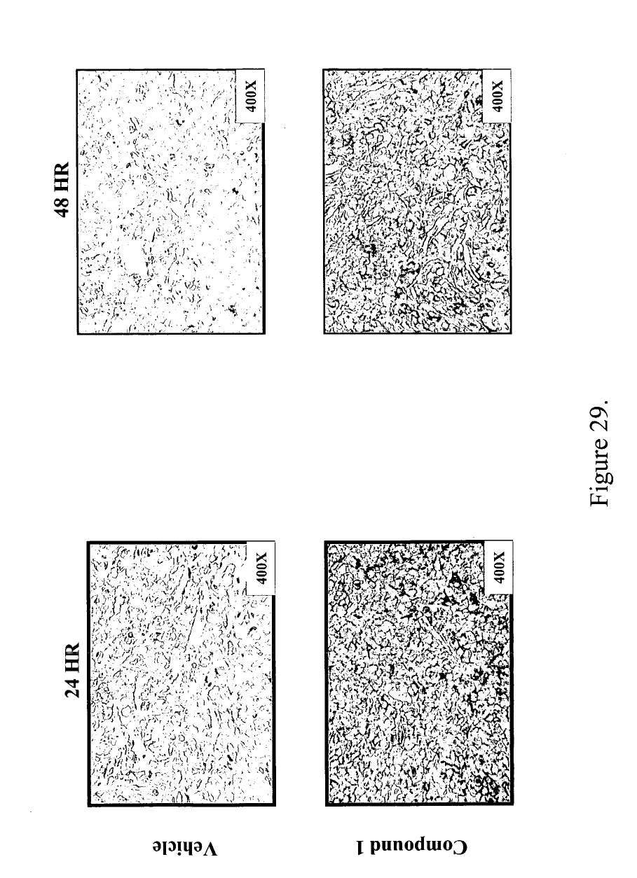

Tissue sections were stained with an antibody that recognizes OB-cadherin 1. As a negative control, adjacent sections were processed similarly but with the omission of a primary antibody. This analysis identified up-regulation of OB-cadherin 1 protein in Colo205 tumors treated with Compound 1 for 24 and 48 hours as compared to vehicle treated samples (FIG. 29).

### Tables

TABLE 1

| | | | | Number |
|---|---|---|---|---|
| | | | | for which |
| | Number of | Number with | | data passed |
| | samples from | RNA yield | Number | Quality Control |
| | which RNA | >1 ug, at both | hybridized to | inspection for |
| | was processed | d1 and d56 | U95A chips | further analysis |
| SU5416 | | | | |
| CR | 0 | 0 | 0 | 0 |
| PR | 13 | 8 | 6 | 6* |
| MR | 6 | 3 | 2 | 1 |
| SD | 6 | 5 | 1 | 1 |
| PD | 10 | 7 | 6 | 5* |
| Control | | | | |
| CR | 1 | 1 | 1 | |
| PR | 9 | 5 | 5 | 5* |
| MR | 4 | 1 | 1 | 0 |
| SD | 3 | 2 | 2 | 2 |
| PD | 11 | 9 | 7 | 6* |
| Total: | 63 | 41 | 31 | 27 |

*These samples were included in the dataset used in detailed analysis

TABLE 2

| Affymetrix | Gene name/ | Putative | Increased in | Increased in |
|---|---|---|---|---|
| number | Symbol | function(s) | SU5416 arm | Control arm |
| 34546_at | Defensin α | Corticostatic, | 10 of 11 | 6 of 12 |
| | 4 | Ca channel | | |
| | | regulator | | |
| 33530_at | CEA CAM 8 | Tumor antigen, | 9 of 11 | 4 of 12 |
| | | integral | | |
| | | membrane | | |
| | | protein. | | |
| 37054_at | BPI | Anti-pathogen | 9 of 11 | 4 of 12 |
| | | response | | |
| 31859_at | MMP-9 | Protease; ECM | 8 of 11 | 2 of 12 |
| | | maintainence | | |
| 32821_at | Lipocalin 2 | Anti-pathogen | 10 of 11 | 5 of 12 |
| | | response; | | |
| | | apoptosis | | |
| 34319_at | S100 P | Ca-binding | 9 of 11 | 3 of 12 |
| | | protein | | |
| 41249_at | Hypothetic. | unknown | 7 of 11 | 1 of 12 |
| | Protein | | | |
| | FLJ13052 | | | |
| 1962_at | Liver | Amino acid | 9 of 11 | 3 of 12 |
| | arginase | metabolism | | |
| 266_s_at | CD24 | Anti-pathogen | 9 of 11 | 0 of 12 |
| | antigen | response; | | |
| | | differentiation | | |
| | | of B cells | | |
| 31506_s_at | Defensin α 3 | Chemotaxis; | 10 of 11 | 4 of 12 |
| | | anti-microbial | | |
| | | response | | |
| 32275_at | Antileuko- | Secreted | 9 of 11 | 4 of 12 |
| | protease | inhibitor of | | |
| | | serine proteases | | |
| 115_at | Thromo- | Blood clotting; | 9 of 11 | 3 of 12 |
| | bospondin 1 | angiogenesis | | |
| 37149_s_at | Lactoferrin | Iron transport; | 11 of 11 | 5 of 12 |
| | | putative | | |
| | | protease | | |

TABLE 3

| | | |
|---|---|---|
| Gene | Forward Primer | Reverse Primer |

| | | | | |
|---|---|---|---|---|
| Thrombospondin 1 | TTGGCTACCAGTCCAGCAGC | (SEQ ID NO: 1) | GGGTTGGTGTCCCAGTAGGA | (SEQ ID NO: 2) |
| MMP-9 | CCCGGAGTGAGTTGAACCA | (SEQ ID NO: 3) | CCTAGTCCTCAGGGCACTGC | (SEQ ID NO: 4) |
| Defensin α 3 | CCCAGAAGTGGTTGTTTCCCT | (SEQ ID NO: 5) | GTCCATGTTTTTCCTTGAGCCT | (SEQ ID NO: 6) |
| Lactoferrin | CTGGAAGCCTGTGAATTCC | (SEQ ID NO: 7) | GAATGGCTGAGGCTTTCTTGG | (SEQ ID NO: 8) |
| Lipocalin-2 | GCTGACTTCGGAACTAAAGGAGAA | (SEQ ID NO: 9) | TGGGACAGGGAAGACGATGT | (SEQ ID NO: 10) |
| CD24 | CTGCCTCGACACACATAAACCTT | (SEQ ID NO: 11) | CATCTAAGCATCAGTGTGTGACC | (SEQ ID NO: 12) |
| | | | A | |

TABLE 4
P-value of Mann-Whitney U Test

| | Affymetrix | SYBR Green RT-PCR |
|---|---|---|
| Gene | (n = 23) | (n = 31) |
| MMP-9 | 0.0025 | 0.0748 |
| Thrombospondin 1 | 0.0267 | 0.7186 |
| CD24 | 0.0006 | 0.0057 |
| Defensin α 3 | 0.0002 | 0.2196 |
| Lactoferrin | 0.0002 | 0.0065 |
| Lipocalin-2 (LCN2) | 0.0005 | 0.0057 |

TABLE 5

| | | Rank Sum | Rank Sum | | |
|---|---|---|---|---|---|
| Gene | n | (Treatment) | (Control) | Mann-Whitney U | p-value |
| MMP-9 | 36 | 415 | 251 | 0.0095 | |
| CD24 | 36 | 443 | 223 | 0.0005 | |
| Lactoferrin | 36 | 460 | 206 | 0.0001 | |
| LCN2 | 36 | 419 | 247 | 0.0065 | |

TABLE 6
Predictor Gene Set for discriminating between the
control and Compound B arms: LCN2, CD24, Lactoferrin

| | | |
|---|---|---|
| Control | Treatment | % Correct |

1. All cases pooled (67 cases from both trials)

| | | | |
|---|---|---|---|
| Control | 26 | 5 | 84 |
| Treatment | 6 | 30 | 83 |
| Total | 32 | 35 | 84 |

2. Jackknifed classification matrix for
all cases pooled (67 cases from both trials)

| | | | |
|---|---|---|---|
| Control | 26 | 5 | 84 |
| Treatment | 8 | 28 | 78 |
| Total | 34 | 33 | 81 |

3. Prediction subset (randomly selected 34 cases)
from all cases pooled (67 cases in both trials)

| | | | |
|---|---|---|---|
| Control | 13 | 1 | 93 |
| Treatment | 4 | 16 | 80 |
| Total | 17 | 17 | 85 |

4. Validation subset (randomly selected 33 cases)
from all cases pooled (67 cases in both trials)

| | | | |
|---|---|---|---|
| Control | 11 | 6 | 65 |
| Treatment | 5 | 11 | 69 |
| Total | 16 | 17 | 67 |

TABLE 7
Trial C patients 1-23 PLGF plasma level ratios

| Patient # | d1 (6 hr):d1 (0 hr) | d29:d1 | d42:d1 |
|---|---|---|---|
| 1 | 0.695512 | 1.871238 | 0.398897 |
| 2 | 2.050289 | 11.96579 | 1.040025 |
| 3 | 1.965517 | 3.586207 | 1.206897 |
| 4 | 1.985061 | 24.72922 | 1.985061 |
| 5 | 1.09557 | 11.3316 | 1.09557 |
| 6 | 1.800672 | 11.02117 | 1.365586 |
| 8 | 1.16493 | 12.38985 | 1.157115 |
| 10 | 1.622462 | >10 | 2.652309 |
| 11 | 1.250022 | 7.511615 | 1.386382 |
| 13 | 1.038442 | 1.817441 | NA |
| 15 | 0.896403 | 6.651554 | 1.189041 |
| 17 | 0.907692 | 19.21308 | 1.134385 |
| 18 | 1.007357 | 12.30822 | 1.105295 |
| 20 | 1.2261 | 11.29078 | 1.598445 |
| 21 | 1.518564 | 14.84205 | 0.955559 |
| 22 | 1 | 2.423462 | 0.815385 |
| Average | 1.326537 | 10.19689 | 1.272397 |

***Note:
d15:D1 ratio is 6.4 for pt. 13

TABLE 8

| MIG | | | | | IP-10 | | |
|---|---|---|---|---|---|---|---|
| Patient | day 1 | day 15 | end C1 dosing | Ratio | day 1 | end C1 dosing | Ratio |
| 11(B) | 41.927 | | 739.71 | 17.64281 | 55.617 | >500 | >9 |
| 1 | 48.375 | | 1066.2 | 22.04031 | 64.847 | >500 | >7.7 |
| 11 | 34.432 | | 344.93 | 10.01772 | 65.32 | 384.06 | 5.879669 |
| 17 | 166.8 | | 907.09 | 5.438189 | 72.29 | >500 | >6.9 |
| 24 | 80.751 | | 314.2 | 3.890973 | | | |
| 26 | 80.751 | | 995.47 | 12.32765 | 64.296 | >500 | >7.7 |
| 27 | 80.826 | | 81.439 | 1.007584 | | | |
| 7 | 106.04 | | 145.64 | 1.373444 | 139.2 | 240.31 | 1.726365 |
| 20 | 161.91 | | 698.23 | 4.312458 | 73.67 | >500 | >6.9 |
| 22 | 37.685 | 339.16 | 8.999867 | | | | |
| 9 (A) | 60.393 | | 138.56 | 2.294306 | | | |
| I-TAC | | | | | | | |
| 11(B) | 428.83 | | >4000.0 | >9 | | | |
| 1 | | | | | | | |
| 11 | | | | | | | |
| 17 | | | | | | | |
| 24 | 259.38 | | 771.04 | 2.972627 | | | |
| 26 | 97.917 | | 701.46 | 7.163822 | | | |
| 27 | 139.94 | | 315.69 | 2.255895 | | | |
| 7 | | | | | | | |
| 20 | | | | | | | |
| 22 | 190.76 | 2020.2 | | 10.59027 | | | |
| 9(A) | 59.975 | | 212.26 | 3.539141 | | | |

TABLE 9

| | PLGF Ratio | VEGFR2 ratio | |
|---|---|---|---|
| Patient # | (end dosing:d1) | (end dosing:d1) | Primary Diagnosis |
| Trial C | | | |
| 1 | 1.871237941 | 0.265856292 | Synovial Sarcoma |
| 2 | 11.96579454 | 0.25171334 | Rectal |
| 3 | 3.586206897 | 0.5673112 | Gall-bladder |
| 4 | 24.72921991 | 0.34236691 | Hepatocellular |
| 5 | 11.33159926 | 0.406890612 | Melanoma |
| 6 | 11.02116835 | 0.572980623 | Breast |
| 7 | 23.86685363 | 0.404286499 | Ovary |
| 8 | 12.38984817 | 0.318366334 | Small Cell Lung |
| 10 | 10 | 0.45614753 | Melanoma |
| 11 | 7.511615487 | 0.323681006 | Met. Colon |
| 13 | 1.817440506 | 0.460416464 | Renal Cell Carcinoma |
| 14 | 3.080408542 | 0.575703582 | Met. Melanoma |
| 15 | 6.651553529 | 0.506347193 | Renal Cell Carcinoma |
| 17 | 19.21307692 | 0.177452364 | NSCLC |
| 18 | 12.30822285 | 0.271285002 | NSCLC |
| 20 | 11.29078149 | 0.385479698 | Colon |
| 21 | 14.84205128 | 0.369637606 | Breast |
| 22 | 2.423461538 | 0.479139734 | Sarcoma |
| 23 | 1 | 0.504789782 | Sarcoma |
| 24 | 0.99016936 | 0.457140878 | met. Rectal carcinoma |
| 25 | 12.03862173 | 0.250133543 | Retropero Sarcoma |
| 26 | 13.29469461 | 0.493391074 | Met Pelvis Sarcoma |
| 29 | 5.237072177 | 0.59927457 | SCCR R) Parotid |
| 30 | | 0.519969363 | Colon AdenoCA |
| 31 | | 0.330647033 | Lung AdenoCA |
| Trial A | | | |
| 1 | | 0.565173104 | Renal Cell Carcinoma |
| 3 | | 0.597994214 | Bronchial adeno. |
| 4 | | 0.685465839 | breast carcinoma |
| 5 | 12.97391648 | 0.182557005 | uterine |
| 6 | 25.082632 | 0.458079657 | pelvic angiosarcoma |
| 7 | | 0.648790016 | pleural mesothelioma |
| 8 | | 0.64392508 | uterine |
| 9 | | 0.38520981 | Bronchial adeno. |
| 10 | 5.301660143 | 0.44915001 | colorectal |
| 13 | | 0.297438475 | neuroendocrine |
| Trial D | | | |
| 1 | | 0.502083475 | GIST |
| 3 | 2.98130415 | 0.670742516 | GIST |
| 4 | 5.228142589 | 0.972905837 | GIST |
| 5 | 1.351061278 | 0.616277438 | GIST |
| 6 | 7.055260831 | 0.684932856 | GIST |
| 13 | 4.095209935 | 0.600072917 | GIST |
| 14 | 4.786806356 | 0.685754939 | GIST |
| 15 | 22.29951691 | 0.767346939 | GIST |
| 16 | 3.034877351 | 0.727153597 | GIST |
| 18 | 16.89889246 | 0.471077781 | GIST |
| 19 | 2.782095462 | 0.542935245 | GIST |
| 20 | 12.47129736 | 0.598602839 | GIST |
| 21 | 11.56450225 | 0.351218422 | GIST |
| 22 | 2.996492067 | 0.644054653 | GIST |
| Trial B | | | |
| 4 | | 0.67109839 | Head & Neck |
| 5 | | 0.678411145 | CRC |
| 6 | | 0.4130696 | thymic |
| 7 | | 0.301532905 | CRC |
| 8 | | 0.456886687 | thyroid |
| 9 | | 0.597322954 | thyroid |

TABLE 10

| MIG | | | | | |
|---|---|---|---|---|---|
| | | | end | | |
| Patient# | day 1 | day 15 | C1 dosing | Ratio | Cancer Type |
| 11(B) | 41.927 | | 739.71 | 17.64281 | Pancreatic |
| 1 | 48.375 | | 1066.2 | 22.04031 | Synovial Sarcoma |
| 11 | 34.432 | | 344.93 | 10.01772 | Met. Colon |
| 17 | 166.8 | | 907.09 | 5.438189 | NSCLC |
| 24 | 80.751 | | 314.2 | 3.890973 | Met. Rectal |
| 26 | 80.751 | | 995.47 | 12.32765 | Pelvis Sarcoma |
| 20 | 161.91 | | 698.23 | 4.312458 | Colon |
| 22 | 37.685 | 339.16 | 8.999867 | Sarcoma | |
| 9 (A) | 60.393 | | 138.56 | 2.294306 | Bronchial Adeno. |

TABLE 11A

| | | | Time | Fold |
|---|---|---|---|---|
| | | Accession | Point | Change |
| Transcript Name | Putative Role | No. | (hrs) | Increase |
| Basic transcription | Transcription | M90354 | 6 | 2.1 |
| factor 3 homologue | factor | | | |
| c-jun proto oncogene | Transcription | J04111 | 6 | 2.5 |
| | factor | | | |
| c-fos cellular oncogene | Transcription | K00650 | 6 | 4.2 |
| | factor | | | |
| Tyrosine phosphatase | Protein | NM_080422 | 6 | 2.2 |
| non-receptor type 2 | phosphatase | | | |
| cdc2-related protein | Cell cycle | M68520 | 6 | 19 |
| kinase | regulation | | | |
| Cyclin C | Cell cycle | M74091 | 6 | 2.5 |
| | regulation | | | |
| DNA polymerase gamma | DNA | U60325 | 6 | 7.3 |
| | polymerase | | | |
| Basic transcription factor | Transcription | M90354 | 24 | 2.2 |
| 3 homologue | factor | | | |
| Protein kinase C alpha | Protein kinase | X52479 | 24 | 3.0 |
| Lipocortin II/annexin A2 | Ca⁺⁺-regulated | D00017 | 24 | 2.3 |
| | membrane | | | |
| | binding protein | | | |
| Histone H2B, member R | Transcriptional | AF531293 | 24 | 3.0 |
| | regulation | | | |
| Amphiregulin | Growth factor | NM_001657 | 24 | 6.1 |
| Ephrin receptor EphB4 | Tyrosine kinase | NM_004444 | 6 | 2.5 |
| | receptor | | | |
| Hanukah factor/ | Serine protease | M18737 | 24 | 2.3 |
| Granzyme A | | | | |
| von Hippel-Lindau | Tumor | NM_000551 | 24 | 3.7 |
| (VHL) tumor suppressor | suppressor | | | |
| OB-cadherin 1 | Ca⁺⁺-dependent | D21254 | 24 | 2.2 |
| | cell adhesion | | | |
| | protein | | | |
| OB-cadherin 2 | Ca⁺⁺-dependent | D21255 | 24 | 2.0 |
| | cell adhesion | | | |
| | protein | | | |
| Phosphoinositol | Phospho- | NM_014935 | 24 | 2.1 |
| 3-phosphate-binding | inositide- | | | |
| protein-3 (PEPP3) | binding protein | | | |
| Phosphoinositol 3-kinase, | Proliferation | M61906 | 24 | 2.2 |
| p85 subunit | | | | |
| Mucin 1 | Adhesion, | J05582 | 24 | 2.5 |
| | cell—cell | | | |
| | interaction | | | |
| Hepatitis C-associated | Interferon- | Exon 1-9 | 24 | 2.0 |
| microtubular aggregate | induced | D28908, | | |
| p44 | protein | D28909, | | |
| | | D28910, | | |
| | | D28911, | | |
| | | D28912, | | |
| | | D28913, | | |
| | | D28914, | | |
| | | D28915 | | |
| ErbB3/HER3 receptor | Growth factor | M29366 | 24 | 2.1 |
| tyrosine kinase | receptor | | | |

TABLE 11B

| | | | Time | Fold |
|---|---|---|---|---|
| | | | Point | Change |
| Transcript Name | Putative Role | Accession No. | (hrs) | Increase |
| Vinculin | Cell adhesion | M33308 | 4 | 2.5 |
| Basic transcription | Transcription | M90357 | 24 | 2.2 |
| factor 3 | factor | | | |
| Phosphoinositol 3- | Proliferation | NM_006219 | 24 | 4.5 |
| kinase, p110 subunit | | | | |
| | | | Time | Fold |
| | | | Point | Change |
| Transcript Name | Putative Role | Accession No. | (hrs) | Decrease |
| Gelsolin | Actin binding | X04412 | 4 | 2.1 |
| | protein | | | |
| Cyclin D2 | Transcription | NM_001759 | 4 | 2.2 |

TABLE 12

| | | Relative | Relative |
|---|---|---|---|
| | | Expression | Expression |
| Transcript Name | Accession No. | Level (6 hr) | Level (24 hr) |
| Amphiregulin | NM_001657 | 1.9 | 2.5 |
| Cdc2-related protein kinase | M68520 | 0.43 | 0.55 |
| Phosphoinositol 3-kinase, | NM_006219 | 0.59 | 1.6 |
| p110 subunit | | | |
| Cyclin C | M74091 | 842 | 22.3 |
| OB-cadherin 1 | D21254 | 0.35 | 23.8 |
| OB-cadherin 2 | D21255 | 0.40 | 0.51 |
| Phosphoinositol 3-kinase, | M61906 | 1.0 | 2.30 |
| p85 subunit | | | |
| Mucin 1 | J05582 | 0.32 | 1.13 |
| von Hippel-Lindau tumor | NM_000551 | 0.9 | 0.55 |
| suppressor | | | |
| Ephrin receptor, EphB4 | NM_004444 | 3.5 | 3.1 |
| Gelsolin | X04412 | 4.0 | 0.04 |

TABLE 13

| | GenBank | | | |
|---|---|---|---|---|
| Transcripts | Accession No. | Forward Primer (5′-3′) | Reverse Primer (5′-3′) | Taqman Probe (5′-3′) |
| Amphiregulin | NM_001657 | ATGATGAGTCGGTCCTCT | TGACAATTGAAAGTTTAA | TCCATTGTCTTATGA |
| | | TTCC | AACCATCATA | TCCAC |
| | | (SEQ ID NO: 13) | (SEQ ID NO: 14) | (SEQ ID NO: 15) |
| CDK-2 related | M68520 | AGTTAGAAGTTAGGGTTT | TACCCATGCCCTCACTCA | AAGTGTCAGCATTCT |
| protein | | AGGCATCATT | ATC | CAA |
| | | (SEQ ID NO: 16) | (SEQ ID NO: 17) | (SEQ ID NO: 18) |
| PI3-kinase, | NM_006219 | CCAGTGTTGTGAGGATGC | CAGTCAACATCAGCGCAA | ATTCCCATGCCGTCG |
| p110 | | ATATC | AGA | TA |
| | | (SEQ ID NO: 19) | (SEQ ID NO: 20) | (SEQ ID NO: 21) |
| PI3-kinase, p85 | M61906 | CAAACCTACTGTATCTCT | GACAGAGATGATTATCCC | AGCGCTCACCTTTG |
| | | AATACAGTGTGACT | TTTAAACCA | (SEQ ID NO: 24) |
| | | (SEQ ID NO: 22) | (SEQ ID NO: 23) | |
| Cyclin C | M74091 | CCTACAGACAGACATACA | ATTATGCTTCATGTTTCCT | CCAAATTAAGAAAT |
| | | TAGACATTTCAA | GGCTTA | ATTATACTAATCA |
| | | (SEQ ID NO: 25) | (SEQ ID NO: 26) | (SEQ ID NO: 27) |
| OB-cadherin 1 | D21254 | GACAACAGTTCTGAGCTG | TGGGTTTAAGCTGGCTGA | ACTCTGGACACTCTA |
| | | TAATTTCG | ATATTAT | TATGT |
| | | (SEQ ID NO: 28) | (SEQ ID NO: 29) | (SEQ ID NO: 30) |
| OB-cadherin 2 | D21255 | TCAGCCAGCTTAAACCCA | TGGCACGTATTAGTTTAA | CTTGTTACTGCTGAT |
| | | TACAA | GATGAAAGTAG | TCT |
| | | (SEQ ID NO: 31) | (SEQ ID NO: 32) | (SEQ ID NO: 33) |
| Mucin 1 | J05582 | TTCAGAGGCCCCACCAAT | CCCACATGAGCTTCCACA | TCTCGGACACTTCTC |
| | | T | CA | (SEQ ID NO: 36) |
| | | (SEQ ID NO: 34) | (SEQ ID NO: 35) | |
| VHL tumor | NM_000551 | TGAGGCAGGGACAAGTCT | ACCCTGACTGAAGGCTCA | CTCTTTGAGACCCCA |
| suppressor | | TTCT | TGA | GTGC |
| | | (SEQ ID NO: 37) | (SEQ ID NO: 38) | (SEQ ID NO: 39) |
| EphB4 | NM_004444 | TCTACCGTCCTTGTCATA | ATGATGATGGGCCCCTGT | CCTTTGCCCAAGTTG |
| | | ACTTTGTG | T | (SEQ ID NO: 42) |
| | | (SEQ ID NO: 40) | (SEQ ID NO: 41) | |
| Gelsolin | X04412 | TGGACGTTTTGTGATCGA | AAGTCAAGGCTTCTGTCT | CTTGAGAATCCTTTC |
| | | AGAG | TTTCTTCT | CAACC |
| | | (SEQ ID NO: 43) | (SEQ ID NO: 44) | (SEQ ID NO: 45) |

TABLE 14

| | |
|---|---|
| Gel No. 1 | VEGF + DMSO − 48 hr |
| Gel No. 2 | Compound 1 + VEGF + DMSO − 48 hr |
| Gel No. 3 | VEGF + DMSO − 48 hr |

TABLE 15

| Spot #1202 | | |
|---|---|---|
| Sample | Run #1 | Run #2 |
| baseline | 126 | 22.5 |
| VEGF at 24 h | 437 | 192.4 |
| VEGF at 48 h | 812 | 540 |
| VEGF and compound 1 (1 uM) at 24 h | 270 | 484.7 |
| VEGF and compound 1 (1 uM) at 48 h | 869 | 158 |

TABLE 16

| | | MALDI Mass | MS-Fit | Sequence |
|---|---|---|---|---|
| SSP | Well | Mapping result | MOWSE Score | Coverage |
| 1202 | A6 | Interstitial | 3.64E+07 | 31% |
| | | Collagenase | | |
| | | Precursor | | |

TABLE 17

| | | | | | |
|---|---|---|---|---|---|
| | | | | | MASCOT |
| SSP | Well | Confirmed Peptide | File Name | MS/MS result | Score |

| | | | | | |
|---|---|---|---|---|---|
| 1202 | A6 | DIYSSFGFPR | spotA6- | MM01_HUMAN, Interstitial Collagenase Precursor P03956 | 34 |
| | | (SEQ ID NO: 46) | 1188.wiff | 53973/6.4 | |
| 1202 | A6 | DGFFYFFHGTR | spotA6prod1393 | - MM01_HUMAN, Interstitial Collagenase Precursor P03956 | 22 |
| | | (SEQ ID NO: 47) | 2.wiff | 53973/6.4 | |

TABLE 18

| | Average | Standard |
|---|---|---|
| HUVEC SAMPLE¹ | pro-MMP1 (ng/ml) | Deviation |
| VEGF 10 min | 4.66 | 0.3079 |
| DMSO 10 min | 4.64 | 0.1003 |
| compound 1 @ 10 nM 10 min | 5.41 | 0.1224 |
| Compound 1 @ 100 nM 10 min | 5.78 | 0.3158 |
| Compound 1 @ 1 uM 10 min | 5.04 | 0.331 |
| VEGF 8 hr | 16.47 | 1.0048 |
| DMSO 8 hr | 17.63 | 1.2563 |
| Compound 1 @ 10 nM 8 hr | 14.93 | 1.1245 |
| Compound 1 @ 100 nM 8 hr | 12.75 | 0.6686 |
| Compound 1 @ 1 uM 8 hr | 14.48 | 1.0551 |
| VEGF 24 hr | 45.71 | 3.06 |
| DMSO 24 hr | 79.94 | 4.50 |
| Compound 1 @ 10 nM 24 hr | 70.21 | 4.82 |
| Compound 1 @ 100 nM 24 hr | 50.26 | 1.24 |
| Compound 1 @ 1 uM 24 hr | 50.42 | 2.42 |
| VEGF 48 hr | 244.74 | 3.91 |
| DMSO 48 hr | 234.74 | 10.85 |
| Compound 1 @ 10 nM 48 hr | 135.35 | 1.04 |
| Compound 1 @ 100 nM 48 hr | 128.75 | 11.05 |
| Compound 1 @ 1 uM 48 hr | 103.09 | 3.60 |

¹Time points indicated (10 min, 8 h, 24 h, 48 h) refer to the period of time post-VEGF treatment after which samples were isolated.

TABLE 19

| | Pro-MMP1 (ng/ml) | FC vs d1 Pre¹ | % Change vs d1 Pre |
|---|---|---|---|
| Pt 3 d1 Pre² | 0.3115 | | |
| d1 24hr | 0.2837 | −1.097990835 | −8.924558587 |
| d13 Pre | 0.6756 | 2.168860353 | 116.8860353 |
| d13 12 hr | 0.6235 | 2.001605136 | 100.1605136 |
| d13 24 hr | 0.4035 | 1.295345104 | 29.53451043 |
| Pt 4 d1 Pre | 0.5214 | | |
| d1 24 hr | 0.8938 | 2.869341894 | 71.42309168 |
| d13 Pre | 0.6246 | 2.005136437 | 19.79286536 |
| d13 12 hr | 0.4579 | 1.469983949 | −12.17874952 |
| d13 24 hr | 0.4514 | 1.449117175 | −13.42539317 |
| Pt 5 d1 Pre | 0.5739 | | |
| d1 24 hr | 0.323 | 1.036918138 | −43.71841784 |
| d13 Pre | 0.7269 | 2.333547352 | 26.65969681 |
| d13 12 hr | 0.6874 | 2.206741573 | 19.77696463 |
| d13 24 hr | 0.4171 | 1.339004815 | −27.32183307 |
| Pt 6 d1 Pre | 0.2969 | | |
| d1 24 hr | 0.6818 | 2.188764045 | 129.6396093 |
| d13 Pre | 0.7597 | 2.438844302 | 155.8773998 |
| d13 12 hr | 0.7992 | 2.56565008 | 169.1815426 |
| d13 24 hr | 1.066 | 3.422150883 | 259.043449 |
| Pt 7 d1 Pre | 0.5743 | | |
| d1 24 hr | 0.7334 | 2.354414125 | 27.70329096 |
| d13 Pre | 0.7374 | 2.367255217 | 28.39979105 |
| d13 12 hr | 0.5154 | 1.654574639 | −10.25596378 |
| d13 24 hr | 0.7203 | 2.312359551 | 25.42225318 |
| Pt 8 d1 Pre | 0.2879 | | |
| d1 24 hr | 0.3664 | 1.176243981 | 27.26641195 |
| d13 Pre | 1.7166 | 5.510754414 | 496.2486975 |
| d13 12 hr | 1.1071 | 3.554093098 | 284.5432442 |
| d13 24 hr | 0.8494 | 2.726805778 | 195.0329976 |
| Pt 9 d1 Pre | 0.7786 | | |
| d1 24 hr | 0.4816 | 1.546067416 | −38.14538916 |
| d13 Pre | 0.4931 | 1.582985554 | −36.66837914 |
| d13 12 hr | 1.047 | 3.361155698 | 34.47212946 |
| d13 24 hr | 2.6022 | 8.353772071 | 234.2152582 |
| Pt 10 d1 Pre | 0.3613 | | |
| d1 24 hr | 0.2396 | −1.300083472 | −33.68391918 |
| d13 Pre | 1.2937 | 4.153130016 | 258.0680875 |
| d13 12 hr | 1.4224 | 4.566292135 | 293.6894547 |
| d13 24 hr | 1.0684 | 3.429855538 | 195.7099363 |
| Pt 11 d1 Pre | 0.299 | | |
| d1 24 hr | 0.2866 | −1.08688067 | −4.147157191 |
| d13 Pre | 0.6931 | 2.225040128 | 131.8060201 |
| d13 12 hr | 0.4496 | 1.443338684 | 50.36789298 |
| d13 24 hr | 1.1685 | 3.751203852 | 290.8026756 |
| Pt 12 d1 Pre | 0.8587 | | |
| d1 24 hr | 0.5418 | 1.739325843 | −36.90462327 |
| d13 Pre | 2.1689 | 6.962760835 | 152.5794806 |
| d13 12 hr | 2.1494 | 6.900160514 | 150.308606 |
| d13 24 hr | 5.9226 | 19.01316212 | 589.7170141 |

¹Fold change of pro-MMP1 levels are indicated by “FC vs d1 pre”. These levels were calculated by dividing the levels of pro-MMP1 after drug treatment by the MMP1 levels present before drug treatment (d1 pre).
²Patient number is indicated (Pt), time point of sampling is indicated pre-treatment (d1 pre), 24 hours post first treatment (d1 24 h), after 13 days of treatment (d13 pre), after 13 days and 12 hours post-treatment (d13 12 h), and 13 days and 24 hours of treatment (d13 24 h).

TABLE 20

| | | | | | | | | |
|---|---|---|---|---|---|---|---|---|
| 5000 ng./mL | 4000 ng/mL | BLANK | AR | BDNF | FGF-6 | Flt3Lig | G-CSF | HCC4 |
| Bio-mlgG | Bio-mlgG | | | | | | | |
| 5000 ng./mL | 4000 ng/mL | BLANK | AR | BDNF | FGF-6 | Flt3Lig | G-CSF | HCC4 |
| Bio-mlgG | Bio-mlgG | | | | | | | |
| 5000 ng./mL | 4000 ng/mL | BLANK | AR | BDNF | FGF-6 | Flt3Lig | G-CSF | HCC4 |
| Bio-mlgG | Bio-mlgG | | | | | | | |
| 5000 ng./mL | 4000 ng/mL | BLANK | AR | BDNF | FGF-6 | Flt3Lig | G-CSF | HCC4 |
| Bio-mlgG | Bio-mlgG | | | | | | | |
| 1000 ng/mL | 800 ng/mL | 600 ng/mL | 400 ng/mL | 300 ng/mL | 200 ng/mL | 100 ng/mL | 80 ng/mL Bio | 60 ng/mL Bio |
| Bio-mlgG | Bio-mlgG | Bio-mlgG | Bio-mlgG | Bio-mlgG | Bio-mlgG | Bio-mlgG | mlgG | mlgG |
| 1000 ng/mL | 800 ng/mL | 600 ng/mL | 400 ng/mL | 300 ng/mL | 200 ng/mL | 100 ng/mL | 80 ng/mL Bio | 60 ng/mL Bio |
| Bio-mlgG | Bio-mlgG | Bio-mlgG | Bio-mlgG | Bio-mlgG | Bio-mlgG | Bio-mlgG | mlgG | mlgG |
| 1000 ng/mL | 800 ng/mL | 600 ng/mL | 400 ng/mL | 300 ng/mL | 200 ng/mL | 100 ng/mL | 80 ng/mL Bio | 60 ng/mL Bio |
| Bio-mlgG | Bio-mlgG | Bio-mlgG | Bio-mlgG | Bio-mlgG | Bio-mlgG | Bio-mlgG | mlgG | mlgG |
| 1000 ng/mL | 800 ng/mL | 600 ng/mL | 400 ng/mL | 300 ng/mL | 200 ng/mL | 100 ng/mL | 80 ng/mL Bio | 60 ng/mL Bio |
| Bio-mlgG | Bio-mlgG | Bio-mlgG | Bio-mlgG | Bio-mlgG | Bio-mlgG | Bio-mlgG | mlgG | mlgG |
| GCF-2 | NT3 | NT4 | PARC | Rantes | SCF | SDF-1α | sgp130 | TARC |
| GCP-2 | NT3 | NT4 | PARC | Rantes | SCF | SDF-1α | sgp130 | TARC |
| GCP-2 | NT3 | NT4 | PARC | Rantes | SCF | SDF-1α | sgp130 | TARC |
| GCF-2 | NT3 | NT4 | PARC | Rantes | SCF | SDF-1α | sgp130 | TARC |
| Blank | IL-2 | IL-6sR | IL-11 | IL-12 p70 | IL-16 | IP-17 | IP-10 | LIF |
| Blank | IL-2 | IL-6sR | IL-11 | IL-12 p70 | IL-16 | IP-17 | IP-10 | LIF |
| Blank | IL-2 | IL-6sr | Il-11 | IL-12 p70 | IL-16 | IP-17 | IP-10 | LIF |
| Blank | IL-2 | IL-6sr | IL-11 | IL-12 p20 | IL-16 | IP-17 | IP-10 | LIF |

| | | | | | | | |
|---|---|---|---|---|---|---|---|
| 5000 ng./mL | I-309 | IL-1α | IL-1β | IL-1sR1 | 0 mg/mL Bio- | 3000 mg/ml | 2000 ng/mL |
| Bio-mlgG | mlgG | Bio-mlgG | Bio-mlG | | | | |
| 5000 ng./mL | I-309 | IL-1α | IL-1β | IL-1sR1 | 0 mg/mL Bio- | 3000 mg/ml | 2000 ng/mL |
| Bio-mlgG | mlgG | Bio-mlgG | Bio-mlG | | | | |
| 5000 ng./mL | I-309 | IL-1α | IL-1β | IL-1sR1 | 0 mg/mL Bio- | 3000 mg/ml | 2000 ng/mL |
| Bio-mlgG | mlgG | Bio-mlgG | Bio-mlG | | | | |
| 5000 ng./mL | I-309 | IL-1α | IL-1β | IL-1sR1 | 0 mg/mL Bio- | 3000 mg/ml | 2000 ng/mL |
| Bio-mlgG | | | | | mlgG | Bio-mlgG | Bio-mlG |
| 1000 ng/mL | 50 ng/mL Bio | 40 ng/mL Bio | 30 ng/mL Bio | 20 ng/mL Bio | 10 ng/mL Bio- | 5 ng/mL Bio | Blank |
| Bio-mlgG | mlgG | mlgG | mlgG | mlgG | mlgG | mlgG | |
| 1000 ng/mL | 50 ng/mL Bio | 40 ng/mL Bio | 30 ng/mL Bio | 20 ng/mL Bio | 10 ng/mL Bio- | 5 ng/mL Bio | Blank |
| Bio-mlgG | mlgG | mlgG | mlgG | mlgG | mlgG | mlgG | |
| 1000 ng/mL | 50 ng/mL Bio | 40 ng/mL Bio | 30 ng/mL Bio | 20 ng/mL Bio | 10 ng/mL Bio- | 5 ng/mL Bio | Blank |
| Bio-mlgG | mlgG | mlgG | mlgG | mlgG | mlgG | mlgG | |
| 1000 ng/mL | 50 ng/mL Bio | 40 ng/mL Bio | 30 ng/mL Bio | 20 ng/mL Bio | 10 ng/mL Bio- | 5 ng/mL Bio | Blank |
| Bio-mlgG | mlgG | mlgG | mlgG | mlgG | mlgG | mlgG | |
| GCF-2 | TGF-β1 | TNF-α | TNF-β | TNF-R1 | TNF-RII | VEGF | Blank |
| GCP-2 | TGF-β1 | TNF-α | TNF-β | TNF-R1 | TNF-RII | VEGF | Blank |
| GCP-2 | TGF-β1 | TNF-α | TNF-β | TNP-R1 | TNF-RII | VEGF | Blank |
| GCF-2 | TGF-β1 | TNP-α | TNF-β | TNP-R1 | TNF-RII | VEGF | Blank |
| Blank | MCP-1 | M-CSF | MDC | MIG | MIP-1β | MIP-1δ | NAP-2 |
| Blank | MCP-1 | M-CSF | MDC | MIG | MIP-1β | MIP-1δ | NAP-2 |
| Blank | MCP-1 | M-CSF | MDC | MIG | MIP-1β | MIP-1δ | NAP-2 |
| Blank | MCP-1 | M-CSF | MDC | MIG | MIP-1β | MIP-1δ | NAP-2 |

TABLE 21

| Patient | 1, | 2, | 3 | Patient | 1, | 2, | 3 |
|---|---|---|---|---|---|---|---|
| ENA-78 | (−) | (−) | ⇓ | TNFR1 | ↑ | ↑ | (−) |
| MPIF-1 | (−) | (−) | ⇓ | VEGF | ↑ | ↑ | (−) |
| GCP-2 | ↑ | (−) | (−) | F1t3L | ↑ | ↑ | ↑ |
| Amphireg | ↑ | (−) | (−) | PLGF | ↑ | ↑ | (−) |
| IL-1α | ↑ | ↑ | (−) | IL6 | ↑ | ↑ | (−) |
| IL-1β | ↑ | ↑ | (−) | MCP-1 | ↑ | ↑ | (−) |
| IL-2 | ↑ | ↑ | (−) | TNFα | ↑ | ↑ | (−) |
| MIG | (−) | ⇓ | (−) | TARC | ↑ | (−) | (−) |
| NT4 | ↑ | (−) | ↑ | MMP7 | ↑ | ↑ | ⇓ |
| GCP-2 | ↑ | ↑ | (−) | MMP9 | (−) | (−) | ↑ |
| IGFBP-1 | ↑ | ↑ | ↑ | leptin | (−) | ↑ | (−) |
| GRO-β | ↑ | (−) | ↑ | | | | |

TABLE 22

| | Patient 1 | | Patient 2 | | Patient 3 | |
|---|---|---|---|---|---|---|
| | ELISA | Ab Chip | ELISA | Ab Chip | ELISA | Ab Chip |
| VEGF | 32 | 2.7 | 72 | 4.3 | 3 | 1.8 |
| PLGF | 13 | 4.6 | 25.1 | 21.7 | 5.3 | 1.6 |
| IL-6 | 29 | 2.9 | 11.6 | 3.7 | 0.9 | 0.99 |
| IL-8 | 2 | 1.5 | 2.7 | 1.8 | 0.77 | 1.7 |
| FLT3 L | 10.3 | 13.9 | 6.7 | 7.7 | 2.6 | 6.2 |
| MCP-1 | 2.2 | 2.5 | 1.93 | 2 | 1.0 | 1.4 |

We claim:

1. A method for determining whether a test compound inhibits tyrosine kinase activity in a mammal, comprising:
(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;
(b), exposing the mammal to the test compound; and
(c) following the exposing of step (b), measuring in the mammal the level of at least one of the proteins and/or mRNA transcripts measured in step (a),
wherein a difference in the level of said protein and/or mRNA transcript measured in (c), compared to the level of protein and/or mRNA transcript measured in step (a) indicates that the test compound is an inhibitor of tyrosine kinase in the mammal.

2. A method for determining whether a test compound inhibits tyrosine kinase activity in a mammal, comprising:
(a) exposing the mammal to the test compound; and
(b) following the exposing of step (a), measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1,
wherein a difference in the level of said protein and/or mRNA measured in (b), compared to the level of protein and/or mRNA in a mammal that has not been exposed to said test compound, indicates that the compound is an inhibitor of tyrosine kinase in the mammal.

3. A method for determining whether a mammal has been exposed to a test compound that inhibits tyrosine kinase activity, comprising:
(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;
(b), exposing the mammal to the test compound; and
(c) following the exposing of step (b), measuring in the mammal the level of at least one of the proteins and/or mRNA transcripts measured in step (a),
wherein a difference in the level of said protein and/or mRNA measured in (c), compared to the level of protein and/or mRNA in step (a) indicates that the mammal has been exposed to a test compound that inhibits tyrosine kinase activity.

4. A method for determining whether a mammal has been exposed to a test compound that inhibits tyrosine kinase activity, comprising
(a) exposing the mammal to the test compound; and
(b) following the exposing of step (a), measuring in a mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1,
wherein a difference in the level of said protein and/or mRNA measured in (b), compared to the level of protein and/or mRNA in a mammal that has not been exposed to said test compound, indicates that the mammal has been exposed to a test compound that is an inhibitor of tyrosine kinase.

5. A method for determining whether a mammal is responding to a compound that inhibits tyrosine kinase activity, comprising:
(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;
(b), exposing the mammal to the compound; and
(c) following the exposing of step (b), measuring in the mammal the level of at least one of the proteins and/or mRNA transcripts measured in step (a),
wherein a difference in the level of said protein and/or mRNA transcripts measured in (c), compared to the level of protein and/or mRNA transcript for said protein in step (a) indicates that that the mammal is responding to the compound that inhibits tyrosine kinase activity.

6. A method for determining whether a mammal is responding to a compound that inhibits tyrosine kinase activity, comprising:
(a) exposing the mammal to the compound; and
(b) following the exposing step (a), measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1,
wherein a difference in the level of said protein and/or mRNA measured in (b), compared to the level of protein and/or mRNA in a mammal that has not been exposed to said compound, indicates that the mammal is responding to the compound that inhibits tyrosine kinase.

7. A method for identifying a mammal that will respond therapeutically to a method of treating cancer comprising administering at least one inhibitor of a VEGFR and/or PDGFR tyrosine kinase, wherein the method for identifying the mammal comprises:
(a) measuring in the mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;
(b) exposing the mammal to at least one inhibitor of a VEGFR and/or PDGFR tyrosine kinase; and
(c) following the exposing of step (b), measuring in the mammal the level of at least one of the proteins and/or mRNA transcripts measured in step (a),
wherein a difference in the level of said protein and/or mRNA transcripts measured in (c), compared to the level of protein and/or mRNA transcript for said protein in step (a) indicates that that the mammal will respond therapeutically to a method of treating cancer comprising administering at least one inhibitor of a VEGFR and/or PDGFR tyrosine kinase.

8. A method for testing or predicting whether a mammal will respond therapeutically to a method of treating cancer comprising administering at least one inhibitor of a VEGFR and/or PDGFR tyrosine kinase, wherein the method for testing or predicting comprises:
(a) measuring in a mammal with cancer the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;
(b) measuring in the same type of mammal without cancer, the level of at least one of the same proteins and/or mRNA transcripts measured in step (a);
(c) comparing levels of said proteins and/or mRNA transcripts measured in (a) and (b);
wherein a difference in the level of said protein and/or mRNA in the mammal with cancer as measured in step (a), compared to the level of said protein and/or mRNA in the mammal without cancer as measured in step (b), indicates that the mammal will respond therapeutically to at least one inhibitor of a VEGFR and/or PDGFR tyrosine kinase.

9. The method of any one of claims 1-8, wherein the mammal is a human, rat, mouse, dog, rabbit, pig, sheep, cow, horse, cat, primate or monkey.

10. The method of any one of claims 1-8, wherein the method is an in vitro method, and wherein the protein and/or mRNA is measured in at least one mammalian biological tissue from the mammal.

11. The method of claim 10, wherein the biological tissue comprises a biological, fluid that is selected from the group consisting of whole fresh blood, peripheral blood mononuclear cells, frozen whole blood, fresh plasma, frozen plasma, urine and saliva.

12. The method of claim 10, wherein the tissue is selected from the group consisting of buccal mucosa tissue, skin, hair follicles, tumor tissue and bone marrow.

13. The method of any one of claims 1-8, wherein the mammal has cancer.

14. The method of any one of claims 1-8, wherein the compound that inhibits tyrosine kinase activity is an indolinone compound.

15. The method of any one of claims 1-8, wherein the compound that inhibits tyrosine kinase activity is:
a pyrrole substituted 2-indolinone having the formula: 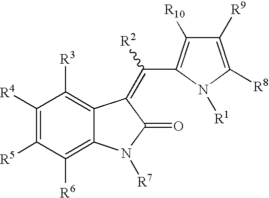
wherein:
R¹, R² and R⁷ are hydrogen;
R³, R⁴, R⁵, and R⁶ are independently selected from the group consisting of hydrogen, hydroxy, halo, unsubstituted lower alkyl, lower alkyl substituted with a carboxylic acid, unsubstituted lower alkoxy, carboxylic acid, unsubstituted aryl, aryl substituted with one or more unsubstituted lower alkyl alkoxy, and morpholino;
R⁸ is unsubstituted lower alkyl;
R⁹ is —(CH₂)(CH₂)C(═O)OH; and
R₁₀ is unsubstituted lower alkyl;
or a pharmaceutically acceptable salt thereof; or
a compound having the formula: 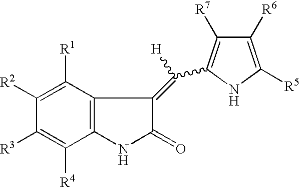
wherein:
R¹ is selected from the group consisting of hydrogen, halo, alkyl, cyclkoalkyl, aryl, heteroaryl, heteroalicyclic, hydroxy, alkoxy, —(CO)R¹⁵, —NR¹³R¹⁴, —(CH₂)ᵣR¹⁶ and —C(O)NR⁸R⁹;
R² is selected from the group consisting of hydrogen, halo, alkyl, trihalomethyl, hydroxy, alkoxy, cyano, —NR¹³R¹⁴, —NR¹³C(O)R¹⁴, —C(O)R¹⁵, aryl, heteroaryl, and —S(O)₂NR¹³R¹⁴;
R³ is selected from the group consisting of hydrogen, halogen, alkyl, trihalomethyl, hydroxy, alkoxy, —(CO)R¹⁵, —NR¹³R¹⁴, aryl, heteroaryl, —NR¹³S(O)₂R¹⁴, —S(O)₂NR¹³R¹⁴, —NR¹³C(O)R¹⁴, —NR¹³C(O)OR¹⁴ and —SO₂R²⁰ (wherein R²⁰ is alkyl, aryl, aralkyl, heteroaryl and heteroaralkyl);
R⁴ is selected from the group consisting of hydrogen, halogen, alkyl, hydroxy, alkoxy and —NR¹³R¹⁴;
R⁵ is selected from the group consisting of hydrogen, alkyl and —C(O)R¹⁰;
R⁶ is selected from the group consisting of hydrogen, alkyl and —C(O)R¹⁰;
R⁷ is selected from the group consisting of hydrogen, alkyl, aryl, heteroaryl, —C(O)R¹⁷ and —C(O)R₁₀; or
R⁶ and R⁷ may combine to form a group selected from the group consisting of —(CH₂)₄—, —(CH₂)₅— and —(CH₂)₆—;
with the proviso that at least one of R⁵, R⁶ or R⁷ must be —C(O)R¹⁰;
R⁸ and R⁹ are independently selected from the group consisting of hydrogen, alkyl and aryl;
R₁₀ is selected from the group consisting of hydroxy, alkoxy, aryloxy, —N(R¹¹)(CH₂)ₙR¹², and —NR¹³R¹⁴;
R¹¹ is selected from the group consisting of hydrogen and alkyl;
R¹² is selected from the group consisting of —NR¹³R¹⁴, hydroxy, —C(O)R¹⁵, aryl, heteroaryl, —N⁺(O⁻)R¹³R¹⁴, —N(OH)R¹³, and —NHC(O)R^{a} (wherein R^{a} is unsubstituted alkyl, haloalkyl, or aralkyl);
R¹³ and R¹⁴ are independently selected from the group consisting of hydrogen, alkyl, lower alkyl substituted with hydroxyalkylamino, cyanoalkyl, cycloalkyl, aryl and heteroaryl; or
R¹³ and R¹⁴ may combine to form a heterocyclo group;
R¹⁵ is selected from the group consisting of hydrogen, hydroxy, alkoxy and aryloxy;
R¹⁶ is selected from the group consisting of hydroxy, —C(O)R¹⁵, —NR¹³R¹⁴ and —C(O)NR¹³R¹⁴;
R¹⁷ is selected from the group consisting of alkyl, cycloalkyl, aryl and heteroaryl;
R²⁰ is alkyl, aryl, aralkyl or heteroaryl; and
n and r are independently 1, 2, 3, or 4;
or a pharmaceutically acceptable salt thereof; or
a compound having the formula: 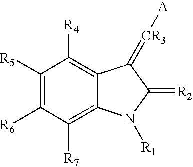
wherein:
R₁ is H;
R₂ is O or S;
R₃ is hydrogen;
R_{4,} R₅, R_{6,} and R₇ are each independently selected from the group consisting of hydrogen, alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R, and CONRR′;
A is a five membered heteroaryl ring selected from the group consisting of thiophene, pyrrole, pyrazole, imidazole, 1,2,3-triazole, 1,2,4-triazole, oxazole, isoxazole, thiazole, isothiazole, 2-sulfonylfuran, 4-alkylfuran, 1,2,3-oxadiazole, 1,2,4-oxadiazole, 1,2,5-oxadiazole, 1,3,4-oxadiazole, 1,2,3,4-oxatriazole, 1,2,3,5-oxatriazole, 1,2,3-thiadiazole, 1,2,4-thiadiazole, 1,2,5-thiadiazole, 1,3,4-thiadiazole, 1,2,3,4-thiatriazole, 1,2,3,5-thiatriazole, and tetrazole, optionally substituted at one or more positions with alkyl, alkoxy, aryl, aryloxy, alkaryl, alkaryloxy, halogen, trihalomethyl, S(O)R, SO₂NRR′, SO₃R, SR, NO₂, NRR′, OH, CN, C(O)R, OC(O)R, NHC(O)R, (CH₂)ₙCO₂R or CONRR′;
n is 0-3;
R is H, alkyl or aryl; and
R′ is H, alkyl or aryl;
or a pharmaceutically acceptable salt thereof; or
a compound having the formula: 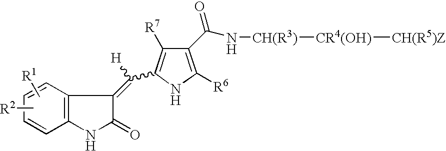
wherein:
R¹ is selected from the group consisting of hydrogen, halo, alkyl, haloalkoxy, cycloalkyl, heteroalicyclic, hydroxy, alkoxy, —C(O)R⁸, —NR⁹R¹⁰ and —C(O)NR¹²R¹³;
R² is selected from the group consisting of hydrogen, halo, alkyl, trihalomethyl, hydroxy, alkoxy, cyano, —NR⁹R¹⁰, —NR⁹C(O)R¹⁰, —C(O)R⁸, —S(O)₂NR⁹R¹⁰ and —SO₂R¹⁴ (wherein R¹⁴ is alkyl, aryl, aralkyl, heteroaryl and heteroaralkyl);
R³, R⁴ and R⁵ are independently hydrogen or alkyl;
Z is aryl, heteroaryl, heterocycle, or —NR¹⁵R¹⁶ wherein R¹⁵ and R¹⁶ are independently hydrogen or alkyl; or R¹⁵ and R¹⁶ together with the nitrogen atom to which they are attached from a heterocycloamino group;
R⁶ is selected from the group consisting of hydrogen or alkyl;
R⁷ is selected from the group consisting of hydrogen, alkyl, aryl, heteroaryl, and —C(O)R¹⁷ as defined below;
R⁸ is selected from the group consisting of hydroxy, alkoxy and aryloxy;
R⁹ and R₁₀ are independently selected from the group consisting of hydrogen, alkyl, cyanoalkyl, cycloalkyl, aryl and heteroaryl; or
R⁹ and R₁₀ combine to form a heterocycloamino group;
R¹² and R¹³ are independently selected from the group consisting of hydrogen, alkyl, hydroxyalkyl, and aryl; or R¹² and R¹³ together with the nitrogen atom to which they are attached form a heterocycloamino;
R¹⁷ is selected from the group consisting of alkyl, cycloalkyl, aryl, hydroxy and heteroaryl;
or a pharmaceutically acceptable salt thereof.

16. The method of any one of claims 1-8, wherein the compound that inhibits tyrosine kinase activity is 3-[2,4-dimethyl-5-(2-oxo-1,2-dihydro-indol-3-ylidenemethyl)-1H-pyrrol-3-yl]-propionic acid (Compound A) or a pharmaceutically acceptable salt thereof.

17. The method of any one of claims 1-8, wherein the compound that inhibits tyrosine kinase activity is 3-(3,5-dimethyl-1H-pyrrol-2-ylmethylene)-1,3-dihydro-indol-2-one (Compound B) or a pharmaceutically acceptable salt thereof.

18. The method of any one of claims 1-8, wherein the compound that inhibits tyrosine kinase activity is a compound of Formula I: 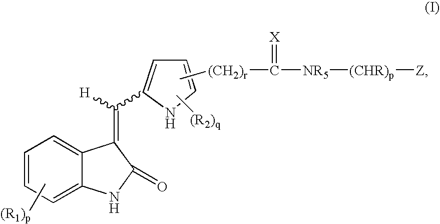
wherein:
R is independently H, OH, alkyl, aryl, cycloalkyl, heteroaryl, alkoxy, heterocyclic and amino;
each R₁ is independently selected from the group consisting of alkyl, halo, aryl, alkoxy, haloalkyl, haloalkoxy, cycloalkyl, heteroaryl, heterocyclic, hydroxy, —C(O)—R₈, —NR₉R₁₀, —NR₉C(O)—R₁₂ and —C(O)NR₉R₁₀;
each R₂ is independently selected from the group consisting of alkyl, aryl, heteroaryl, —C(O)—R₈, and SO₂R″, where R″ is alkyl, aryl, heteroaryl, NR₉N₁₀ or alkoxy;
each R₅ is independently selected from the group consisting of hydrogen, alkyl, aryl, haloalkyl, cycloalkyl, heteroaryl, heterocyclic, hydroxy, —C(O)—R₈ and (CHR)ᵣR₁₁;
X is O or S;
p is 0-3;
q is 0-2;
r is 0-3;
R₈ is selected from the group consisting of —OH, alkyl, aryl, heteroaryl, alkoxy, cycloalkyl and heterocyclic;
R₉ and R₁₀ are independently selected from the group consisting of H, alkyl, aryl, aminoalkyl, heteroaryl, cycloalkyl and heterocyclic, or R₉ and R₁₀ together with N may form a ring, where the ring atoms are selected from the group consisting of C, N, O and S;
R₁₁ is selected from the group consisting of —OH, amino, monosubstituted amino, disubstituted amino, alkyl, aryl, heteroaryl, alkoxy, cycloalkyl and heterocyclic
R₁₂ is selected from the group consisting of alkyl, aryl, heteroaryl, alkoxy, cycloalkyl and heterocyclic;
Z is OH, O-alkyl, or —NR₃R_{4,} where R₃ and R₄ are independently selected from the group consisting of hydrogen, alkyl, aryl, heteroaryl, cycloalkyl, and heterocyclic, or R₃ and R₄ may combine with N to form a ring where the ring atoms are selected from the group consisting of CH₂, N, O and S or 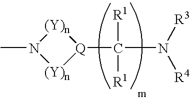
wherein Y is independently CH₂, O, N or S,
Q is C or N;
n is independently 0-4; and
m is 0-3;
or a pharmaceutically acceptable salt thereof.

19. The method of any one of claims 1-8, wherein the compound that inhibits tyrosine kinase activity is a compound of Formula II: 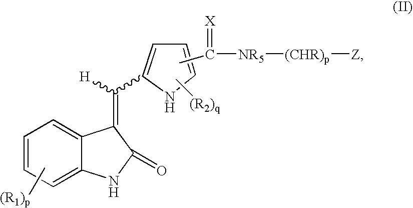
wherein:
R is independently H, OH, alkyl, aryl, cycloalkyl, heteroaryl, alkoxy, heterocyclic and amino;
each R₁ is independently selected from the group consisting of alkyl, halo, aryl, alkoxy, haloalkyl, haloalkoxy, cycloalkyl, heteroaryl, heterocyclic, hydroxy, —C(O)—R₈, —NR₉R₁₀, —NR₉C(O)—R₁₂ and —C(O)NR₉R₁₀;
each R₂ is independently selected from the group consisting of alkyl, aryl, heteroaryl, —C(O)—R₈, and SO₂R″, where R″ is alkyl, aryl, heteroaryl, NR₉N₁₀ or alkoxy;
each R₅ is independently selected from the group consisting of hydrogen, alkyl, aryl, haloalkyl, cycloalkyl, heteroaryl, heterocyclic, hydroxy, —C(O)—R₈ and (CHR)ᵣR₁₁;
X is O or S;
p is 0-3;
q is 0-2;
r is 0-3;
R₈ is selected from the group consisting of —OH, alkyl, aryl, heteroaryl, alkoxy, cycloalkyl and heterocyclic;
R₉ and R₁₀ are independently selected from the group consisting of H, alkyl, aryl, aminoalkyl, heteroaryl, cycloalkyl and heterocyclic, or R₉ and R₁₀ together with N may form a ring, where the ring atoms are selected from the group consisting of C, N, O and S;
R₁₁ is selected from the group consisting of —OH, amino, monosubstituted amino, disubstituted amino, alkyl, aryl, heteroaryl, alkoxy, cycloalkyl and heterocyclic
R₁₂ is selected from the group consisting of alkyl, aryl, heteroaryl, alkoxy, cycloalkyl and heterocyclic;
Z is OH, O-alkyl, or —NR₃R_{4,} where R₃ and R₄ are independently selected from the group consisting of hydrogen, alkyl, aryl, heteroaryl, cycloalkyl, and heterocyclic, or R₃ and R₄ may combine with N to form a ring where the ring atoms are selected from the group consisting of CH₂, N, O and S or 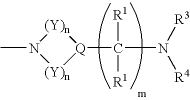
wherein Y is independently CH₂, O, N or S,
Q is C or N;
n is independently 0-4; and
m is 0-3;
or a pharmaceutically acceptable salt thereof.

20. The method of claim 18, wherein the compound that inhibits tyrosine kinase activity is selected from the group consisting of: 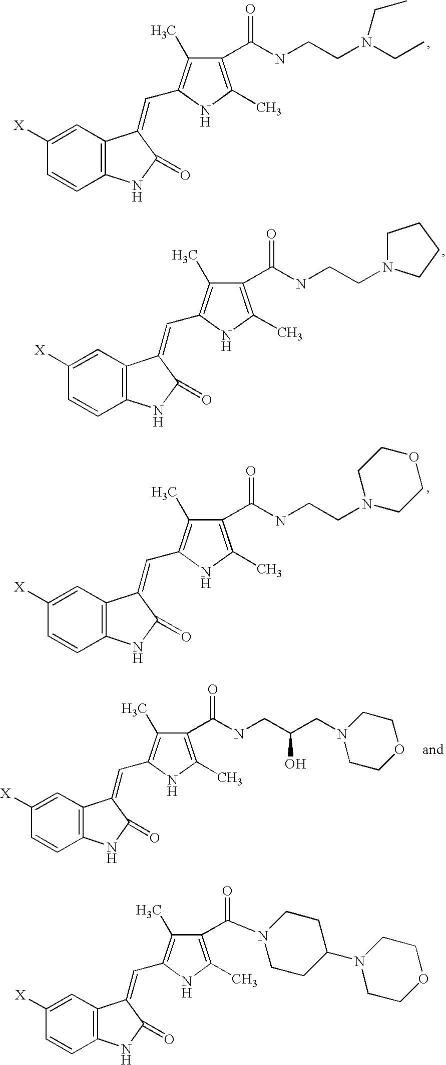
wherein X is F, Cl, I or Br;
or a pharmaceutically acceptable salt thereof.

21. The method of claim 18, wherein the compound of Formula I is 5-(5-Fluoro-2-oxo-1,2-dihydro-indol-3-ylidenemethyl)-2,4-dimethyl-1H-pyrrole-3-carboxylic acid (2-diethylamino-ethyl)-amide (Compound 1).

22. A kit comprising:
(a) antibody and/or nucleic acid for detecting the presence of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1; and
(b) instructions for determining whether or not a mammal will respond therapeutically to a method of treating cancer comprising administering a compound that inhibits tyrosine kinase activity.

23. A kit of claim 22, wherein said instructions comprise the steps of:
(i) measuring in a mammal the level of at least one of the following proteins and/or mRNA transcripts for such proteins and/or genes: PAI-1, TIMP-1, vinculin, VEGF, PLGF, VEGF/PLGF heterodimers, MIG, IP-10, I-TAC, eucaryotic initiation factor 4A11, human (clone 5) orphan G protein-coupled receptor (Genbank Accession No. L06797; CXCR4), Homo sapiens thymosin beta-10 gene, Homo sapiens hnRNPcore protein A1, human leucocyte antigen (CD37), human MHC class II HLA-DR beta-1, Homo sapiens translation initiation factor elF3 p66 subunit, Homo sapiens nm23-H2 gene, human acidic ribosomal phosphoprotein P0, human cyclophilin, GenBank Accession No. AI541256 (Homo sapiens cDNA), human T-cell receptor active beta chain, human MHC class II lymphocyte antigen (HLA-DP) beta chain, Homo sapiens MAP kinase kinase 3 (MKK3), human RLIP76 protein, MMP-9, lactoferrin, lipocalin-2, CD24 antigen, basic transcription factor 3 homologue, c-jun proto-oncogene, c-fos cellular oncogene, tyrosine phosphatase non-receptor type 2, cdc2 related protein kinase, cyclin C, DNA polymerase gamma, protein kinase C alpha, lipocortin II/annexin A2, histone H2B member R, amphiregulin, basic transcription factor 3, phosphoinositol 3-kinase p110 subunit, GCP-2, IL-1α, IL-1β, IL-2, NT4, GCP-2, IGFBP-1, GRO-β, TNFR1, FLT3L, IL-6, IL-8, C-reactive protein, MCP-1, TNFα, TARC, MMP7, leptin, pro-MMP1 (interstitial collegenase precursor), ITIH4, soluble VEGF receptor 2 (sVEGFR2), human KIAA0195, human beta-tubulin class III isotype (beta-3), human tropomyosin, 1-phosphatidyl inositol-4-phosphate-5-kinase isoform C; human MLC emb gene for embryonic myosin alkaline light chain, Homo sapiens glyoxalase II, Homo sapiens trans-golgi network glycoprotein 48, histone H2B, Genbank Accession No. W26677 (Homo sapiens cDNA), human PMI gene for a putative receptor protein, human DNA-binding protein A (dbpA), ephrin receptor EphB4, hanukah factor/granzyme A, von Hippel-Lindau (VHL) tumor suppressor, OB-cadherin 1, OB-cadherin 2, phosphoinositol 3-phosphate-binding protein-3 (PEPP3), phosphoinositol 3-kinase p85 subunit, mucin 1, hepatitis C-associated microtubular aggregate p44, ErbB3/HER3 receptor tyrosine kinase, gelsolin, cyclin D2, ENA-78 and MPIF-1;
(ii) exposing the mammal to a compound that inhibits tyrosine kinase activity; and
(iii) following the exposing step of (ii), measuring in the mammal the level of at least one of the proteins and/or mRNA transcripts for such proteins measured in step (i);
wherein a difference in the level of said proteins and/or mRNA transcripts measured in (iii), compared to the level of proteins and or mRNA transcripts measured in step (i) indicates that the mammal will respond therapeutically to a method of treating cancer comprising administering the compound that inhibits tyrosine kinase activity.

24. A method for testing or predicting whether a mammal will experience an adverse event in response to a method of treating cancer comprising administering a tyrosine kinase inhibitor, wherein the method for testing or predicting comprises:
(a) measuring in the mammal the level of IL-6 or C-reactive protein (CRP) protein and/or mRNA transcript for such protein and/or gene before administering the tyrosine kinase inhibitor;
(b) measuring in the mammal the level of IL-6 or CRP protein and/or mRNA transcript for such protein and/or gene after administering the tyrosine kinase inhibitor;
(c) comparing levels of said IL-6 or CRP protein and/or mRNA transcript measured in (a) and (b);
wherein a level of two-fold or greater of said protein and/or mRNA transcript as measured in step (b), compared to the level of said protein and/or mRNA transcript as measured in step (a), indicates that the mammal will experience fatigue in response to the method of treating cancer comprising administering the tyrosine kinase inhibitor.

25. The method of claim 24, wherein the tyrosine kinase inhibitor is a compound of Formula I or salt thereof.

26. The method of claim 25, wherein the compound of Formula I or salt thereof is 5-(5-Fluoro-2-oxo-1,2-dihydro-indol-3-ylidenemethyl)-2,4-dimethyl-1H-pyrrole-3-carboxylic acid (2-diethylamino-ethyl)-amide (Compound 1) or salt thereof.

27. A method of claim 24, wherein the adverse event is debilitating fatigue.

28. The method of claim 24, wherein the method is an in vitro method, and wherein the protein and/or mRNA is measured in at least one biological tissue from the mammal.

29. The method of claim 24, wherein the biological tissue comprises a biological fluid that is selected from the group consisting of whole fresh blood, peripheral blood mononuclear cells, frozen whole blood, fresh plasma, frozen plasma, urine and saliva.

30. The method of claim 24, wherein the tissue is selected from the group consisting of buccal mucosa tissue, skin, hair follicles, tumor tissue and bone marrow.


SEQUENCE LISTING (185 sequences)

SEQ ID NO: 1 - DNA, 20 bp, Artificial Sequence
  Description of Artificial Sequence  Primer
ttggctaccagtccagcagc

SEQ ID NO: 2 - DNA, 20 bp, Artificial Sequence
  Description of Artificial Sequence  Primer
gggttggtgtcccagtagga

SEQ ID NO: 3 - DNA, 19 bp, Artificial Sequence
  Description of Artificial Sequence  Primer
cccggagtgagttgaacca

SEQ ID NO: 4 - DNA, 20 bp, Artificial Sequence
  Description of Artificial Sequence  Primer
cctagtcctcagggcactgc

SEQ ID NO: 5 - DNA, 21 bp, Artificial Sequence
  Description of Artificial Sequence  Primer
cccagaagtggttgtttccct

SEQ ID NO: 6 - DNA, 22 bp, Artificial Sequence
  Description of Artificial Sequence  Primer
gtccatgtttttccttgagcct

SEQ ID NO: 7 - DNA, 19 bp, Artificial Sequence
  Description of Artificial Sequence  Primer
ctggaagcctgtgaattcc

SEQ ID NO: 8 - DNA, 21 bp, Artificial Sequence
  Description of Artificial Sequence  Primer
gaatggctgaggctttcttgg

SEQ ID NO: 9 - DNA, 24 bp, Artificial Sequence
  Description of Artificial Sequence  Primer
gctgacttcggaactaaaggagaa

SEQ ID NO: 10 - DNA, 20 bp, Artificial Sequence
  Description of Artificial Sequence  Primer
tgggacagggaagacgatgt

SEQ ID NO: 11 - DNA, 23 bp, Artificial Sequence
  Description of Artificial Sequence  Primer
ctgcctcgacacacataaacctt

SEQ ID NO: 12 - DNA, 24 bp, Artificial Sequence
  Description of Artificial Sequence  Primer
catctaagcatcagtgtgtgacca

SEQ ID NO: 13 - DNA, 22 bp, Artificial Sequence
  Description of Artificial Sequence  Primer
atgatgagtcggtcctctttcc

SEQ ID NO: 14 - DNA, 28 bp, Artificial Sequence
  Description of Artificial Sequence  Primer
tgacaattgaaagtttaaaaccatcata

SEQ ID NO: 15 - DNA, 20 bp, Artificial Sequence
  Description of Artificial Sequence  PrOBE
tccattgtcttatgatccac

SEQ ID NO: 16 - DNA, 28 bp, Artificial Sequence
  Description of Artificial Sequence  Primer
agttagaagttagggtttaggcatcatt

SEQ ID NO: 17 - DNA, 21 bp, Artificial Sequence
  Description of Artificial Sequence  Primer
tacccatgccctcactcaatc

SEQ ID NO: 18 - DNA, 18 bp, Artificial Sequence
  Description of Artificial Sequence  Probe
aagtgtcagcattctcaa

SEQ ID NO: 19 - DNA, 23 bp, Artificial Sequence
  Description of Artificial Sequence  Primer
ccagtgttgtgaggatgcatatc

SEQ ID NO: 20 - DNA, 21 bp, Artificial Sequence
  Description of Artificial Sequence  Primer
cagtcaacatcagcgcaaaga

SEQ ID NO: 21 - DNA, 17 bp, Artificial Sequence
  Description of Artificial Sequence  Probe
attcccatgccgtcgta

SEQ ID NO: 22 - DNA, 32 bp, Artificial Sequence
  Description of Artificial Sequence  Primer
caaacctactgtatctctaatacagtgtgact

SEQ ID NO: 23 - DNA, 27 bp, Artificial Sequence
  Description of Artificial Sequence  Primer
gacagagatgattatccctttaaacca

SEQ ID NO: 24 - DNA, 14 bp, Artificial Sequence
  Description of Artificial Sequence  Probe
agcgctcacctttg

SEQ ID NO: 25 - DNA, 30 bp, Artificial Sequence
  Description of Artificial Sequence  Primer
cctacagacagacatacatagacatttcaa

SEQ ID NO: 26 - DNA, 25 bp, Artificial Sequence
  Description of Artificial Sequence  Primer
attatgcttcatgtttcctggctta

SEQ ID NO: 27 - DNA, 27 bp, Artificial Sequence
  Description of Artificial Sequence  Probe
ccaaattaagaaatattatactaatca

SEQ ID NO: 28 - DNA, 26 bp, Artificial Sequence
  Description of Artificial Sequence  Primer
gacaacagttctgagctgtaatttcg

SEQ ID NO: 29 - DNA, 25 bp, Artificial Sequence
  Description of Artificial Sequence  Primer
tgggtttaagctggctgaatattat

SEQ ID NO: 30 - DNA, 20 bp, Artificial Sequence
  Description of Artificial Sequence  Probe
actctggacactctatatgt

SEQ ID NO: 31 - DNA, 23 bp, Artificial Sequence
  Description of Artificial Sequence  Primer
tcagccagcttaaacccatacaa

SEQ ID NO: 32 - DNA, 29 bp, Artificial Sequence
  Description of Artificial Sequence  Primer
tggcacgtattagtttaagatgaaagtag

SEQ ID NO: 33 - DNA, 18 bp, Artificial Sequence
  Description of Artificial Sequence  Probe
cttgttactgctgattct

SEQ ID NO: 34 - DNA, 19 bp, Artificial Sequence
  Description of Artificial Sequence  Primer
ttcagaggccccaccaatt

SEQ ID NO: 35 - DNA, 20 bp, Artificial Sequence
  Description of Artificial Sequence  Primer
cccacatgagcttccacaca

SEQ ID NO: 36 - DNA, 15 bp, Artificial Sequence
  Description of Artificial Sequence  Probe
tctcggacacttctc

SEQ ID NO: 37 - DNA, 22 bp, Artificial Sequence
  Description of Artificial Sequence  Primer
tgaggcagggacaagtctttct

SEQ ID NO: 38 - DNA, 21 bp, Artificial Sequence
  Description of Artificial Sequence  Primer
accctgactgaaggctcatga

SEQ ID NO: 39 - DNA, 19 bp, Artificial Sequence
  Description of Artificial Sequence  Probe
ctctttgagaccccagtgc

SEQ ID NO: 40 - DNA, 26 bp, Artificial Sequence
  Description of Artificial Sequence  Primer
tctaccgtccttgtcataactttgtg

SEQ ID NO: 41 - DNA, 19 bp, Artificial Sequence
  Description of Artificial Sequence  Primer
atgatgatgggcccctgtt

SEQ ID NO: 42 - DNA, 15 bp, Artificial Sequence
  Description of Artificial Sequence  Probe
cctttgcccaagttg

SEQ ID NO: 43 - DNA, 22 bp, Artificial Sequence
  Description of Artificial Sequence  Primer
tggacgttttgtgatcgaagag

SEQ ID NO: 44 - DNA, 26 bp, Artificial Sequence
  Description of Artificial Sequence  Primer
aagtcaaggcttctgtcttttcttct

SEQ ID NO: 45 - DNA, 20 bp, Artificial Sequence
  Description of Artificial Sequence  Probe
cttgagaatcctttccaacc

SEQ ID NO: 46 - protein, 10 aa, Homo sapiens
DIYSSFGFPR

SEQ ID NO: 47 - protein, 11 aa, Homo sapiens
DGFFYFFHGTR

SEQ ID NO: 48 - protein, 114 aa, Homo sapiens
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 49 - protein, 120 aa, Homo sapiens
MKVSVAALSCLMLVTALGSQARVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 50 - protein, 902 aa, Homo sapiens
MSEFRIHHDVNELLSLLRVHGGDGAEVYIDLLQKNRTPYVTTTVSAHSAKVKIAEFSRTPEDFLKKYDELKSKNTRNLDPLVYLLSKLTEDKETLQYLQQNAKERAELAAAAVGSSTTSINVPAAASKISMQELEELRKQLGSVATGSTLQQSLELKRKMLRDKQNKKNSGQHLPIFPAWVYERPALIGDFLIGAGISTDTALPIGTLPLASQESAVVEDLLYVLVGVDGRYVSAQPLAGRQSRTFLVDPNLDLSIRELVHRILPVAASYSAVTRFIEEKSSFEYGQVNHALAAAMRTLVKEHLILVSQLEQLHRQGLLSLQKLWFYIQPAMRTMDILASLATSVDKGECLGGSTLSLLHDRSFSYTGDSQAQELCLYLTKAASAPYFEVLEKWIYRGIIHDPYSEFMVEEHELRKERIQEDYNDKYWDQRYTIVQQQIPSFLQKMADKILSTGKYLNVVRECGHDVTCPVAKEIIYTLKERAYVEQIEKAFNYASKVLLDFLMEEKELVAHLRSIKRYFLMDQGDFFVHFMDLAEEELRKPVEDITPPRLEALLELALRMSTANTDPFKDDLKIDLMPHDLITQLLRVLAIETKQEKAMAHADPTELALSGLEAFSFDYIVKWPLSLIINRKALTRYQMLFRHMFYCKHVERQLCSVWISNKTAKQHSLHSAQWFAGAFTLRQRMLNFVQNIQYYMMFEVMEPTWHILEKNLKSASNIDDVLGHHTGFLDTCLKDCMLTNPELLKVFSKLMSVCVMFTNCMQKFTQSMKLDGELGGQTLEHSTVLGLPAGAEERARKELARKHLAEHADTVQLVSGFEATINKFDKNFSAHLLDLLARLSIYSTSDCEHGMASVISRLDFNGFYTERLERLSAERSQKATPQVPVLRGPPAPAPRVAVTAQ

SEQ ID NO: 51 - protein, 252 aa, Homo sapiens
MRAPLLPPAPVVLSLLILGSGHYAAGLDLNDTYSGKREPFSGDHSADGFEVTSRSEMSSGSEISPVSEMPSSSEPSSGADYDYSEEYDNEPQIPGYIVDDSVRVEQVVKPPQNKTESENTSDKPKRKKKGGKNGKNRRNRKKKNPCNAEFQNFCIHGECKYIEHLEAVTCKCQQEYFGERCGEKSMKTHSMIDSSLSKIALAAIAAFMSAVILTAVAVITVQLRRQYVRKYEGEAEERKKLRQENGNVHAIA

SEQ ID NO: 52 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 53 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 54 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 55 - protein, 125 aa, Homo sapiens
MKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 56 - protein, 210 aa, Homo sapiens
MLPLPSCSLPILLLFLLPSVPIESQPPPSTLPPFLAPEWDLLSPRVVLSRGAPAGPPLLFLLEAGAFRESAGAPANRSRRGVSETAPASRRGELAVCDAVSGWVTDRRTAVDLRGREVEVLGEVPAAGGSPLRQYFFETRCKADNAEEGGPGAGGGGCRGVDRRHWVSECKAKQSYVRALTADAQGRVGWRWIRIDTACVCTLLSRTGRA

SEQ ID NO: 57 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 58 - protein, 107 aa, Homo sapiens
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 59 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 60 - protein, 235 aa, Homo sapiens
MTVLAPAWSPTTYLLLLLLLSSGLSGTQDCSFQHSPISSDFAVKIRELSDYLLQDYPVTVASNLQDEELCGALWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 61 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 62 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 63 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 64 - protein, 94 aa, Homo sapiens
MAPLKMLALVTLLLGASLQHIHAARGTNVGRECCLEYFKGAIPLRKLKTWYQTSEDCSRDAIVFVTVQGRAICSDPNNKRVKNAVKYLQSLERS

SEQ ID NO: 65 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 66 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTRDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 67 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 68 - DNA, 2619 bp, Homo sapiens
gactcctaggggcttgcagacctagtgggagagaaagaacatcgcagcagccaggcagaaccaggacaggtgaggtgcaggctggctttcctctcgcagcgcggtgtggagtcctgtcctgcctcagggcttttcggagcctggatcctcaaggaacaagtagacctggccgcggggagtggggagggaaggggtgtctattgggcaacagggcggcaaagccctgaataaaggggcgcagggcaggcgcaagtgcagagccttcgtttgccaagtcgcctccagaccgcagacatgaaacttgtcttcctcgtcctgctgttcctcggggccctcggactgtgtctggctggccgtaggagaaggagtgttcagtggtgcgccgtatcccaacccgaggccacaaaatgcttccaatggcaaaggaatatgagaaaagtgcgtggccctcctgtcagctgcataaagagagactcccccatccagtgtatccaggccattgcggaaaacagggccgatgctgtgacccttgatggtggtttcatatacgaggcaggcctggccccctacaaactgcgacctgtagcggcggaagtctacgggaccgaaagacagccacgaactcactattatgccgtggctgtggtgaagaagggcggcagctttcagctgaacgaactgcaaggtctgaagtcctgccacacaggccttcgcaggaccgctggatggaatgtccctacagggacacttcgtccattcttgaattggacgggtccacctgagcccattgaggcagctgtggccaggttcttctcagccagctgtgttcccggtgcagataaaggacagttccccaacctgtgtcgcctgtgtgcggggacaggggaaaacaaatgtgccttctcctcccaggaaccgtacttcagctactctggtgccttcaagtgtctgagagacggggctggagacgtggcttttatcagagagagcacagtgtttgaggacctgtcagacgaggctgaaagggacgagtatgagttactctgcccagacaacactcggaagccagtggacaagttcaaagactgccatctggcccgggtcccttctcatgccgttgtggcacgaagtgtgaatggcaaggaggatgccatctggaatcttctccgccaggcacaggaaaagtttggaaaggacaagtcaccgaaattccagctctttggctcccctagtgggcagaaagatctgctgttcaaggactctgccattgggttttcgagggtgcccccgaggatagattctgggctgtaccttggctccggctacttcactgccatccagaacttgaggaaaagtgaggaggaagtggctgcccggcgtgcgcgggtcgtgtggtgtgcggtgggcgagcaggagctgcgcaagtgtaaccagtggagtggcttgagcgaaggcagcgtgacctgctcctcggcctccaccacagaggactgcatcgccctggtgctgaaaggagaagctgatgccatgagtttggatggaggatatgtgtacactgcatgcaaatgtggtttggtgcctgtcctggcagagaactacaaatcccaacaaagcagtgaccctgatcctaactgtgtggatagacctgtggaaggatatcttgctgtggcggtggttaggagatcagacactagccttacctggaactctgtgaaaggcaagaagtcctgccacaccgccgtggacaggactgcaggctggaatatccccatgggcctgctcttcaaccagacgggctcctgcaaatttgatgaatatttcagtcaaagctgtgcccctgggtctgacccgagatctaatctctgtgctctgtgtattggcgacgagcagggtgagaataagtgcgtgcccaacagcaacgagagatactacggctacactggggctttccggtgcctggctgagaatgctggagacgttgcatttgtgaaagatgtcactgtcttgcagaacactgatggaaataacaatgaggcatgggctaaggatttgaagctggcagactttgcgctgctgtgcctcgatggcaaacggaagcctgtgactgaggctagaagctgccatcttgccatggccccgaatcatgccgtggtgtctcggatggataaggtggaacgcctgaaacaggtgctgctccaccaacaggctaaatttgggagaaatggatctgactgcccggacaagttttgcttattccagtctgaaaccaaaaaccttctgttcaatgacaacactgagtgtctggccagactccatggcaaaacaacatatgaaaaatatttgggaccacagtatgtcgcaggcattactaatctgaaaaagtgctcaacctcccccctcctggaagcctgtgaattcctcaggaagtaaaaccgaagaagatggcccagctccccaagaaagcctcagccattcactgcccccagctcttctccccaggtgtgttggggccttggctcccctgctgaaggtggggattgcccatccatctgcttacaattccctgctgtcgtcttagcaagaagtaaaatgagaaattttgttgatattcaaaaaaaa

SEQ ID NO: 69 - protein, 711 aa, Homo sapiens
MKLVFLVLLFLGALGLCLAGRRRRSVQWCAVSQPEATKCFQWQRNMRKVRGPPVSCIKRDSPIQCIQAIAENRADAVTLDGGFIYEAGLAPYKLRPVAAEVYGTERQPRTHYYAVAVVKKGGSFQLNELQGLKSCHTGLRRTAGWNVPTGTLRPFLNWTGPPEPIEAAVARFFSASCVPGADKGQFPNLCRLCAGTGENKCAFSSQEPYFSYSGAFKCLRDGAGDVAFIRESTVFEDLSDEAERDEYELLCPDNTRKPVDKFKDCHLARVPSHAVVARSVNGKEDAIWNLLRQAQEKFGKDKSPKFQLFGSPSGQKDLLFKDSAIGFSRVPPRIDSGLYLGSGYFTAIQNLRKSEEEVAARRARVVWCAVGEQELRKCNQWSGLSEGSVTCSSASTTEDCIALVLKGEADAMSLDGGYVYTACKCGLVPVLAENYKSQQSSDPDPNCVDRPVEGYLAVAVVRRSDTSLTWNSVKGKKSCHTAVDRTAGWNIPMGLLFNQTGSCKFDEYFSQSCAPGSDPRSNLCALCIGDEQGENKCVPNSNERYYGYTGAFRCLAENAGDVAFVKDVTVLQNTDGNNNEAWAKDLKLADFALLCLDGKRKPVTEARSCHLAMAPNHAVVSRMDKVERLKQVLLHQQAKFGRNGSDCPDKFCLFQSETKNLLFNDNTECLARLHGKTTYEKYLGPQYVAGITNLKKCSTSPLLEACEFLRK

SEQ ID NO: 70 - DNA, 597 bp, Homo sapiens
atgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatatctgggcctccctgaaaaccacatcgtcttccctgtcccaatcgaccagtgtatcgacggctga

SEQ ID NO: 71 - protein, 198 aa, Homo sapiens
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKYLGLPENHIVFPVPIDQCIDG

SEQ ID NO: 72 - DNA, 2334 bp, Homo sapiens
agacacctctgccctcaccatgagcctctggcagcccctggtcctggtgctcctggtgctgggctgctgctttgctgcccccagacagcgccagtccacccttgtgctcttccctggagacctgagaaccaatctcaccgacaggcagctggcagaggaatacctgtaccgctatggttacactcgggtggcagagatgcgtggagagtcgaaatctctggggcctgcgctgctgcttctccagaagcaactgtccctgcccgagaccggtgagctggatagcgccacgctgaaggccatgcgaaccccacggtgcggggtcccagacctgggcagattccaaacctttgagggcgacctcaagtggcaccaccacaacatcacctattggatccaaaactactcggaagacttgccgcgggcggtgattgacgacgcctttgcccgcgccttcgcactgtggagcgcggtgacgccgctcaccttcactcgcgtgtacagccgggacgcagacatcgtcatccagtttggtgtcgcggagcacggagacgggtatcccttcgacgggaaggacgggctcctggcacacgcctttcctcctggccccggcattcagggagacgcccatttcgacgatgacgagttgtggtccctgggcaagggcgtcgtggttccaactcggtttggaaacgcagatggcgcggcctgccacttccccttcatcttcgagggccgctcctactctgcctgcaccaccgacggtcgctccgacggcttgccctggtgcagtaccacggccaactacgacaccgacgaccggtttggcttctgccccagcgagagactctacacccgggacggcaatgctgatgggaaaccctgccagtttccattcatcttccaaggccaatcctactccgcctgcaccacggacggtcgctccgacggctaccgctggtgcgccaccaccgccaactacgaccgggacaagctcttcggcttctgcccgacccgagctgactcgacggtgatggggggcaactcggcgggggagctgtgcgtcttccccttcactttcctgggtaaggagtactcgacctgtaccagcgagggccgcggagatgggcgcctctggtgcgctaccacctcgaactttgacagcgacaagaagtggggcttctgcccggaccaaggatacagtttgttcctcgtggcggcgcatgagttcggccacgcgctgggcttagatcattcctcagtgccggaggcgctcatgtaccctatgtaccgcttcactgaggggccccccttgcataaggacgacgtgaatggcatccggcacctctatggtcctcgccctgaacctgagccacggcctccaaccaccaccacaccgcagcccacggctcccccgacggtctgccccaccggaccccccactgtccacccctcagagcgccccacagctggccccacaggtcccccctcagctggccccacaggtccccccactgctggcccttctacggccactactgtgcctttgagtccggtggacgatgcctgcaacgtgaacatcttcgacgccatcgcggagattgggaaccagctgtatttgttcaaggatgggaagtactggcgattctctgagggcagggggagccggccgcagggccccttccttatcgccgacaagtggcccgcgctgccccgcaagctggactcggtctttgaggagccgctctccaagaagcttttcttcttctctgggcgccaggtgtgggtgtacacaggcgcgtcggtgctgggcccgaggcgtctggacaagctgggcctgggagccgacgtggcccaggtgaccggggccctccggagtggcagggggaagatgctgctgttcagcgggcggcgcctctggaggttcgacgtgaaggcgcagatggtggatccccggagcgccagcgaggtggaccggatgttccccggggtgcctttggacacgcacgacgtcttccagtaccgagagaaagcctatttctgccaggaccgcttctactggcgcgtgagttcccggagtgagttgaaccaggtggaccaagtgggctacgtgacctatgacatcctgcagtgccctgaggactagggctcccgtcctgctttgcagtgccatgtaaatccccactgggaccaaccctggggaaggagccagtttgccggatacaaactggtattctgttctggaggaaagggaggagtggaggtgggctgggccctctcttctcacctttgttttttgttggagtgtttctaataaacttggattctctaaccttt

SEQ ID NO: 73 - DNA, 2116 bp, Homo sapiens
cggttctccaagcacccagcatcctgctagacgcgccgcgcaccgacggaggggacatgggcagagcaatggtggccaggctggggctggggctgctgctgctggcactgctcctacccacgcagatttattccagtgaaacaacaactggaacttcaagtaactcctcccagagtacttccaactctgggttggccccaaatccaactaatgccaccaccaaggcggctggtggtgccctgcagtcaacagccagtctcttcgtggtctcactctctcttctgcatctctactcttaagagactcaggccaagaaacgtcttctaaatttccccatcttctaaacccaatccaaatggcgtctggaagtccaatgtggcaaggaaaaacaggtcttcatcgaatctactaattccacaccttttattgacacagaaaatgttgagaatcccaaatttgattgatttgaagaacatgtgagaggtttgactagatgatgaatgccaatattaaatctgctggagtttcatgtacaagatgaaggagaggcaacatccaaaatagttaagacatgatttccttgaatgtggcttgagaaatatggacacttaatactaccttgaaaataagaatagaaataaaggatgggattgtggaatggagattcagttttcattggttcattaattctataaggccataaaacaggtaatataaaaagcttccatcgatctatttatatgtacatgagaaggaatccccaggtgttactgtaattcctcaacgtattgtttcgacggcactaatttaatgccgatatactctagatgaatgtttacattgttgagctattgctgttctcttgggaactgaactcactttcctcctgaggctttggatttgacattgcatttgaccttttaggtagtaattgacatgtgccagggcaatgatgaatgagaatctaccccagatccaagcatcctgagcaactcttgattatccatattgagtcaaatggtaggcatttcctatcacctgtttccattcaacaagagcactacattcttttagctaaacggattccaaagagtagaattgcattgaccacgactaatttcaaaatgctttttattattattattttttagacagtctcactttgtcgcccaggccggagtgcagtggtgcgatctcagatcagtgtaccatttgcctcccgggctcaagcgattctcctgcctcagcctcccaagtagctgggattacaggcacctgccaccatgcccggctaatttttgtaattttagtagagacagggtttcaccatgttgcccaggctggtttagaactcctgacctcaggtgatccacccgcctcggcctcccaaagtgctgggattacaggcttgagcccccgcgcccagccatcaaaatgctttttatttctgcatatgtttgaatactttttacaatttaaaaaaatgatctgttttgaaggcaaaattgcaaatcttgaaattaagaaggcaaaatgtaaaggagtcaaactataaatcaagtatttgggaagtgaagactggaagctaatttgcataaattcacaaacttttatactctttctgtatatacattttttttctttaaaaaacaactatggatcagaatagcaacatttagaacactttttgttatcagtcaatatttttagatagttagaacctggtcctaagcctaaaagtgggcttgattctgcagtaaatcttttacaactgcctcgacacacataaacctttttaaaaatagacactccccgaagtcttttgtttgtatggtcacacactgatgcttagatgttccagtaatctaatatggccacagtagtcttgatgaccaaagtcctttttttccatctttagaaaactacatgggaacaaacagatcgaacagttttgaagctactgtgtgtgtgaatgaacactcttgctttattccagaatgctgtacatctattttggattgtatattgtggttgtgtatttacgctttgattcatagtaacttcttatggaattgatttgcattgaacgacaaactgtaaataaaaagaaacggtg

SEQ ID NO: 74 - protein, 80 aa, Homo sapiens
MGRAMVARLGLGLLLLALLLPTQIYSSETTTGTSSNSSQSTSNSGLAPNPTNATTKAAGGALQSTASLFVVSLSLLHLYS

SEQ ID NO: 75 - DNA, 1864 bp, Homo sapiens
gtggtttttcggatcatgtctggtggctccgcggattataacagagaacatggcggcccagagggaatggaccccgatggtgtcatcgagagcaactggaatgagattgttgataactttgatgatatgaatttaaaggagtctctccttcgtggcatctatgcttacggttttgagaagccttccgctattcagcagagagctattattccctgtattaaagggtatgatgtgattgctcaagctcagtcaggtactggcaagacagccacatttgctatttccatcctgcaacagttggagattgagttcaaggagacccaagcactagtattggcccccaccagagaactggctcaacagatccaaaaggtaattctggcacttggagactatatgggagccacttgtcatgcctgcattggtggaacaaatgttcgaaatgaaatgcaaaaactgcaggctgaagcaccacatattgttgttggtacacccgggagagtgtttgatatgttaaacagaagatacctttctccaaaatggatcaaaatgtttgttttggatgaagcagatgaaatgttgagccgtggttttaaggatcaaatctatgagattttccaaaaactaaacacaagtattcaggttgtgtttgcttctgccacaatgccaactgatgtgttggaagtgaccaaaaaattcatgagagatccaattcgaattctggtgaaaaaggaagaattgacccttgaaggaatcaaacagttttatattaatgttgagagagaggaatggaagttggatacactttgtgacttgtacgagacactgaccattacacaggctgttatttttctcaatacgaggcgcaaggtggactggctgactgagaagatgcatgccagagacttcacagtttctgctctgcatggtgacatggaccagaaggagagagatgttatcatgagggaattccggtcagggtcaagtcgtgttctgatcactactgacttgttggctcgcgggattgatgtgcaacaagtgtctttggttataaattatgatctacctaccaatcgtgaaaactatattcacagaattggcagagggggtcgatttgggaggaaaggtgtggctataaactttgttactgaagaagacaagaggattcttcgtgacattgagactttctacaatactacagtggaggagatgcccatgaatgtggctgaccttatttaattcctgggatgagagttttggatgcagtgctcgctgttgctgaataggcgatcacaacgtgcattgtgcttctttctttgggaatatttgaatcttgtctcaatgctcataacggatcagaaatacagattttgatagcaaagcgacgttagtcgtgagctcttgtgaggaaagtcattggctttatcctctttagagttagactgttggggtgggtataaaagatggggtctgtaaaatctttctttcttagaaatttatttcctagttctgtagaaatggttgtattagatgttctctatcatttaataatatacttgtggactaaaagatataagtgctgtataaaatcagccaattatgttaaactagcatatctgcctttattgtgtttgtcattagcctgagtagaaaggcctttaaaatttttttagaaagcatttgaatgcattttgtttggtattgtatttattcaataaagtatttaattagtgctaagtgtgaactggaccctgttgctaagccccagcaagcaatcctaggtagggtttaatccccagtaaaattgccatattgcacatgtcttaatgaagtttgaatgttaaataaattgtatattcacttt

SEQ ID NO: 76 - protein, 407 aa, Homo sapiens
MSGGSADYNREHGGPEGMDPDGVIESNWNEIVDNFDDMNLKESLLRGIYAYGFEKPSAIQQRAIIPCIKGYDVIAQAQSGTGKTATFAISILQQLEIEFKETQALVLAPTRELAQQIQKVILALGDYMGATCHACIGGTNVRNEMQKLQAEAPHIVVGTPGRVFDMLNRRYLSPKWIKMFVLDEADEMLSRGFKDQIYEIFQKLNTSIQVVFASATMPTDVLEVTKKFMRDPIRILVKKEELTLEGIKQFYINVEREEWKLDTLCDLYETLTITQAVIFLNTRRKVDWLTEKMHARDFTVSALHGDMDQKERDVIMREFRSGSSRVLITTDLLARGIDVQQVSLVINYDLPTNRENYIHRIGRGGRFGRKGVAINFVTEEDKRILRDIETFYNTTVEEMPMNVADLI

SEQ ID NO: 77 - DNA, 1670 bp, Homo sapiens
cggcacgaggcaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaagagacttttttttatacgataaataacttttttttaagttacacatttttcagatataaaagactgaccaatattgtacagtttttattgcttgttggatttttgctcttgtgtttctttagtttttcgtgaaggtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaa

SEQ ID NO: 78 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 79 - DNA, 1262 bp, Homo sapiens
  misc_feature (53)..(53): n is a, c, g, or t
cgtcctacatctcgcgcatacacgcccacgtgcgcacatcactgggggtgccncgggagacagagccgctggtagcctaaggngggggggcagccaggagaaagccccgccgctgctcgtcccgcccctcgggtgccagcaccgcccctgctgcggcgggtgaggggcggggcggggccgcggcgtatataaggctaggcggggcgccgctcttttgtttcttgctgcagcaacgcgagtgggagcaccaggatctcgggctcggaacgagactgcacggtgacgtgacggccgggcgggggcccagggtgtggtcggatccggtgcaccgcgggcgcgcaacccggacaggcgcttctcggaccggacgcaggggccgcgaccacgccctgggaccgagaagaggggtgcggacgcgcccagatcctcggccttggggctgctcggcagccttggcgcgagtgccacgtcgagaggcgtcggcggggagcgcggaaggggacggcctgcgcccaggcccaggtcaagcgccttggtttgcccactaggattgttttaagaaaatggcagacaaaccagacatgggggaaatcgccagcttcgataaggccaagctgaagaaaacggagacgcaggagaagaacaccctgccgaccaaagagagtgagtgtgcctcggtctccgcgccccagcccagcccctcaccctgctcttccttgcaaacccactcctccaccccccaccccgccgttgtccccggtgtgggcggccccggcactctttcagtttcacaaagcgccttgtttctccccagccccaagcttccttctaaatcccacacctcgtggtgctcatcacaccgggaagcacctcggttgcgggtggggggttgcagcncccctccagcgccccgttccgtctcaagccattgagcaggagaagcggagtgaaatttcctaagatcctggaggatttcctaccccccgtctctcggagcaccccagtcgctgatgtggagaagagccaccctgcaagatggacacgagtccacaagctgcactgtgaacctgcgagcccgcgccgatgccaccggcctgtggtcgtctgaagggacccccccccaatcggactgccaaattctcggtttgccccgggatattatagaaaattatttgtatgaataatgaaaataaaacacacctcgttggcatggctggcggtggtctgagtgttttagttagtatgggtgcagtccactgcag

SEQ ID NO: 80 - protein, 49 aa, Homo sapiens
DCFKKMADKPDMGEIASFDKAKLKKTETQEKNTLPTKETIEQEKRSEIS

SEQ ID NO: 81 - DNA, 1198 bp, Homo sapiens
ttaaagtctctcttcaccctgccgtcatgtctaagtcagagtctcctaaagagcccgaacagctgaggaagctcttcattggagggttgagctttgaaacaactgatgagagcctgaggagccattttgagcaatggggaacgctcacggactgtgtggtaatgagagatccaaacaccaagcgctctaggggctttgggtttgtcacatatgccactgtggaggaggtggatgcagctatgaatgcaaggccacacaaggtggatggaagagttgtggaaccaaagagagctgtctccagagaagattctcaaagaccaggtgcccacttaactgtgaaaaagatatttgttggtggcattaaagaagacactgaagaacatcacctaagagattattttgaacagtatggaaaaattgaagtgattgaaatcatgactgaccgaggcagtggcaagaaaaggggctttgcctttgtaacctttgacgaccatgactccgtggataagattgtcattcagaaataccatactgtgaatggccacaactgtgaagttagaaaagccctgtcaaagcaagagatggctagtgcttcatccagccaaagaggtcgaagtggttctggaaactttggtggtggtcgtggaggtggtttcggtgggaatgacaacttcggtcgtggaggaaacttcagtggtcgtggtggctttggtggcagccgtggtggtggtggatatggtggcagtggggatggctataatggatttggcaatgatggaagcaattttggaggtggtggaagctacaatgattttgggaattacaacaatcagtcttcaaattttggacccatgaagggaggaaattttggaggcagaagctctggcccctatggcggtggaggccaatactttgcaaaaccacgaaaccaaggtggctatggcggttccagcagcagcagtagctatggcagtggcagaagattttaattagggaggagtctgctactagtcttatcagctcttaaaaacagaaactcatctgtccaagttcgtggcagaaaggaacgtccttgtgaagacctttatctgagccactgtacttcgttatcacgccatgcagtttacatgagctgttctgcagctcgaaattccattttgtgaatgggtttttttttttaataaactgtatttaactt

SEQ ID NO: 82 - protein, 320 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGSNFGGGGSYNDFGNYNNQSSNFGPMKGGNFGGRSSGPYGGGGQYFAKPRNQGGYGGSSSSSSYGSGRRF

SEQ ID NO: 83 - DNA, 1125 bp, Homo sapiens
gtctcccccactgtcagcacctcttctgtgtggtgagtggaccgcttaccccactaggtgaagatgtcagcccaggagagctgcctcagcctcatcaagtacttcctcttcgttttcaacctcttcttcttcgtcctcggcagcctgatcttctgcttcggcatctggatcctcatcgacaagaccagcttcgtgtcctttgtgggcttggccttcgtgcctctgcagatctggtccaaagtcctggccatctcaggaatcttcaccatgggcatcgccctcctgggttgtgtgggggccctcaaggagctccgctgcctcctgggcctgtattttgggatgctgctgctcctgtttgccacacagatcaccctgggaatcctcatctccactcagcgggcccagctggagcgaagcttgcgggacgtcgtagagaaaaccatccaaaagtacggcaccaaccccgaggagaccgcggccgaggagagctgggactatgtgcagttccagctgcgctgctgcggctggcactacccgcaggactggttccaagtcctcatcctgagaggtaacgggtcggaggcgcaccgcgtgccctgctcctgctacaacttgtcggcgaccaacgactccacaatcctagataaggtgatcttgccccagctcagcaggcttggacacctggcgcggtccagacacagtgcagacatctgcgctgtccctgcagagagccacatctaccgcgagggctgcgcgcagggcctccagaagtggctgcacaacaaccttatttccatagtgggcatttgcctgggcgtcggcctactcgagctcgggttcatgacgctctcgatattcctgtgcagaaacctggaccacgtctacaaccggctcgctcgataccgttaggccccgccctccccaaagtcccgccccgcccccgtcacgtgcgctgggcacttccctgctgcctgtaaatatttgtttaatccccagttcgcctggagccctccgccttcacattcccctggggacccacgtggctgcgtgcccctgctgctgtcacctctcccacgggacctggggctttcgtccacagcttcctgtccccatctgtcggcctac

SEQ ID NO: 84 - protein, 281 aa, Homo sapiens
MSAQESCLSLIKYFLFVFNLFFFVLGSLIFCFGIWILIDKTSFVSFVGLAFVPLQIWSKVLAISGIFTMGIALLGCVGALKELRCLLGLYFGMLLLLFATQITLGILISTQRAQLERSLRDVVEKTIQKYGTNPEETAAEESWDYVQFQLRCCGWHYPQDWFQVLILRGNGSEAHRVPCSCYNLSATNDSTILDKVILPQLSRLGHLARSRHSADICAVPAESHIYREGCAQGLQKWLHNNLISIVGICLGVGLLELGFMTLSIFLCRNLDHVYNRLARYR

SEQ ID NO: 85 - DNA, 1216 bp, Homo sapiens
agttctccctgagtgagacttgcctgctcctctggcccctggtcctgtcctgttctccagcatggtgtgtctgaagctccctggaggttcctacatggcagtgctgacagtgacactgatggtgctgagctccccactggctttggctggggacacccgaccatgtttcttgcagcaggataagtatgagtgtcatttcttcaacgggacggagcgggtgcggttcctgcacagaggcatctataaccaacaggagaacgtgcgcttcgacagcgacgtgggggagtaccgggcggtgacggagctggggcggcctgacgctgagtactggaacagccagaaggacatcctggagcaggcgcgggccgcggtggacacctactgcagacacaactacggggctgtggagagcttcacagtgcagcggcgagttgagcctaaggtgactgtgtatcctgcaaggacccagaccctgcagcaccacaacctcctggtctgctctgtgaatggtttctatccaggcagcattgaagtcaggtggttccggaacggccaggaagagaaggctggggtggtgtccacaggcctgattcagaatggagactggaccttccagattctggtgatgctggaaacagttcctcggagtggagaggtttacacctgccaagtggagcacccaagcgtgacgagccctctcacagtggaatggagagcacagtctgaatctgcacagagcaagatgctgagtggaatcgggggctttgtgctgggcctgctcttccttggggccgggctattcatctacttcaagaatcagaaagggcactctggacttcacccaacaggactcgtgagctgaagtgcagatgaccacattcaagggggaaccttctgccccagctttgcatgatgaaaagctttcctgcttggctcttattcttccacaagagaggactttctcaggccctggttgctaccggttcagcaactctgcagaaaatgtccatccttgtggcttcctcagctcctgcccttggcctgaagtcccagcattgatggcagtgcctcatcttcaactttagtgctcccctttacctaaccctacggcctcccatgcatctgtactccccctgtgccacaaatggactacgttattaaatttttctgaagcccagagttaaaaatcatctgtccacctggcaccaaagacaaa

SEQ ID NO: 86 - protein, 266 aa, Homo sapiens [sequence omitted]

SEQ ID NO: 87 - DNA, 1881 bp, Homo sapiens
gaattcggcacgagctaacgcggtccccggcacgcaccatctgttgccatcccggccggccgaggccattgcagattttggaagatggcaaagttcatgacacccgtgatccaggacaacccctcaggctggggtccctgtgcggttcccgagcagtttcgggatatgccctaccagccgttcagcaaaggagatcggctaggaaaggttgcagactggacaggagccacataccaagataagaggtacacaaataagtactcctctcagtttggtggtggaagtcaatatgcttatttccatgaggaggatgaaagtagcttccagctggtggatacagcgcgcacacagaagacggcctaccagcggaatcgaatgagatttgcccagaggaacctccgcagagacaaagatcgtcggaacatgttgcagttcaacctgcagatcctgcctaagagtgccaaacagaaagagagagaacgcattcgactgcagaaaaagttccagaaacaatttggggttaggcagaaatgggatcagaaatcacagaaaccccgagactcttcagttgaagttcgtagtgattgggaagtgaaagaggaaatggattttcctcagttgatgaagatgcgctacttggaagtatcagagccacaggacattgagtgttgtggggccctagaatactacgacaaagcctttgaccgcatcaccacgaggagtgagaagccactgcggagcatcaagcgcatcttccacactgtcaccaccacagacgaccctgtcatccgcaagctggcaaaaactcaggggaatgtgtttgccactgatgccatcctggccacgctgatgagctgtacccgctcagtgtattcctgggatattgtcgtccagagagttgggtccaaactcttctttgacaagagagacaactctgactttgacctcctgacagtgagtgagactgccaatgagccccctcaagatgaaggtaattccttcaattcaccccgcaacctggccatggaggcaacctacatcaaccacaatttctcccagcagtgcttgagaatggggaaggaaagatacaacttccccaacccaaacccgtttgtggaggacgacatggataagaatgaaatcgcctctgttgcgtaccgttaccgcaggtggaagcttggagatgatattgaccttattgtccgttgtgagcacgatggcgtcatgactggagccaacggggaagtgtccttcatcaacatcaagacactcaatgagtgggattccaggcactgtaatggcgttgactggcgtcagaagctggactctcagcgaggggctgtcattgccacggagctgaagaacaacagctacaagttggcccggtggacctgctgtgctttgctggctggatctgagtacctcaagcttggttatgtgtctcggtaccacgtgaaagactcctcacgccacgtcatcctaggcacccagcagttcaagcctaatgagtttgccagccagatcaacctgagcgtggagaatgcctggggcattttacgctgcgtcattgacatctgcatgaagctggaggagggcaaatacctcatcctcaaggaccccaacaagcaggtcatccgtgtctacagcctccctgatggcaccttcagctctgatgaagatgaggaggaagaggaggaggaagaagaggaagaagaagaggaagaaacttaaaccagtgatgtggagctggagtttgtccttccaccgagactacgagggcctttgatgcttagtggaatgtgtgtctaacttgctctctgacatttagcagatgaaataaaatatatatctgtttagtcttttaaaaaaaaaaaaaaaaaa

SEQ ID NO: 88 - protein, 548 aa, Homo sapiens
MAKFMTPVIQDNPSGWGPCAVPEQFRDMPYQPFSKGDRLGKVADWTGATYQDKRYTNKYSSQFGGGSQYAYFHEEDESSFQLVDTARTQKTAYQRNRMRFAQRNLRRDKDRRNMLQFNLQILPKSAKQKERERIRLQKKFQKQFGVRQKWDQKSQKPRDSSVEVRSDWEVKEEMDFPQLMKMRYLEVSEPQDIECCGALEYYDKAFDRITTRSEKPLRSIKRIFHTVTTTDDPVIRKLAKTQGNVFATDAILATLMSCTRSVYSWDIVVQRVGSKLFFDKRDNSDFDLLTVSETANEPPQDEGNSFNSPRNLAMEATYINHNFSQQCLRMGKERYNFPNPNPFVEDDMDKNEIASVAYRYRRWKLGDDIDLIVRCEHDGVMTGANGEVSFINIKTLNEWDSRHCNGVDWRQKLDSQRGAVIATELKNNSYKLARWTCCALLAGSEYLKLGYVSRYHVKDSSRHVILGTQQFKPNEFASQINLSVENAWGILRCVIDICMKLEEGKYLILKDPNKQVIRVYSLPDGTFSSDEDEEEEEEEEEEEEEEET

SEQ ID NO: 89 - DNA, 670 bp, Homo sapiens
cggccacgaggcggaatcccttctgctctcccagcgcagcgccgccgcccggcccctccagcttcccggaccatggccaacctggagcgcaccttcatcgccatcaagccggacggcgtgcagcgcggcctggtgggcgagatcatcaagcgcttcgagcagaagggattccgcctcgtggccatgaagttcctccgggcctctgaagaacacctgaagcagcactacattgacctgaaagaccgaccattcttccctgggctggtgaagtacatgaactcagggccggttgtggccatggtctgggaggggctgaacgtggtgaagacaggccgagtgatgcttggggagaccaatccagcagattcaaagccaggcaccattcgtggggacttctgcattcaggttggcaggaacatcattcatggcagtgattcagtaaaaagtgctgaaaaagaaatcagcctatggtttaagcctgaagaactggttgactacaagtcttgtgctcatgactgggtctatgaataagaggtggacacaacagcagtctccttcagcacggcgtggtgtgtccctggacacagctcttcattccattgacttagaggcaacaggattgatcattcttttatagagcatatttgccaataaagcttttggaagccgg

SEQ ID NO: 90 - protein, 152 aa, Homo sapiens
MANLERTFIAIKPDGVQRGLVGEIIKRFEQKGFRLVAMKFLRASEEHLKQHYIDLKDRPFFPGLVKYMNSGPVVAMVWEGLNVVKTGRVMLGETNPADSKPGTIRGDFCIQVGRNIIHGSDSVKSAEKEISLWFKPEELVDYKSCAHDWVYE

SEQ ID NO: 91 - DNA, 1097 bp, Homo sapiens
cttctctcgccaggcgtcctcgtggaagtgacatcgtctttaaaccccctcgtggcaatccctgacgcaccgccgtgatgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaatcaccaaaaagcaaccaacttagccagttttatttgcaaaacaaggaaataaaggcttacttcttt

SEQ ID NO: 92 - protein, 317 aa, Homo sapiens
MPREDRATWKSNYFLKIIQLLDDYPKCFIVGADNVGSKQMQQIRMSLRGKAVVLMGKNTMMRKAIRGHLENNPALEKLLPHIRGNVGFVFTKEDLTEIRDMLLANKVPAAARAGAIAPCEVTVPAQNTGLGPEKTSFFQALGITTKISRGTIEILSDVQLIKTGDKVGASEATLLNMLNISPFSFGLVIQQVFDNGSIYNPEVLDITEETLHSRFLEGVRNVASVCLQIGYPTVASVPHSIINGYKRVLALSVETDYTFPLAEKVKAFLADPSAFVAAAPVAAATTAAPAAAAAPAKVEAKEESEESDEDMGFGLFD

SEQ ID NO: 93 - DNA, 6711 bp, Homo sapiens
gaattcccttgtaaggttttcttaacaaaacaccagtcacataagtgcattttattttatatttttgtttatttatttgagacggagtctcttgtctctcaggctggagtgcagtggcgccatctctgctcgctgcaacctccacctcctgggttccagcgattctcctgcctcagcctcccgagggggtagctgggactacaggtgcgcaccaccatgcccagctaattttgtatttttcgtagagatggggtttcaccatgttgtccaggctggtcttgaactcctgacctcaggtgatcctcccgcctcggcctcccaaagtgctggaattacaggcgtgatccaccgcacccggcctattttttgagagagggtcacactctgtcgtcccggctggaatgcagtgatgcgatcaccgcccactacagcctcgacctccgggctcaagcaatcctccccgcccagcctcctgagtagcgagcgcctcgacgcccagctaatttttatttttatttatttttttgtagagacggcgtctctctaagatgcccaggctggtggccggtgtcgaactcctaagatgaagcgatcctccccggccttggcctccgcgcctcctaaagcgccaggtatgagccaccgcgcctggcctacaagtgcattttaattaaagtattattaatgtctttgcctgaagaaattcgcttttaaattgtgacttatctttcacccaaaaatcaaagcacaattcagccccgaggcgggggcggtaggagctgggcggggcgggggcagggaaagaccaggagcagagattcaaaaagagtaagagggcaaaatgtgcataatgcatcttcacaggtaagagcctggccaggctcctgttttaatggcttcctcctgaagaagattcaagcagagtgtaagatattttcggaaagtagagcattttgaaagcatttcataatctctcaaaaccggagactgctcctgtcccacctcgttagagaaaacagcgatgctcaaaggcaacctccttcctgacattgcctggtaggacgcgacgtggtgtttgcccgcgcggaatgcggacgcaaggctgctcctaggtctcggggacgcgccatccccatttccgctcgcggaggcgtagggtccgggcgcgggaccccagtcgaccttgactggcggcgcgaccttgaggcctgcgttcgcctcagttgccccctctgtgcaatggggagacgcgcctcatcgcttgacaacggccgaagagccgccgcgcttccgtctcccgcgtgcgcgcgccatgctgcccacccccgttccgcactgaccctcccccgtgccccgcgtcccgtactgccgccccgccccgagtcccatgccgcagccaccgcgacggagcccgcaggcgggaacctgcctccgcgcgttagcgcgcacgcgcgcctcatgtgtcgtccccatcagcgccggcttccgtctataggccagatgcactgtcactctggcgaagtcgcagacccgattggccgggacggaggcgcgagaccgggttgcgggcggggccgaacgtggtataaaacgggcgggaggccaggctcgtgccgttttgcagacgccaccgccgaggaaaaccgtgtactattagccatggtcaaccccaccgtgttcttcgacattgccgtcgacggcgagcccttgggccgcgtctcctttgaggtcgggcgggcggcggcgtgcgggaatggggcccagaaagtgggccggggtcggggtgggtggtagcgccccaaaggcccgggcgcggggcgaccctgcttgaggggcgagcgcgggcgggctgcggcgccatttcctgacgaggggccattttgggaggtccgcgagtcgcgggaggaggccgggacgcggcggacaaaggcaggcggggcggctgcgaggccgttgggggagggggcccgcgtccgcccgcccgcctcatgtggccgcgccctgtcctgtccgacgcacgtgctcggcggccgcgctcaggtccgcgccttgagagtcgttgtccgccctagcttggcctgggcgccgcagaccggagccagaagcacgctcgcgggggcttgcgaccgccttcctgggaagctgtcccctggcaggcatgggtgctttacatcctgagctgggaagctgtttgcttgagggtttttctcaaggatcgaggcgcggtgtgagcccgtccatgctcggtcctgtagatcccgggaggccatgttataaaaggagacttgctgggatgtgacgggttgccacttgaaatatcttccatttggataaagtaggaatatttatacatgtgccccaaacgtccctccgtgtcccccacccccaagcggaaatgtgaaaatgggccttgcctttgctggtgcccaaggaccgccttccactgcagtgacggcgctggcgggggaggcgctcttgagcccctcccgattgtccctctgcctagcaagcaagttgcgactggccacaaggcaggcctcttccgaccaaggtggattaccagtgattacctaattagttttgagagcgttaaatgagttcttaaagatcagttgtaattatagcatagtatctaaacttggcgcgtgtcttcaaagttaaatattgagtacgattccgttccagttaacatggatagaccttagggagtagcgaaataggatgttagtggttttattcctttaaatcacatctcaaaaggccaccaatggctagtttggatcttattccgaaaatagattgatcctcatgcagtcttcgtgaggacagagcgatttccttgttgcctaccctgtccatagtgcctggcacataggcactgaaacactgcatgttaatccacaccccaccccacctatgagtgtagtcaaagctggtaagtgacaagggctttcgtggaaacttggcctgacctaatgttgggcatcaggttacccaaagagcttcagggaaatgagaaaggacttgcaggtcttgatgagaatggaggggtaactgccaatgagggctttggctttagcgaaagtctgaaagggaagccataggaacttaaacgtaccgactataaagctctgagaaaagctgatgttttagaaagaccatacattctaggtacaaatacctaaaaactaaaaaataagtacgttggccaggcgggcggatcacgaagtcaggagattgagaccatcctgggcccctggtgaaaccccacctctattaaaaatacaaaaattagctgggcgtggtggcgcttgcctgtaatctcagctactctagaggctgaggcaggagatcgcttgaaccccggaggcggaggctgcagtgagccgagatcgtgccactgcactccagcctggtgacagcgagactcttgtctcaaaaaaaaaaaagtacattgctataagagaagtgcacacggatactagtagttaattcagtcacatctgtgaaatagcttataaaatgctacttttaaacaagctgtttttatgaaagggcttgtaaatgtttatggtatttaagctacctctctagccataacgtattatacattcaagaaaggttcaaaaccagatatactagaaaccaatctttattttttaccccactactaggtaagggcctggataccaagaagtgactgctcatctaatccataaagctatgttaacagattggaggtagtagcattttcattacaagtgactaaaagaacagctgtttacccctgatcgtgcagcagtgcttgctgttccttagaattttgccttgtaagttctagctcaagttggggggtggtgatagacatttaagaagccatatatcttttcagaagtaggtgtgatgtactaaaagtttgagacactttctagaagtctcactatttaagttatgactagtattggatttttggcatgtctttgggtttcatgtttcttaacccaactgcctgcagggccttatggctgtcaggagcagttcttgggaattaaagtaattactgaagaagtattctagtgagaaaatgaatttatgactcagaagcccctaaagacatgggtactaagcaacaaaataagcagatgttaattaactgtaattttctcttacagctgtttgcagacaaggtcccaaagacagcaggttggtccattttctaagtttaacaaagatgttccaattgtgacagtttgtgtgtgtgtgtgtatatatatatttttatgtatgtatatatgtgtttaatttttttttaaacagaaaattttcgtgctctgagcactggagagaaaggatttggttataagggttcctgctttcacagaattattccagggtttatgtgtcaggtacgaaatttactgaattttattttatttgggttgctcccttcatttgggattgagccagaatatttcaggatacacatatctgaactgttactctaccatttcggttctatttaacccttctattcagtttgaacttgggtttaaagtttgaaccttgcagatttggcacacttcatggttatgttgtcagaagtgacatttttcctatatgttgacagggtggtgacttcacacgccataatggcactggtggcaagtccatctatggggagaaatttgaagatgagaacttcatcctaaagcatacgggtcctggcatcttgtccatggcaaatgctggacccaacacaaatggttcccagtttttcatctgcactgccaagactgagtggtaagggtacaacatggcacactaaccacctgactaaatgaaaagttgccctggggggaacggaacaaacactacttttcttcaacctttgcttccacagactttttcatccctaagatactagaagaagagcatacataaatgacaaatatagccaatgtgatacagaatgtcagatactatgatagaaacttggcccttagctgggtggttgaattaggtgctacttttttgagatggagttttgctctgttgccaggttggagtgcagtggcacaatctgggctcactgcaacctctgcctcctgggttcaagcgattctcctgccttggcctcctgagtagctgagaatacagatgtgtgccagcatgcctggctaattttttgtatttttgtggagacggggtttcatcatgttggccaagctggtcttgaactcgtgacttaaggtgaaccacctgccttggccccccaaagtgctgggatttcaggcatgagccactgcgcccaaccaattaagtgcttttttttttttttttcttttctcagactggatctcgctcttatctcccaggttggagtgcagtggtgccatctcagctcactgcaacctcctcccgggttcaagcaattcttctgcctcagcctctcaagtagctggaactacaggcatgcaccaccactcccagctaaattgtgtattattagtagagcgggatttaccatgttgtccaggctggtctcgaactcctgggctcaagtgatctgcctgccttgacccccccgaagtgctgggattacaggcatgagccactgtgcccacccaattaagtgctgcttttatgttactattaataacatgcggttggttgggttttttgtttctttggggtttttgttttgttttgtttgtttttgggggaggggggcgcaattcattctatatgtgtaactcttttttgagatggagtttcgctctgtcgcccaggctggagtgcagtggcgcgatctcggctcactgcaagctccgcctcccaggttcacgccattctcctgcctcagcctcccgagtagctgggactataggcacatgccaccatgcccggctaattttttgtatttttagtagagacagggtttcaccgtgttagccaggatggtctcgatctcctgacctcgtgatccgcccgccttggcctcccaaagtgctgggattacaggcgtgagccaccgcacccggcctatatgtgtaactctttaatggtaattggagaatcatgtttaatgacatttagtacaaaaggcttcagttaaaaaaaaaaaaaaaaagctacctttctcgtcttggttcatgacacatggaggctgcttgtttgtggttgccagtcataatgattgttcttccttttcaaggttggatggcaagcatgtggtgtttggcaaagtgaaagaaggcatgaatattgtggaggccatggagcgctttgggtccaggaatggcaagaccagcaagaagatcaccattgctgactgtggacaactcgaataagtttgacttgtgttttatcttaaccaccagatcattccttctgtagctcaggagagcacccctccaccccatttgctcgcagtatcctagaatctttgtgctctcgctgcagttccctttgggttccatgttttccttgttccctcccatgcctagctggattgcagagttaagtttatgattatgaaataaaaactaaataacaattgtcctcgtttgagttaagtgttgatgtaggctttattttaagcagtaatgggttacttctgaaacatcacttgtttgcttaattctacacagtacttagattttttttactttccagtcccaggaagtgtcaatgtttgttgagtggaatatt

SEQ ID NO: 94 - protein, 165 aa, Homo sapiens
MVNPTVFFDIAVDGEPLGRVSFELFADKVPKTAENFRALSTGEKGFGYKGSCFHRIIPGFMCQGGDFTRHNGTGGKSIYGEKFEDENFILKHTGPGILSMANAGPNTNGSQFFICTAKTEWLDGKHVVFGKVKEGMNIVEAMERFGSRNGKTSKKITIADCGQLE

SEQ ID NO: 95 - DNA, 717 bp, Homo sapiens
  misc_feature (109)..(109): n is a, c, g, or t
gaattcggcacgaggcgcggtgaggttgtctagtccacgctcggagccatgccgtccaagggcccgctgcagtctgtgcaggtcttcggacgcaagaagacagcgacanttggcgcactgcaaacgcggcaatggtctcatcaaggtgaacgggcggnccctggagatgattgagccgcgcacgctacagtacaagctgctggagccagttctgcttctcggnaaggagcgatttgctggtgtagacatccgtgtccgtgtaaagggtggtggtcacgtggcccagatttatgctatccgtcagtccatctccaaagccctggtggcctattaccagaaatatgtggatgaggttccaagaagnngatcaaagacatcctcatcccagtatgaccggaccctgctggtagttgaccctcgtcnctncgagtccaaaaagtttgagggcctngttcccgnggtnggtaccagaaaatctaccgtaagccatngtgactnnaaacttacttgtataataaacagttttttnggggtttaaagttaaaaanattaaaannntgnnntnnntttnnnntnnntntnnnnnnnnnntnnnnntnnnnnntnntttgggggggccnttnccntttgctttgggggggtttaattattggnttgttttannggggntngnancctggggtnccnttttttgntttgnnntnccttttggggtgn

SEQ ID NO: 96 - DNA, 1116 bp, Homo sapiens
gtgtgaggccatcacggaagatgctgctgcttctgctgcttctggggctagcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagagtcgactagcgatccaaaaaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctagctccaaaaccatcccaggtcattcttcatcctcacccaggattctcctgtacctgctcccaatctgtgttcctaaaagtgattctcactctgcttctcatctcctacttacatgaatacttctctcttttttctgtttccctgaagattgagctccc

SEQ ID NO: 97 - protein, 312 aa, Homo sapiens [sequence omitted]

SEQ ID NO: 98 - DNA, 1501 bp, Homo sapiens
agcgagtccttcttttcctgactgcagctcttttcattttgccatccttctccagctccatgatggttctgcaggtttctgcggccccccggacagtggctctgacggcgttactgatggtgctgctcacatctgtggtccagggcagggccactccagagaattacgtgtaccagggacggcaggaatgctacgcgtttaatgggacacagcgcttcctggagagatacatctacaaccgggaggagtacgcgcgcttcgacagcgacgtgggggagttccgggcggtgacggagctggggcggcctgctgcggagtactggaacagccagaaggacatcctggaggagaagcgggcagtgccggacagggtatgcagacacaactacgagctggacgaggccgtgaccctgcagcgccgagtccagcctaaggtgaacgtttccccctccaagaaggggcccctgcagcaccacaacctgcttgtctgccacgtgacagatttctacccaggcagcattcaagtccgatggttcctgaatggacaggaggaaacagctggggtcgtgtccaccaacctgatccgtaatggagactggaccttccagatcctggtgatgctggaaatgaccccccagcagggagacgtctacatctgccaagtggagcacaccagcctggacagtcctgtcaccgtggagtggaaggcacagtctgattctgcccagagtaagacattgacgggagctgggggcttcgtgctggggctcatcatctgtggagtgggcatcttcatgcacaggaggagcaagaaagttcaacgaggatctgcataaacagggttcctgacctcaccgaaaagactaatgtgccttagaacaagcatttgctgtgttttgttaacacctggttccaggacagaccctcagcttcccaagaggatactgctgccaagaagttgctctgaagtcagtttctatcgttctgctctttgattcaaagcactgtttctctcactgggcctccaaccatgttcccttcttcttagcaccacaaataatcaaaacccaacataagtgtttgctttcctttaaaaatatgcatcaaatcgtctctcattacttttctctgagggttttagtaaacagtaggagttaataaagaagttcattttggtttacacgtaggaaagaagagaagcatcaaagtggagatatgttaactattgtataatgtggcctgttatacatgacactcttctgaattgactgtatttcagtgagctgcccccaaatcaagtttagtgccctcatccatttatgtctcagaccgctattcttaactattcaatggtgagcagactgcaaatctgcctgataggacccatattcccacagcactaattcaacatatatcttactgagagcatgttttatcattaccattaagaagttaaatgaacatcagaatttaaaatcataaatataatctaatacacttt

SEQ ID NO: 99 - protein, 258 aa, Homo sapiens [sequence omitted]

SEQ ID NO: 100 - DNA, 5022 bp, Homo sapiens
cggacatggctgcggcccccggaggaggggacgtgaagtgaggagggggttgggaggggagaggacgcgggcgaggaagaccagccccggggccccgatgttgtgactgtgacagactcactggggtttgtacatgctggggaggagccttcctttcaggggtgaccacattcatctgggcatgcctgcagtactcttggcccatggacctgaaggagaagcacctgggcgagcctccctcagccctgggcctgtccacgcggaaggccctcagcgtcctgaaggagcagctggaggcagtgctggaaggacatctcagggagcggaagaagtgtctgacgtggaaggaggtgtggagaagcagcttcctccaccacagtaaccgctgctcctgcttccactggccgggggcctcactcatgctactggccgtgctgctgctgctgggctgctgcgggggacagccagccgggagccgtggggtggggctggtgaatgcctcggccttgttcctgttactgcttctcaaccttgtgctcatcgggcggcaagaccggctgaagcgtcgggaggtagagcggaggctgcgagggatcattgaccaaatccaagatgccctcagggatggcagggagatccagtggcccagtgccatgtatccagacctccacatgccttttgcgccatcctggtccttgcactgggcctacagagacggacacctggtcaacctgccagtcagcctgctggttgaaggagacatcatagctttgaggcctggccaggaatcgtttgcttctctgagggggatcaaggatgacgagcacatcgtcctggagccgggagacctcttcccccccttctcccctccaccctcaccccggggagaagtggagagagggccacagagcccccagcagcaccggcttttccgtgtccttgagacccctgtgattgacaacatcagatggtgcctggacatggccctgtcccgaccagtcactgccctggacaatgagcggttcacagtgcagtcggtgatgctacactatgctgtgcccgtggtcctggccggcttcctcatcaccaatgccctgcgcttcatcttcagtgccccgggggtcacttcctggcagtacaccctcctccagctccaggtgaatggcgtcctgcccatcctccccctgctctttccagtcctctgggttctggcaactgcctgtggagaggcccgtgtcctggcccagatgagcaaggcctcacccagctccctgctggctaagttctcagaggatactctcagcagctatacggaggctgtctcctctcaggaaatgctgcgctgcatttggggccacttcctgagggtgctcggggggacatcgccaacgctgagccacagttccagcctgctgcacagcctgggctctgtcacggtcctgtgctgtgtggacaaacaggggatcctgtcatggccaaatcccagcccagagactgtactgttcttcagcgggaaggtggagccccctcacagcagccatgaggacctcaccgatggcctatccacccgctccttctgccatcccgagccccatgaacgagacgccctcctggctggctccctgaacaacaccctgcacctttccaatgagcaggagcgtggcgactggcctggcgaggctcccaagccccccgagccctattcacaccacaaagcgcatggccgcagcaaacacccatctggctccaacgtgagcttcagcagggacaccgagggtggtgaagaagagcccagcaagacccagcctgggatggagagcgacccctacgaagcagaggactttgtgtgtgactaccacctggagatgctgagcctgtcccaggaccagcagaacccctcctgcatccagtttgatgactccaactggcagctgcacctcacctccctcaaacccctgggcctcaatgtgctgctgaacctgtgtgatgccagcgtcaccgagcgcctgtgccgattctccgaccacctgtgcaacattgccctgcaagagagccacagcgccgtgctgcccgtccatgtgccctggggcctctgcgagcttgcccgcctcattggcttcactcctggggccaaggagcttttcaagcaggagaaccatctggcgctgtaccgcctccccagtgccgagacaatgaaggagacatcgctggggcggctctcctgtgtcaccaagcggcggcctcccctcagccacatgatcagcctcttcattaaagacaccaccaccagcacagagcagatgctgtcccatggcaccgctgatgtggtcttagaggcctgcacagacttctgggacggagctgacatctaccctctctcgggatctgacagaaagaaagtgctggacttctaccagcgagcctgcctgtctgggtattgctctgccttcgcctacaagcccatgaactgcgccctgtcctctcagctcaatggcaagtgcatcgagctggtacaggtgcccggccaaagcagcatcttcaccatgtgcgagctgcccagcaccatccccatcaagcagaacgcccgccgcagcagctggagctctgacgaagggatcggggaggtgctggagaaggaagactgcatgcaggccctgagcggccagatcttcatgggcatggtgtcctcccagtaccaggcccggctggacatcgtgcgcctcattgatgggcttgtcaacgcctgcatccgctttgtctacttctctttggaggatgagctcaaaagcaaggtgtttgcagaaaaaatgggcctggagacaggctggaactgccacatctccctcacacccaatggtgacatgcctggctccgagatccccccctccagccccagccacgcaggctccctgcatgatgacctgaatcaggtgtcccgagatgatgcagaagggctcctcctcatggaggaggagggccactcggacctcatcagcttccagcctacggacagcgacatccccagcttcctggaggactccaaccgggccaagctgccccggggtatccaccaagtgcggccccacctgcagaacattgacaacgtgcccctgctagtgccccttttcaccgactgcaccccagagaccatgtgtgagatgataaagatcatgcaagagtacggggaggtgacctgctgcctgggcagctctgccaacctgcggaacagctgcctcttcctccagagcgacatcagcattgccctggatcccctgtacccatcccgttgctcctgggagacctttggctacgccaccagcatcagcatggcccaggcctcggatggcctttctcccctgcagctgtcagggcagctcaacagcctgccctgttccctgacctttcgccaggaggagaccatcagcatcatccggcttatcgaacaggctcggcatgccacctatggcatccgtaagtgcttcctcttcctgctgcagtgccagctgactcttgtggtcatccagttcctttcttgcctggtccagctgccgccactcctgagtaccaccgacatcctgtggctgtcctgcttttgctaccctctgctcagcatctctctgctggggaagcccccccatagctccatcatgtctatggcaacggggaaaaacctccagtccattcccaagaagacccagcactacttcctgctctgcttcctgctcaagttcagcctcaccatcagctcctgcctcatctgctttggcttcacactgcagagcttctgtgacagctcccgggaccgcaacctcaccaactgctcctccgtcatgctgcccagcaacgacgacagggctccagcctggtttgaggactttgccaatggactgctgtcggctcagaagctcacggccgccctgattgtcctgcacactgtcttcatttccatcacccatgtgcatcgcaccaagcccctgtggagaaagagccccttgaccaacctctggtgggccgtgacagtgcctgtggtgctgctgggtcaggtggtccagacggctgtggacctgcagctgtggacacacagggacagccacgtccactttggcctggaggacgtgcccctgctgacatggctcctgggctgcctgtccctggtccttgtggtggtgaccaatgagatcgtgaagctacatgagattcgggtccgagtccgctaccagaagcgacagaagctgcagtttgaaactaagctgggcatgaactctcccttctgagccactggctgtggtggctgtagttgcccccgtccctggggctaaagccagacccatttctgaacaggggagtttgtatcatgaatgtttccaggtttgctcctgcacccgtggcactggaaacccagctccccgtgtcagaccccgctgtcttcctgagccctggggctcactgtggaggagctgacggcctgggcccttggccagtcctggctcttccctgggcctcaccagggacactcttgaatgtatggcctcaggcgctccctagaggggccctaaaccccctcacctgtgagctaccccctttagggatcccttgcccccttggagatcccttgccccccagtgcctctgctcgtgggtccctggacacggccttgaagccaaccttctttggaggagcaacagcagcagccttggccgacgcgtccaactcccaaggctgccgtggagggcaggggggtggtgcttgcctggatgtggccccgagtgcctcccctccctccctctgtgggggagtctcccgcctgaacctgaagatggagcagggcccccgcttcgccctggagcctcttcctgtgcctggctcaagctggctgcctgtcagtcttggggaatctggcccaggtctcctcagcctctgccccagttctgggagaagtttctactggtgtatattttttactggaaatgagccttttaggaatgaatgtagactggtttgtattaaaatgtgtcaattgct

SEQ ID NO: 101 - protein, 1356 aa, Homo sapiens
MDLKEKHLGEPPSALGLSTRKALSVLKEQLEAVLEGHLRERKKCLTWKEVWRSSFLHHSNRCSCFHWPGASLMLLAVLLLLGCCGGQPAGSRGVGLVNASALFLLLLLNLVLIGRQDRLKRREVERRLRGIIDQIQDALRDGREIQWPSAMYPDLHMPFAPSWSLHWAYRDGHLVNLPVSLLVEGDIIALRPGQESFASLRGIKDDEHIVLEPGDLFPPFSPPPSPRGEVERGPQSPQQHRLFRVLETPVIDNIRWCLDMALSRPVTALDNERFTVQSVMLHYAVPVVLAGFLITNALRFIFSAPGVTSWQYTLLQLQVNGVLPILPLLFPVLWVLATACGEARVLAQMSKASPSSLLAKFSEDTLSSYTEAVSSQEMLRCIWGHFLRVLGGTSPTLSHSSSLLHSLGSVTVLCCVDKQGILSWPNPSPETVLFFSGKVEPPHSSHEDLTDGLSTRSFCHPEPHERDALLAGSLNNTLHLSNEQERGDWPGEAPKPPEPYSHHKAHGRSKHPSGSNVSFSRDTEGGEEEPSKTQPGMESDPYEAEDFVCDYHLEMLSLSQDQQNPSCIQFDDSNWQLHLTSLKPLGLNVLLNLCDASVTERLCRFSDHLCNIALQESHSAVLPVHVPWGLCELARLIGFTPGAKELFKQENHLALYRLPSAETMKETSLGRLSCVTKRRPPLSHMISLFIKDTTTSTEQMLSHGTADVVLEACTDFWDGADIYPLSGSDRKKVLDFYQRACLSGYCSAFAYKPMNCALSSQLNGKCIELVQVPGQSSIFTMCELPSTIPIKQNARRSSWSSDEGIGEVLEKEDCMQALSGQIFMGMVSSQYQARLDIVRLIDGLVNACIRFVYFSLEDELKSKVFAEKMGLETGWNCHISLTPNGDMPGSEIPPSSPSHAGSLHDDLNQVSRDDAEGLLLMEEEGHSDLISFQPTDSDIPSFLEDSNRAKLPRGIHQVRPHLQNIDNVPLLVPLFTDCTPETMCEMIKIMQEYGEVTCCLGSSANLRNSCLFLQSDISIALDPLYPSRCSWETFGYATSISMAQASDGLSPLQLSGQLNSLPCSLTFRQEETISIIRLIEQARHATYGIRKCFLFLLQCQLTLVVIQFLSCLVQLPPLLSTTDILWLSCFCYPLLSISLLGKPPHSSIMSMATGKNLQSIPKKTQHYFLLCFLLKFSLTISSCLICFGFTLQSFCDSSRDRNLTNCSSVMLPSNDDRAPAWFEDFANGLLSAQKLTAALIVLHTVFISITHVHRTKPLWRKSPLTNLWWAVTVPVVLLGQVVQTAVDLQLWTHRDSHVHFGLEDVPLLTWLLGCLSLVLVVVTNEIVKLHEIRVRVRYQKRQKLQFETKLGMNSPF

SEQ ID NO: 102 - DNA, 2030 bp, Homo sapiens
tggctggcaatggccttgctgacctcgagccgggcccacgtggggacctttggagcacagcctacgatcctggtgcaaggccggtggatgcagaggccagtccatataccacccaggcctgcgaggagcgtggtccccacccatccagcccatatgtgcaagtgcccttgacagagaggctggtcatatccatggtgaccatttatgggccacaacaggtccccatctgcgcagtgaaccctgtgctgagcaccttgcagacgtgatcttgcttcgtcctgcagcactgtgcggggcaggaaaatccaagaggaagaaggatctacggatatcctgcatgtccaagccacccgcacccaaccccacacccccccggaacctggactcccggaccttcatcaccattggagacagaaactttgaggtggaggctgatgacttggtgaccatctcagaactgggccgtggagcctatggggtggtagagaaggtgcggcacgcccagagcggcaccatcatggccgtgaagcggatccgggccaccgtgaactcacaggagcagaagcggctgctcatggacctggacatcaacatgcgcacggtcgactgtttctacactgtcaccttctacggggcactattcagagagggagacgtgtggatctgcatggagctcatggacacatccttggacaagttctaccggaaggtgctggataaaaacatgacaattccagaggacatccttggggagattgctgtgtctatcgtgcgggccctggagcatctgcacagcaagctgtcggtgatccacagagatgtgaagccctccaatgtccttatcaacaaggagggccatgtgaagatgtgtgactttggcatcagtggctacttggtggactctgtggccaagacgatggatgccggctgcaagccctacatggcccctgagaggatcaacccagagctgaaccagaagggctacaatgtcaagtccgacgtctggagcctgggcatcaccatgattgagatggccatcctgcggttcccttacgagtcctgggggaccccgttccagcagctgaagcaggtggtggaggagccgtccccccagctcccagccgaccgtttctcccccgagtttgtggacttcactgctcagtgcctgaggaagaaccccgcagagcgtatgagctacctggagctgatggagcaccccttcttcaccttgcacaaaaccaagaagacggacattgctgccttcgtgaagaagatcctgggagaagactcataggggctgggcctcggaccccactccggccctccagagccccacagccccatctgcgggggcagtgctcacccacaccataagctactgccatcctggcccagggcatctgggaggaaccgagggggctgctcccacctggctctgtggcgagccatttgtcccaagtgccaaagaagcagaccattggggctcccagccaggcccttgtcggccccaccagtgcctctccctgctgctcctaggacccgtctccagctgctgagatcctggactgagggggcctggatgccccctgtggatgctgctgcccctgcacagcaggctgccagtgcctgggtggatgggccaccgccttgcccagcctggatgccatccaagttgtatatttttttaatctctcgactgaatggactttgcacactttggcccagggtggccacacctctatcccggctttggtgcggggtacacaagaggggatgagttgtgtgaataccccaagactcccatgagggagatgccatgagccgcccaaggccttcccctggcactggcaaacagggcctctgcggagcacactggctcacccagtcctgcccgccaccgttatcggtgtcattcacctttcgtgttttttttaatttatcctctgttgattttttcttttgctttatgggtttggcttgtttttcttgcatggtttggagctgatcgcttctcccccaccccctagggg

SEQ ID NO: 103 - protein, 318 aa, Homo sapiens [sequence omitted]

SEQ ID NO: 104 - DNA, 1648 bp, Homo sapiens
atgcgggagatcgtgcacatccaggccggccagtgcggcaaccagatcggggccaagttctgggaagtcatcagtgatgagcatggcatcgaccccagcggcaactacgtgggcgactcggacttgcagctggagcggatcagcgtctactacaacgaggcctcttctcacaagtacgtgcctcgagccattctggtggacctggaacccggaaccatggacagtgtccgctcaggggcctttggacatctcttcaggcctgacaatttcatctttggtcagagtggggccggcaacaactgggccaagggtcactacacggagggggcggagctggtggattcggtcctggatgtggtgcggaaggagtgtgaaaactgcgactgcctgcagggcttccagctgacccactcgctggggggggggacgggctccggcatgggcacgttgctcatcagcaaggtgcgtgaggagtatcccgaccgcatcatgaacaccttcagcgtcgtgccctcacccaaggtgtcagacacggtggtggaaccctacaacgccacgctgtccatccaccagctggtggaaaacacggatgaaacctactgcatcgacaacgaggcgctctacgacatctgcttccgcaccctcaagctggccacgcccacctacggggacctcaaccacctggtatcggccaccatgagcggagtcaccacctccttgcgcttcccgggccagctcaacgctgacctgcgcaagctggccgtcaacatggtgcccttcccgcgcctgcacttcttcatgcccggcttcgcccccctcaccaggcggggcagccagcagtaccgggccctgaccgtgcccgagctcacccagcagatgttcgatgccaagaacatgatggccgcctgcgacccgcgccacggccgctacctgacggtggccaccgtgttccggggccgcatgtccatgaaggaggtggacgagcagatgctggccatccagagcaagaacagcagctacttcgtggagtggatccccaacaacgtgaaggtggccgtgtgtgacatcccgccccgcggcctcaagatgtcctccaccttcatcgggaacagcacggccatccaggagctgttcaagcgcatctccgagcagttcacggccatgttccggcgcaaggccttcctgcactggtacacgggcgagggcatggacgagatggagttcaccgaggccgagagcaacatgaacgacctggtgtccgagtaccagcagtaccaggacgccacggccgaggaagagggcgagatgtacgaagacgacgaggaggagtcggaggcccagggccccaagtgaaactgctcgcagctggagtgagaggcaggtggcggccggggccgaagccagcagtgtctaaacccccggagccatcttgctgccgacaccctgctttccccatcgccctagggctcccttgccgccctcctgcagtatttatggcctcgtcctcccccacctaggccacgtgtgagctgctcctgtctctgtcttattgcagctccaggcctgacgttttacggttttgttttttactggtttgtgtttatattttcggggatacttaataaatctattgctgtcagataccctt

SEQ ID NO: 105 - protein, 450 aa, Homo sapiens
MREIVHIQAGQCGNQIGAKFWEVISDEHGIDPSGNYVGDSDLQLERISVYYNEASSHKYVPRAILVDLEPGTMDSVRSGAFGHLFRPDNFIFGQSGAGNNWAKGHYTEGAELVDSVLDVVRKECENCDCLQGFQLTHSLGGGTGSGMGTLLISKVREEYPDRIMNTFSVVPSPKVSDTVVEPYNATLSIHQLVENTDETYCIDNEALYDICFRTLKLATPTYGDLNHLVSATMSGVTTSLRFPGQLNADLRKLAVNMVPFPRLHFFMPGFAPLTRRGSQQYRALTVPELTQQMFDAKNMMAACDPRHGRYLTVATVFRGRMSMKEVDEQMLAIQSKNSSYFVEWIPNNVKVAVCDIPPRGLKMSSTFIGNSTAIQELFKRISEQFTAMFRRKAFLHWYTGEGMDEMEFTEAESNMNDLVSEYQQYQDATAEEEGEMYEDDEEESEAQGPK

SEQ ID NO: 106 - DNA, 1633 bp, Homo sapiens
cagaatctccggcagtttttgtacctcaagaagtaagtggaacacctttccctgtcatagttattttcatccagacatctggtggaagcatcagattccttacagatataagagaggcatcatttaaaaggtagaacaggatcgacaaacaaggatttatgtcaggatctctcagcctctgtgttaccgagggcatttctaacagtcttcttactacggcctccgccgaccgcgcgctcgccccgccgctcctgctgcagccccagggcccctcgccgccgccaccatggacgccatcaagaagaagatgcagatgctgaagctcgacaaggagaacgccttggatcgagctgagcaggcggaggccgacaagaaggcggcggaagacaggagcaagcagctggaagatgagctggtgtcactgcaaaagaaactcaagggcaccgaagatgaactggacaaatactctgaggctctcaaagatgcccaggagaagctggagctggcagagaaaaaggccaccgatgctgaagccgacgtagcttctctgaacagacgcatccagctggttgaggaagagttggatcgtgcccaggagcgtctggcaacagctttgcagaagctggaggaagctgagaaggcagcagatgagagtgagagaggcatgaaagtcattgagagtcgagcccaaaaagatgaagaaaaaatggaaattcaggagatccaactgaaagaggcaaagcacattgctgaagatgccgaccgcaaatatgaagaggtggcccgtaagctggtcatcattgagagcgacctggaacgtgcagaggagcgggctgagctctcagaaggccaagtccgacagctggaagaacaattaagaataatggatcagaccttgaaagcattaatggctgcagaggataagtactcgcagaaggaagacagatatgaggaagagatcaaggtcctttccgacaagctgaaggaggctgagactcgggctgagtttgcggagaggtcagtaactaaattggagaaaagcattgatgacttagaagagaaagtggctcatgccaaagaagaaaaccttagtatgcatcagatgctggatcagactttactggagttaaacaacatgtgaaaacctccttagctgcgaccacattctttcattttgttttgttttgttttgtttttaaacacctgcttaccccttaaatgcaatttatttacttttaccactgtcacagaaacatccacaagataccagctaggtcagggggtggggaaaacacatacaaaaagcaagcccatgtcagggcgatcctggttcaaatgtgccatttcccgggttgatgctgccacactttgtagagagtttagcaacacagtgtgcttagtcagcgtaggaatcctcactaaagcaggagaagttccattcaaagtgccaatgatagagtcaacaaggaaggttaatgttggaaacacaatcaggtgtggattggtgctactttgaacaaaaggtccccctgtggtcttttgttcaacattgtacaatgtagaactctgtccaacactaatttattttgtcttgagttttactacaagatgagactatggatcccgcatgcct

SEQ ID NO: 107 - protein, 284 aa, Homo sapiens
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGQVRQLEEQLRIMDQTLKALMAAEDKYSQKEDRYEEEIKVLSDKLKEAETRAEFAERSVTKLEKSIDDLEEKVAHAKEENLSMHQMLDQTLLELNNM

SEQ ID NO: 108 - DNA, 1835 bp, Homo sapiens
  misc_feature (44)..(44): n is a, c, g, or t
ttacactttatacttccggctcgaatattgtgtggaattgtgancggataacaatttcacacaggaaacanctatgaccttgattacgccaagctcgaaattaaccctcactaaagggaacaaaagctggagctcgcgcgcctgcaggtcgacactagtggatccaaagaattcggcacgaggcgacgggcggagcggagcgcggcgcgccggggccgccgccggggggatcggctgcctccccgggccgggtgtagagagggcgggtccccggcctcgggagcacggcggtggaggggacataggaggcggccatggcgacccccggcaacctagggtcctccgtcctggcgagcaagaccaagaccaagaagaagcacttcgtagcgcagaaagtgaagctgtttcgggccagcgacccgctgctcagcgtcctcatgtggggggtaaaccactcgatcaatgaactgagccatgttcaaatccctgttatgttgatgccagatgacttcaaagcctattcaaaaataaaggtggacaatcacctttttaacaaagaaaacatgccgagccatttcaagtttaaggaatactgcccgatggtcttccgtaactgcgggaagaggtttggaattgatgttcaagatttccagaattccctgaccaggagcgcacccctccccaacgactcccaggcccgcagtggagctcgttttcacacttcctacgacaaaagatacatgatcaagactattaccagtgaagacgtggccgaaatgcacaacatcctgaagaaataccaccagtacatagtggaatgtcatgggatcacccttcttccccacttgttgggcatgtaccggcttaatgttgatggagttgaaatatatgtgatagttacaagaaatgtattcagccaccgtttgtctgtgtataggaaatacgacttaaagggctctacagtggctagagaagctagtgacaaagaaaaggccaaagaactgccaactctgaaagataatgatttcattaatgagggccaaaagatttatattgatgacaacagcaagaaggtcttcctggaaaaactaaaaaaggatgttgagtttctggcccagctgaagctcatggactacagtctgctggtgggaattcatgatgtggagagagccgaacaggaggaagtggagtgtgaggagaacgatggggaggaggagggcgagagcgatggcacccacccggtgggaacccccccagatagccccgggaatacactgaacagctcaccacccctggctcccggggagttcgagccgaacatcgacgtctatggaattaagtgccatgaaaactcgcctaggaaggaggtgtacttcatggcaattattgacatccttactcattatgatgcaaaaaagaaagctgcccatgctgcaaaaactgttaaacatggcgctggcgcggagatctccaccgtgaacccagaacagtattcaaagcgctttttggactttattggccacatcttgacgtaacctcctgcgcayctcggacagcatgaacattggatggacagaggtggcttcggtgtaggaaaaatgaaaaccaaactcagtgaagtactcatcttgcaggaagcaaacctccttgtttacatcttcaggccaagatgactgatttgggggctactcgctttacagctacctgattttcccagcatcgttctagctatttctgactttgtgtatatgtgtgtgtgtgtgtgttgggggggggtgagtgtgtgcccgcgtgtgcatttaaagcataaattaattaaacagccacttcggtca

SEQ ID NO: 109 - protein, 406 aa, Homo sapiens [sequence omitted]

SEQ ID NO: 110 - DNA, 2572 bp, Homo sapiens
aacagaattagttggcccagctctgcctataagtagctgaatgtcttgaggcaacttcaacgtttccctggaccttagattccttgcctgtaaaacagcatggggccagatgatctctaagggtccttctggctctgaaggcaatgatctggggcatggaacctgtagttagagagctgggaaatgggcagatgtgggctccagggcacccagaattgcaggcttaggagctaacagcaaccaggattctgtagtctagcaatcttgctttacaggtgaggaaactgggcctagaaaggcgaagtgatttttttgcctctctcagctttattcctcttttcctctgaactgtagagtctaaagattcagcacaaagcagttttgtgtagtggatacataagcttttttgttgttatttttctgaattattttgttgactttcaaagttttttttacataaacagtaaatgctcgttataaaaatttccattaatacaggaagtgaaaaaagtaaaaaactgcaaattgctgttatcctcccacccttaccccctaggtccccagaggtgtctctgttaacagttcagcgtgtatccatcctgactcctccaataaatgcagaaacttgtatgtctctccccgacaactggattatcatatacattattcaagcataggctttagactcagacatatctacatctaatcccagcttatagctaattatttgagtgaccttggccaagttgttcatccagttttagtctcaatctccccatgtgtaaaatgaaaataataatagtatctacctggcctggcgtggcggcttatgcctataatcccagccctttggaagaccgaggctagtggatcccttgagctcaggagttcaagaccaacctgggcaatttagcaagacctcatctctactgaaaaacaaaaaacaaaaaaactcccccaaaattagccaagtgtgctggtgtgcacctgtagtcccagctactcggaaggctgaggtgggagaatcgcttgagccaggaaagacgaggctgcagtgagctgtgattgcaccactgcactgcaacctgagcaacagagcgataccttgtttctgttaaaacaaacaaacaaacaaaatagtatctaccttataggatcatggtgaagatttaatgagattttatatgaatagcacttaacagttcctggtactgatagtagtaagcactacacacacacacacacacacacacacacacacacacagagcacagaatgagttagaggtaaagtgaaaactaaaccccaagttttctgaccctcagtctcctcgactttctaccacatctctctgcttctctcctaggtgcctaggcatgggttcagtgctcactacttgttgaatgaatgactgaggttgtgtgtaagggggtagatctagggatctgaggtctgtggagttcctgggatgcctgctctggaaaatggaggctttcatcctgtgagttgggagggtgtggggcagtgtgggttggctggaccagctgttgcttcagagctccatgcctggagagttgggcctctaggcagagctgagggcccagagtggctctcagcttaaaggatcttggcttagaaggaatgtgcagtgggctgcctctgctcgggaggggctaaaaaaagcctcaccctcccctgggctttgtgtgaggcttatcaactgctcaagtcagctcatctctctggctgctccggcatatttgagaaggtctgtttccctggtccttctgggtttccaccaattggcaagaagggatcagcctgtcctagaggtgaagagagagctgtggcatgaaggggagggggctggtggccccaaacctggtgacaatacacagttgtcagctgtaccctgctggcgtttcttccttttatagtcagcagcagttgctcttgctttcacccagcccctctgtggggctcctgcccaggataaaagggaagggaggcagcccaggctcctatctcatctcccagacgccacgtctctcggtttcttcttagatcactcctctgccaaagatcccaacaagacaacatggctcccaagaagcctgagcctaagaaggaggcagccaagccagctccagctccagctccagcccctgcaccagcccctgccccagctcctgaggctcccaaggaacctgcctttgaccccaagagtgtaaaggtaagtgaggctcagccattgggatagaggtggggatgacattgagagtccttttgctctggagcttagcgatctactttatgtgggctggactgggatgaggactagggtgtccatgccccagatcgcagtcccatggggcagtggagtgggtgttggggctgatgagggggagattgagtcataaaccttttccgtcaagaatgaggtgctgctttgagggagccctgtcctgctaccctagatttgtgcagctaagttgggaatggggggaggtacaaccaaccatccatccacccttttataaggcattaatgaggaccaccatagcaaagtaaa

SEQ ID NO: 111 - protein, 197 aa, Homo sapiens
MAPKKPEPKKEAAKPAPAPAPAPAPAPAPAPEAPKEPAFDPKSVKIDFTADQIEEFKEAFSLFDRTPTGEMKITYGQCGDVLRALGQNPTNAEVLRVLGKPKPEEMNVKMLDFETFLPILQHISRNKEQGTYEDFVEGLRVFDKESNGTVMGAELRHVLATLGEKMTEAEVEQLLAGQEDANGCINYEAFVKHIMSG

SEQ ID NO: 112 - DNA, 1011 bp, Homo sapiens
gatttgcggaagaacctgaccgtggacgagggcaccatgaaggtagaggtgctgcctgccctgaccgacaactacatgtacctggtcattgatgatgagaccaaggaggctgccattgtggatccggtgcagccccagaaggtcgtggacgcggcgagaaagcacggggtgaaactgaccacagtgctcaccacccaccaccactgggaccatgctggcgggaatgagaaactggtcaagctggagtcgggactgaaggtgtacgggggtgacgaccgtatcggggccctgactcacaagatcactcacctgtccacactgcaggtggggtctctgaacgtcaagtgcctggcgaccccgtgccacacttcaggacacatttgttacttcgtgagcaagcccggaggctcggagccccctgccgtgttcacaggtgacaccttgtttgtggctggctgcgggaagttctatgaagggactgcggatgagatgtgtaaagctctgctggaggtcttgggccggctccccccggacacaagagtctactgtggccacgagtacaccatcaacaacctcaagtttgcacgccacgtggagcccggcaatgccgccatccgggagaagctggcctgggccaaggagaagtacagcatcggggagcccacagtgccatccaccctggcagaggagtttacctacaaccccttcatgagagtgagggagaagacggtgcagcagcacgcaggtgagacggacccggtgaccaccatgcgggccgtgcgcagggagaaggaccagttcaagatgccccgggactgaggccgccctgcaccttcagcggatttggggattaggctcttttaggtaactggctttcctgctggtccgtgcgggaaattcagtcttgatttaaccttaattttacagcccttggcttgtgttatcggacattctaatgcatatttataagagaagtttaacaagtatttattcccataaaaaaaaaaaaaa

SEQ ID NO: 113 - protein, 260 aa, Homo sapiens [sequence omitted]

SEQ ID NO: 114 - DNA, 2233 bp, Homo sapiens
agaggggccccgcgcgcggatctcgcgagagcattagagggcggaagcgctatccgagcaggatgcggttcgtggttgccttggtcctcctgaacgtcgcagcggcgggagccgtgccgctcttggccaccgaaagcgtcaagcaagaagaagctggagtacggccttctgcaggaaacgtctccacccaccccagcttgagccaacggcctggaggctctaccaagtcgcatccggagccgcagactccaaaagacagccctagcaagtcgagtgcggaggcgcagaccccagaagacacccccaacaagtcgggtggggaggcaaagaccctaaaagacagctccaacaagtcgggtgcggaggcacagacccccaaaggcagcactagcaagtcgggttcggaggcgcagaccacaaaagacagcactagtaagtcgcatccggagctgcagactccaaaagacagcactggcaaatcgggtgcggaggcgcagaccccagaagacagccccaacaggtcgggtgcggagccaaagacccaaaaagacagccctagcaagtcaggttcggaggcgcagaccacaaaagatgtccctaataagtcgggtgcggacggccagaccccaaaagacggctccagcaagtcgggtgcggaggatcagaccccaaaagacgtccctaacaagtcgggtgcggagaagcagactccaaaagacggctctaacaagtccggtgcagaggagcagggcccaatagacgggcccagcaagtcgggtgcggaggagcagacctcaaaagacagccctaacaaggtggttccagagcagccttcccggaaagaccattccaagcccatctccaacccttctgataacaaggagctccccaaggctgacacaaaccagcttgctgacaaagggaagctttctcctcatgctttcaaaaccgaatctggggaggaaactgacctcatttctcccccgcaggaggaagttaagtcttcagagcctactgaggatgtggggcccaaagaggctgaagatgatgatacaggacccgaggagggctcaccgcccaaagaagagaaagaaaagatgtccggttctgcctccagtgagaaccgtgaagggacactttcggattccacgggtagcgagaaggatgacctttatccgaacggttctggaaatggcagcgcggagagcagccacttctttgcatatctggtgactgcagccattcttgtggctgtcctctatatcgctcatcacaacaagcggaagatcattgcttttgtcctggaaggaaaaagatctaaagtcacccggcggccaaaggccagtgactaccaacgtttggaccagaagatcttttctcccccaagtcctaacagaatggtatattcctctggaaaaagatgaacgtcaccaatggattgtgctgctctcgtttcagctttgatttttttgtccttgagaaccttgtcctccctgctgatttgtttctaaatcaaaagaaatgaagaaaaaagtactgtgacctgagagacaccctcctctagaatttagtggcgggtctgggctggcagaggtagggggctgctttgggctttgcacctgcactttggtgacattgttcttctgtgttccctttatttatgctggtggcttccatccgttctcctctggggtgagtggaggggtatatggaaacacggctatgaccaaagggagatcccagcctgggcagcctgcgctgctgaccaccctccctggggcccgggctctgtaggaaagttggtccttgactgtggcattgcactctgcactgtttctctctgcagacctaggggaaaactgcaggtggaagtgcttttctactaaggcctcttactttgggggggatgtgccctacagaagacatagaagatggggaaatgccaatgggcaaagagctactttgaatacataattctcttcaaagacttcagcagcaaacctaaacagcaggttaaaaaaaaagatgcttttttgggtgcaagtctaacctgtctagcatgagatcttcttgattttctgattattttatgtagcttgagacaaagtgaatcaacttccacttagttgtaccgagcataaaacagaacttgggcttcctggcagtgaggccactgtcccatcacagatttttaaaataaatatgatttgaagtagtgtgatctttcacacaa

SEQ ID NO: 115 - protein, 453 aa, Homo sapiens
MRFVVALVLLNVAAAGAVPLLATESVKQEEAGVRPSAGNVSTHPSLSQRPGGSTKSHPEPQTPKDSPSKSSAEAQTPEDTPNKSGGEAKTLKDSSNKSGAEAQTPKGSTSKSGSEAQTTKDSTSKSHPELQTPKDSTGKSGAEAQTPEDSPNRSGAEPKTQKDSPSKSGSEAQTTKDVPNKSGADGQTPKDGSSKSGAEDQTPKDVPNKSGAEKQTPKDGSNKSGAEEQGPIDGPSKSGAEEQTSKDSPNKVVPEQPSRKDHSKPISNPSDNKELPKADTNQLADKGKLSPHAFKTESGEETDLISPPQEEVKSSEPTEDVGPKEAEDDDTGPEEGSPPKEEKEKMSGSASSENREGTLSDSTGSEKDDLYPNGSGNGSAESSHFFAYLVTAAILVAVLYIAHHNKRKIIAFVLEGKRSKVTRRPKASDYQRLDQKIFSPPSPNRMVYSSGKR

SEQ ID NO: 116 - DNA, 829 bp, Homo sapiens
gcggttcgccttcaacatgccggaaccagcgaagtccgctcccgcgcccaagaagggctcgaagaaagccgtgactaaggcgcagaagaaggacggtaagaagcgcaagcgcagccgcaaggagagctactccgtatacgtgtacaaggtgctgaagcaggtccaccccgacaccggcatctcctctaaggccatgggaatcatgaactccttcgtcaacgacatcttcgaacgcatcgcgggtgaggcttcccgcctggcgcattacaacaagcgctcgaccatcacctccagggagatccagacggccgtgcgcctgctgctgcccggggagttggccaagcacgccgtgtccgagggcaccaaggccgtcaccaagtacaccagcgctaagtaaacttgccaaggagggactttctctggaatttcctgatatgaccaagaaagcttcttatcaaaagaagcacaattgccttcggttacctcattatctactgcagaaaagaagacgagaatgcaaccatacctagatggacttttccacaagctaaagctggcctcttgatctcattcagattccaaagagaatcatttacaagttaatttctgtctccttggtccattccttctctttaataatcatttactgttcctcaaagaattgtttacattacccatctcctcttttgctctgagaaagagtatataagcttctgtaccccactggggggttggggtaatattctgtggtcctcagccctgtaccttaataaatttgtatgcctttttttttaaaaaaaaaaaaaaaaaaaaaaaaaaaaaaaaaaaa

SEQ ID NO: 117 - protein, 126 aa, Homo sapiens
MPEPAKSAPAPKKGSKKAVTKAQKKDGKKRKRSRKESYSVYVYKVLKQVHPDTGISSKAMGIMNSFVNDIFERIAGEASRLAHYNKRSTITSREIQTAVRLLLPGELAKHAVSEGTKAVTKYTSAK

SEQ ID NO: 118 - DNA, 3858 bp, Homo sapiens
agtctggtttaactggttggaacgactaaagcacgctggcgcaaggaaagctctcaacttcgggagctgaggcgcaggctggccagagcgtggagaggaaagccctttccatcctcaaggccgttgcaggagatgcccgcgagccaccttcgccagcaccacaccggggtgtaatggataggtaacagagaagacctcgtcccttcctagtcagggcatcagcatgactgagtgcttcctgccccccaccagcagccccagtgaacaccgcagggtggagcatggcagcgggcttacccggacccccagctctgaagagatcagccctactaagtttcctggattgtaccgcactggcgagccctcacctccccatgacatcctccatgagcctcctgatgtagtgtctgatgatgagaaagatcatgggaagaaaaaagggaaatttaagaaaaaggaaaagaggactgaaggctatgcagcctttcaggaagatagctctggagatgaggcagaaagtccttctaaaatgaagaggtccaagggaatccatgttttcaagaagcccagcttttctaaaaagaaggaaaaggattttaaaataaaagagaaacccaaagaagaaaagcataaagaagaaaagcacaaagaagaaaaacataaagagaagaagtcaaaagacttgacagcagctgatgttgttaaacagtggaaggaaaagaagaaaaagaaaaagccaattcaggagccagaggtgcctcagattgatgttccaaatctcaaacccatttttggaattcctttggctgatgcagtagagaggaccatgatgtatgatggcattcggctgccagccgttttccgtgaatgtatagattacgtagagaagtatggcatgaagtgtgaaggcatctacagagtatcaggaattaaatcaaaggtggatgagctaaaagcagcctatgaccgggaggagtctacaaacttggaagactatgagcctaacactgtagccagtttgctgaagcagtatttgcgagaccttccagagaatttgcttaccaaagagcttatgcccagatttgaagaggcttgtgggaggaccacggagactgagaaagtgcaggaattccagcgtttactcaaagaactgccagaatgtaactatcttctgatttcttggctcattgtgcacatggaccatgtcattgcaaaggaactggaaacaaaaatgaatatacagaacatttctatagtgctcagcccaactgtgcagatcagcaatcgagtcctgtatgtgtttttcacacatgtgcaagaactctttggaaatgtggtactaaagcaagtgatgaaacctctgcgatggtctaacatggccacgatgcccacgctgccagagacccaggcgggcatcaaggaggagatcaggagacaggagtttcttttgaattgtttacatcgagatctgcagggtgggataaaggatttgtctaaagaagaaagattatgggaagtacaaagaattttgacagccctcaaaagaaaactgagagaagctaaaagacaggagtgtgaaaccaagattgcacaagagatagccagtctttcaaaagaggatgtttccaaagaagagatgaatgaaaatgaagaagttataaatattctccttgctcaggagaatgagatcctgactgaacaggaggagctcctggccatggagcagtttctgcgccggcagattgcctcagaaaaagaagagattgaacgcctcagagctgagattgctgaaattcagagtcgccagcagcacggccgaagtgagactgaggagtactcctccgagagcgagagcgagagtgaggatgaggaggagctgcagatcattctggaagacttacagagacagaacgaagagctggaaataaagaacaatcatttgaatcaagcaattcatgaggagcgcgaggccatcatcgagctgcgcgtgcagctgcggctgctccagatgcagcgagccaaggccgagcagcaggcgcaggaggacgaggagcctgagtggcgcgggggtgccgtccagccgcccagagacggcgtccttgagccaaaagcagctaaagagcagccaaaggcaggcaaggagccggcaaagccatcgcccagcagggataggaaggagacgtccatctgagcagcctgcgtggccgtctggagtccgtgagactgaaaggacccgtgcatcttactgtaacccgggggccaggccggctctctcgctgtacattctgtaaaggtgtcttctcttctcagactcttcctctgtcacacgtctgactccttcacgtcaggctcaggttccatgggaggacgaagcagtggacgcattgtgggctttagggacagatgagttttccagatagtgtcagcttatttgaagattaattttctttgttaacttaaaataactattttaacccttgagtggcttctttttaaaccaaaaaccgtctttctttgcttttttatcacagcagaatcaggatctctttctcattcaaggggggaaccacaccaggtcagcgctgcgcctgctgtggccgccgcgagccacgccctctgggatctctggtaccgtcactcttgcttgtgccttccacaccttctcggtgcagatccctatgggggagctgcctcacgttctctgactggtcagagcagcgcctggtgggtgttccctggcccactctcctctctccttctgcagttctaaaccacagtctataagcccgagtcaccaggacggcctgtctggccacagacaggggctgcctgtggagcctgcccaccggcccccggcagtgcagtccagcggggaggaggctgcccgttcctgccagttcctcactgcggggaccagcaaaggccttctcactgggttggtcaaaggtagtcaccttggcctggtgcatccacagaggatgttgttcaaaccagaaatcttttaaacgactgaccttccttaaaaacagaatgactccgattgcttgcttgggctagaatgtacacgtctccttgcctgaataagccatatatatgctcttaaacaaaagtttgaaattatccatatcatctcagtgaacctactggtggactcccaattgacaagattgagcaatagaaaaaaattcctttcctttgaatgatagctgtgattcaccccaccccattttcttgtttctggtccatccgatgagacggatgctctgatgctctgaggcttctgggaggctgggccctggaggcaacgtgctgcaggcgcactctgtcagagtgaacagcaccgcgagacaggccaggctcgtggctcggaagacaaaccccacacacactcaaggggtcgaaaacaaaccccacacgagggctctcacctccttctcctaggtagtatttattttcagcacctgtttgatgcagtttttaatcctctacctattgcactgttgtgactcgttggccattatttgattttggtacgaaaaaaagctttgttatagaaatcagcatactatttttttaaatctggagagaagatattctggtgactgaaagtatggtcgggtgtcagatataaatgtgcaaatgccttcttgctgtcctgtcggtctcagtacgttcactttatagctgctggcaatatcgaaggttccttttttgtttgtgtaaactctaatttctatcaaggtgtcatggatttttaaaattagtatttcattacaaatgtctcagcattggttaactaattttgggcaggaccattattgatcaagcaaataaattcaacagccatttgggaaaaag

SEQ ID NO: 119 - protein, 655 aa, Homo sapiens
MTECFLPPTSSPSEHRRVEHGSGLTRTPSSEEISPTKFPGLYRTGEPSPPHDILHEPPDVVSDDEKDHGKKKGKFKKKEKRTEGYAAFQEDSSGDEAESPSKMKRSKGIHVFKKPSFSKKKEKDFKIKEKPKEEKHKEEKHKEEKHKEKKSKDLTAADVVKQWKEKKKKKKPIQEPEVPQIDVPNLKPIFGIPLADAVERTMMYDGIRLPAVFRECIDYVEKYGMKCEGIYRVSGIKSKVDELKAAYDREESTNLEDYEPNTVASLLKQYLRDLPENLLTKELMPRFEEACGRTTETEKVQEFQRLLKELPECNYLLISWLIVHMDHVIAKELETKMNIQNISIVLSPTVQISNRVLYVFFTHVQELFGNVVLKQVMKPLRWSNMATMPTLPETQAGIKEEIRRQEFLLNCLHRDLQGGIKDLSKEERLWEVQRILTALKRKLREAKRQECETKIAQEIASLSKEDVSKEEMNENEEVINILLAQENEILTEQEELLAMEQFLRRQIASEKEEIERLRAEIAEIQSRQQHGRSETEEYSSESESESEDEEELQIILEDLQRQNEELEIKNNHLNQAIHEEREAIIELRVQLRLLQMQRAKAEQQAQEDEEPEWRGGAVQPPRDGVLEPKAAKEQPKAGKEPAKPSPSRDRKETSI

SEQ ID NO: 120 - DNA, 746 bp, Homo sapiens
  misc_feature (2)..(6): n is a, c, g, or t
tnnnnnttnnnnnnttnnccttgctcagcattggntntgatgtgctggtggagaaccacgaagaatgnattgctgaggggagacctggtccagggtcttctcccctgtaatccagggccacactgatgagntctgggggntctgcacacacccctcccagaaccgnttcctcacctgcggccacgaccggnagttctgcctgtgggatggggagagccatgcactggcctggagcatcgacctcaaggagactggtctctgtgctgacttccacccgagtggggcagttgtggccgnaggactgaacacggggaggtggttggttttggncacagagaccagagagatcgtgtctgatgtcattgatggcaatnagcagctctcagtggtccggtacagnccagatgggttggtcctggcccaattggttccccatnacaacntnatnttcaatcttttngnggtttccaggggatggtgcccaattccagnccnttttgggccntttgtntttgggtcaacncccagnttcaaccactcaatnttggagtaggttcaannnttngnnttaccagttgnnnttntccaannnnnnnnnnnnntntnnnntnnttnttcttttncntnanncnnnnnnnnnnncnnntctncntnttnntcaanccnnntnnnnnnncnnncnnnnncntntnnctncntnnnnncnntnnnnctnntnnncnnnnnctnnnnntnnncnnnnnnnn

SEQ ID NO: 121 - DNA, 1211 bp, Homo sapiens
ggccccccccccccctagaaatgctcgaaccaggacggctcctggagtcctcgcgccctcgcagaaggactacgggccccggcgaccccgggggcggggcttccggcgcgctgccttgtgggcacggtagttccgccgggtctggcttccgcctgccgagcggccccggaccgcaggccggactacacttcccgtcggcccgcctgctctcccgatgccgccttggcgcgagacgttggcaagcagagtgtctccaagatggccgcttggggaaggaggcgtcttggcccgggcagcagtggcggcagcgcccgagagagggtgagcttgtcggccacagactgctacattgtgcatgagatctacaatggggagaatgcccaagaccagtttgagtacgagctggagcaggccctggaagcccagtacaagtacattgtgattgagcccactcgcattggcgacgagacagcccgctggatcaccgtgggcaactgcctgcacaagacggccgtgctggcgggcaccgcctgcctcttcaccccgttggcgctgcccttagattattcccactacatttccctgcccgctggtgtgctgagcctggcctgctgcaccctctatgggatctcctggcagtttgacccttgctgcaagtaccaagtggagtacgacgcctataaactgtcgcgcctgcctctgcacacactcacctcctccaccccggtggtgctggtccggaaggacgacctgcacagaaagagactgcacaacacgatagcactggccgccctggtgtactgtgtaaagaagatttacgaactctatgccgtatgatttcagtagaacagggagcgaagcaaaaccacccggcccacaagagacaacagagtattcagatcgccacactctgtgaggcagcagagcctgggcaggtgtttggcttagtatttgttatttttaaaaaataacagatcacgggtgtacccagggtttttcagctcattacactaagatgtggatttccataacccaagaggggggtctgaggctgtggaagtccgactgggcagtggaatgctgatggaggcagacgctgccgagggggtgtggacgtgctttgggggaggtctttaagtctattgtttaactgtaccatccagagcccaccagaagctattgatcattaaaattatgagaatttcaactcc

SEQ ID NO: 122 - protein, 192 aa, Homo sapiens
MAAWGRRRLGPGSSGGSARERVSLSATDCYIVHEIYNGENAQDQFEYELEQALEAQYKYIVIEPTRIGDETARWITVGNCLHKTAVLAGTACLFTPLALPLDYSHYISLPAGVLSLACCTLYGISWQFDPCCKYQVEYDAYKLSRLPLHTLTSSTPVVLVRKDDLHRKRLHNTIALAALVYCVKKIYELYAV

SEQ ID NO: 123 - DNA, 1568 bp, Homo sapiens
gaattcgggcgggggagcccaaggagcgagcgcgccagacgaagctcgagccgcctccgccagcgcgaccccacctcggccgccggcctgcgccgcgagatccgccccggcctccccgagagcgagccccggccgccgcgaccaccagccgcgctaaccgccgaccaaccgccaccgaggcgcctgagcgagagcagaggaggaggaggcatgagtgaggcgggcgaggccaccaccaccaccaccaccaccctcccgcaggctccgacggaggcggccgccgcggctccccaggaccccgcgcccaagagcccggtgggcagcggtgcgccccaggccgcggccccggcgcccgccgcccacgtcgcaggaaaccccggtggggacgcggcccctgcagccacgggcaccgcggccgccgcctctttagccgccgccgccggcagcgaagacgcggagaaaaaagttctcgccaccaaagtccttggcactgtcaaatggttcaacgtcagaaatggatatggatttataaatcgaaatgacaccaaagaagatgtatttgtacatcagactgccatcaagaagaataacccacggaaatatctgcgcagtgtaggagatggagaaactgtagagtttgatgtggttgaaggagagaagggtgcagaagctgccaatgtgactggcccggatggagttcctgtggaagggagtcgttacgctgcagatcggcgccgttacagacgtggctactatggaaggcgccgtggccctccccggaattacgctggggaggaggaggaggaagggagcggcagcagtgaaggatttgacccccctgccactgataggcagttctctggggcccggaatcagctgcgccgcccccagtatcgccctcagtaccggcagcggcggttcccgccttaccacgtgggacagacctttgaccgtcgctcacgggtcttaccccatcccaacagaatacaggctggtgagattggagagatgaaggatggagtcccagagggagcacaacttcagggaccggttcatcgaaatccaacttaccgcccaaggtaccgtagcaggggacctcctcgcccacgacctgccccagcagttggagaggctgaagataaagaaaatcagcaagccaccagtggtccaaaccagccgtctgttcgccgtggataccggcgtccctacaattaccggcgtcgcccgccgtcctcctaacgctccttcacaagatggcaaagaggccaaggcaggtgaagcaccaactgagaaccctgctccacccacccagcagagcagtgtgagtaacaccaggctcctcaggcaccttcaccatcggcaggtggacctaaagaattagatgaccattcagaaataaagcaaaaagcaggccacataccttaaccaacaccaaagaaacatccaagcaataaagtggaagactaaccaagatttggacattggaatgtttactgttattctttaagaaacaactacaaaaagaaaatgtcaacaaatttttccagcaagctgagaacctggaattc

SEQ ID NO: 124 - protein, 412 aa, Homo sapiens
EFGRGSPRSERARRSSSRLRQRDPTSAAGLRREIRPGLPESEPRPPRPPAALTADQPPPRRLSESRGGGGMSEAGEATTTTTTTLPQAPTEAAAAAPQDPAPKSPVGSGAPQAAAPAPAAHVAGNPGGDAAPAATGTAAAASLAAAAGSEDAEKKVLATKVLGTVKWFNVRNGYGFINRNDTKEDVFVHQTAIKKNNPRKYLRSVGDGETVEFDVVEGEKGAEAANVTGPDGVPVEGSRYAADRRRYRRGYYGRRRGPPRNYAGEEEEEGSGSSEGFDPPATDRQFSGARNQLRRPQYRPQYRQRRFPPYHVGQTFDRRSRVLPHPNRIQAGEIGEMKDGVPEGAQLQGPVHRNPTYRPRYRSRGPPRPRPAPAVGEAEDKENQQATSGPNQPSVRRGYRRPYNYRRRPPSS

SEQ ID NO: 125 - DNA, 2963 bp, Homo sapiens
gtgagaagcctcctggcagacactggagccacgatgaagcccccaaggcctgtccgtacctgcagcaaagttctcgtcctgctttcactgctggccatccaccagaccactactgccgaaaagaatggcatcgacatctacagcctcaccgtggactccagggtctcatcccgatttgcccacacggtcgtcaccagccgagtggtcaatagggccaatacggtacaggaggccaccttccagatggagctgcccaagaaagccttcatcaccaacttctccatgaacatcgatggcatgacctacccagggatcatcaaggagaaggctgaagcccaggcacagtacagcgcagcagtggccaagggaaagaacgctggcctcgtcaaggccaccgggagaaacatggagcagttccaggtgtcggtcagtgtggctcccaatgccaagatcacctttgagctggtctatgaggagctgctcaagcggcgtttgggggtgtacgagctgctgctgaaagtgcggccccagcagctggtcaagcacctgcagatggacattcacatcttcgagccccagggcatcagctttctggagacagagagcaccttcatgaccaaccagctggtagacgccctcaccacctggcagaataagaccaaggctcacatccggttcaagccaacactttcccagcagcaaaagtccccagagcagcaagaaacagtcctggacggcaacctcattatccgctatgatgtggaccgggccatctccgggggctccattcagatcgagaacggctactttgtacactactttgcccccgagggcctaaccacaatgcccaagaatgtggtctttgtcattgacaagagcggctccatgagtggcaggaaaatccagcagacccgggaagccctaatcaagatcctggatgacctcagccccagagaccagttcaacctcatcgtcttcagtacagaagcaactcagtggaggccatcactggtgccagcctcagccgagaacgtgaacaaggccaggagctttgctgcgggcatccaggccctgggagggaccaacatcaatgatgcaatgctgatggctgtgcagttgctggacagcagcaaccaggaggagcggctgcccgaagggagtgtctcactcatcatcctgctcaccgatggcgaccccactgtgggggagactaaccccaggagcatccagaataacgtgcgggaagctgtaagtggccggtacagcctcttctgcctgggcttcggtttcgacgtcagctatgccttcctggagaagctggcactggacaatggcggcctggcccggcgcatccatgaggactcagactctgccctgcagctccaggacttctaccaggaagtggccaacccactgctgacagcagtgaccttcgagtacccaagcaatgccgtggaggaggtcactcagaacaacttccggctcctcttcaagggctcagagatggtggtggctgggaagctccaggaccgggggcctgatgtgctcacagccacagtcagtgggaagctgcctacacagaacatcactttccaaacggagtccagtgtggcagagcaggaggcggagttccagagccccaagtatatcttccacaacttcatggagaggctctgggcatacctgactatccagcagctgctggagcaaactgtctccgcatccgacgctgatcagcaggccctccggaaccaagcgctgaatttatcacttgcctacagctttgtcacgcctctcacatctatggtagtcaccaaacccgatgaccaagagcagtctcaagttgctgagaagcccatggaaggcgaaagtagaaacaggaatgtccactcaggttccactttcttcaaatattatctccagggagcaaaaataccaaaaccagaggcttccttttctccaagaagaggatggaatagacaagctggagctgctggctcccggatgaatttcagacctggggttctcagctccaggcaacttggactcccaggacctcctgatgttcctgaccatgctgcttaccaccccttccgccgtctggccatcttgcctgcttcagcaccaccagccacctcaaatcctgatccagctgtgtctcgtgtcatgaatatgaaaatcgaagaaacaaccatgacaacccaaaccccagcccccatacaggctccctctgccatcctgccactgcctgggcagagtgtggagcggctctgtgtggaccccagacaccgccaggggccagtgaacctgctctcagaccctgagcaaggggttgaggtgactggccagtatgagagggagaaggctgggttctcatggatcgaagtgaccttcaagaaccccctggtatgggttcacgcatcccctgaacacgtggtggtgactcggaaccgaagaagctctgcgtacaagtggaaggagacgctattctcagtgatgcccggcctgaagatgaccatggacaagacgggtctcctgctgctcagtgacccagacaaagtgaccatcggcctgttgttctgggatggccgtggggaggggctccggctccttctgcgtgacactgaccgcttctccagccacgttggagggacccttggccagttttaccaggaggtgctctggggatctccagcagcatcagatgacggcagacgcacgctgagggttcagggcaatgaccactctgccaccagagagcgcaggctggattaccaggaggggcccccgggagtggagatttcctgctggtctgtggagctgtagttctgatggaaggagctgtgcccaccctgtacacttggcttccccctgcaactgcagggccgcttctggggcctggaccaccatggggaggaagagtcccactcattacaaataaagaaaggtggtgtgagcctggg

SEQ ID NO: 126 - protein, 930 aa, Homo sapiens
MKPPRPVRTCSKVLVLLSLLAIHQTTTAEKNGIDIYSLTVDSRVSSRFAHTVVTSRVVNRANTVQEATFQMELPKKAFITNFSMNIDGMTYPGIIKEKAEAQAQYSAAVAKGKNAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTNINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSGSTFFKYYLQGAKIPKPEASFSPRRGWNRQAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 127 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCSNDEGLECVPTEESNITMQIMRIKPHQGQHIGEMSFLQHNKCECRPKKDRARQENPCGPCSERRKHLFVQDPQTCKCSCKNTHSRCKARQLELNERTCRCDKPRR

SEQ ID NO: 128 - protein, 221 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRHSPGRQSPDMPGDFRADAPSFLPPRRSLPMLFRMEWGCALTGSQSAVWPSSPVPEEIPRMHPGRNGKKQQRKPLREKMKPERCGDAVPRR

SEQ ID NO: 129 - protein, 1356 aa, Homo sapiens
MQSKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLEIIILVGTAVIAMFFWLLLVIILRTVKRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 130 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKEMSKRSP

SEQ ID NO: 131 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 132 - DNA, 5102 bp, Homo sapiens
gaattccacttctctgtcgcccgcggttcgccgccccgctcgccgccgcgatgccagtgtttcatacgcgcacgatcgagagcatcctggagccggtggcacagcagatctcccacctggtgataatgcacgaggagggcgaggtggacggcaaagccattcctgacctcaccgcgcccgtggccgccgtgcaggcggccgtcagcaacctcgtccgggttggaaaagagactgttcaaaccactgaggatcagattttgaagagagatatgccaccagcatttattaaggttgagaatgcttgcaccaagcttgtccaggcagctcagatgcttcagtcagacccttactcagtgcctgctcgagattatctaattgatgggtcaaggggcatcctctctggaacatcagacctgctccttaccttcgatgaggctgaggtccgtaaaattattagagtttgcaaaggaattttggaatatcttacagtggcagaggtggtggagactatggaagatttggtcacttacacaaagaatcttgggccaggaatgactaagatggccaagatgattgacgagagacagcaggagctcactcaccaggagcaccgagtgatgttggtgaactcgatgaacaccgtgaaagagttgctgccagttctcatttcagctatgaagatttttgtaacaactaaaaactcaaaaaaccaaggcatagaggaagctttaaaaaatcgcaattttactgtagaaaaaatgagtgctgaaattaatgagataattcgtgtgttacaactcacctcttgggatgaagatgcctgggccagcaaggacactgaagccatgaagagagcattggcctccatagactccaaactgaaccaggccaaaggttggctccgtgaccctagtgcctccccaggggatgctggtgagcaggccatcagacagatcttagatgaagctggaaaagttggtgaactctgtgcaggcaaagaacgcagggagattctgggaacttgcaaaatgctagggcagatgactgatcaagtggctgacctccgtgccagaggacaaggatcctcaccggtggccatgcagaaagctcagcaggtatctcagggtctggatgtgctcacagcaaaagtggaaaatgcagctcgcaagctggaagccatgaccaactcaaagcagagcattgcaaagaagatcgatgctgctcagaactggcttgcagatccaaatggtggaccggaaggagaagagcagattcgaggtgctttggctgaagctcggaaaatagcagaattatgtgatgatcctaaagaaagagatgacattctacgttcccttggggaaatatctgctctgacttctaaattagcagatctacgaagacaggggaaaggagattctccagaggctcgagccttggccaaacaggtggccacggccctgcagaacctgcagaccaaaaccaaccgggctgtggccaacagcagaccggccaaagcagctgtacaccttgagggcaagattgagcaagcacagcggtggattgataatcccacagtggatgaccgtggagtcggtcaggctgccatccgggggcttgtggccgaagggcatcgtctggctaatgttatgatggggccttatcggcaagatcttctcgccaagtgtgaccgagtggaccagctgacagcccagctggctgacctggctgccagaggggaaggggagagtcctcaggcacgagcacttgcatctcagctccaagactccttaaaggatctaaaagctcggatgcaggaggccatgactcaggaagtgtcagatgttttcagcgataccacaactcccatcaagctgttggcagtggcagccacggcgcctcctgatgcgcctaacagggaagaggtatttgatgagagggcagctaactttgaaaaccattcaggaaagcttggtgctacggccgagaaggcggctgcggttggtactgctaataaatcaacagtggaaggcattcaggcctcagtgaagacggcccgagaactcacaccccaggtggtctcggctgctcgtatcttacttaggaaccctggaaatcaagctgcttatgaacattttgagaccatgaagaaccagtggatcgataatgttgaaaaaatgacagggctggtggacgaagccattgataccaaatctctgttggatgcttcagaagaagcaattaaaaaagacctggacaagtgcaaggtagctatggccaacattcagcctcagatgctggttgctggggcaaccagtattgctcgtcgggccaaccggatcctgctggtggctaagagggaggtggagaattccgaggatcccaagttccgtgaggctgtgaaagctgcctctgatgaattgagcaaaaccatctccccaatggtgatggatgcaaaagctgtggctggaaacatttccgaccctggactgcaaaagagcttcctggactcaggatatcggatcctgggagctgtggccaaggtcagagaagccttccaacctcaggagcctgacttcccgccgcctccaccagaccttgaacaactccgactaacagatgagcttgctcctcccaaaccacctctgcctgaaggtgaggtccctccacctaggcctccaccaccagaggaaaaggatgaagagttccctgagcagaaggccggggaggtgattaaccagccaatgatgatggctgccagacagctccatgatgaagctcgcaaatggtccagcaagggcaatgacatcattgcagcagccaagcgcatggctctgctgatggctgagatgtctcggctggtaagagggggcagtggtaccaagcgggcactcattcagtgtgccaaggacatcgccaaggcctcagatgaggtgactcggttggccaaggaggttgccaagcagtgcacagataaacggattagaaccaacctcttacaggtatgtgagcgaatcccaaccataagcacccagctcaaaatcctgtccacagtgaaggccaccatgctgggccggaccaacatcagtgatgaggagtctgagcaggccacagagatgctggttcacaatgcccagaacctcatgcagtctgtgaaggagactgtgcgggaagctgaagctgcttcaatcaaaattcgaacagatgctggatttacactgcgctgggttagaaagactccctggtaccagtaggcacctggctgagcctggctggcacagaaacctctactaaaaagaaggaaaatgatctgagtcccaggagctgcccagagttgctgggagctgaaaaatcacatcctggcctggcacatcagaaaggaatgggggcctcttcaaattagaagacatttatactcttttttcatggacactttgaaatgtgtttctgtataaagcctgtattctcaaacacagttacacttgtgcaccctctatcccaataggcagactgggtttctagcccatggacttcacataagctcagaatccaagtgaacactagccagacactctgctctgcccttgttccctaggggacacttccctctgtttctctttccttggctcccattcactcttccagaatcccaagacccagggcccaggcaaatcagttactaagaagaaaattgctgtgcctcccaaaattgttttgagctttccatgttgctgccaaccataccttccttccctgggctgtgctacctgggtccttttcagaagtgagctttgctgctacaggggaaggtggcctctgtggagccccagcatatgggggcctggattcatttcctgcccttcctcagtttaatccttctagtttcccacaatataaaactgtacttcactgtcaggaagaaatcacagaatcatatgattctgcttttaccatgcccctgagcaatgtctgtgctagggaaactccccgtcccatatcctgcctcagcccgccaaggtagccatcccatgaacacactgtgtcctggtgctctctgccactggaagggcagagtagccagggtgtggccctgccatcttcccagcagggccactcccggcactccatgcttagtcactgcctgcagaggtctgtgctgaggccttatcattcattcttagctcttaattgttcattttgagctgaaatgctgcattttaattttaaccaaaacatgtctcctatatcctggtttttgtagccttcctccacatcctttctaaacaagattttaaagacatgtaggtgtttgttcatctgtaactctaaaagatcctttttaaattcagtcctaagaaagaggagtgcttgtcccctaagagtgtttaatggcaaggcagccctgtctgaaggacacttcctgcctaagggagagtggtatttgcagactagaattctagtgctgctgaagatgaatcaatgggaaatactactcctgtaattcctacctccctgcaaccaactacaaccaagctctctgcatctactcccaagtatggggttcaagagagtaatgggtttcatatttcttatcaccacagtaagttcctactaggcaaaatgagagggcagtgtttcctttttggtacttattactgctaagtatttcccagcacatgaaaccttattttttccaaagccagaaccagatgagtaaaggagtaagaaccttgcctgaacatccttccttcccacccatcgctgtgtgttagttcccaacatcgaatgtgtacaacttaagttggtcctttacactcaggctttcactatttcctttaaaatgaggatgattattttcaaggccctcagcatatttgtatagttgcttgcctgatataaatgcaatattaatgcctttaaagtatgaatctatgccaaagatcacttgttgttttactaaagaaagattacttagaggaaataagaaaaatcatgtttgctctcccggttcttccagtggtttgagacactggtttacactttatgccggatgtgcttttctccaatatcagtgctcgagacacagtgaagcaaattaaaaaaaa

SEQ ID NO: 133 - DNA, 1450 bp, Homo sapiens
aagcttttagttccctttaatcataaaagccacttgctaactaaaactagagatagctcaagctatctgattttaaaggcttagtctcaatgtgtcccttccctgaaatcccagtagagtagccaattgtctgaaacccgcttggatttagcaatgaaacacctcagtcctggccaaaccaagacagtgggtctcaggaaacattcttcatcctaataaaggcaaattaactacagttgacccttgaacaacatgggggttatgggtgctgacttcccatgcagtaaaaaatctgggtataactttcgattccacaaaaactttgctaatagccttaactgttaactggaagccttaccaataacacatacagttgattaacacatatattgtatgtattatattacaataaattaagctagagaaaagaaaatgtattccttttgctgccatcctgcctccctgtgtgcttctaatctcagctggtctcacccgggactccccaagtgccaaccctagtcccccacgcagccccttttccactcagataagatgaaagaaacaatcatgaaccaaaaactcaccaaacggcaagcagaagtgcacactggtcggaaaggaactgctcacaggaaaaaggtggttcacagcatctgagacgctgtgtttgagggcaagtaggcccctttgacacctttggtgttgaactcatgaggttttgaatgtccagggacattggccaatatcaaaagaactttaaaagtcagtttggtaaggtacttcttgacttcagggacaaaacagcaatggaaccaatccagaaaaagggttctcattgtccaggccttctggtacaaccaaaagactggcagctggcatttatcttgtcccttcaaggctcagaggttaacggttttatacataagggtagtcctgatcataaacctagcgacagcagaggataaaaaatttcagttctccttaaagaaattaggggtaaacaatatccctggtattgaagaggtgaatatgtttacacaccaaggaacagtgattcactttaacaaccctgaagttcaggcatcgctggcagcaaacactttcaccatgacaggccatgctgagacaaagcagctgacagaaatgctactcagcatcgatcataaaccagtgctgcagatggtctgactagttcaaagagactggctaacactgcccaaacaatctgtgggtggaaaagcaccacttgctactggagaggatgatgaagttctagatcttgtggagaattctgatgaggcttccaacaatgaggcaaactgaattaagtcaacttctgaagaagataaaacttgaagtagttactgagagctgctgttttatgttatgactgctttttaaaaatgtttttgtttacagatcttaataaaatctagatctctaatatttt

SEQ ID NO: 134 - DNA, 3622 bp, Homo sapiens
cccggggaggggaccggggaacagagggccgagaggcgtgcggcaggggggagggtaggagaaagaagggcccgactgtaggagggcagcggagcattacctcatcccgtgagcctccgcgggcccagagaagaatcttctagggtggagtctccatggtgacgggcgggcccgcccccctgagagcgacgcgagccaatgggaaggccttggggtgacatcatgggctatttttaggggttgactggtagcagataagtgttgagctcgggctggataagggctcagagttgcactgagtgtggctgaagcagcgaggcgggagtggaggtgcgcggagtcaggcagacagacagacacagccagccagccaggtcggcagtatagtccgaactgcaaatcttattttcttttcaccttctctctaactgcccagagctagcgcctgtggctcccgggctggtggttcgggagtgtccagagagccttgtctccagccggccccgggaggagagccctgctgcccaggcgctgttgacagcggcggaaagcagcggtaccccacgcgcccgccgggggacgtcggcgagcggctgcagcagcaaagaactttcccggcggggaggaccggagacaagtggcagagtcccggagcgaacttttgcaagcctttcctgcgtcttaggcttctccacggcggtaaagaccagaaggcggcggagagccacgcaagagaagaaggacgtgcgctcagcttcgctcgcaccggttgttgaacttgggcgagcgcgagccgcggctgccgggcgccccctccccctagcagcggaggaggggacaagtcgtcggagtccgggcggccaagacccgccgccggccggccactgcagggtccgcactgatccgctccgcggggagagccgctgctctgggaagtgagttcgcctgcggactccgaggaaccgctgcgcccgaagagcgctcagtgagtgaccgcgacttttcaaagccgggtagcgcgcgcgagtcgacaagtaagagtgcgggaggcatcttaattaaccctgcgctccctggagcgagctggtgaggagggcgcagcggggacgacagccagcgggtgcgtgcgctcttagagaaactttccctgtcaaaggctccggggggcgcgggtgtcccccgcttgccagagccctgttgcggccccgaaacttgtgcgcgcacgccaaactaacctcacgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggaggggttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttgaagagagaccgtcgggggctgaggggcaacgaagaaaaaaaataacacagagagacagacttgagaacttgacaagttgcgacggagagaaaaaagaagtgtccgagaactaaagccaagggtatccaagttggactgggttcggtctgacggcgcccccagtgtgcacgagtgggaaggacttggtcgcgccctcccttggcgtggagccagggagcggccgcctgcgggctgccccgctttgcggacgggctgtccccgcgcgaacggaacgttggactttcgttaacattgaccaagaactgcatggacctaacattcgatctcattcagtattaaaggggggagggggagggggttacaaactgcaatagagactgtagattgcttctgtagtactccttaagaacacaaagcggggggagggttggggaggggcggcaggagggaggtttgtgagagcgaggctgagcctacagatgaactctttctggcctgctttcgttaactgtgtatgtacatatatatattttttaatttgattaaagctgattactgtcaataaacagcttcatgcctttgtaagttatttcttgtttgtttgtttgggtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatcctagtctatctaggtggagttgaaagagttaagaatgctcgataaaatcactctcagtgcttcttactattaagcagtaaaaactgttctctattagacttagaaataaatgtacctgatgtacctgatgctatgtcaggcttcatactccacgctcccccagcgtatctatatggaattgcttaccaaaggctagtgcgatgtttcaggaggctggaggaaggggggttgcagtggagagggacagcccactgagaagtcaaacatttcaaagtttggattgcatcaagtggcatgtgctgtgaccatttataatgttagaaattttacaataggtgcttattctcaaagcaggaattggtggcagattttacaaaagatgtatccttccaatttggaatcttctctttgacaattcctagataaaaagatggcctttgtcttatgaatatttataacagcattctgtcacaataaatgtattcaaataccaataacagatcttgaattgcttccctttactacttttttgttcccaagttatatactgaagtttttatttttagttgctgaggtt

SEQ ID NO: 135 - DNA, 6210 bp, Homo sapiens
gcaggaacagtgctagtattgctcgagcccgagggctggaggttaggggatgaaggtctgcttccacgctttgcactgaattagggctagaattggggatgggggtaggggcgcattccttcgggagccgaggcttaagtcctcggggtcctgtactcgatgccgtttctcctatctctgagcctcagaactgtcttcagtttccgtacaagggtaaaaaggcgctctctgccccatcccccccgacctcgggaacaagggtccgcattgaaccaggtgcgaatgttctctctcattctgcgccgttcccgcctcccctcccccagccgcggcccccgcctccccccgcactgcaccctcggtgttggctgcagcccgcgagcagttcccgtcaatccctccccccttacacaggatgtccatattaggacatctgcgtcagcaggtttccacggcctttccctgtagccctggggggagccatccccgaaacccctcatcttggggggcccacgagacctctgagacaggaactgcgaaatgctcacgagattaggacacgcgccaaggcgggggcagggagctgcgagcgctggggacgcagccgggcggccgcagaagcgcccaggcccgcgcgccacccctctggcgccaccgtggttgagcccgtgacgtttacactcattcataaaacgcttgttataaaagcagtggctgcggcgcctcgtactccaaccgcatctgcagcgagcaactgagaagccaagactgagccggcggccgcggcgcagcgaacgagcagtgaccgtgctcctacccagctctgcttcacagcgcccacctgtctccgcccctcggcccctcgcccggctttgcctaaccgccacgatgatgttctcgggcttcaacgcagactacgaggcgtcatcctcccgctgcagcagcgcgtccccggccggggatagcctctcttactaccactcacccgcagactccttctccagcatgggctcgcctgtcaacgcgcaggtaaggctggcttcccgtcgccgcggggccgggggcttggggtcgcggaggaggagacaccgggcgggacgctccagtagatgagtagggggctcccttgtgcctggagggaggctgccgtggccggagcggtgccggctcgggggctcgggacttgctctgagcgcacgcacgcttgccatagtaagaattggttcccccttcgggaggcaggttcgttctgagcaacctctggtctgcactccaggacggatctctgacattagctggagcagacgtgtcccaagcacaaactcgctaactagagcctggcttcttcggggaggtggcagaaagcggcaatcccccctcccccggcagcctggagcacggaggagggatgagggaggagggtgcagcgggcgggtgtgtaaggcagtttcattgataaaaagcgagttcattctggagactccggagcggcgcctgcgtcagcgcagacgtcagggatatttataacaaaccccctttcaagcaagtgatgctgaagggataacgggaacgcagcggcaggatggaagagacaggcactgcgctgcggaatgcctgggaggaaaagggggagacctttcatccaggatgagggacatttaagatgaaatgtccgtggcaggatcgtttctcttcactgctgcatgcggcactgggaactcgccccacctgtgtccggaacctgctcgctcacgtcggctttccccttctgttttgttctaggacttctgcacggacctggccgtctccagtgccaacttcattcccacggtcactgccatctcgaccagtccggacctgcagtggctggtgcagcccgccctcgtctcctctgtggccccatcgcagaccagagcccctcaccctttcggagtccccgccccctccgctggggcttactccagggctggcgttgtgaagaccatgacaggaggccgagcgcagagcattggcaggaggggcaaggtggaacaggtgaggaactctagcgtactcttcctgggaatgtgggggctgggtgggaagcagccccggagatgcaggagcccagtacagaggatgaagccactgatggggctggctgcacatccgtaactgggagccctggctccaagcccattccatcccaactcagactctgagtctcaccctaagaagtactctcatagtttcttccctaagtttcttaccgcatgctttcagactgggctcttctttgttctcttgctgaggatcttattttaaatgcaagtcacacctattctgcaactgcaggtcagaaatggtttcacagtggggtgccaggaagcagggaagctgcaggagccagttctactggggtgggtgaatggaggtgatggcagacacttttactgaatgtcggtctttttttgtgattattctagttatctccagaagaagaagagaaaaggagaatccgaagggaaaggaataagatggctgcagccaaatgccgcaaccggaggagggagctgactgatacactccaagcggtaggtactctgtgggttgctcctttttaaaacttaagggaaagttggagattgagcataagggcccttgagtaagactgtgtcttatgctttcctttatccctctgtatacaggagacagaccaactagaagatgagaagtctgctttgcagaccgagattgccaacctgctgaaggagaaggaaaaactagagttcatcctggcagctcaccgacctgcctgcaagatccctgatgacctgggcttcccagaagagatgtctgtggcttcccttgatctgactgggggcctgccagaggttgccaccccggagtctgaggaggccttcaccctgcctctcctcaatgaccctgagcccaagccctcagtggaacctgtcaagagcatcagcagcatggagctgaagaccgagccctttgatgacttcctgttcccagcatcatccaggcccagtggctctgagacagcccgctccgtgccagacatggacctatctgggtccttctatgcagcagactgggagcctctgcacagtggctccctggggatggggcccatggccacagagctggagcccctgtgcactccggtggtcacctgtactcccagctgcactgcttacacgtcttccttcgtcttcacctaccccgaggctgactccttccccagctgtgcagctgcccaccgcaagggcagcagcagcaatgagccttcctctgactcgctcagctcacccacgctgctggccctgtgagggggcagggaaggggaggcagccggcacccacaagtgccactgcccgagctggtgcattacagagaggagaaacacatcttccctagagggttcctgtagacctagggaggaccttatctgtgcgtgaaacacaccaggctgtgggcctcaaggacttgaaagcatccatgtgtggactcaagtccttacctcttccggagatgtagcaaaacgcatggagtgtgtattgttcccagtgacacttcagagagctggtagttagtagcatgttgagccaggcctgggtctgtgtctcttttctctttctccttagtcttctcatagcattaactaatctattgggttcattattggaattaacctggtgctggatattttcaaattgtatctagtgcagctgattttaacaataactactgtgttcctggcaatagtgtgttctgattagaaatgaccaatattatactaagaaaagatacgactttattttctggtagatagaaataaatagctatatccatgtactgtagtttttcttcaacatcaatgttcattgtaatgttactgatcatgcattgttgaggtggtctgaatgttctgacattaacagttttccatgaaaacgttttattgtgtttttaatttatttattaagatggattctcagatatttatatttttattttatttttttctaccttgaggtcttttgacatgtggaaagtgaatttgaatgaaaaatttaagcattgtttgcttattgttccaagacattgtcaataaaagcatttaagttgaatgcgaccaaccttgtgctcttttcattctggaagtcttgtaagtttctgaaaggtattattggagaccagtttgtcaagaagggtagctgctggagggggacacaccctctgtctgatcccttatcaaagaggacaaggaaactatagagctgattttagaatattttacaaatacatgccttccattggaatgctaagattttctactgcttctggggacgggaaaccgctgtgtaacagcttttgtgggaatacattttttctgtttcagtactcgcagggggaaatatttaaattttgttgtgctaatattaaattcagatgttttgatcttaaaggaaccctttaagcaaacagaacctagctttgtacagactattttaactttttattctcacaaaatcacgtggagggttattctacttcaaagatgagcaaattgaagaatggttagaataaacaactttcttgatattccgttatcggcattagaatcttcctgctcgttatcgtatccagcaggctgaactgcctcttgatacttggttaaaaaaaattttcaggccgggcgcggtggcccatgcctgtaatcctagcactttgggaggccgaggcaggcggatcacctgaggtcgggagttcgagaccagcctgaccaacatggagaaaccccgtctttactaaaaatacaaaattagcctggtgtggtggtgcatgcctgtaatcctagctacttgagaggctgagacaggaaaatcacttgaactcgggaggcggatgttgcagcgaactgagattgcgccattgcactccagcctgggcaacaagattgaaactctgtttaaaaaaaaaagttttcactaatgtgtacatttttttgtactcttttattctcgaaagggaaggagggctattgccctatcccttattaataaatgcattgtggtttctggtttctctaataccatatgcccttcattcagtttatagtgggcggaagtgggggagaaaaagttgctcagaaatcaaaagatatctcaaacagcacaaataatggctgatcgttctgcaaacaaaaagttacataatagctcaagaaggagaagtcaacatgactctgaacaagctttaacttagaaactttatcatcttaaggaagaacgtgacctttgtccaggacgtctctggtaatggggcacttacacacacatgcacacgtacaaaccacagggaaaggagaccgcccttctgcctctgctcgcgagtatcacgcaggcaccatgcactatgttttcacacacactgggtggaagaagagcttcagcgccagtcttctaatgctttggtgataatgaaaatcactgggtgcttatggggtgtcatattcaatcgagttaaaagttttaattcaaaatgacagttttactgaggttgatgttctcgtctatgatatctctgcccctcccataaaaatggacatttaaaagcaacttaccgctctttagatcactcctatatcacacaccacttggggtgctgtttctgctagacttgtgatgacagtggccttaggatccctgtttgctgttcaaagggcaaatattttatagcctttaaatatacctaaactaaatacagaattaatataactaacaaacacctggtctgaaataacaaggtgatctaccctggaaggaacccagctggtgggccaggagcggtggctcacacctgtaattccagcactttgggaggctgagacaggaggatcactggagtccaggagtttgagaccagcctgggcaacatggcaaaacccagtgtgcttctgttgtcccagctacactactcaggaggctgaggcaggagtatgacttgagcctgggagggggaggttgcagagaactgatattgcaccaccactgcactccagcctgggtgacacagcaaaaccctatctcaaaaaaaaaaaaaaaaaaaaggaacccagctggttcctgtaggtgtgcaataataacaaccagaggaagaaaaggaagacgatttcccagatgaagaagggcagctggaccttcggac

SEQ ID NO: 136 - DNA, 1714 bp, Homo sapiens
gctcgggcgccgagtctgcgcgctgacgtccgacgctccaggtactttccccacggccgacagggcttggcgtgggggcggggcgcggcgcgcagcgcgcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggaaattcgaaatgagtcccatgactatcctcatagagtggccaagtttccagaaaacagaaatcgaaacagatacagagatgtaagcccatatgatcacagtcgtgttaaactgcaaaatgctgagaatgattatattaatgccagtttagttgacatagaagaggcacaaaggagttacatcttaacacagggtccacttcctaacacatgctgccatttctggcttatggtttggcagcagaagaccaaagcagttgtcatgctgaaccgcattgtggagaaagaatcggttaaatgtgcacagtactggccaacagatgaccaagagatgctgtttaaagaaacaggattcagtgtgaagctcttgtcagaagatgtgaagtcgtattatacagtacatctactacaattagaaaatatcaatagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttgatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagagtgctctacggaaacgtattcgagaggacagaaaggccaccacagctcagaaggtgcagcagatgaaacagaggctaaatgagaatgaacgaaaaagaaaaaggccaagattgacagacacctaatattcatgacttgagaatattctgcagctataaattttgaaccattgatgtgcaaagcaagacctgaagcccactccggaaactaaagtgaggctcgctaaccctctagattgcctcacagttgtttgtttacaaagtaaactttacatccaggggatgaagagcacccaccagcagaagactttgcagaacctttaattggatgtgttaagtgtttttaatgagtgtatgaaatgtagaaagatgtacaagaaataaattaggagagattactttgtattgtactgccattcctactgtatttttatactttttggcagcattaaatatttttgttaaataaaaaaaaaaaaaaa

SEQ ID NO: 137 - DNA, 2213 bp, Homo sapiens
ggaggcggcaacattgtttcaagttggccaaattgacaagagcgagaggtatactgcgttccatcccgacccgggccacggtactgggccctgtttccccctcctcggcccccgagagccagggtccgccttctgcagggttcccaggcccccgctccagggccgggctgacccgactcgctggcgcttcatggagaacttccaaaaggtggaaaagatcggagagggcacgtacggagttgtgtacaaagccagaaacaagttgacgggagaggtggtggcgcttaagaaaatccgcctggacactgagactgagggtgtgcccagtactgccatccgagagatctctctgcttaaggagcttaaccatcctaatattgtcaagctgctggatgtcattcacacagaaaataaactctacctggtttttgaatttctgcaccaagatctcaagaaattcatggatgcctctgctctcactggcattcctcttcccctcatcaagagctatctgttccagctgctccagggcctagctttctgccattctcatcgggtcctccaccgagaccttaaacctcagaatctgcttattaacacagagggggccatcaagctagcagactttggactagccagagcttttggagtccctgttcgtacttacacccatgaggtggtgaccctgtggtaccgagctcctgaaatcctcctgggctgcaaatattattccacagctgtggacatctggagcctgggctgcatctttgctgagatggtgactcgccgggccctattccctggagattctgagattgaccagctcttccggatctttcggactctggggaccccagatgaggtggtgtggccaggagttacttctatgcctgattacaagccaagtttccccaagtgggcccggcaagattttagtaaagttgtacctcccctggatgaagatggacggagcttgttatcgcaaatgctgcactacgaccctaacaagcggatttcggccaaggcagccctggctcaccctttcttccaggatgtgaccaagccagtaccccatcttcgactctgatagccttcttgaagcccccagccctaatctcaccctctcctccagtgtgggcttgaccaggcttgcgcttgggctatttggactcaggtgggccctctgaacttgccttaaacactcaccttctagtcttggccagccaactctgggaatacaggggtgaaaggggggaaccagtgaaaatgaaaggaagtttcagtattagatgcacttaagttagcctccaccaccctttcccccttctcttagttattgctgaagagggttggtataaaaataattttaaaaaagccttcctacacgttagatttgccgtaccaatctctgaatgccccataattattatttccagtgtttgggatgaccaggatcccaagcctcctgctgccacaatgtttataaaggccaaatgatagcgggggctaagttggtgcttttgagaaccaagtaaaacaaaaccactgggaggagtctattttaaagaattcggttgaaaaaaatagatccaatcagtttataccctagttagtgttttgcctcacctaataggctgggagactgaagactcagcccgggtggctgcagaaaaatgattggccccagtccccttgtttgtcccttctacaggcatgaggaatctgggaggccctgagacagggattgtgcttcattccaatctattgcttcaccatggccttatgaggcaggtgagagatgtttgaatttttctcttccttttagtattcttagttgttcagttgccaaggatccctgatcccattttcctctgacgtccacctcctaccccataggagttagaagttagggtttaggcatcattttgagaatgctgacactttttcagggctgtgattgagtgagggcatgggtaaaaatatttctttaaaagaaggatgaacaattatatttatatttcaggttatatccaatagtagagttggctttttttttttttttttggtcatagtgggtggatttgttgccatgtgcaccttggggttttgtaatgacagtgctaaaaaaaaagcattttttttttatgatttgtctctgtcacccttgtccttgagtgctcttgctattaacgttatttgtaatttagtttgta

SEQ ID NO: 138 - DNA, 1508 bp, Homo sapiens
gagcgcggttaccggacgggctgggtctatggtcgctccgcggccgctccgccgcgtggtgcttttttatcagggcaagctgtgttccatggcagggaacttttggcagagctcccactatttgcaatggattttggataaacaagatctgttgaaggagcgccaaaaggatttaaagtttctctcagaggaagaatattggaagttacaaatattttttacaaatgttatccaagcattaggtgaacatcttaaattaagacaacaagttattgccactgctacggtatatttcaagagattctatgccaggtattctctgaaaagtatagatcctgtattaatggctcctacatgtgtgtttttggcatccaaagtagaggaatttggagtagtttcaaatacaagattgattgctgctgctacttctgtattaaaaactagattttcatatgcctttccaaaggaatttccttataggatgaatcatatattagaatgtgaattctatctgttagaactaatggattgttgcttgatagtgtatcatccttatagacctttgctccagtatgtgcaggacatgggccaagaagacatgttgcttccccttgcatggaggatagtgaatgatacctacagaacggatctttgcctactgtatcctcctttcatgatagctttagcttgcctacatgtagcctgtgttgtacagcagaaagatgccaggcaatggtttgctgagctttctgtggatatggaaaagattttggaaataatcagggttattttaaaactatatgagcagtggaagaatttcgatgagagaaaagagatggcaaccattcttagtaagatgccaaaaccaaaaccacctccaaacagtgaaggagagcagggtccaaatggaagtcagaactctagctacagccaatcttaaaacattccgaagaattccatagtggaccacttggaaataaaccattggacagatttcagtaatgtcttcagtggaacacaaatgaaaatgaatagcttgtttctgtcaagcatattggaaagtgattttatttttgcaaatagtttttctttaatatgattctagtacataattgattgattaaatctcttgattataaatgtttggaaaggttctaaggggacctacagacagacatacatagacatttcaaaattaatagcttttgattagtataatatttcttaatttggataataaaaattgtagctttttattaagccaggaaacatgaagcataatttgtttaaaattctctttggtcattgagggaccaaaaaaggacgtaaaatttacagtcaatctatgagggtttttttccctccataagtttaactttaaaactgtatttaaggaatcaaatcttacaaaatcctggaagattttggtaatgatgttgataatttcagggaaattaatcaagtaccgtatattgatttaaaagtgtattttattcagtagtttgagg

SEQ ID NO: 139 - DNA, 4320 bp, Homo sapiens
aggatttggggtggaaggcaggcatggtcaacccatgtcactgacaggagagcagagacagacgtgtctctctccacgtcttccagccagtaaaagaagccaagctggagcccaaagccaggtgttctgactcccagcgtgggggtccctgcaccaaccatgagccgcctgctctggaggaaggtggccggcgccaccgtcgggccagggccggttccagctccggggcgctgggtctccagctccgtccccgcgtccgaccccagcgacgggcagcggcggcggcagcagcagcagcagcagcagcagcagcagcaacagcagcctcagcagccgcaagtgctatcctcggagggcgggcagctgcggcacaacccattggacatccagatgctctcgagagggctgcacgagcaaatcttcgggcaaggaggggagatgcctggcgaggccgcggtgcgccgcagcgtcgagcacctgcagaagcacgggctctgggggcagccagccgtgcccttgcccgacgtggagctgcgcctgccgcccctctacggggacaacctggaccagcacttccgcctcctggcccagaagcagagcctgccctacctggaggcggccaacttgctgttgcaggcccagctgcccccgaagcccccggcttgggcctgggcggagggctggacccggtacggccccgagggggaggccgtacccgtggccatccccgaggagcgggccctggtgttcgacgtggaggtctgcttggcagagggaacttgccccacattggcggtggccatatccccctcggcctggtattcctggtgcagccagcggctggtggaagagcgttactcttggaccagccagctgtcgccggctgacctcatccccctggaggtccctactggtgccagcagccccacccagagagactggcaggagcagttagtggtggggcacaatgtttcctttgaccgagctcatatcagggagcagtacctgatccagggttcccgcatgcgtttcctggacaccatgagcatgcacatggccatctcagggctaagcagcttccagcgcagtctgtggatagcagccaagcagggcaaacacaaggtccagccccccacaaagcaaggccagaagtcccagaggaaagccagaagaggcccagcgatctcatcctgggactggctggacatcagcagtgtcaacagtctggcagaggtgcacagactttatgtaggggggcctcccttagagaaggagcctcgagaactgtttgtgaagggcaccatgaaggacattcgtgagaacttccaggacctgatgcagtactgtgcccaggacgtgtgggccacccatgaggttttccagcagcagctaccgctcttcttggagaggtgtccccacccagtgactctggccggcatgctggagatgggtgtctcctacctgcctgtcaaccagaactgggagcgttacctggcagaggcacagggcacttatgaggagctccagcgggagatgaagaagtcgttgatggatctggccaatgatgcctgccagctgctctcaggagagaggtacaaagaagacccctggctctgggacctggagtgggacctgcaagaatttaagcagaagaaagctaagaaggtgaagaaggaaccagccacagccagcaagttgcccatcgagggggctggggcccctggtgatcccatggatcaggaagacctcggcccctgcagtgaggaggaggagtttcaacaagatgtcatggcccgcgcctgcttgcagaagctgaaggggaccacagagctcctgcccaagcggccccagcaccttcctggacaccctggatggtaccggaagctctgcccccggctagacgaccctgcatggaccccgggccccagcctcctcagcctgcagatgcgggtcacacctaaactcatggcacttacctgggatggcttccctctgcactactcagagcgtcatggctggggctacttggtgcctgggcggcgggacaacctggccaagctgccgacaggtaccaccctggagtcagctggggtggtctgcccctacagagccatcgagtccctgtacaggaagcactgtctcgaacaggggaagcagcagctgatgccccaggaggccggcctggcggaggagttcctgctcactgacaatagtgccatatggcaaacggtagaagaactggattacttagaagtggaggctgaggccaagatggagaacttgcgagctgcagtgccaggtcaacccctagctctgactgcccgtggtggccccaaggacacccagcccagctatcaccatggcaatggaccttacaacgacgtggacatccctggctgctggtttttcaagctgcctcacaaggatggtaatagctgtaatgtgggaagcccctttgccaaggacttcctgcccaagatggaggatggcaccctgcaggctggcccaggaggtgccagtgggccccgtgctctggaaatcaacaaaatgatttctttctggaggaacgcccataaacgtatcagctcccagatggtggtgtggctgcccaggtcagctctgccccgtgctgtgatcaggcaccccgactatgatgaggaaggcctctatggggccatcctgccccaagtggtgactgccggcaccatcactcgccgggctgtggagcccacatggctcaccgccagcaatgcccggcctgaccgagtaggcagtgagttgaaagccatggtgcaggccccacctggctacacccttgtgggtgctgatgtggactcccaagagctgtggattgcagctgtgcttggagacgcccactttgccggcatgcatggctgcacagcctttgggtggatgacactgcagggcaggaagagcaggggcactgatctacacagtaagacagccactactgtgggcatcagccgtgagcatgccaaaatcttcaactacggccgcatctatggtgctgggcagccctttgctgagcgcttactaatgcagtttaaccaccggctcacacagcaggaggcagctgagaaggcccagcagatgtacgctgccaccaagggcctccgctggtatcggctgtcggatgagggcgagtggctggtgagggagttgaacctcccagtggacaggactgagggtggctggatttccctgcaggatctgcgcaaggtccagagagaaactgcaaggaagtcacagtggaagaagtgggaggtggttgctgaacgggcatggaaggggggcacagagtcagaaatgttcaataagcttgagagcattgctacgtctgacataccacgtaccccggtgctgggctgctgcatcagccgagccctggagccctcggctgtccaggaagagtttatgaccagccgtgtgaattgggtggtacagagctctgctgttgactacttacacctcatgcttgtggccatgaagtggctgtttgaagagtttgccatagatgggcgcttctgcatcagcatccatgacgaggttcgctacctggtgcgggaggaggaccgctaccgcgctgccctggccttgcagatcaccaacctcttgaccaggtgcatgtttgcctacaagctgggtctgaatgacttgccccagtcagtcgcctttttcagtgcagtcgatattgaccggtgcctcaggaaggaagtgaccatggattgtaaaaccccttccaacccaactgggatggaaaggagatacgggattccccagggtgaagcgctggatatttaccagataattgaactcaccaaaggctccttggaaaaacgaagccagcctggaccatagcactgcctggaggctctgtatttgctcccgtggagcttcatcggggtggtgcaggctcccaaactcaggctttcagctgtgctttttgcaaaagggcttgctaaggccagccatttttcagtagcaggacctgccaagaagattccttctaactgaaggtgcagttgaattcagtgggttcagaaccaagatgccaacatcggtgtggactacaggacaaggggcattgttgcttgttgggtaaaaatgaagcagaagccccaaagttcacattaactcaggcatttcatttattttttccttttcttcttggctggttctttgttctgtcccccatgctctgatgcagtgccctagaaggggaaagaattaatgctctaacgtgataaacctgctccaaggcagtggaaataaaaagaaggaaaaaaaaaaaaaaaaaaa

SEQ ID NO: 140 - DNA, 2245 bp, Homo sapiens
ggagcaagaggtggttggggggggaccatggctgacgttttcccgggcaacgactccacggcgtctcaggacgtggccaaccgcttcgcccgcaaaggggcgctgaggcagaagaacgtgcacgaggtgaaggaccacaaattcatcgcgcgcttcttcaagcagcccaccttctgcagccactgcaccgacttcatctgggggtttgggaaacaaggcttccagtgccaagtttgctgttttgtggtccacaagaggtgccatgaatttgttactttttcttgtccgggtgcggataagggacccgacactgatgaccccaggagcaagcacaagttcaaaatccacacttacggaagccccaccttctgcgatcactgtgggtcactgctctatggacttatccatcaagggatgaaatgtgacacctgcgatatgaacgttcacaagcaatgcgtcatcaatgtccccagcctctgcggaatggatcacactgagaagagggggcggatttacctaaaggctgaggttgctgatgaaaagctccatgtcacagtacgagatgcaaaaaatctaatccctatggatccaaacgggctttcagatccttatgtgaagctgaaacttattcctgatcccaagaatgaaagcaagcaaaaaaccaaaaccatccgctccacactaaatccgcagtggaatgagtcctttacattcaaattgaaaccttcagacaaagaccgacgactgtctgtagaaatctgggactgggatcgaacaacaaggaatgacttcatgggatccctttcctttggagtttcggagctgatgaagatgccggccagtggatggtacaagttgcttaaccaagaagaaggtgagtactacaacgtacccattccggaaggggacgaggaaggaaacatggaactcaggcagaaattcgagaaagccaaacttggccctgctggcaacaaagtcatcagtccctctgaagacaggaaacaaccttccaacaaccttgaccgagtgaaactcacggacttcaatttcctcatggtgttgggaaaggggagttttggaaaggtgatgcttgccgacaggaagggcacagaagaactgtatgcaatcaaaatcctgaagaaggatgtggtgattcaggatgatgacgtggagtgcaccatggtagaaaagcgagtcttggccctgcttgacaaacccccgttcttgacgcagctgcactcctgcttccagacagtggatcggctgtacttcgtcatggaatatgtcaacggtggggacctcatgtaccacattcagcaagtaggaaaatttaaggaaccacaagcagtattctatgcggcagagatttccatcggattgttctttcttcataaaagaggaatcatttatagggatctgaagttagataacgtcatgttggattcagaaggacatatcaaaattgctgactttgggatgtgcaaggaacacatgatggatggagtcacgaccaggaccttctgtgggactccagattatatcgccccagagataatcgcttatcagccgtatggaaaatctgtggactggtgggcctatggcgtcctgttgtatgaaatgcttgccgggcagcctccatttgatggtgaagatgaagacgagctatttcagtctatcatggagcacaacgtttcctatccaaaatccttgtccaaggaggctgtttctatctgcaaaggactgatgaccaaacacccagccaagcggctgggctgtgggcctgagggggagagggacgtgagagagcatgccttcttccggaggatcgactgggaaaaactggagaacagggagatccagccaccattcaagcccaaagtgtgtggcaaaggagcagagaactttgacaagttcttcacacgaggacagcccgtcttaacaccacctgatcagctggttattgctaacatagaccagtctgattttgaagggttctcgtatgtcaacccccagtttgtgcaccccatcttacagagtgcagtatgaaactcaccagcgagaacaaacacctccccagcccccagccctccccgcagtggaagtgaatccttaaccctaaaattttaaggccacggcttgtgtctgattccatatggaggcctgaaaattgtagggttattagtccaaatgtgatcaactgttcagggtctctctcttacaaccaagaacattatcttagtggaag

SEQ ID NO: 141 - DNA, 1362 bp, Homo sapiens
catttggggacgctctcagctctcggcgcacggcccagcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtacttt

SEQ ID NO: 142 - DNA, 1137 bp, Homo sapiens
aagagcgagtcttggccttagcgcgggctttgcctccctgcttgccacgtccagacatagcgagcgcaactcactacgagcaaccacaaagtgaacgggaaaggcggcgctttttataaacactattgggcgcgaaaaagaagacgtgttgttggttagggctgcagtttaatttcaaccaatagtagtgcgtcttctggatttgcgaatcctgattgggcagacctgacctctgacgttaccctgaataactaccaatcagacacaagacttcaactcttcaccttatttgcataagcgattctatataaaagcgccttgtcataccctgctcacgctgtttttccttttcgttggcgctttatagctacacagtgctatgccagagccagcgaagtctgctcccgccccgaaaaagggctccaagaaggcggtgactaaggcgcagaagaaagacggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccgtcaccaagtacaccagcgctaagtaaacagtgagttggttgcaaactctcaaccctaacggctcttttaagagccacccatgttctcaaagaaagagctggtgcttgtattcctcctctgctggccactgacaaacccttgtaacttgctactgtgttttttggtctgaagtagagcagttatttaactaatccttagtgactttttttttttagatctgccattctaatcttagagttaagtaaggagatgggaaattttctattataagttcgaaaccaattaaaatacgttagaaaccaattaaaatactcgtcggtcccccgtcggttagtgatttggaacagtgccaagttgcagcggttgtcagtttgaatttgcccgggcaacgcccgcccttcct

SEQ ID NO: 143 - DNA, 1270 bp, Homo sapiens
agacgttcgcacacctgggtgccagcgccccagaggtcccgggacagcccgaggcgccgcgcccgccgccccgagctccccaagccttcgagagcggcgcacactcccggtctccactcgctcttccaacacccgctcgttttggcggcagctcgtgtcccagagaccgagttgccccagagaccgagacgccgccgctgcgaaggaccaatgagagccccgctgctaccgccggcgccggtggtgctgtcgctcttgatactcggctcaggccattatgctgctggattggacctcaatgacacctactctgggaagcgtgaaccattttctggggaccacagtgctgatggatttgaggttacctcaagaagtgagatgtcttcagggagtgagatttcccctgtgagtgaaatgccttctagtagtgaaccgtcctcgggagccgactatgactactcagaagagtatgataacgaaccacaaatacctggctatattgtcgatgattcagtcagagttgaacaggtagttaagcccccccaaaacaagacggaaagtgaaaatacttcagataaacccaaaagaaagaaaaagggaggcaaaaatggaaaaaatagaagaaacagaaagaagaaaaatccatgtaatgcagaatttcaaaatttctgcattcacggagaatgcaaatatatagagcacctggaagcagtaacatgcaaatgtcagcaagaatatttcggtgaacggtgtggggaaaagtccatgaaaactcacagcatgattgacagtagtttatcaaaaattgcattagcagccatagctgcctttatgtctgctgtgatcctcacagctgttgctgttattacagtccagcttagaagacaatacgtcaggaaatatgaaggagaagctgaggaacgaaagaaacttcgacaagagaatggaaatgtacatgctatagcataactgaagataaaattacaggatatcacattggagtcactgccaagtcatagccataaatgatgagtcggtcctctttccagtggatcataagacaatggaccctttttgttatgatggttttaaactttcaattgtcactttttatgctatttctgtatataaaggtgcacgaaggtaaaaagtattttttcaagttgtaaataatttatttaatatttaatggaagtgtatttattttacagctcattaaacttttttaaccaaacagaaaaaaaaaaaaaaaaaaaaaaaaaaaaaaaaa

SEQ ID NO: 144 - DNA, 3953 bp, Homo sapiens
ttatggttaatgttcttatagacatccaaaggtcagaaactattcccatttgaaaaatatctgttgtggtataaatgtgctgttttctttcctcttctccctgactttagggaactgctcgcagaaagaagaaggtggttcatagaacagccacagcagatgacaaaaaacttcagttctccttaaagaagttaggggtaaacaatatctctggtattgaagaggcaagtatcaaattttgttactttaaaaaacaagatttggctgggaaaagttaacgttaatgcattaaatgggttgttgggttttttttaacttagggacttcaaagtccctaagatgtgtttctaccataaattaataaatatcagggagctcattaagtctgaatgctattagaatacatattccattccaggcaaaatttcacctgtgcttacacgtgaaatactagttagccagagctagtttaataaaacatttgtttttaaagagactggtcagcattgctaatttaaatttttcttttcttaataggtgaatatgtttacaaaccaaggaacagtgatccactttaacaaccctaaagttcaggcatctctggcagcgaacactttcaccattacaggccatgctgagacaaagcagctgacagaaatgctacccagcatcttaaaccagcttggtgcggatagtctgactagtttaaggagactggccgaagctctgcccaaacaatgtgagtttcctagtaatggttttaccagggaattactcatttagcagctgatttctgatctcagggctcagaatggatatgagtatttttaagtttggaaatgcaagctttaaaaataacagatttgtaactgattttaagcaactgtccttgctcaagtttgcagtaattgatgtagcgtgccatgattgttacacttgattttgtggaatgttttctacttacttgatttggatcagatacttttattaactagaaatgatgaaaatgttaatttggtgctttgccaataactacttgtaagtttggaattgaaaaaaaaattagtgtaaattatgaaattacttcagtttcatctatatagttcgtattaccagtaatcttttaaaaatggcttgccagtattctggcattttaattacagtgtgatagggatttattcggggcagaaaatagtgtagctgaatatacatctgaggatgtggcagtgttatgctgttttctgtgcttaaaattttgaagaataggaatgcaggaggaagtcagaggcttatatatggctctttagttacccatgtttttctaggtattgacttaatctgcctcaattttcatttttattatcacattgagttgcaggttctaaactgtcagggctttcagagctgaaataggcttttgaagtatcccactgatgcctgtatgggcctagtacataactctcctgtgtacgttcatattcttgtgtgataaaggagagtggatgcttaccactcacagactctttaatttttttactttaacttttttcatttcagtaagtggttgttgagcatcacccttatgccacacacagagtagttgagaaaatggcatcttcatttgtctcccaaaatctcaccatgatttggtatgtgggttttacctgcactctaagagttccctactgcccttatactacctcaggcctatggtggccagaggattgaaagagtggtatggaatttgtttgttggcgttcctagtattttaacccatttgtagacattagaatatcatgttattgatagtatcataggataaaatcccaaatgtcccttatcatggaaataagttgtaacaacacttggcatttcatctgttctttttttttttttttttttttttttggtgaatatttattaaaaacctagacaaataatgtttacattttcctttcatagctgtggatggaaaagcaccacttgctactggagaggatgatgatgatgaagttccaggtaggaacgtttgcttgtggttaacctagagaatcttagccaagggagaataagaaatctttgtaggaaaaactacccagggaagaggggtggtaagttaagatggacatagatcttacttagaatgagaaaaataatgcagtattaggtaattgagaattatgtttatagacttgacttggcttgtttctgtttgggatcccaaggatgtgtaggtatctaaccttaaatattgaataaataagtatatatatatagtaccctaaatataactattacctgcagagcactaatgacccttgctccctactttgaaactcatgaatttacaagaaggtgtggagttgttcaggtatcttgggatatatatatgcattctaaaatctgtagcagcataactccttttgggaatcagaggattttgtctcttacctgttattggataaatttacgttcttctaaaatatttattgggcaggagaatcactggactcataaatattcccactttgcatagacaggtatccttaggaatcaggaaaattttaacattgtgtgtcattgtattctttggttctgctcccccactattgaccaatgtagagatgggaagaggggggcatttttttctcttttttttttttttgcattcttgttcttggggctatgacacagtatttatcatcattggcaaatgaatgctctttccttcatccccttttaatatctgataattatttgtagattggcttttttaagaatttctactctttttcttttcctagatcttgtggagaattttgatgaggcttccaagaatgaggcaaactgaattgagtcaacttctgaagataaaacctgaagaagtttactgggagctgctattttatattatgactgctttttaagaaaatttttgtttatggatctgataaaatctagatctctaatatttttaagcccaagccccttggacactgcagctcttttcagtttttgcttatacacaattcattctttgcagctaattaagccgaagaagcctgggaatcaagtttgaaacaaagattaataaagttcttttgcctagtatacagttttatttttttatttattgacaccgatctgtacacagtaaaaaaaattgcttatagaaagctaatcatggcatgtaatatggctgataacctttggaatttgattaaagatttaaaatcacggtgtaagtgtaacaaaggtggtataaagttctcaggtttgaaaactttgtctccaacagtccttagtgcttccatgatttatatggtgggtgtaaatatgagaatagagtattccttagtggataaacagacatttctccctgatattctctattgtaagcatatgttaagtgccttttatgaattaccctcggtgttatcttcctttattcctcaatttgtgaagaactaatagctccattttgtagatgtaacctgaggtttagaacttctaaaaagtaaaagtaatctccagatcccttctttgtaggatattttataaggtgacttggaaaaggtagtgtttagaataggagtggctcctgggtcattgtcttttccttaagtgtaacacctaataaatgaatagggttatgtttttatttaataaaaaatatacagtaaaattgagcatatacagttaaaagaatttataatgtctgccactataaccaggcttaccagacagtttcatggtccagaaaatccctaaacatagggttacttttaaacattttacaaattacaatgaaacaattgtgtaatctgaaccaaggccatttgaggagaaatagttctacttgtatggtatttatttttaaatttttcatagcaatttgcaagtaccttttgaaagtattatcagttgtatctaaaatgcactattaaccgtgg

SEQ ID NO: 145 - DNA, 3213 bp, Homo sapiens
atgtgcttcagtttcataatgcctcctgctatggcagacatccttgacatctgggcggtggattcacagatagcatctgatggctccatacctgtggatttccttttgcccactgggatttatatccagttggaggtacctcgggaagctaccatttcttatattaagcagatgttatggaagcaagttcacaattacccaatgttcaacctccttatggatattgactcctatatgtttgcatgtgtgaatcagactgctgtatatgaggagcttgaagatgaaacacgaagactctgtgatgtcagaccttttcttccagttctcaaattagtgacaagaagttgtgacccaggggaaaaattagactcaaaaattggagtccttataggaaaaggtctgcatgaatttgattccttgaaggatcctgaagtaaatgaatttcgaagaaaaatgcgcaaattcagcgaggaaaaaatcctgtcacttgtgggattgtcttggatggactggctaaaacaaacatatccaccagagcatgaaccatccatccctgaaaacttagaagataaactttatgggggaaagctcatcgtagctgttcattttgaaaactgccaggacgtgtttagctttcaagtgtctcctaatatgaatcctatcaaagtaaatgaattggcaatccaaaaacgtttgactattcatgggaaggaagatgaagttagcccctatgattatgtgttgcaagtcagcgggagagtagaatatgtttttggtgatcatccactaattcagttccagtatatccggaactgtgtgatgaacagagccctgccccattttatacttgtggaatgctgcaagatcaagaaaatgtatgaacaagaaatgattgccatagaggctgccataaatcgaaattcatctaatcttcctcttccattaccaccaaagaaaacacgaattatttctcatgtttgggaaaataacaaccctttccaaattgtcttggttaagggaaataaacttaacacagaggaaactgtaaaagttcatgtcagggctggtctttttcatggtactgagctcctgtgtaaaaccatcgtaagctcagaggtatcagggaaaaatgatcatatttggaatgaaccactggaatttgatattaatatttgtgacttaccaagaatggctcgattatgttttgctgtttatgcagttttggataaagtaaaaacgaagaaatcaacgaaaactattaatccctctaaatatcagaccatcaggaaagctggaaaagtgcattatcctgtagcgtgggtaaatacgatggtttttgactttaaaggacaattgagaactggagacataatattacacagctggtcttcatttcctgatgaactcgaagaaatgttgaatccaatgggaactgttcaaacaaatccatatactgaaaatgcaacagctttgcatgttaaatttccagagaataaaaaacaaccttattattaccctcccttcgataagattattgaaaaggcagctgagattgcaagcagtgatagtgctaatgtgtcaagtcgaggtggaaaaaagtttcttcctgtattgaaagaaatcttggacagggatcccttgtctcaactgtgtgaaaatgaaatggatcttatttggactttgcgacaagactgccgagagattttcccacaatcactgccaaaattactgctgtcaatcaagtggaataaacttgaggatgttgctcagcttcaggcgctgcttcagatttggcctaaactgcccccccgggaggccctagagcttctggatttcaactatccagaccagtacgttcgagaatatgctgtaggctgcctgcgacagatgagtgatgaagaactttctcaatatcttttacaactggtgcaagtgttaaaatatgagccttttcttgattgtgccctctctagattcctattagaaagagcacttggtaatcggaggatagggcagtttctattttggcatcttaggtcagaagtgcacattcctgctgtctcagtacaatttggtgtcatccttgaagcatactgccggggaagtgtggggcacatgaaagtgctttctaagcaggttgaagcactcaataagttaaaaactttaaatagtttaatcaaactgaatgccgtgaagttaaacagagccaaagggaaggaggccatgcatacctgtttaaaacagagtgcttaccgggaagccctctctgacctgcagtcacccctgaacccatgtgttatcctctcagaactctatgttgaaaagtgcaaatacatggattccaaaatgaagcctttgtggctggtatacaataacaaggtatttggtgaggattcagttggagtgatttttaaaaatggtgatgatttacgacaggatatgttgacactccaaatgttgcgcttgatggatttactctggaaagaagctggtttggatcttcggatgttgccttatggctgtttagcaacaggagatcgctctggcctcattgaagttgtgagcacctctgaaacaattgctgacattcagctgaacagtagcaatgtggctgctgcagcagccttcaacaaagatgcccttctgaactggcttaaagaatacaactctggggatgacctggaccgagccattgaggaatttacactgtcctgtgctggctactgtgtagcttcttatgtccttgggattggtgacagacatagtgacaacatcatggtcaaaaaaactggccagctcttccacattgactttggacatattcttggaaatttcaaatctaagtttggcattaaaagggagcgagtgccttttattcttacctatgatttcatccatgtcattcaacaaggaaaaacaggaaatacagaaaagtttggccggttccgccagtgttgtgaggatgcatatctgattttacgacggcatgggaatctcttcatcactctctttgcgctgatgttgactgcagggcttcctgaactcacatcagtcaaagatatacagtatcttaaggactctcttgcattagggaagagtgaagaagaagcactcaaacagtttaagcaaaaatttgatgaggcgctcagggaaagctggactactaaagtgaactggatggcccacacagttcggaaagactacagatcttaa

SEQ ID NO: 146 - DNA, 2602 bp, Homo sapiens
gccgtgtcgccaccatggctccgcaccgccccgcgcccgcgctgctttgcgcgctgtccctggcgctgtgcgcgctgtcgctgcccgtccgcgcggccactgcgtcgcggggggcgtcccaggcgggggcgccccaggggcgggtgcccgaggcgcggcccaacagcatggtggtggaacaccccgagttcctcaaggcagggaaggagcctggcctgcagatctggcgtgtggagaagttcgatctggtgcccgtgcccaccaacctttatggagacttcttcacgggcgacgcctacgtcatcctgaagacagtgcagctgaggaacggaaatctgcagtatgacctccactactggctgggcaatgagtgcagccaggatgagagcggggcggccgccatctttaccgtgcagctggatgactacctgaacggccgggccgtgcagcaccgtgaggtccagggcttcgagtcggccaccttcctaggctacttcaagtctggcctgaagtacaagaaaggaggtgtggcatcaggattcaagcacgtggtacccaacgaggtggtggtgcagagactcttccaggtcaaagggcggcgtgtggtccgtgccaccgaggtacctgtgtcctgggagagcttcaacaatggcgactgcttcatcctggacctgggcaacaacatccaccagtggtgtggttccaacagcaatcggtatgaaagactgaaggccacacaggtgtccaagggcatccgggacaacgagcggagtggccgggcccgagtgcacgtgtctgaggagggcactgagcccgaggcgatgctccaggtgctgggccccaagccggctctgcctgcaggtaccgaggacaccgccaaggaggatgcggccaaccgcaagctggccaagctctacaaggtctccaatggtgcagggaccatgtccgtctccctcgtggctgatgagaaccccttcgcccagggggccctgaagtcagaggactgcttcatcctggaccacggcaaagatgggaaaatctttgtctggaaaggcaagcaggcaaacacggaggagaggaaggctgccctcaaaacagcctctgacttcatcaccaagatggactaccccaagcagactcaggtctcggtccttcctgagggcggtgagaccccactgttcaagcagttcttcaagaactggcgggacccagaccagacagatggcctgggcttgtcctacctttccagccatatcgccaacgtggagcgggtgcccttcgacgccgccaccctgcacacctccactgccatggccgcccagcacggcatggatgacgatggcacaggccagaaacagatctggagaatcgaaggttccaacaaggtgcccgtggaccctgccacatatggacagttctatggaggcgacagctacatcattctgtacaactaccgccatggtggccgccaggggcagataatctataactggcagggtgcccagtctacccaggatgaggtcgctgcatctgccatcctgactgctcagctggatgaggagctgggaggtacccctgtccagagccgtgtggtccaaggcaaggagcccgcccacctcatgagcctgtttggtgggaagcccatgatcatctacaagggcggcacctcccgcgagggcgggcagacagcccctgccagcacccgcctcttccaggtccgcgccaacagcgctggagccacccgggctgttgaggtattgcctaaggctggtgcactgaactccaacgatgcctttgttctgaaaaccccctcagccgcctacctgtgggtgggtacaggagccagcgaggcagagaagacgggggcccaggagctgctcagggtgctgcgggcccaacctgtgcaggtggcagaaggcagcgagccagatggcttctgggaggccctgggcgggaaggctgcctaccgcacatccccacggctgaaggacaagaagatggatgcccatcctcctcgcctctttgcctgctccaacaagattggacgttttgtgatcgaagaggttcctggtgagctcatgcaggaagacctggcaacggatgacgtcatgcttctggacacctgggaccaggtctttgtctgggttggaaaggattctcaagaagaagaaaagacagaagccttgacttctgctaagcggtacatcgagacggacccagccaatcgggatcggcggacgcccatcaccgtggtgaagcaaggctttgagcctccctcctttgtgggctggttccttggctgggatgatgattactggtctgtggaccccttggacagggccatggctgagctggctgcctgaggaggggcagggcccacccatgtcaccggtcagtgccttttggaactgtccttccctcaaagaggccttagagcgagcagagcagctctgctatgagtgtgtgtgtgtgtgtgtgttgtttcttttttttttttttacagtatccaaaaatagccctgcaaaaattcagagtccttgcaaaattgtctaaaatgtcagtgtttgggaaattaaatccaataaaaacattttgaagtgtg

SEQ ID NO: 147 - DNA, 6480 bp, Homo sapiens
agagcgagcaggggagagcgagaccagttttaaggggaggaccggtgcgagtgaggcagccccgaggctctgctcgcccaccacccaatcctcgcctcccttctgctccaccttctctctctgccctcacctctcccccgaaaaccccctatttagccaaaggaaggaggtcaggggaacgctctcccctccccttccaaaaaacaaaaacagaaaaacccttttccaggccggggaaagcaggagggagaggggccgccgggctggccatggagctgctgtgccacgaggtggacccggtccgcagggccgtgcgggaccgcaacctgctccgagacgaccgcgtcctgcagaacctgctcaccatcgaggagcgctaccttccgcagtgctcctacttcaagtgcgtgcagaaggacatccaaccctacatgcgcagaatggtggccacctggatgctggaggtctgtgaggaacagaagtgcgaagaagaggtcttccctctggccatgaattacctggaccgtttcttggctggggtcccgactccgaagtcccatctgcaactcctgggtgctgtctgcatgttcctggcctccaaactcaaagagaccagcccgctgaccgcggagaagctgtgcatttacaccgacaactccatcaagcctcaggagctgctggagtgggaactggtggtgctggggaagttgaagtggaacctggcagctgtcactcctcatgacttcattgagcacatcttgcgcaagctgccccagcagcgggagaagctgtctctgatccgcaagcatgctcagaccttcattgctctgtgtgccaccgactttaagtttgccatgtacccaccgtcgatgatcgcaactggaagtgtgggagcagccatctgtgggctccagcaggatgaggaagtgagctcgctcacttgtgatgccctgactgagctgctggctaagatcaccaacacagacgtggattgtctcaaagcttgccaggagcagattgaggcggtgctcctcaatagcctgcagcagtaccgtcaggaccaacgtgacggatccaagtcggaggatgaactggaccaagccagcacccctacagacgtgcgggatatcgacctgtgaggatgccagttgggccgaaagagagagacgcgtccataatctggtctcttcttctttctggttgtttttgttctttgtgttttagggtgaaacttaaaaaaaaaattctgcccccacctagatcatatttaaagatcttttagaagtgagagaaaaaggtcctacgaaaacggaataataaaaagcatttggtgcctatttgaagtacagcataagggaatcccttgtatatgcgaacagttattgtttgattatgtaaaagtaatagtaaaatgcttacaggaaaacctgcagagtagttagagaatatgtatgcctgcaatatgggaacaaattagaggagacttttttttttcatgttatgagctagcacatacacccccttgtagtataatttcaaggaactgtgtacgccatttatggcatgattagattgcaaagcaatgaactcaagaaggaattgaaataaggagggacatgatggggaaggagtacaaaacaatctctcaacatgattgaaccatttgggatggagaagcacctttgctctcagccacctgttactaagtcaggagtgtagttggatctctacattaatgtcctcttgctgtctacagtagctgctacctaaaaaaagatgttttattttgccagttggacacaggtgattggctcctgggtttcatgttctgtgacatcctgcttcttcttccaaatgcagttcattgcagacaccaccatattgctatctaatggggaaatgtagctatgggccataaccaaaactcacatgaaacggaggcagatggagaccaagggtgggatccagaatggagtcttttctgttattgtatttaaaagggtaatgtggccttggcatttcttcttagaaaaaaactaatttttggtgctgattggcatgtctggttcacagtttagcattgttataaaccattccattcgaaaagcactttgaaaaattgttcccgagcgatagatgggatggtttatgcaagtcatgctgaatactcctcccctcttctcttttgccccctcccttcctgcccccagtctgggttactcttcgcttctggtatctggcgttctttggtacacagttctggtgttcctaccaggactcaagagacaccccttcctgctgacattcccatcacaacattcctcagacaagcctgtaaactaaaatctgttaccattctgatggcacagaaggatcttaattcccatctctatacttctcctttggacatggaaagaaaagttattgctggtgcaaagatagatggctgaacatcagggtgtggcattttgttcccttttccgttttttttttttttattgttgttgttaattttattgcaaagttgtattcagcgtacttgaatttttcttcctctccacttcttagaggcattcagttagcaaagaggttggagcaacaacttttttttttttttttgcacaattgtaattgacaggtaatgaagctatttgttaaaatatttgcctttttaagtaaaaaagaaaaatcagaacagggctatttgaagaattattttatacacagattctgccttgtttcatagtatgagggttgaagacggaaaacaatctaagggtctctcatttttttaattttgttttgttcagtttggtttttttttttttttgcgctgctaagaagctaaagtcatccatccttattcacgttgacagtacctagctgtaatgtttcacagagtgtgctgctattttataaacatttttataatatattattttactgcttaaattccaagtcctgaagtagatggttgagatatgagttcttcgtactggaaaagcccttccgtagtttgttttcttctggtagcatattcatggttgtttttttttttcttttttggttttttggttttttttttttcctctgatcacattcttcaaagacggagtattctttacctcaggtttactggacaaaatcaataactacaaaaggcaatgattcacgcttttgttttcataatacctcacaaccgtacagtttctgcttgggagcccattcgcatgaggaatacagaagcagtgtgagcagggctgactccctctcaggtggaaggcagggcggtctcactcccagggacctttttggtcatggaggccatcgggctcccagttagaccctggtatcctcatcatgatggaaaaaatacattgaaccaagggatcctccctccccttcaaggcagacgttcagtacaaacatttatgcggtaggctcagatgtcgtaatttgcacttaggtaccaggtgtcaggaaacagactaaaaagaattccaccaggctgtttggagatcctcatcttggagctttttcaaaagcggggcttcatctgcaaagggccctttcatcttgaagtttttcccctccgtctttcccctcccctggcatggacaccttgtgtttaggatcatctctgcaggtttcctaggtctgaatctgcgagtagatgaacctgcagcaagcagcgtttatggtgcttccttctccctcctctgtctcaaactgcgcaggcaagcactatgcaagcccaggccctctgctgagcggtactaaacggtcgggttttcaatcacactgaattggcaggataagaaaaataggtcagataagtatgggatgatagttgaagggaggtgaagaggctgcttctctacagaggtgaaattccagatgagtcagtctcttgggaagtgtgtttagaagggttcaggactttgtgagttagcatgaccctaaaattctaggggatttctggtgggacaatgggtggtgaattttgaagttttggagagggaagtggagcagccagcaagtaagctagccagagttttctcaagagccagctttgctcagcacactctcctgggccccaaggagtcccacggaatggggaaagtgggaaccctggagttcttgggaatcttggagcctaaagagaaaccgaggtgcaaattcatttcatggtgactgacccttgagcttaaacagaagcagcaaatgaaagaaccggacaaataaggaagggcacaagcctacccgactctatttacagtctgtaactttccactcttcctgtagtcccgaggcccctgggtccttctagcttttctctttcccatccttggggccttgtgtgatgatgggtgtggggctgccgatgggaaagtcgggggttgttaggcttttctgcctgctcctgcttaaacacaagaaggaatcctggattttgccctctccttagctcttagtctctttggtaggagttttgttccagaggagctctcccccttggatttgaacttgctctttttgttgttgttgttctttctcttctttttcttacctcccactaaaggggttccaaattatcctggtctttttctaccttgttgtgtttctatctcgtctttacttccatctgtttgtttttttctccatcagtgggggccgagttgttcccccagcctgccaaattttgatccttcccctcttttggccaaatcctagggggaagaaatcctagtatgccaaaaatatatgctaagcataattaaactccatgcgggtccataacagccaagaagcctgcaggagaaagccaagggcagttccctccgcagaacaccccatgcgtgctgagaggcgagctccttgaagaaggggctgttcttccaggaggccttattttgaactgcctcaggaccccactggagagcacagcatgccttactactgggtcatccttggtctatgtgctctgtactggaggctctgttctgcctcttatcagccaggtcaggggcacacatggcttaagtgacaaagccagaggagaagacaaccctgacagcatcacgctgcatcccattgctagcaggattggcaactcttcagacggagctgcgcttccctgcagtctagcacctctagggcctctccagactgtgccctgggagctctgggactgaaaggttaagaacataaggcaggatcagatgactctctccaagagggcaggggaattttctctccatgggccacaggggacagggctgggagaagaaatagacttgcaccttatgtcatgtaaataattgattttctagttcaagaagataatattggtagtgtgggaattggaggtaggaaggggaggaagtctgagtaagccagttggcttctaagccaaaaggattcctctttgtttatctctgagacagtccaaccttgagaatagctttaaaagggaaattaatgctgagatgataaagtccccttaagccaacaaaccctctgtagctatagaatgagtgcaggtttctattggtgtggactcagagcaatttacaagagctgttcatgcagccatccatttgtgcaaaatagggtaagaagattcaagaggatatttattacttcctcataccacatggcttttgatgattctggattctaaacaacccagaatggtcatttcaggcacaacgatactacattcgtgtgtgtctgcttttaaacttggctgggctatcagaccctattctcggctcaggttttgagaagccatcagcaaatgtgtacgtgcatgctgtagctgcagcctgcatcccttcgcctgcagcctactttggggaaataaagtgccttactgactgtagccattacagtatccaatgtcttttgacaggtgcctgtccttgaaaaacaaagtttctatttttatttttaattggtttagttcttaactgctggccaactcttacatccccagcaaatcatcgggccattggattttttccattatgttcatcacccttatatcatgtacctcagatctctctctctctcctctctctcagttatatagtttcttgtcttggactttttttttcttttctttttctttttttttttgctttaaaacaagtgtgatgccatatcaagtccatgttattctctcacagtgtactctataagaggtgtgggtgtctgtttggtcaggatgttagaaagtgctgataagtagcatgatcagtgtatgcgaaaaggtttttaggaagtatggcaaaaatgttgtattggctatgatggtgacatgatatagtcagctgccttttaagaggtcttatctgttcagtgttaagtgatttaaaaaaataataacctgttttctgactagtttaaagatggatttgaaaatggttttgaatgcaattaggttatgctatttggacaataaactcaccttgacct

SEQ ID NO: 148 - DNA, 3945 bp, Homo sapiens
cgtccacccgcccagggagagtcagacctgggggggcgagggccccccaaactcagttcggatcctacccgagtgaggcggcgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgaagtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtccggacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggcgtggctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctgattgcgggcacggcagtcgtgggtgtggtcctggtcctggtggtcattgtggtcgcagttctctgcctcaggaagcagagcaatgggagagaagcagaatattcggacaaacacggacagtatctcatcggacatggtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcggctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctgggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcggggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgaggacctgctccgaatcggagtcactctggcgggacaccagaagaaaatcttggccagtgtccagcacatgaagtcccaggccaagccgggaaccccgggtgggacaggaggaccggccccgcagtactgacctgcaggaactccccaccccagggacaccgcctccccattttccggggcagagtggggactcacagaggcccccagccctgtgccccgctggattgcactttgagcccgtggggtgaggagttggcaatttggagagacaggatttgggggttctgccataataggaggggaaaatcaccccccagccacctcggggaactccagaccaagggtgagggcgcctttccctcaggactgggtgtgaccagaggaaaaggaagtgcccaacatctcccagcctccccaggtgcccccctcaccttgatgggtgcgttcccgcagaccaaagagagtgtgactcccttgccagctccagagtgggggggctgtcccagggggcaagaaggggtgtcagggcccagtgacaaaatcattggggtttgtagtcccaacttgctgctgtcaccaccaaactcaatcatttttttcccttgtaaatgcccctcccccagctgctgccttcatattgaaggtttttgagttttgtttttggtcttaatttttctccccgttccctttttgtttcttcgttttgtttttctaccgtccttgtcataactttgtgttggagggaacctgtttcactatggcctcctttgcccaagttgaaacaggggcccatcatcatgtctgtttccagaacagtgccttggtcatcccacatccccggaccccgcctgggacccccaagctgtgtcctatgaaggggtgtggggtgaggtagtgaaaagggcggtagttggtggtggaacccagaaacggacgccggtgcttggaggggttcttaaattatatttaaaaaagtaactttttgtataaataaaagaaaatgggacgtgtcccagctccaggggt

SEQ ID NO: 149 - DNA, 834 bp, Homo sapiens
atgaggaactcctatagatttctggcatcctctctctcagttgtcgtttctctcctgctaattcctgaagatgtctgtgaaaaaattattggaggaaatgaagtaactcctcattcaagaccctacatggtcctacttagtcttgacagaaaaaccatctgtgctggggctttgattgcaaaagactgggtgttgactgcagctcactgtaacttgaacaaaaggtcccaggtcattcttggggctcactcaataaccagggaagagccaacaaaacagataatgcttgttaagaaagagtttccctatccatgctatgacccagccacacgcgaaggtgaccttaaacttttacagctgacggaaaaagcaaaaattaacaaatatgtgactatccttcatctacctaaaaagggggatgatgtgaaaccaggaaccatgtgccaagttgcagggtgggggaggactcacaatagtgcatcttggtccgatactctgagagaagtcaatatcaccatcatagacagaaaagtctgcaatgatcgaaatcactataattttaaccctgtgattggaatgaatatggtttgtgctggaagcctccgaggtggaagagactcgtgcaatggagattctggaagccctttgttgtgcgagggtgttttccgaggggtcacttcctttggccttgaaaataaatgcggagaccctcgtgggcctggtgtctatattcttctctcaaagaaacacctcaactggataattatgactatcaagggagcagtttaaataaccgtttcctttcatttactgtggcttcttaatcttttcaca

SEQ ID NO: 150 - DNA, 4862 bp, Homo sapiens
acgcagctccgccccgcgtccgacccgcggatcccgcggcgtccggcccgggtggtctggatcgcggagggaatgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattgaagatttctgttgaaacttacactgtttcatctcagcttttgatggtactgatgagtcttgatctagatacaggactggttccttccttagtttcaaagtgtctcattctcagagtaaaataggcaccattgcttaaaagaaagttaactgacttcactaggcattgtgatgtttaggggcaaacatcacaaaatgtaatttaatgcctgcccattagagaagtatttatcaggagaaggtggtggcatttttgcttcctagtaagtcaggacagcttgtatgtaaggaggtttatataagtaattcagtgggaattgcagcatatcgtttaattttaagaaggcattggcatctgcttttaatggatgtataatacatccattctacatccgtagcggttggtgacttgtctgcctcctgctttgggaagactgaggcatccgtgaggcagggacaagtctttctcctctttgagaccccagtgcctgcacatcatgagccttcagtcagggtttgtcagaggaacaaaccaggggacactttgttagaaagtgcttagaggttctgcctctatttttgttggggggtgggagaggggaccttaaaatgtgtacagtgaacaaatgtcttaaagggaatcatttttgtaggaagcattttttataattttctaagtcgtgcactttctcggtccactcttgttgaagtgctgttttattactgtttctaaactaggattgacattctacagttgtgataatagcatttttgtaacttgccatccgcacagaaaatacgagaaaatctgcatgtttgattatagtattaatggacaaataagtttttgctaaatgtgagtatttctgttcctttttgtaaatatgtgacattcctgattgatttgggtttttttgttgttgttgttttgttttgttttgtttttttgggatggagkctcactcttgtcacccaggctggagtgcagtggcgccatctcggctcactgcaacctctgcctcctgagttcacgtaatcctcctgagtagctgggattacaggtgcctgccaccacgctggccaatttttgtacttttagtagagacagtgtttcgccatgttggccaggctggtttcaaactcctgacctcaggtgatccgcccacctcagcctcccaaaatggtgggattacaggtgtgtgggccaccgtgcctggctgattcagcattttttatcaggcaggaccaggtggacttccacctccagcctctggtcctaccaatggattcatggagtagcctggactgtttcatagttttctaaatgtacaaattcttataggctagacttagattcattaactcaaattcaatgcttctatcagactcagttttttgtaactaatagatttttttttccacttttgttctactccttccctaatagctttttaaaaaaatctccccagtagagaaacatttggaaaagacagaaaactaaaaaggaagaaaaaagatccctattagatacacttcttaaatacaatcacattaacattttgagctatttccttccagcctttttagggcagattttggttggtttttacatagttgagattgtactgttcatacagttttataccctttttcatttaactttataacttaaatattgctctatgttagtataagcttttcacaaacattagtatagtctcccttttataattaatgtttgtgggtatttcttggcatgcatctttaattccttatcctagcctttgggcacaattcctgtgctcaaaaatgagagtgacggctggcatggtggctcccgcctgtaatcccagtactttgggaagccaaggtaagaggattgcttgagcccagaacttcaagatgagcctgggctcatagtgagaacccgtctatacaaaaaatttttaaaaattagcatggcggcacacatctgtaatcctagctacttggcaggctgaggtgagaagatcattggagtttaggaattggaggcggcagtgagtcatgagtatgccgctgcactccagcctgggggacagagcaagaccctgcctcaaaaaaaaaaaaaaaaaaaattcaggccgggaatggtggttcacgcctgtaatcccagcactttggggggtcgaggtgggcagatcacctgaggtcaggagttcgagaccagcctggccaacatggtaaaaccccatttctactaaaaaatacaagaattagctgggtgtggtggcgcatgcctgtaatcctagctactcaggaggctgaggcaggagaatcacttgaccccaggaggcgaagattgcagtgagctgatatcgcaccattgtactccagcctgtgtgacagagcaatactcttgtcccaaaaaaaaaaaaaattcaaatcagagtgaagtgaatgagacactccagttttccttctactccgaattttagctcctcctttcaacattcaacaaatagtcttttttttttttttttttttttggggatggagtctccctctgttgcccaggctggagtgcagaggtgcgatctctgctcactacaagctctgcctcccgagttcaagtgattctcctggctcaccctcctgagctgggattacaggcgcctgccaccatgcctggctaattttgtgtttttagtggagacggggtttcaccatgttgtccaggatggtcttgatctcctgaccttgtgatccacccacctcagcctcccaaagtggtgggattacaggtgtgagccaccgcgtccagccagctttattattttttttaagctgtctttgtgtcaaaatgatagttcatgctcctcttgttaaaacctgcaggccgagcacagtggctcatgcctgtaatcccagcattttgggagaccaaggcggatggatcacctgaggtcaggagctcaagaccagcctggctaacatggtgaaacctcatctccacttaaaatacaaaaattgccggccgcggcggctcatgcctgtaatcccagcactttgggaggcctaggcgggtggatcacgacgtcaggaaatcgagaccatcctggctaacacgggtgaaaccccgtctctattaaaaaatagaaaaaattaggcgggcgtggtggtgagcgcctgtagtcccagctactcgagagcctgaggcaggagaatggcatgaacctggaaggtggagcttgcagtgagctgagatggtgccactgcactctaacctgggcgacagagtgagactccgtctcaaaaaaaaaaacaaaaaccaaaacttatccaggtgtggcggtgggcgcctgtgaggcaggcgaatctcttgaacccgggaggcggaggttgcagtgagccaagatcacaccattgcactccagcctgggaaacaagagtgaaattccatctcaaaaccaaattttcaaaaaaaaaacatgccgcttgagtactgtgtttttggtgttgtccaaggaaaattaaaacctgtagcatgaataatgtttgttttcatttcgaatcttgtgaatgtattaaatatatcgctcttaagagacggtgaagttcctatttcaagttttttttgttttgttttgtttttaagctgttttttaatacattaaatggtgctgagtaaaggaaataggcagggtgtgttgtgtggtgttttaactaggcgcttctctctcagagagttttgaaacctgtttacataaaggcccaagatgggaaggagatccaaacataagccaccagcctcattccaagtctcttctctttccaaccctggattttttttttttatttaacattgtttcttttagctttatttttcttataaaagaaatgtatcactataaaaaattacacactacagaaaaatattaagaagaaaaacattcacatcggaaacaaagttttttcccatgaaaacagaacccaaaagggtaagtggttagtatttcaccagcaattatgttgagaataaggccaggcgaggtggctcacgcctgtaatctcagcactttgggaggccagggcaggcagatcatctgaggtcaggagtttgagaccagcctggccaacatggtgaaaccctatctctactaaaaattaaa

SEQ ID NO: 151 - DNA, 3661 bp, Homo sapiens
cgcggagagatgccgcgggggccgctcgcagccgccgctgacttgtgaatgggaccgggactggggccgggactgacaccgcagcgcttgccctgcgccagggactggcggctcggaggttgcgtccaccctcaagggccccagaaatcactgtgttttcagctcagcggccctgtgacattccttcgtgttgtcatttgttgagtgaccaatcagatgggtggagtgtgttacagaaattggcagcaagtatccaatgggtgaagaagaagctaactggggacgtgggcagccctgacgtgatgagctcaaccagcagagacattccatcccaagagaggtctgcgtgacgcgtccgggaggccaccctcagcaagaccaccgtacagttggtggaaggggtgacagctgcattctcctgtgcctaccacgtaaccaaaaatgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagagcgtataccagatatctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccaaaagagcctatagcttgaaggtagaggcagccaacgtgcacatcgacccgaagtttatcagcaatggccctttcaaggacactgtgaccgtcaagatcgcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagccaaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctggtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaacaataacgatacaaatttggccttaagaactgtgtctggcgttctcaagaatctagaagatgtgtaaacaggtatttttttaaatcaaggaaaggctcatttaaaacaggcaaagttttacagagaggatacatttaataaaactgcgaggacatcaaagtggtaaatactgtgaaataccttttctcacaaaaaggcaaatattgaagttgtttatcaacttcgctagaaaaaaaaaacacttggcatacaaaatatttaagtgaaggagaagtctaacgctgaactgacaatgaagggaaattgtttatgtgttatgaacatccaagtctttcttcttttttaagttgtcaaagaagcttccacaaaattagaaaggacaacagttctgagctgtaatttcgccttaaactctggacactctatatgtagtgcatttttaaacttgaaatatataatattcagccagcttaaacccatacaatgtatgtacaatacaatgtacaattatgtctcttgagcatcaatcttgttactgctgattcttgtaaatctttttgcttctactttcatcttaaactaatacgtgccagatataactgtcttgtttcagtgagagacgccctatttctatgtcatttttaatgtatctatttgtacaattttaaagttcttattttagtatacatataaatatcagtattctgacatgtaagaaaatgttacggcatcacacttatattttatgaacattgtactgttgctttaatatgagcttcaatataagaagcaatctttgaaataaaaaaagatttttttttaaaaaaaa

SEQ ID NO: 152 - DNA, 3867 bp, Homo sapiens
acaggcccgcgacgctcccctcagctggcggcggccgcggagagatgccgcgggggccgctcgcagccgccgctgacttgtgaatgggaccgggactggggccgggactgacaccgcagcgcttgccctgcgccagggactggcggctcggaggttgcgtccaccctcaagggccccagaaatcactgtgttttcagctcagcggccctgtgacattccttcgtgttgtcatttgttgagtgaccaatcagatgggtggagtgtgttacagaaattggcagcaagtatccaatgggtgaagaagaagctaactggggacgtgggcagccctgacgtgatgagctcaaccagcagagacattccatcccaagagaggtctgcgtgacgcgtccgggaggccaccctcagcaagaccaccgtacagttggtggaaggggtgacagctgcattctcctgtgcctaccacgtaaccaaaaatgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagagcgtataccagatatctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccaaaagagcctatagcttgaaggtagaggcagccaacgtgcacatcgacccgaagtttatcagcaatggccctttcaaggacactgtgaccgtcaagatcgcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagccaaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctgggttgcccaagcttaatggaacccccctctcccagggaagacatgagattgctttatctgggcttccagctgatgctattttcctatgttaaagtaaacagaagattttgtcttctgggggtctttataaaacttcctttcctctatgtggtggctacagagagtccaaccacacttacgtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaacaataacgatacaaatttggccttaagaactgtgtctggcgttctcaagaatctagaagatgtgtaaacaggtatttttttaaatcaaggaaaggctcatttaaaacaggcaaagttttacagagaggatacatttaataaaactgcgaggacatcaaagtggtaaatactgtgaaataccttttctcacaaaaaggcaaatattgaagttgtttatcaacttcgctagaaaaaaaaaacacttggcatacaaaatatttaagtgaaggagaagtctaacgctgaactgacaatgaagggaaattgtttatgtgttatgaacatccaagtctttcttcttttttaagttgtcaaagaagcttccacaaaattagaaaggacaacagttctgagctgtaatttcgccttaaactctggacactctatatgtagtgcatttttaaacttgaaatatataatattcagccagcttaaacccatacaatgtatgtacaatacaatgtacaattatgtctcttgagcatcaatcttgttactgctgattcttgtaaatctttttgcttctactttcatcttaaactaatacgtgccagatataactgtcttgtttcagtgagagacgccctatttctatgtcatttttaatgtatctatttgtacaattttaaagttcttattttagtatacatataaatatcagtattctgacatgtaagaaaatgttacggcatcacacttatattttatgaacattgtactgttgctttaatatgagcttcaatataagaagcaatctttgaaataaaaaaagattttttttt

SEQ ID NO: 153 - DNA, 5047 bp, Homo sapiens
gctggatcctgcagtaaccacaacagcatcctctccctgcgccagggacctgccagccggagagatgactgattagatcagattagatccggagccccgctctgcagaagggggccccaggggcgggggaggaggaccccagctggcctgagctggggggaggggtgccttggggctcgcagagttagagctttccagcgcggggatcacacctcagaagccgccacaatgaaagacggaacacatttctacacccagtgactggccaggtcccagaggaaaacaaaaaatttgacttgaaaatatcgaccttggacatgtccaataaaacaggtgggaaacgcccggctaccaccaacagtgacatacccaaccacaacatggtgtccgaggtccctccagagcggcccagcgtccgggcaactcgcacagcccgcaaagccatcgcctttggcaagcgctcacactccatgaagcggaaccccaatgcacctgtcaccaaggcgggctggctcttcaaacaggccagctccggggttaagcagtggaacaagcgctggttcgtcctggtggatcgctgcctcttctactataaagatgagaaggaagagagtatcctgggcagcatccccctcctgagcttccgggtagccgcagtgcagccctcagacaacatcagccggaaacacacgtttaaggctgagcatgccggggtccgcacctacttcttcagtgccgagagccccgaggagcaagaggcctggatccaggccatgggggaggctgctcgagtacagatccctccagcccagaagtcagtgccccaagctgtgcggcacagccatgagaagccagactcggagaacgtcccacccagcaagcaccaccagcagccaccccacaacagcctccctaagcctgagccagaggccaagactcgaggggagggtgatggccgaggctgtgagaaggcagagagaaggcctgagaggccagaagtcaagaaagagcctccggtgaaagccaatggcctcccagctggaccggagccagcctcagagccgggcagcccttaccccgagggcccaagagtgccagggggtggggaacagcctgcccagcccaatggctggcagtaccactccccaagccggccagggagcacagctttcccgtctcaggatggagagactgggggacaccggcggagtttcccaccacgcaccaaccctgacaaaattgcccagcgcaagagctccatgaaccagcttcagcagtgggtgaatctgcgccggggggtacccccgcctgaagaccttcggagtccctctaggttctatcctgtgtctcgcagggtccctgagtactatggcccctactcctcccagtaccccgatgattatcagtactacccgccaggagtgcggccggagagcatctgttccatgccggcctatgatcggatcagcccgccctgggccctggaggacaagcgccatgccttccgcaatgggggtggccctgcctaccagctgcgagagtggaaggagcccgccagctacgggcggcaggatgccaccgtctggatcccaagcccctcccggcagccagtctattatgatgagctggatgccgcctctagctccctgcgccgcctgtccctgcagccccgctcccactctgtgccccgctcacccagccagggctcctacagccgtgcccgcatttactcccctgtccgctcacccagtgcccgttttgagcggctgccacctcgcagtgaggacatctatgctgaccctgctgcctatgtgatgaggcgatccatcagctcccccaaggtccctccatacccagaagtgttccgggacagcctccacacctacaagttaaacgagcaagacacagataagctgctgggaaaattgtgtgagcagaacaaggtggtgagggagcaggaccggctggtgcagcagctccgagctgagaaggagagcctggaaagtgccttgatggggacccaccaggagctggagatgtttggaagccagcccgcctacccagaaaagctgcgacacaaaaaggattcactgcagaaccagctcatcaacatccgcgtggagctgtctcaggcgaccacggccctgacaaacagcaccatagagtatgagcacctcgagtctgaggtctctgccctgcacgatgacctctgggagcagctcaatttggacacccagaatgaggtgctgaaccggcaaatccaaaaggagatctggaggatccaggacgtgatggaggggctgaggaagaacaacccctcccggggcacggacaccgccaagcacagaggaggacttggcccctcagccacctacagctccaacagcccggccagccccctcagctctgccagcctcaccagccccctgagccccttttcactggtgtcgggctctcaggggtcccccaccaagcctggctccaacgagcccaaggcaaactatgaacaaagcaagaaagacccccaccagacattgcccctggacacccccagagacatcagccttgtgcccaccaggcaagaggtagaggcagagaagcaggcagctctcaacaaagttggcgttgtgccccctcggacaaaatcgcccactgatgatgaggtgaccccatcagcagtggtaagaaggaatgccagtgggctcaccaatggactctcctcccaggaacgccccaagagtgctgtgtttcctggcgaggggaaggtcaagatgagcgtggaggagcagattgaccgaatgcggcggcaccagagtggctccatgaaggagaagcggaggagcctgcagctcccggccagcccggcccccgaccccagtccccggccagcctacaaagtggtgcgccgccaccgcagcatccacgaggtagacatctccaacctggaggcagccctgcgggcagaggagcctggcgggcatgcctacgagacaccccgggaggaaattgcccggcttcgcaaaatggagctagagccccagcattatgacgtggacatcaataaggagctctccactccagacaaagtcctcatccctgaacggtacattgacctggagcctgacactcccctgagccctgaggagttgaaggagaagcagaagaaggtggagaggatcaagacactcattgccaaatccagtatgcagaacgtggtgcccatcggcgagggggactctgtggacgtgccccaggactcagagagccagctgcaggagcaggagaagcggattgaaatctcctgcgccctggcgaccgaggcctcccgcaggggccgcatgctgtctgtgcaatgtgccaccccaagccctcccacctcccctgcttccccggctcctccagcaaaccccctgtcgtctgaatccccacggggcgccgacagcagctataccatgcgggtctgagctctgactgcaagccctggctgaggccaatgctgtgaagctccacagagccacattctgaagccgtcctctgcccacctgaggtcctggctccccaccctggccccctgcccctgcactcccatgggaatgccgcagggagccaggctggggccatgggctgctgccagaggaccgtggatacctcagtgtccacacacccaccatgcccagccctggagccatcactactcacaccgtggtcctgggccagggcctgagatgacagtggggagcaccatcctcattaatgtccaagtcacagggagcctcagccttgccctggctggggttgtggtgactccagtggaacattccctgatgggggacatgccgtggtggagaacacacctgtggctatcttatgtgaggactagaggtgaagaggagatggacactgcctctggagccagcctgacaccaaggacagcacttgtcatcatccctatcctcgtcagccccaccctgctgcctcagctggacccagggctttgacacaaacccagtgctttgcttatgggtgctcgctggggtccggtggagactgaccaccctgcttgagccaaagacaaggtgatgagagatggggagaggccattggctcccagagggaacagtgctggctgtggctagagaacagcaggtctgtgcagtgtctgagggcaggttgggaagggtagcagagagagagagacagaaagagagagagagagagagagagagagagagagagagagagagagatcctcagagtggaaggagggggaagcagcaggacacattggcaagtcaagcaggaaggagggagatggaaaggggatatcagattggtttcccccggtggagccttaggttagtgcccagtgcagtgccagactgtctcctctgctcctcccacctcatccctaggaggacccaccagtggagcacatgcagcctcagtggagatgcttggtgtggggatctgggtgaagggggttgagtagcgactgcctgggagatggctgttagtaggtctgcgcctggtgtctgcctcgccatcctggggtaaggggcagagagaaggacttgtcttatgtagggtgtggtcagccttggggccttacctacccagttccatgatatttcttgccctgttccccctggaatgtgcagtgggccagctgagagtacgccttgaggaggggggatgaggccttaatctgggaggcctatccccctatcccaggcatcccagacgaggactggctgaggctaggcgctctcatgatccacctgccccgggagggcagcggggaagacagagaaaagcaaacacattcctcctcagctccacccacctggagacgaatgtagccagagaggaggaaggagggaaactgaaaacaccgtggcccctcggccttctctctgctagagttgccgctcagaggcttcagcctgacttccagcggtcccaagaacacctactaattcctctccactccttcatggctgggacagttactggttcatatgcaagtaaagatgacaatttactcaac

SEQ ID NO: 154 - DNA, 3372 bp, Homo sapiens
tacaaccaggctcaactgttgcatggtagcagatttgcaaacatgagtgctgaggggtaccagtacagagcgctgtatgattataaaaaggaaagagaagaagatattgacttgcacttgggtgacatattgactgtgaataaagggtccttagtagctcttggattcagtgatggacaggaagccaggcctgaagaaattggctggttaaatggctataatgaaaccacaggggaaaggggggactttccgggaacttacgtagaatatattggaaggaaaaaaatctcgcctcccacaccaaagccccggccacctcggcctcttcctgttgcaccaggttcttcgaaaactgaagcagatgttgaacaacaagctttgactctcccggatcttgcagagcagtttgcccctcctgacattgccccgcctcttcttatcaagctcgtggaagccattgaaaagaaaggtctggaatgttcaactctatacagaacacagagctccagcaacctggcagaattacgacagcttcttgattgtgatacaccctccgtggacttggaaatgatcgatgtgcacgttttggctgacgctttcaaacgctatctcctggacttaccaaatcctgtcattccagcagccgtttacagtgaaatgatttctttagctccagaagtacaaagctccgaagaatatattcagctattgaagaagcttattaggtcgcctagcatacctcatcagtattggcttacgcttcagtatttgttaaaacatttcttcaagctctctcaaacctccagcaaaaatctgttgaatgcaagagtactctctgaaattttcagccctatgcttttcagattctcagcagccagctctgataatactgaaaacctcataaaagttatagaaattttaatctcaactgaatggaatgaacgacagcctgcaccagcactgcctcctaaaccaccaaaacctactactgtagccaacaacggtatgaataacaatatgtccttacaaaatgctgaatggtactggggagatatctcgagggaagaagtgaatgaaaaacttcgagatacagcagacgggacctttttggtacgagatgcgtctactaaaatgcatggtgattatactcttacactaaggaaagggggaaataacaaattaatcaaaatatttcatcgagatgggaaatatggcttctctgacccattaaccttcagttctgtggttgaattaataaaccactaccggaatgaatctctagctcagtataatcccaaattggatgtgaaattactttatccagtatccaaataccaacaggatcaagttgtcaaagaagataatattgaagctgtagggaaaaaattacatgaatataacactcagtttcaagaaaaaagtcgagaatatgatagattatatgaagaatatacccgcacatcccaggaaatccaaatgaaaaggacagctattgaagcatttaatgaaaccataaaaatatttgaagaacagtgccagacccaagagcggtacagcaaagaatacatagaaaagtttaaacgtgaaggcaatgagaaagaaatacaaaggattatgcataattatgataagttgaagtctcgaatcagtgaaattattgacagtagaagaagattggaagaagacttgaagaagcaggcagctgagtatcgagaaattgacaaacgtatgaacagcattaaaccagaccttatccagctgagaaagacgagagaccaatacttgatgtggttgactcaaaaaggtgttcggcaaaagaagttgaacgagtggttgggcaatgaaaacactgaagaccaatattcactggtggaagatgatgaagatttgccccatcatgatgagaagacatggaatgttggaagcagcaaccgaaacaaagctgaaaacctgttgcgagggaagcgagatggcacttttcttgtccgggagagcagtaaacagggctgctatgcctgctctgtagtggtggacggcgaagtaaagcattgtgtcataaacaaaacagcaactggctatggctttgccgagccctataacttgtacagctctctgaaagaactggtgctacattaccaacacacctcccttgtgcagcacaacgactccctcaatgtcacactagcctacccagtatatgcacagcagaggcgatgaagcgcttactctttgatccttctcctgaagttcagccaccctgaggcctctggaaagcaaagggctcctctccagtctgatctgtgaattgagctgcagaaacgaagccatctttctttggatgggactagagctttctttcacaaaaaagaagtaggggaagacatgcagcctaaggctgtatgatgaccacacgttcctaagctggagtgcttatcccttctttttctttttttctttggtttaatttaaagccacaaccacatacaacacaaagagaaaaagaaatgcaaaaatctctgcgtgcagggacaaagaggcctttaaccatggtgcttgttaatgctttctgaagctttaccagctgaaagttgggactctggagagcggaggagagagaggcagaagaaccctggcctgagaaggtttggtccagcctggtttagcctggatgttgctgtgcacggtggacccagacacatcgcactgtggattatttcattttgtaacaaatgaacgatatgtagcagaaaggcacgtccactcacaagggacgctttgggagaatgtcagttcatgtatgttcagaagaaattctgtcatagaaagtgccagaaagtgtttaacttgtcaaaaaacaaaaacccagcaacagaaaaatggagtttggaaaacaggacttaaaatgacattcagtatataaaatatgtacataatattggatgactaactatcaaatagatggatttgtatcaataccaaatagcttctgttttgttttgctgaaggctaaattcacagcgctatgcaattcttaattttcattaagttgttatttcagttttaaatgtaccttcagaataagcttccccaccccagtttttgttgcttgaaaatattgttgtcccggatttttgttaatattcatttttgttatccttttttaaaaataaatgtacaggatgccagtaaaaaaaaaaatggcttcagaattaaaactatgaaatattttacagtttttcttgtacagagtacttgctgttagcccaaggttaaaaagttcataacagattttttttggactgttttgttgggcagtgcctgataagcttcaaagctgctttattcaataaaaaaaaaacccgaattcactgg

SEQ ID NO: 155 - DNA, 4139 bp, Homo sapiens
ccgctccacctctcaagcagccagcgcctgcctgaatctgttctgccccctccccacccatttcaccaccaccatgacaccgggcacccagtctcctttcttcctgctgctgctcctcacagtgcttacagttgttacaggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagctcggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaacggtggcagcagcctctcttacacaaacccagcagtggcagccgcttctgccaacttgtagggcacgtcgccgctgagctgagtggccagccagtgccattccactccactcaggttcttcaggccagagcccctgcaccctgtttgggctggtgagctgggagttcaggtgggctgctcacagcctccttcagaggccccaccaatttctcggacacttctcagtgtgtggaagctcatgtgggcccctgaggctcatgcctgggaagtgttgtgggggctcccaggaggactggcccagagagccctgagatagcggggatcctgaactggactgaataaaacgtggtctcccactg

SEQ ID NO: 156 - DNA, 4879 bp, Homo sapiens
accaattcgccagcggttcaggtggctcttgcctcgatgtcctagcctaggggcccccgggccggacttggctgggctcccttcaccctctgcggagtcatgagggcgaacgacgctctgcaggtgctgggcttgcttttcagcctggcccggggctccgaggtgggcaactctcaggcagtgtgtcctgggactctgaatggcctgagtgtgaccggcgatgctgagaaccaataccagacactgtacaagctctacgagaggtgtgaggtggtgatggggaaccttgagattgtgctcacgggacacaatgccgacctctccttcctgcagtggattcgagaagtgacaggctatgtcctcgtggccatgaatgaattctctactctaccattgcccaacctccgcgtggtgcgagggacccaggtctacgatgggaagtttgccatcttcgtcatgttgaactataacaccaactccagccacgctctgcgccagctccgcttgactcagctcaccgagattctgtcagggggtgtttatattgagaagaacgataagctttgtcacatggacacaattgactggagggacatcgtgagggaccgagatgctgagatagtggtgaaggacaatggcagaagctgtcccccctgtcatgaggtttgcaaggggcgatgctggggtcctggatcagaagactgccagacattgaccaagaccatctgtgctcctcagtgtaatggtcactgctttgggcccaaccccaaccagtgctgccatgatgagtgtgccgggggctgctcaggccctcaggacacagactgctttgcctgccggcacttcaatgacagtggagcctgtgtacctcgctgtccacagcctcttgtctacaacaagctaactttccagctggaacccaatccccacaccaagtatcagtatggaggagtttgtgtagccagctgtccccataactttgtggtggatcaaacatcctgtgtcagggcctgtcctcctgacaagatggaagtagataaaaatgggctcaagatgtgtgagccttgtgggggactatgtcccaaagcctgtgagggaacaggctctgggagccgcttccagactgtggactcgagcaacattgatggatttgtgaactgcaccaagatcctgggcaacctggactttctgatcaccggcctcaatggagacccctggcacaagatccctgccctggacccagagaagctcaatgtcttccggacagtacgggagatcacaggttacctgaacatccagtcctggccgccccacatgcacaacttcagtgttttttccaatttgacaaccattggaggcagaagcctctacaaccggggcttctcattgttgatcatgaagaacttgaatgtcacatctctgggcttccgatccctgaaggaaattagtgctgggcgtatctatataagtgccaataggcagctctgctaccaccactctttgaactggaccaaggtgcttcgggggcctacggaagagcgactagacatcaagcataatcggccgcgcagagactgcgtggcagagggcaaagtgtgtgacccactgtgctcctctgggggatgctggggcccaggccctggtcagtgcttgtcctgtcgaaattatagccgaggaggtgtctgtgtgacccactgcaactttctgaatggggagcctcgagaatttgcccatgaggccgaatgcttctcctgccacccggaatgccaacccatggagggcactgccacatgcaatggctcgggctctgatacttgtgctcaatgtgcccattttcgagatgggccccactgtgtgagcagctgcccccatggagtcctaggtgccaagggcccaatctacaagtacccagatgttcagaatgaatgtcggccctgccatgagaactgcacccaggggtgtaaaggaccagagcttcaagactgtttaggacaaacactggtgctgatcggcaaaacccatctgacaatggctttgacagtgatagcaggattggtagtgattttcatgatgctgggcggcacttttctctactggcgtgggcgccggattcagaataaaagggctatgaggcgatacttggaacggggtgagagcatagagcctctggaccccagtgagaaggctaacaaagtcttggccagaatcttcaaagagacagagctaaggaagcttaaagtgcttggctcgggtgtctttggaactgtgcacaaaggagtgtggatccctgagggtgaatcaatcaagattccagtctgcattaaagtcattgaggacaagagtggacggcagagttttcaagctgtgacagatcatatgctggccattggcagcctggaccatgcccacattgtaaggctgctgggactatgcccagggtcatctctgcagcttgtcactcaatatttgcctctgggttctctgctggatcatgtgagacaacaccggggggcactggggccacagctgctgctcaactggggagtacaaattgccaagggaatgtactaccttgaggaacatggtatggtgcatagaaacctggctgcccgaaacgtgctactcaagtcacccagtcaggttcaggtggcagattttggtgtggctgacctgctgcctcctgatgataagcagctgctatacagtgaggccaagactccaattaagtggatggcccttgagagtatccactttgggaaatacacacaccagagtgatgtctggagctatggtgtgacagtttgggagttgatgaccttcggggcagagccctatgcagggctacgattggctgaagtaccagacctgctagagaagggggagcggttggcacagccccagatctgcacaattgatgtctacatggtgatggtcaagtgttggatgattgatgagaacattcgcccaacctttaaagaactagccaatgagttcaccaggatggcccgagacccaccacggtatctggtcataaagagagagagtgggcctggaatagcccctgggccagagccccatggtctgacaaacaagaagctagaggaagtagagctggagccagaactagacctagacctagacttggaagcagaggaggacaacctggcaaccaccacactgggctccgccctcagcctaccagttggaacacttaatcggccacgtgggagccagagccttttaagtccatcatctggatacatgcccatgaaccagggtaatcttggggagtcttgccaggagtctgcagtttctgggagcagtgaacggtgcccccgtccagtctctctacacccaatgccacggggatgcctggcatcagagtcatcagaggggcatgtaacaggctctgaggctgagctccaggagaaagtgtcaatgtgtagaagccggagcaggagccggagcccacggccacgcggagatagcgcctaccattcccagcgccacagtctgctgactcctgttaccccactctccccacccgggttagaggaagaggatgtcaacggttatgtcatgccagatacacacctcaaaggtactccctcctcccgggaaggcaccctttcttcagtgggtcttagttctgtcctgggtactgaagaagaagatgaagatgaggagtatgaatacatgaaccggaggagaaggcacagtccacctcatccccctaggccaagttcccttgaggagctgggttatgagtacatggatgtggggtcagacctcagtgcctctctgggcagcacacagagttgcccactccaccctgtacccatcatgcccactgcaggcacaactccagatgaagactatgaatatatgaatcggcaacgagatggaggtggtcctgggggtgattatgcagccatgggggcctgcccagcatctgagcaagggtatgaagagatgagagcttttcaggggcctggacatcaggccccccatgtccattatgcccgcctaaaaactctacgtagcttagaggctacagactctgcctttgataaccctgattactggcatagcaggcttttccccaaggctaatgcccagagaacgtaactcctgctccctgtggcactcagggagcatttaatggcagctagtgcctttagagggtaccgtcttctccctattccctctctctcccaggtcccagccccttttccccagtcccagacaattccattcaatctttggaggcttttaaacattttgacacaaaattcttatggtatgtagccagctgtgcactttcttctctttcccaaccccaggaaaggttttccttattttgtgtgctttcccagtcccattcctcagcttcttcacaggcactcctggagatatgaaggattactctccatatcccttcctctcaggctcttgactacttggaactaggctcttatgtgtgcctttgtttcccatcagactgtcaagaagaggaaagggaggaaacctagcagaggaaagtgtaattttggtttatgactcttaaccccctagaaagacagaagcttaaaatctgtgaagaaagaggttaggagtagatattgattactatcataattcagcacttaactatgagccaggcatcatactaaacttcacctacattatctcacttagtcctttatcatccttaaaacaattctgtgacatacatattatctcattttacacaaagggaagtcgggcatggtggctcatgcctgtaatctcagcactttgggaggctgaggcagaaggattacctgaggcaaggagtttgagaccagcttagccaacatagtaagacccccatctcttt

SEQ ID NO: 157 - DNA, 1611 bp, Homo sapiens
gaattctgaatataggacacgaatttatgatccttagcaatgtgaagttagagaaggggttttattgtgaaattgacacaggttgttttatatcttataaatgaagtctcctcattttcctgtggtcagaagagagggggcaagcagaaaagcagaggaacaaatttggaggctaaaataacattctacataaggaactatactacagtagaattaattgatagcagggattaagagatgtaaatgaatttgagatacatattctagaggtagaatgtgcaatactttttgtatgtccatatacagaaattggttgcattttccttaaataaaaagattttttaaaagtcagtgagctgttatgttttcttccctctgacttcaattccttgattctttcaatttttttaatataaatttactgtctaaaagctggatcagcttatgctcctttgttgagagaagttggcatgctgtcaagtgggctgggcacactgagtttcagtttcctttctctgagtctttgaagcttcaaggctgctgaataatttccttctcccattttgtgcctgcctagctatccagacagagcagctaccctcagctctagctgatactacagacagtacaacaggtaaatgtctttctgcttttcatttttcctagctagcattagtctctctctgtctctctcaggtgacagtgtccattgcaatctcagtttttgttttaatttaaaaaacaataatttatagtaaaaaattagctaatgatttttttgctttctgttcatcctttgttttgtcattttttgtattatgtagagtatataagaggcataaatgcaaattttataactacatattatctgttttttaatatttaatggaaaatatatatgatttgccactagatcaagaagtatggcagtgacaactcgtttgacatggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcagaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaaggtaggtttaattagataatcctgtagagagttctcccttgcatgtttggtaggtttgaaccaattcatctctttaaggaaaaatgaacttttcacttgtcaataatttggatgattcagactgaaacctggatacagattgtttgctaagagacaaccatggtcaataaaatgtatatttatgataagaacccttaacgtaagatttatcctcttagcacattttaagtac

SEQ ID NO: 158 - DNA, 155 bp, Homo sapiens
gaattcactgatattcattcattcattcagccaattattcgacaacttctaatctacattattctttgattatttccccagattcactggatgaaagaaagataaaaggggtcattgagtaagtcaatgtttttaagattctattactctcttca

SEQ ID NO: 159 - DNA, 312 bp, Homo sapiens
tttgcgacctaacctcagtcaattgttaaaaacggtcatgtctaaacaggctcaggaagagcttactgtctgccttgagaacttatgaaccatatggatccctggttcaacaaatacgaattctcctcctgggtccaattggagctcccaagtccagctttttcaactcagtgaggtctgttttccaagggcatgtaacgcatcaggctttggtgggcactaatacaactgggatatctgagaaggtaagcacatttgaggccacctagcctttgcttctctgttcaaatcaattatatttcaaaagctttt

SEQ ID NO: 160 - DNA, 447 bp, Homo sapiens
ggccacctagcctttgcttctctgttcaaatcaattatatttcaaaagccttttgcagatcaactttattacatatagacttcatctcaatttataataaaaaatgaatctttaaaattgcttttctcccctctacagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccaggtaatatttgactaatgagaaattataactgatttttaaaatgcttatttttgtacaaatgtatcagcgtttatcttcttaaattatacttgctcaagatcctttgtctcttttagattttttttttcaaaaagaataaaaacatctcgagggctcttc

SEQ ID NO: 161 - DNA, 341 bp, Homo sapiens
ttgtgctcataaatatttgttgaattaatatcttgctttatgtctaccttacagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcaaagggagttggtaaacgctggtgagtctcattccactttgctaagggtaataccactaagggtaattgactagactgtattttagaatgctttttggacaggataaagaacttaagtcattgcatatttcaatct

SEQ ID NO: 162 - DNA, 288 bp, Homo sapiens
gatctttccaaatctgaaattgttccataggttgcctattacataattgatagttaaataacttgaaaatactgatgctctctaaaatgatttaaaaaattctgtttggcataggtgtggtacatgtggctttgctcactcatgtggatagcatggatttgattacaaaaggtgaccttatagaaatagagagatgtgagcctgtgaggtccaaggtaatgaatgatgcccttcgtaaacacattttctggggtatgttactacaatcacatactagtgtgtataaaa

SEQ ID NO: 163 - DNA, 372 bp, Homo sapiens
tttttttccaatggaaattattgcaagttcctacatcttgatattgctttcataatttatactaacataaaataatatttttcactgttttgcaatgtctttttaatttctgtattgcagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaataggtagatggtttggtggtgtggaagcttggaagcggtcaggtagttggctactttctgcttggatctattaaatactg

SEQ ID NO: 164 - DNA, 483 bp, Homo sapiens
cctctggttgcctttcctgagataatccactaagaatattttgtgtttcttttctcagggaatctaagggaggaaattatcaactgtgcacaaggaaaaaaatagatatgtgaaaggttcacgtaaatttcctcacatcacagaagattaaaattcagaaaggagaaaacacagaccaaagagaagtatctaagaccaaagggatgtgttttattaatgtctaggatgaagaaatgcatagaacattgtagtacttgtaaataactagaaataacatgatttagtcataattgtgaaaaataataataatttttcttggatttatgttctgtatctgtgaaaaaataaatttcttataaaactcgggtctaacttgagagtgtgtgtgattttggaaaaattatgatttgtcagcatcttctgatattcactgctttcatcttaattttgccttctgattttatttctaaagtatgtgatttt

SEQ ID NO: 165 - DNA, 25 bp, Artificial Sequence
  Description of Artificial Sequence Primer
gctctcttatttgtaccggtttttg

SEQ ID NO: 166 - DNA, 24 bp, Artificial Sequence
  Description of Artificial Sequence Primer
aagctagtgactgtcaccgatcag

SEQ ID NO: 167 - DNA, 16 bp, Artificial Sequence
  Description of Artificial Sequence Probe
tcatgtttccaatctc

SEQ ID NO: 168 - DNA, 30 bp, Artificial Sequence
  Description of Artificial Sequence Primer
cctgatataaatgcaatattaatgccttta

SEQ ID NO: 169 - DNA, 21 bp, Artificial Sequence
  Description of Artificial Sequence Primer
aagaaccgggagagcaaacat

SEQ ID NO: 170 - DNA, 20 bp, Artificial Sequence
  Description of Artificial Sequence Probe
atctatgccaaagatcactt

SEQ ID NO: 171 - DNA, 17 bp, Artificial Sequence
  Description of Artificial Sequence Primer
ggagcaccgcctgtgaa

SEQ ID NO: 172 - DNA, 20 bp, Artificial Sequence
  Description of Artificial Sequence Primer
tgtgcgttgcctgaatgaac

SEQ ID NO: 173 - DNA, 16 bp, Artificial Sequence
  Description of Artificial Sequence Probe
accaacctgaagacac

SEQ ID NO: 174 - DNA, 22 bp, Artificial Sequence
  Description of Artificial Sequence Primer
tctcgactgaatggactttgca

SEQ ID NO: 175 - DNA, 18 bp, Artificial Sequence
  Description of Artificial Sequence Primer
ttgtgtaccccgcaccaa

SEQ ID NO: 176 - DNA, 17 bp, Artificial Sequence
  Description of Artificial Sequence Probe
cacacctctatcccggc

SEQ ID NO: 177 - DNA, 20 bp, Artificial Sequence
  Description of Artificial Sequence Primer
gctgcatgtggatcctgaga

SEQ ID NO: 178 - DNA, 25 bp, Artificial Sequence
  Description of Artificial Sequence Primer
tgagtagccagaataatcaccatca

SEQ ID NO: 179 - DNA, 18 bp, Artificial Sequence
  Description of Artificial Sequence Probe
cttcaagctcctgggtaa

SEQ ID NO: 180 - DNA, 136 bp, Homo sapiens
ggtgccgccagctgccgcaccagcccgaacaccattgagggagctgggagaccctccccacagtgccacccatgcagctgctccccaggccaccccgctgatggagccccaccttgtctgctaaataaacatgtgc

SEQ ID NO: 181 - protein, 1066 aa, Homo sapiens
MPVFHTRTIESILEPVAQQISHLVIMHEEGEVDGKAIPDLTAPVAAVQAAVSNLVRVGKETVQTTEDQILKRDMPPAFIKVENACTKLVQAAQMLQSDPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNSKNQGIEEALKNRNFTVEKMSAEINEIIRVLQLTSWDEDAWASKDTEAMKRALASIDSKLNQAKGWLRDPSASPGDAGEQAIRQILDEAGKVGELCAGKERREILGTCKMLGQMTDQVADLRARGQGSSPVAMQKAQQVSQGLDVLTAKVENAARKLEAMTNSKQSIAKKIDAAQNWLADPNGGPEGEEQIRGALAEARKIAELCDDPKERDDILRSLGEISALTSKLADLRRQGKGDSPEARALAKQVATALQNLQTKTNRAVANSRPAKAAVHLEGKIEQAQRWIDNPTVDDRGVGQAAIRGLVAEGHRLANVMMGPYRQDLLAKCDRVDQLTAQLADLAARGEGESPQARALASQLQDSLKDLKARMQEAMTQEVSDVFSDTTTPIKLLAVAATAPPDAPNREEVFDERAANFENHSGKLGATAEKAAAVGTANKSTVEGIQASVKTARELTPQVVSAARILLRNPGNQAAYEHFETMKNQWIDNVEKMTGLVDEAIDTKSLLDASEEAIKKDLDKCKVAMANIQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEQLRLTDELAPPKPPLPEGEVPPPRPPPPEEKDEEFPEQKAGEVINQPMMMAARQLHDEARKWSSKGNDIIAAAKRMALLMAEMSRLVRGGSGTKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 182 - DNA, 1666 bp, Homo sapiens
ctccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 183 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 184 - DNA, 2480 bp, Homo sapiens
tttgcttcccctcttcccgaagctctgacacctgccccaacaagcaatgttggaaaattatttacatagtggcgcaaactcccttactgctttggatataaatccaggcaggaggaggtagctctaaggcaagagatctgggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacaggtaagggccaccccaggctatgggagagttttgatctgaggtatgggggtggggtctaagactgcatgaacagtctcaaaaaaaaaaaaaaaagactgtatgaacagaacagtggagcatccttcatggtgtgtgtgtgtgtgtgtgtgtgtgtgtgtggtgtgtaactggagaaggggtcagtctgtttctcaatcttaaattctatacgtaagtgaggggatagatctgtgtgatctgagaaacctctcacatttgcttgtttttctggctcacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggccagctgtgggtcctgaaggtacctcccggttttttacaccgcatgggccccacgtctctgtctctggtacctcccgcttttttacactgcatggttcccacgtctctgtctctgggcctttgttcccctatatgcattgaggcctgctccaccctcctcagcgcctgagaatggaggtaaagtgtctggtctgggagctcgttaactatgctgggaaatggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgagtgagtccaagctgttttctgatagttgctttagaagttgtgactaacttctctatgacctttgaa

SEQ ID NO: 185 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP